(19) United States
(12) Patent Application Publication
(10) Pub. No.: US 2025/0066371 A1
(43) Pub. Date: Feb. 27, 2025

(54) FLUOROALKOXYALKYLENE-DIHYDROIMIDAZO[5,1-D]TETRAZINONE COMPOUNDS AND RELATED COMPOUNDS AND THEIR USE IN TREATING MEDICAL CONDITIONS

(71) Applicant: Modifi Biosciences, Inc., Westport, CT (US)

(72) Inventor: Kyle Thomas Tarantino, Killingworth, CT (US)

(21) Appl. No.: 18/792,835

(22) Filed: Aug. 2, 2024

Related U.S. Application Data
(60) Provisional application No. 63/517,715, filed on Aug. 4, 2023.
(60) Provisional application No. 63/544,054, filed on Oct. 13, 2023.

(51) Int. Cl. C07D 487/04; A61K 31/495; A61P 35/00; C07B 59/00

(52) U.S. Cl. CPC C07D 487/04; A61K 31/495; A61P 35/00; C07B 59/004; C07B 2200/05

(57) ABSTRACT

The invention provides fluoroalkoxyalkylene dihydroimidazo[5,1-d]tetrazinone compounds and related compounds, pharmaceutical compositions, and their use in treating cancer.

### CROSS-REFERENCE TO RELATED APPLICATIONS

This application claims the benefit of and priority to U.S. Provisional Patent Application Ser. No. 63/517,715, filed Aug. 4, 2023, and U.S. Provisional Patent Application Ser. No. 63/544,054, filed Oct. 13, 2023; the contents of each of which are hereby incorporated by reference in their entirety.

### FEDERALLY SPONSORED RESEARCH OR DEVELOPMENT

The invention was made with government support under grant number R44CA271994 awarded by the National Cancer Institute which is part of the National Institutes of Health. The government has certain rights in the invention.

### FIELD OF THE INVENTION

The invention provides fluoroalkoxyalkylene dihydroimidazo[5,1-d]tetrazinone compounds and related compounds, pharmaceutical compositions, and their use in treating cancer

### BACKGROUND

Cancer continues to be a significant health problem despite the substantial research efforts and scientific advances reported in the literature for treating this disease. Solid tumors, such as prostate cancer, colon, rectum, skin cancer, breast cancer, and lung cancer remain highly prevalent among the world population. Existing therapies for treating cancer include localized therapies, such as surgery, radiation therapy, cryotherapy, and systemic therapies (e.g., chemotherapy, hormonal therapy, immune therapy, and targeted therapy) used alone or in combination. Support therapies are also used in some contexts, where supportive therapies are additional treatments that do not directly treat cancer but are used to reduce side effects and address patient quality of life. However, current treatment options for cancer are not effective for all patients and/or can have substantial adverse side effects. New therapies are needed to address this unmet need in cancer therapy.

Certain imidazotetrazinone compounds are described in international patent applications WO 2023/049806 and WO 2009/077741, U.S. Pat. No. 5,266,291, and by Moody et al. in Pharmaceuticals (2014) vol. 7, pages 797-838. Temozolomide, described in U.S. Pat. No. 5,266,291, is marketed for the treatment of newly diagnosed glioblastoma multiforme cancers and refractory anaplastic astrocytoma in patients who have experienced disease progression on a drug regimen containing nitrosourea and procarbazine. Additional new compounds are needed to provide therapies that have superior efficacy and/or reduced adverse side effects.

Accordingly, the need exists for new compounds and therapeutic methods for treating cancer. The present invention addresses the foregoing needs and provides other related advantages.

### SUMMARY

The invention provides fluoroalkoxyalkylene dihydroimidazo[5,1-d]tetrazinone compounds and related compounds, pharmaceutical compositions, and their use in treating cancer. In particular, one aspect of the invention provides a collection of fluoroalkoxyalkylene dihydroimidazo[5,1-d]tetrazinone compounds, such as a compound represented by Formula I:

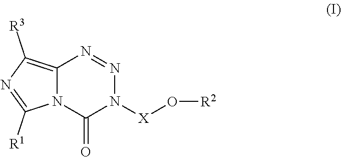

or a pharmaceutically acceptable salt thereof, where the variables are as defined in the detailed description. In a more specific embodiment, the compound of Formula I is a compound represented by Formula I-A:

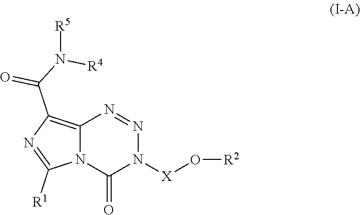

or a pharmaceutically acceptable salt thereof, where the variables are as defined in the detailed description.

Another aspect of the invention provides a collection of fluoroalkoxyalkylene dihydroimidazo[5,1-d]tetrazinone compounds, such as a compound represented by Formula I-aa:

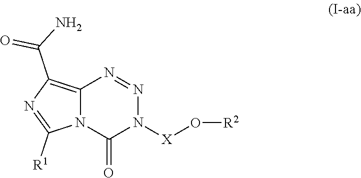

or a pharmaceutically acceptable salt thereof, where the variables are as defined in the detailed description.

Another aspect of the invention provides a collection of fluoroalkoxyalkylene dihydroimidazo[5,1-d]tetrazinone compounds, such as a compound represented by Formula II:

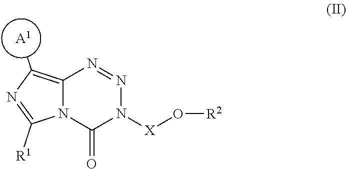

or a pharmaceutically acceptable salt thereof, where the variables are as defined in the detailed description.

Another aspect of the invention provides a collection of fluoroalkoxyalkylene imidazotriazene compounds, such as a compound represented by Formula IIIa or Formula IIIb:

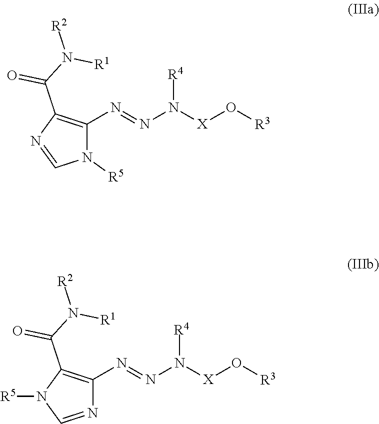

or a pharmaceutically acceptable salt thereof, where the variables are as defined in the detailed description.

Another aspect of the invention provides a collection of fluoroalkoxyalkylene nitrosourea compounds, such as a compound represented by Formula IV:

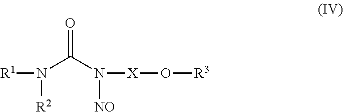

or a pharmaceutically acceptable salt thereof, where the variables are as defined in the detailed description.

Another aspect of the invention provides a collection of fluoroalkoxyalkylene hydrazine compounds, such as a compound represented by Formula V:

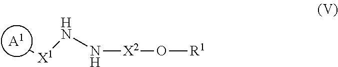

or a pharmaceutically acceptable salt thereof, where the variables are as defined in the detailed description.

Further description of additional collections of fluoroalkoxyalkylene dihydroimidazo[5,1-d]tetrazinone and related compounds are described in the detailed description. All of the foregoing compounds may be part of a pharmaceutical composition comprising a pharmaceutically acceptable carrier.

Another aspect of the invention provides a method of treating cancer. The method comprises administering to a subject in need thereof a therapeutically effective amount of a compound described herein, such as a compound of Formula I, I-A, I-aa, II, III, IV, or V, to treat the cancer, as further described in the detailed description. In certain embodiments, the cancer is MGMT deficient.

Another aspect of the invention provides a method of producing a DNA lesion in a subject. The method comprises administering to a subject an effective amount of a compound described herein, such as a compound of Formula I, I-A, I-aa, II, III, IV, or V, to produce a DNA lesion in the subject, as further described in the detailed description. In certain embodiments, the subject has cancer.

Another aspect provides a method of treating a MGMT-deficient cancer in a patient, comprising exposing a MGMT-deficient cancer cell in the patient in need thereof to 2-(C₁₋₄ fluoroalkoxy)ethane-1-diazonium, to thereby treat the MGMT-deficient cancer.

Another aspect provides a method of treating a MGMT-deficient cancer in a patient, comprising exposing a MGMT-deficient cancer cell in the patient in need thereof to 2-(trifluoromethoxy)ethane-1-diazonium, to thereby treat the MGMT-deficient cancer.

Another aspect provides a method of treating a MGMT-deficient cancer in a patient, comprising forming 2-(trifluoromethoxy)ethane-1-diazonium in a MGMT-deficient cancer cell in the patient in need of treatment, to thereby treat the MGMT-deficient cancer.

Another aspect provides a method of treating a MGMT-deficient cancer in a patient, comprising forming a 2-(C₁₋₄ fluoroalkoxy)ethane-1-DNA adduct in a MGMT-deficient cancer cell in the patient in need thereof, to thereby treat the MGMT-deficient cancer.

Another aspect provides a method of treating a MGMT-deficient cancer in a patient, comprising forming a 2-(trifluoromethoxy)ethane-1-DNA adduct in a MGMT-deficient cancer cell in the patient in need thereof, to thereby treat the MGMT-deficient cancer.

Another aspect provides a method of treating a MGMT-deficient cancer in a patient, comprising exposing DNA to 2-(trifluoromethoxy)ethane-1-diazonium to thereby form a 2-(trifluoromethoxy)ethane-1-DNA adduct in a MGMT-deficient cancer cell in the patient in need thereof, to thereby treat the MGMT-deficient cancer.

Another aspect provides a method, comprising exposing DNA to 2-(trifluoromethoxy)ethane-1-diazonium in a cancer cell to thereby form a 2-(trifluoromethoxy)ethane-1-DNA adduct in the cancer cell.

Another aspect provides a method of treating a MGMT-deficient cancer in a patient, comprising administering to the patient in need thereof a compound comprising a 2-(trifluoromethoxy)ethanyl group, to thereby treat the patient, wherein said compound undergoes conversion in vivo to 2-(trifluoromethoxy)ethane-1-diazonium.

Another aspect provides a method of forming a 2-(trifluoromethoxy)ethane-1-DNA adduct, comprising exposing DNA to a compound comprising a 2-(trifluoromethoxy)ethanyl group to thereby form a 2-(trifluoromethoxy)ethane-1-DNA adduct, wherein said compound undergoes conversion in vivo to 2-(trifluoromethoxy)ethane-1-diazonium.

Another aspect provides a method of forming a 2-(trifluoromethoxy)ethane-1-DNA adduct, comprising exposing DNA to 2-(trifluoromethoxy)ethane-1-diazonium to thereby form a 2-(trifluoromethoxy)ethane-1-DNA adduct. In certain embodiments, the method comprises exposing DNA in a cancer patient to 2-(trifluoromethoxy)ethane-1-diazonium. In certain embodiments, the cancer patient has a cancer that is MGMT-deficient.

Another aspect provides a DNA adduct, comprising DNA covalently bonded to one or more occurrences of

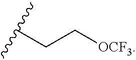 [SMILES: *CCOC(F)(F)F]

In certain embodiments, the DNA adduct comprises DNA covalently bonded to 1 to 10 occurrences of

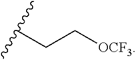 [SMILES: *CCOC(F)(F)F]

### DETAILED DESCRIPTION

The invention provides fluoroalkoxyalkylene dihydroimidazo[5,1-d]tetrazinone compound and related compounds, pharmaceutical compositions, and their use in treating cancer. Compounds described herein provide advantages over compounds described in the literature. For example, the literature compound 3-(2-fluoroethyl)-N-methyl-4-oxo-3,4-dihydroimidazo[5,1-d][1,2,3,5]tetrazine-8-carboxamide (referred to as compound A1; structure shown below) has been found to have the adverse event of lethal toxicity in animal model testing. For example, compound A1 resulted in the death of 100% of rats when administered to the rats at a dose of 10 mg/kg. The compound A1 resulted in the death of 100% of dogs when administered to the dogs at a dose of 2 mg/kg.

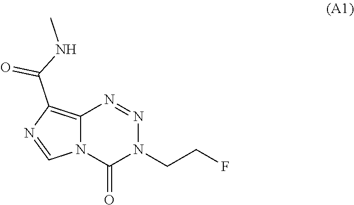 [SMILES: CNC(=O)c1ncn2c(=O)n(CCF)nnc12]

Without wishing to be bound by theory, it has been discovered that the fluoroethyl component of compound A1 gives rise to the lethal toxicity adverse side effect. By contrast, compound I-1 described herein has been tested in rats and dogs, and no adverse clinical signs were observed in the tested dose amounts which exceeded the dose amounts at which compound A1 caused the 100% mortality rate described above.

Another exemplary benefit of compounds described herein is their superior potency in causing the death of cancer cells. For example, compound I-1 demonstrated high potency in killing LN229 gliobastoma cells that were engineered to be MGMT negative/MMR positive, and high potency in killing LN229 gliobastoma cells that were engineered to be MGMT negative/MMR negative. In both instances, compound I-1 had an IC₅₀ less than 20 μM in the assay for causing the death of the aforementioned LN229 gliobastoma cells. This high potency stands in contrast to that observed for compound A2 (structure shown below) which had no detectable anti-cancer activity against a LN229 isogenic cell line set, even when the compound was used at a concentration up to 200 μM. This result demonstrates the superior anti-cancer effects of compound I-1 compared to compound A2.

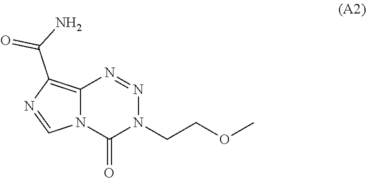 [SMILES: COCCn1nnc2c(C(N)=O)ncn2c1=O]

The practice of the present invention employs, unless otherwise indicated, conventional techniques of organic chemistry, pharmacology, molecular biology (including recombinant techniques), cell biology, biochemistry, and immunology. Such techniques are explained in the literature, such as in “Comprehensive Organic Synthesis” (B. M. Trost & I. Fleming, eds., 1991-1992); “Handbook of experimental immunology” (D. M. Weir & C. C. Blackwell, eds.); “Current protocols in molecular biology” (F. M. Ausubel et al., eds., 1987, and periodic updates); and “Current protocols in immunology” (J. E. Coligan et al., eds., 1991), each of which is herein incorporated by reference in its entirety.

Various aspects of the invention are set forth below in sections; however, aspects of the invention described in one particular section are not to be limited to any particular section. Further, when a variable is not accompanied by a definition, the previous definition of the variable controls.

### Definitions

Compounds of the present invention include those described generally herein, and are further illustrated by the classes, subclasses, and species disclosed herein. As used herein, the following definitions shall apply unless otherwise indicated. These definitions apply regardless of whether a term is used by itself or in combination with other terms, unless otherwise indicated. Hence, the definition of “alkyl” applies to “alkyl” as well as the “alkyl” portions of “—O-alkyl” etc. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “cycloaliphatic”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

As used herein, the term “bicyclic ring” or “bicyclic ring system” refers to any bicyclic ring system, i.e., carbocyclic or heterocyclic, saturated or having one or more units of unsaturation, having one or more atoms in common between the two rings of the ring system. Thus, the term includes any permissible ring fusion, such as ortho-fused or spirocyclic. As used herein, the term “heterobicyclic” is a subset of “bicyclic” that requires that one or more heteroatoms are present in one or both rings of the bicycle. Such heteroatoms may be present at ring junctions and are optionally substituted, and may be selected from nitrogen (including N-oxides), oxygen, sulfur (including oxidized forms such as sulfones and sulfonates), phosphorus (including oxidized forms such as phosphates), boron, etc. In some embodiments, a bicyclic group has 7-12 ring members and 0-4 heteroatoms independently selected from nitrogen, oxygen, or sulfur. As used herein, the term “bridged bicyclic” refers to any bicyclic ring system, i.e., carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, or sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally or alternatively, any substitutable nitrogen of a bridged bicyclic group is optionally substituted. Exemplary bicyclic rings include:

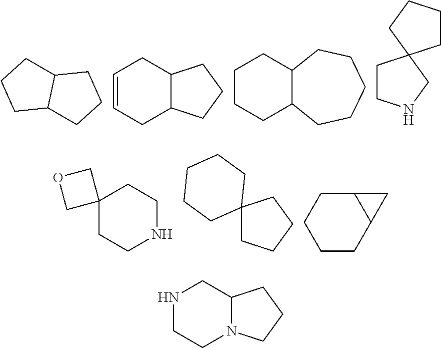

Exemplary bridged bicyclics include.

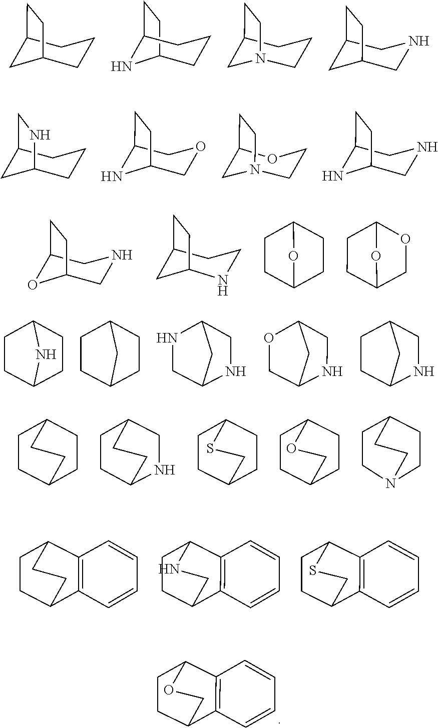

The term “lower alkyl” refers to a C₁₋₄ straight or branched alkyl group. Exemplary lower alkyl groups are methyl, ethyl, propyl, isopropyl, butyl, isobutyl, and tert-butyl.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR^{●} (as in N-substituted pyrrolidinyl)).

The term “unsaturated,” as used herein, means that a moiety has one or more units of unsaturation.

As used herein, the term “bivalent C₁₋₈ (or C₁₋₆) saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term deutero alkylene refers to an alkylene group that is substituted with at least one deuterium (D). In certain embodiments, the deutero alkylene contains 1, 2, 3, or 4 deuterium (D). In certain embodiments, the deutero alkylene contains 2 or 3 deuterium (D). In certain embodiments, the deutero alkylene contains 4 deuterium (D). Deuterium-enriched compounds refers to the feature that the compound has a quantity of deuterium that is greater than in naturally occurring compounds or synthetic compounds prepared from substrates having the naturally occurring distribution of isotopes. The threshold amount of deuterium enrichment is specified in certain instances in this disclosure, and all percentages given for the amount of deuterium present are mole percentages. Deuterium (H) is a stable, non-radioactive isotope of ¹H hydrogen and has an atomic weight of 2.014. Hydrogen naturally occurs as a mixture of the isotopes ¹H hydrogen (i.e., protium), deuterium (²H), and tritium (³H). The natural abundance of deuterium is 0.015%. One of ordinary skill in the art recognizes that in all chemical compounds with an H atom, the H atom actually represents a mixture of ¹H hydrogen, deuterium (H), and tritium (³H), where about 0.015% is deuterium. Thus, compounds with a level of deuterium that has been enriched to be greater than its natural abundance of 0.015% are considered unnatural and, as a result, novel over their non-enriched counterparts.

Unless indicated otherwise, when a D is specifically recited at a position or is shown in a formula, this D represents a mixture of hydrogen and deuterium where the amount of deuterium is about 100% (i.e., the abundance of deuterium ranges from at least 90% up to 100%). In certain embodiments, the abundance of deuterium in D is from 95% to 100%, or from 97% to 100%. In certain embodiments, the abundance of deuterium in D is at least 91%, 92%, 93%, 94%, 95%, 96%, 97%, 98%, or 99%.

The term “—(C₀ alkylene)-” refers to a bond. Accordingly, the term “—(C₀₋₃ alkylene)-” encompasses a bond (i.e., C₀) and a —(C₁₋₃ alkylene)- group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

The term “halogen” means F, Cl, Br, or I.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present invention, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where unless otherwise specified, the radical or point of attachment is on the heteroaromatic ring or on one of the rings to which the heteroaromatic ring is fused. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, and tetrahydroisoquinolinyl. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle,” “heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, 2-oxa-6-azaspiro[3.3]heptane, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be mono- or bicyclic. The term “heterocyclylalkyl” refers to an alkyl group substituted by a heterocyclyl, wherein the alkyl and heterocyclyl portions independently are optionally substituted. The term “oxo-heterocyclyl” refers to a heterocyclyl substituted by one or more oxo group. The term “heterocyclylene” refers to a multivalent heterocyclyl group having the appropriate number of open valences to account for groups attached to it. For example, “heterocyclylene” is a bivalent heterocyclyl group when it has two groups attached to it; “heterocyclylene” is a trivalent heterocyclyl group when it has three groups attached to it. The term “oxo-heterocyclylene” refers to a multivalent oxo-heterocyclyl group having the appropriate number of open valences to account for groups attached to it.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Each optional substituent on a substitutable carbon is a monovalent substituent independently selected from halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR—, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄₀S(O)₂R^{∘}; —S(O)₂NR^{∘}₂; —S(O)(NR^{∘})R^{∘}; —S(O)₂N=C(NR^{∘}₂)₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂.

Each R^{∘} is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono-or bicyclic ring having 0-4 heteroatoms independently selected from nitrogen, oxygen, or sulfur, which may be substituted by a divalent substituent on a saturated carbon atom of R^{∘} selected from =O and =S; or each R^{∘} is optionally substituted with a monovalent substituent independently selected from halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●}.

Each R^{●} is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, and wherein each R^{●} is unsubstituted or where preceded by halo is substituted only with one or more halogens; or wherein an optional substituent on a saturated carbon is a divalent substituent independently selected from ═O, ═S, ═NNR*₂, =NNHC(O)R*, =NNHC(O)OR*, =NNHS(O)₂R*, =NR*, =NOR*, —O(C(R*₂))₂₋₃O—, or —S(C(R*₂))₂₋₃S—, or a divalent substituent bound to vicinal substitutable carbons of an “optionally substituted” group is —O(CR*₂)₂₋₃O—, wherein each independent occurrence of R* is selected from hydrogen, C₁₋₆ aliphatic or an unsubstituted 5-6-membered saturated, partially unsaturated, or aryl ring having 0-4 heteroatoms independently selected from nitrogen, oxygen, or sulfur.

When R* is C₁₋₆ aliphatic, R* is optionally substituted with halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, and wherein each R^{●} is unsubstituted or where preceded by halo is substituted only with one or more halogens.

An optional substituent on a substitutable nitrogen is independently —R^{†}, —NR⁵⁵⁴₂, —C(O)R^{†}, —C(O)OR^{†}, —C(O)C(O)R^{†}, —C(O)CH₂C(O)R^{†}, —S(O)₂R⁵⁵⁴, —S(O)₂NR⁵⁵⁴₂, —C(S)NR^{†}₂, —C(NH)NR^{†}₂, or —N(R^{†})S(O)₂R⁵⁵⁴; wherein each R^{†} is independently hydrogen, C₁₋₆ aliphatic, unsubstituted —OPh, or an unsubstituted 5-6-membered saturated, partially unsaturated, or aryl ring having 0-4 heteroatoms independently selected from nitrogen, oxygen, or sulfur, or, two independent occurrences of R⁵⁵⁴, taken together with their intervening atom(s) form an unsubstituted 3-12-membered saturated, partially unsaturated, or aryl mono- or bicyclic ring having 0-4 heteroatoms independently selected from nitrogen, oxygen, or sulfur; wherein when R^{†} is C₁₋₆ aliphatic, R^{†} is optionally substituted with halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, and wherein each R^{●} is unsubstituted or where preceded by halo is substituted only with one or more halogens.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Further, acids which are generally considered suitable for the formation of pharmaceutically useful salts from basic pharmaceutical compounds are discussed, for example, by P. Stahl et al., Camille G. (eds.) Handbook of Pharmaceutical Salts. Properties, Selection and Use. (2002) Zurich: Wiley-VCH; S. Berge et al., Journal of Pharmaceutical Sciences (1977) 66(1) 1-19; P. Gould, International J. of Pharmaceutics (1986) 33 201-217; Anderson et al., The Practice of Medicinal Chemistry (1996), Academic Press, New York; and in The Orange Book (Food & Drug Administration, Washington, D.C. on their website). These disclosures are incorporated herein by reference.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. The invention includes compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention.

Diastereomeric mixtures can be separated into their individual diastereomers on the basis of their physical chemical differences by methods known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Alternatively, a particular enantiomer of a compound of the present invention may be prepared by asymmetric synthesis. Still further, where the molecule contains a basic functional group (such as amino) or an acidic functional group (such as carboxylic acid) diastereomeric salts are formed with an appropriate optically-active acid or base, followed by resolution of the diastereomers thus formed by fractional crystallization or chromatographic means known in the art, and subsequent recovery of the pure enantiomers.

Individual stereoisomers of the compounds of the invention may, for example, be substantially free of other isomers, or may be admixed, for example, as racemates or with all other, or other selected, stereoisomers. Chiral center(s) in a compound of the present invention can have the S or R configuration as defined by the IUPAC 1974 Recommendations. Further, to the extent a compound described herein may exist as an atropisomer (e.g., substituted biaryls), all forms of such atropisomer are considered part of this invention.

Chemical names, common names, and chemical structures may be used interchangeably to describe the same structure. If a chemical compound is referred to using both a chemical structure and a chemical name, and an ambiguity exists between the structure and the name, the structure predominates. It should also be noted that any carbon as well as heteroatom with unsatisfied valences in the text, schemes, examples and tables herein is assumed to have the sufficient number of hydrogen atom(s) to satisfy the valences.

The terms “a” and “an” as used herein mean “one or more” and include the plural unless the context is inappropriate.

The term “alkyl” refers to a saturated straight or branched hydrocarbon, such as a straight or branched group of 1-12, 1-10, or 1-6 carbon atoms, referred to herein as C₁-C₁₂ alkyl, C₁-C₁₀ alkyl, and C₁-C₆ alkyl, respectively. Exemplary alkyl groups include, but are not limited to, methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, heptyl, octyl, etc. The term fluoroalkyl refers to an alkyl group that is substituted with at least one fluoro. In certain embodiments, the fluoroalkyl contains 1, 2, or 3 fluoro groups. In certain embodiments, the fluoroalkyl contains 2 or 3 fluoro groups. In certain embodiments, the fluoroalkyl contains 3 fluoro groups.

The term “cycloalkyl” refers to a monovalent saturated cyclic, bicyclic, or bridged cyclic (e.g., adamantyl) hydrocarbon group of 3-12, 3-8, 4-8, or 4-6 carbons, referred to herein, e.g., as “C₃-C₆ cycloalkyl,” derived from a cycloalkane. Exemplary cycloalkyl groups include cyclohexyl, cyclopentyl, cyclobutyl, and cyclopropyl. The term “cycloalkylene” refers to a bivalent cycloalkyl group.

The terms “alkenyl” and “alkynyl” are art-recognized and refer to unsaturated aliphatic groups analogous in length and possible substitution to the alkyls described above, but that contain at least one double or triple bond respectively.

The terms “alkoxyl” or “alkoxy” are art-recognized and refer to an alkyl group, as defined above, having an oxygen radical attached thereto. Representative alkoxyl groups include methoxy, ethoxy, propyloxy, tert-butoxy and the like. The term “haloalkoxyl” refers to an alkoxyl group that is substituted with at least one halogen. Exemplary haloalkoxyl groups include —OCH₂F, —OCHF₂, —OCF₃, —OCH₂CF₃, —OCF₂CF₃, and the like.

The term “oxo” is art-recognized and refers to a “=O” substituent. For example, a cyclopentane substituted with an oxo group is cyclopentanone.

The symbol “ [symbol] ” indicates a point of attachment.

When any substituent or variable occurs more than one time in any constituent or the compound of the invention, its definition on each occurrence is independent of its definition at every other occurrence, unless otherwise indicated.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

As used herein, the terms “subject” and “patient” are used interchangeably and refer to organisms to be treated by the methods of the present invention. Such organisms preferably include, but are not limited to, mammals (e.g., murines, simians, equines, bovines, porcines, canines, felines, and the like), and most preferably includes humans.

The term “IC₅₀” is art-recognized and refers to the concentration of a compound that is required to achieve 50% inhibition of the target.

The abbreviation “MGMT” means O⁶-methylguanine-DNA-methyltransferase.

As used herein, the term “effective amount” refers to the amount of a compound sufficient to effect beneficial or desired results (e.g., a therapeutic, ameliorative, inhibitory or preventative result). An effective amount can be administered in one or more administrations, applications or dosages and is not intended to be limited to a particular formulation or administration route. As used herein, the term “treating” includes any effect, e.g., lessening, reducing, modulating, ameliorating or eliminating, that results in the improvement of the condition, disease, disorder, and the like, or ameliorating a symptom thereof.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see e.g., Martin, Remington's Pharmaceutical Sciences, 15th Ed., Mack Publ. Co., Easton, PA [1975].

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

In addition, when a compound of the invention contains both a basic moiety (such as, but not limited to, a pyridine or imidazole) and an acidic moiety (such as, but not limited to, a carboxylic acid) zwitterions (“inner salts”) may be formed. Such acidic and basic salts used within the scope of the invention are pharmaceutically acceptable (i.e., non-toxic, physiologically acceptable) salts. Such salts of the compounds of the invention may be formed, for example, by reacting a compound of the invention with an amount of acid or base, such as an equivalent amount, in a medium such as one in which the salt precipitates or in an aqueous medium followed by lyophilization.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

As a general matter, compositions specifying a percentage are by weight unless otherwise specified.

### I. Fluoroalkoxyalkylene dihydroimidazo[5,1-d]tetrazinone and Related Compounds

One aspect of the invention provides fluoroalkoxyalkylene dihydroimidazo[5,1-d]tetrazinone compounds and related compounds. The compounds may be used in the pharmaceutical compositions and therapeutic methods described herein. Exemplary compounds are described in the following sections, along with exemplary procedures for making the compounds.

One aspect of the invention provides a compound represented by Formula I:

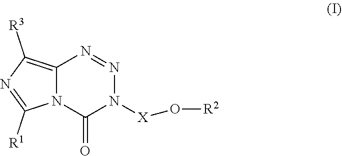

or a pharmaceutically acceptable salt thereof, wherein:
R¹ is hydrogen or C₁₋₄ alkyl;
R² is C₁₋₄ fluoroalkyl;
R³ is —C(O)N(R⁴)(R⁵), —CO₂R⁵, —C(O)SR⁴, —C(S)N(R⁴)(R⁵), —C(═NR⁷)OR⁴, —C(═NR⁷)SR⁴, —C(═NR⁷)N(R⁴)(R⁵), —C(O)-(halo), —C(O)—(C₁₋₄ alkyl), —CN, halo, or C₁₋₄ alkyl;
R⁴ is hydrogen, C₁₋₄ alkyl, or C₃₋₆ cycloalkyl;
R⁵ is hydrogen, C₁₋₄ alkyl, C₁₋₄ alkenyl, C₁₋₄ alkynyl, —(C₀₋₄ alkylene)-R⁶, —(C₁₋₄ alkylene)-C(O)—R⁶, —(C₁₋₄ alkylene)-C(O)—(C₁₋₄ alkyl), —(C₁₋₄ alkylene)-OR⁷, or —(C₁₋₄ alkylene)-N(R⁷)(R⁸); or R⁴ and R⁵ are taken together with the nitrogen atom to which they are attached to form a 3-7 membered heterocyclic ring containing the nitrogen atom and 0 or 1 additional heteroatom selected from nitrogen, oxygen, and sulfur, wherein the 3-7 membered heterocyclic ring is substituted with 0, 1, 2, or 3 occurrences of R⁹;
R⁶ is C₃₋₆ cycloalkyl, phenyl, or 5-6 membered heteroaryl; each of which is substituted with 0, 1, 2, or 3 occurrences of R⁹;
R⁷ and R⁸ are independently for each occurrence hydrogen, C₁₋₄ alkyl, or C₃₋₆ cycloalkyl; or R⁷ and R⁸ are taken together with the nitrogen atom to which they are attached to form a 3-7 membered heterocyclic ring containing the nitrogen atom;
R⁹ represents independently for each occurrence C₁₋₄ alkyl, C₃₋₆ cycloalkyl, halo, —OR⁷, or —N(R⁷)(R⁸); and
X is C₁₋₃ alkylene or C₁₋₃ deuteroalkylene.

The definitions of variables in Formula I above encompass multiple chemical groups. The application contemplates embodiments where, for example, i) the definition of a variable is a single chemical group selected from those chemical groups set forth above, ii) the definition of a variable is a collection of two or more of the chemical groups selected from those set forth above, and iii) the compound is defined by a combination of variables in which the variables are defined by (i) or (ii).

In certain embodiments, the compound is a compound of Formula I.

As defined generally above, R¹ is hydrogen or C₁₋₄ alkyl. In certain embodiments, R¹ is hydrogen or methyl. In certain embodiments, R¹ is hydrogen. In certain embodiments, R¹ is C₁₋₄ alkyl. In certain embodiments, R¹ is methyl. In certain embodiments, R¹ is selected from the groups depicted in the compounds in Table 1 below.

As defined generally above, R² is C₁₋₄ fluoroalkyl. In certain embodiments, R² is C₁₋₂ fluoroalkyl. In certain embodiments, R² is C₁ fluoroalkyl. In certain embodiments, the fluoroalkyl contains at least three fluorine atoms. In certain embodiments, R² is C₁₋₂ trifluoroalkyl. In certain embodiments, R² is trifluoromethyl. In certain embodiments, R² is selected from the groups depicted in the compounds in Table 1 below.

As defined generally above, R³ is —C(O)N(R⁴)(R⁵), —CO₂R⁵, —C(O)SR⁴, —C(S)N(R⁴)(R⁵), —C(═NR⁷)OR⁴, —C(═NR⁷)SR⁴, —C(═NR⁷)N(R⁴)(R⁵), —C(O)-(halo), —C(O)—(C₁₋₄ alkyl), —CN, halo, or C₁₋₄ alkyl. In certain embodiments, R³ is —C(O)N(R⁴)(R⁵). In certain embodiments, R³ is —CO₂R⁵, —C(O)SR⁴, or —C(S)N(R⁴)(R⁵). In certain embodiments, R³ is —C(═NR⁷)OR⁴, —C(═NR⁷)SR⁴, or —C(═NR⁷)N(R⁴)(R⁵). In certain embodiments, R³ is —C(O)-(halo), —C(O)—(C₁₋₄ alkyl), —CN, halo, or C₁₋₄ alkyl. In certain embodiments, R³ is —C(O)Cl, —C(O)CH₃, —CN, chloro, fluoro, or —CH₃.

In certain embodiments, R³ is —C(O)N(R⁴)(R⁵). In certain embodiments, R³ is —CO₂R⁵. In certain embodiments, R³ is —C(O)SR⁴. In certain embodiments, R³ is —C(S)N(R⁴)(R⁵). In certain embodiments, R³ is —C(═NR⁷)OR⁴. In certain embodiments, R³ is —C(═NR⁷)SR⁴. In certain embodiments, R³ is —C(═NR⁷)N(R⁴)(R⁵). In certain embodiments, R³ is —C(O)-(halo). In certain embodiments, R³ is —C(O)Cl. In certain embodiments, R³ is —C(O)—(C₁₋₄ alkyl). In certain embodiments, R³ is —C(O)CH₃. In certain embodiments, R³ is —CN. In certain embodiments, R³ is halo. In certain embodiments, R³ is chloro or fluoro. In certain embodiments, R³ is C₁₋₄ alkyl. In certain embodiments, R³ is —CH₃. In certain embodiments, R³ is selected from the groups depicted in the compounds in Table 1 below.

As defined generally above, R⁴ is hydrogen, C₁₋₄ alkyl, or C₃₋₆ cycloalkyl; or R⁴ and R⁵ are taken together with the nitrogen atom to which they are attached to form a 3-7 membered heterocyclic ring containing the nitrogen atom and 0 or 1 additional heteroatom selected from nitrogen, oxygen, and sulfur; wherein the 3-7 membered heterocyclic ring is substituted with 0, 1, 2, or 3 occurrences of R⁹. In certain embodiments, R⁴ is hydrogen, C₁₋₄ alkyl, or C₃₋₆ cycloalkyl. In certain embodiments, R⁴ is hydrogen or C₁₋₄ alkyl. In certain embodiments, R⁴ is hydrogen or methyl. In certain embodiments, R⁴ is C₁₋₄ alkyl or C₃₋₆ cycloalkyl.

In certain embodiments, R⁴ is hydrogen. In certain embodiments, R⁴ is C₁₋₄ alkyl. In certain embodiments, R⁴ is methyl. In certain embodiments, R⁴ is C₃₋₆ cycloalkyl. In certain embodiments, R⁴ is selected from the groups depicted in the compounds in Table 1 below.

As defined generally above, R⁵ is hydrogen, C₁₋₄ alkyl, C₁₋₄ alkenyl, C₁₋₄ alkynyl, —(C₀₋₄ alkylene)-R⁶, —(C₁₋₄ alkylene)-C(O)—R⁶, —(C₁₋₄ alkylene)-C(O)—(C₁₋₄ alkyl), —(C₁₋₄ alkylene)-OR⁷, or —(C₁₋₄ alkylene)-N(R⁷)(R⁸); or R⁴ and R⁵ are taken together with the nitrogen atom to which they are attached to form a 3-7 membered heterocyclic ring containing the nitrogen atom and 0 or 1 additional heteroatom selected from nitrogen, oxygen, and sulfur, wherein the heterocyclic ring is substituted with 0, 1, 2, or 3 occurrences of R⁹.

In certain embodiments, R⁵ is hydrogen, C₁₋₄ alkyl, C₁₋₄ alkenyl, C₁₋₄ alkynyl, —(C₀₋₄ alkylene)-R⁶, —(C₁₋₄ alkylene)-C(O)—R⁶, —(C₁₋₄ alkylene)-C(O)—(C₁₋₄ alkyl), —(C₁₋₄ alkylene)-OR⁷, or —(C₁₋₄ alkylene)-N(R⁷)(R⁸). In certain embodiments, R⁵ is hydrogen, C₁₋₄ alkyl, or —(C₀₋₄ alkylene)-R⁶. In certain embodiments, R⁵ is hydrogen, C₁₋₄ alkyl, or —R⁶. In certain embodiments, R⁵ is hydrogen, C₁₋₄ alkyl, C₃₋₆ cycloalkyl, phenyl, or 5-6 membered heteroaryl; wherein the phenyl is substituted with 0 or 1 occurrence of R⁹.

In certain embodiments, R⁵ is hydrogen, C₁₋₄ alkyl, C₁₋₄ alkenyl, C₁₋₄ alkynyl, —(C₁₋₄ alkylene)-OR⁷, or —(C₁₋₄ alkylene)-N(R⁷)(R⁸). In certain embodiments, R⁵ is hydrogen, C₁₋₄ alkyl, C₁₋₄ alkenyl, or C₁₋₄ alkynyl. In certain embodiments, R⁵ is hydrogen or C₁₋₄ alkyl. In certain embodiments, R⁵ is C₁₋₄ alkyl, C₁₋₄ alkenyl, or C₁₋₄ alkynyl.

In certain embodiments, R⁵ is hydrogen. In certain embodiments, R⁵ is C₁₋₄ alkyl. In certain embodiments, R⁵ is C₁₋₄ alkenyl. In certain embodiments, R⁵ is C₁₋₄ alkynyl.

In certain embodiments, R⁵ is —(C₀₋₄ alkylene)-R⁶, —(C₁₋₄ alkylene)-C(O)—R⁶, or —(C₁₋₄ alkylene)-C(O)—(C₁₋₄ alkyl). In certain embodiments, R⁵ is —(C₀₋₄ alkylene)-R⁶ or —(C₁₋₄ alkylene)-C(O)—R⁶. In certain embodiments, R⁵ is —(C₀₋₄ alkylene)-R⁶. In certain embodiments, R⁵ is —(C₁₋₄ alkylene)-C(O)—R⁶. In certain embodiments, R⁵ is —(C₁₋₄ alkylene)-C(O)—(C₁₋₄ alkyl).

In certain embodiments, R⁵ is —(C₁₋₄ alkylene)-R⁶, —(C₁₋₄ alkylene)-OR⁷, or —(C₁₋₄ alkylene)-N(R⁷)(R⁸). In certain embodiments, R⁵ is —(C₁₋₄ alkylene)-R⁶.

In certain embodiments, R⁵ is —R⁶. In certain embodiments, R⁵ is C₃₋₆ cycloalkyl, phenyl, or 5-6 membered heteroaryl; each of which is substituted with 0, 1, 2, or 3 occurrences of R⁹. In certain embodiments, R⁵ is C₃₋₆ cycloalkyl, phenyl, or 5-6 membered heteroaryl; wherein the phenyl is substituted with 0 or 1 occurrence of R⁹. In certain embodiments, R⁵ is C₃₋₆ cycloalkyl, phenyl, or 6-membered heteroaryl; wherein the phenyl is substituted with 0 or 1 occurrence of R⁹. In certain embodiments, R⁵ is C₃₋₆ cycloalkyl, phenyl, or 5-6 membered heteroaryl. In certain embodiments, R⁵ is C₃₋₆ cycloalkyl, phenyl, or 6-membered heteroaryl. In certain embodiments, R⁵ is C₃₋₆ cycloalkyl. In certain embodiments, R⁵ is phenyl substituted with 0 or 1 occurrence of R⁹. In certain embodiments, R⁵ is phenyl. In certain embodiments, R⁵ is 5-6 membered heteroaryl. In certain embodiments, R⁵ is 6-membered heteroaryl.

In certain embodiments, R⁵ is —(C₁₋₄ alkylene)-OR⁷ or —(C₁₋₄ alkylene)-N(R⁷)(R⁸). In certain embodiments, R⁵ is —(CH₂)₂—OR⁷ or —(CH₂)₂—N(R⁷)(R⁸). In certain embodiments, R⁵ is —(C₁₋₄ alkylene)-OR⁷. In certain embodiments, R⁵ is —(CH₂)₂—OR⁷. In certain embodiments, R⁵ is —(C₁₋₄ alkylene)-N(R⁷)(R⁸). In certain embodiments, R⁵ is —(CH₂)₂—N(R⁷)(R⁸).

In certain embodiments, R⁵ is selected from the groups depicted in the compounds in Table 1 below.

In certain embodiments, R⁴ and R⁵ are taken together with the nitrogen atom to which they are attached to form a 3-7 membered heterocyclic ring containing the nitrogen atom and 0 or 1 additional heteroatom selected from nitrogen, oxygen, and sulfur, wherein the 3-7 membered heterocyclic ring is substituted with 0, 1, 2, or 3 occurrences of R⁹. In certain embodiments, R⁴ and R⁵ are taken together with the nitrogen atom to which they are attached to form a 3-7 membered heterocyclic ring containing the nitrogen atom and 0 or 1 additional heteroatom selected from nitrogen, oxygen, and sulfur, wherein the additional nitrogen atom is optionally substituted with C₁₋₄ alkyl. In certain embodiments, R⁴ and R⁵ are taken together with the nitrogen atom to which they are attached to form a 3-7 membered heterocyclic ring containing the nitrogen atom and 0 or 1 additional nitrogen atom; wherein the additional nitrogen atom is optionally substituted with C₁₋₄ alkyl. In certain embodiments, R⁴ and R⁵ are taken together with the nitrogen atom to which they are attached to form a 3-7 membered heterocyclic ring containing the nitrogen atom and no additional heteroatoms; wherein the 3-7 membered heterocyclic ring is substituted with 0, 1, 2, or 3 occurrences of R⁹.

In certain embodiments, R⁴ and R⁵ are taken together with the nitrogen atom to which they are attached to form a 3-7 membered heterocyclic ring containing the nitrogen atom and 0 or 1 additional heteroatom selected from nitrogen, oxygen, and sulfur. In certain embodiments, R⁴ and R⁵ are taken together with the nitrogen atom to which they are attached to form a 3-7 membered heterocyclic ring containing the nitrogen atom and 0 or 1 additional nitrogen atom. In certain embodiments, R⁴ and R⁵ are taken together with the nitrogen atom to which they are attached to form a 3-7 membered heterocyclic ring containing the nitrogen atom and no additional heteroatoms.

In certain embodiments, R⁴ and R⁵ are selected from the groups depicted in the compounds in Table 1 below.

As defined generally above, R⁶ is C₃₋₆ cycloalkyl, phenyl, or 5-6 membered heteroaryl; each of which is substituted with 0, 1, 2, or 3 occurrences of R⁹. In certain embodiments, R⁶ is C₃₋₆ cycloalkyl, phenyl, or 6-membered heteroaryl; each of which is substituted with 0, 1, 2, or 3 occurrences of R⁹. In certain embodiments, R⁶ is phenyl or 5-6 membered heteroaryl, each of which is substituted with 0, 1, 2, or 3 occurrences of R⁹. In certain embodiments, R⁶ is phenyl or 6-membered heteroaryl, each of which is substituted with 0, 1, 2, or 3 occurrences of R⁹.

In certain embodiments, R⁶ is C₃₋₆ cycloalkyl, phenyl, or 5-6 membered heteroaryl; wherein the phenyl is substituted with 0 or 1 occurrence of R⁹. In certain embodiments, R⁶ is C₃₋₆ cycloalkyl, phenyl, or 6-membered heteroaryl; wherein the phenyl is substituted with 0 or 1 occurrence of R⁹. In certain embodiments, R⁶ is phenyl or 5-6 membered heteroaryl, wherein the phenyl is substituted with 0 or 1 occurrence of R⁹. In certain embodiments, R⁶ is phenyl or 6-membered heteroaryl, wherein the phenyl is substituted with 0 or 1 occurrence of R⁹.

In certain embodiments, R⁶ is C₃₋₆ cycloalkyl, phenyl, or 5-6 membered heteroaryl. In certain embodiments, R⁶ is C₃₋₆ cycloalkyl, phenyl, or 6-membered heteroaryl. In certain embodiments, R⁶ is phenyl or 5-6 membered heteroaryl. In certain embodiments, R⁶ is phenyl or 6-membered heteroaryl.

In certain embodiments, R⁶ is C₃₋₆ cycloalkyl substituted with 0, 1, 2, or 3 occurrences of R⁹. In certain embodiments, R⁶ is C₃₋₆ cycloalkyl. In certain embodiments, R⁶ is phenyl substituted with 0, 1, 2, or 3 occurrences of R⁹. In certain embodiments, R⁶ is phenyl. In certain embodiments, R⁶ is 5-6 membered heteroaryl substituted with 0, 1, 2, or 3 occurrences of R⁹. In certain embodiments, R⁶ is 6-membered heteroaryl substituted with 0, 1, 2, or 3 occurrences of R⁹. In certain embodiments, R⁶ is 5-6 membered heteroaryl. In certain embodiments, R⁶ is 6-membered heteroaryl.

As defined generally above, R⁷ and R⁸ are independently for each occurrence hydrogen, C₁₋₄ alkyl, or C₃₋₆ cycloalkyl; or R⁷ and R⁸ are taken together with the nitrogen atom to which they are attached to form a 3-7 membered heterocyclic ring containing the nitrogen atom. In certain embodiments, R⁷ and R⁸ are independently for each occurrence hydrogen, C₁₋₄ alkyl, or C₃₋₆ cycloalkyl. In certain embodiments, R⁷ and R⁸ are independently for each occurrence hydrogen or C₁₋₄ alkyl. In certain embodiments, R⁷ and R⁸ are independently for each occurrence hydrogen or methyl. In certain embodiments, R⁷ and R⁸ are independently for each occurrence C₁₋₄ alkyl or C₃₋₆ cycloalkyl.

In certain embodiments, R⁷ and R⁸ are hydrogen. In certain embodiments, R⁷ and R⁸ are independently for each occurrence C₁₋₄ alkyl. In certain embodiments, R⁷ and R⁸ are methyl. In certain embodiments, R⁷ and R⁸ are independently for each occurrence C₃₋₆ cycloalkyl. In certain embodiments, R⁷ and R⁸ are independently for each occurrence selected from the groups depicted in the compounds in Table 1 below.

As defined generally above, R⁹ represents independently for each occurrence C₁₋₄ alkyl, C₃₋₆ cycloalkyl, halo, —OR⁷, or —N(R⁷)(R⁸). In certain embodiments, R⁹ represents independently for each occurrence C₁₋₄ alkyl, fluoro, chloro, —OH, or —NH₂. In certain embodiments, R⁹ is C₁₋₄ alkyl, fluoro, chloro, —OH, or —NH₂. In certain embodiments, R⁹ represents independently for each occurrence C₁₋₄ alkyl, C₃₋₆ cycloalkyl, or halo. In certain embodiments, R⁹ represents independently for each occurrence C₁₋₄ alkyl or C₃₋₆ cycloalkyl. In certain embodiments, R⁹ represents independently for each occurrence C₁₋₄ alkyl or halo. In certain embodiments, R⁹ represents independently for each occurrence methyl or halo. In certain embodiments, R⁹ represents independently for each occurrence —OR⁷ or —N(R⁷)(R⁸). In certain embodiments, R⁹ represents independently for each occurrence —OH or —NH₂.

In certain embodiments, R⁹ represents independently for each occurrence C₁₋₄ alkyl. In certain embodiments, R⁹ is methyl. In certain embodiments, R⁹ represents independently for each occurrence C₃₋₆ cycloalkyl. In certain embodiments, R⁹ represents independently for each occurrence halo. In certain embodiments, R⁹ represents independently for each occurrence fluoro or chloro. In certain embodiments, R⁹ represents independently for each occurrence —OR⁷. In certain embodiments, R⁹ is —OH. In certain embodiments, R⁹ represents independently for each occurrence —N(R⁷)(R⁸). In certain embodiments, R⁹ is —NH₂. In certain embodiments, R⁹ is selected from the groups depicted in the compounds in Table 1 below.

As defined generally above, X is C₁₋₃ alkylene or C₁₋₃ deuteroalkylene. In certain embodiments, X is —CH₂CH₂— or —CH₂CH(CH₃)—. In certain embodiments, X is —CH₂CH₂—. In certain embodiments, X is —CH₂CH₂CH₂—. In certain embodiments, X is —CH₂CH(CH₃)—. In certain embodiments, X is —CH₂—. In certain embodiments, X is C₁₋₃ deuteroalkylene.

In certain embodiments, X is —CZ₂CZ₂—, wherein each Z is hydrogen or deuterium, provided that the abundance of deuterium in Z is at least 75%. In certain embodiments, the abundance of deuterium in Z is at least 90%. In certain embodiments, the abundance of deuterium in Z is at least 95%. In certain embodiments, X is —CD₂CH₂—. In certain embodiments, X is —CH₂CD₂—. In certain embodiments, X is —CD₂CD₂-.

In certain embodiments, X is selected from the groups depicted in the compounds in Table 1 below.

The description above describes multiple embodiments relating to compounds of Formula I. The patent application specifically contemplates all combinations of the embodiments.

Another aspect of the invention provides a compound represented by Formula I-A:

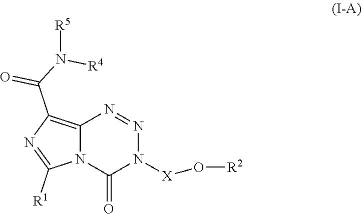

or a pharmaceutically acceptable salt thereof, wherein:
R¹ is hydrogen or methyl;
R² is C₁₋₂ fluoroalkyl;
R⁴ is hydrogen or C₁₋₄ alkyl;
R⁵ is hydrogen, C₁₋₄ alkyl, C₃₋₆ cycloalkyl, phenyl, or 5-6 membered heteroaryl; wherein the phenyl is substituted with 0 or 1 occurrence of R⁹; or R⁴ and R⁵ are taken together with the nitrogen atom to which they are attached to form a 3-7 membered saturated heterocyclic ring containing the nitrogen atom and 0 or 1 additional nitrogen atom; wherein the additional nitrogen atom is optionally substituted with C₁₋₄ alkyl;
R⁹ is C₁₋₄ alkyl, fluoro, chloro, —OH, or —NH₂; and
X is C₁₋₃ alkylene.

The definitions of variables in Formula I-A above encompass multiple chemical groups. The application contemplates embodiments where, for example, i) the definition of a variable is a single chemical group selected from those chemical groups set forth above, ii) the definition of a variable is a collection of two or more of the chemical groups selected from those set forth above, and iii) the compound is defined by a combination of variables in which the variables are defined by (i) or (ii).

In certain embodiments, the compound is a compound of Formula I-A.

As defined generally above, R¹ is hydrogen or methyl. In certain embodiments, R¹ is hydrogen. In certain embodiments, R¹ is methyl.

As defined generally above, R² is C₁₋₂ fluoroalkyl. In certain embodiments, R² is trifluoromethyl. In certain embodiments, R² is C₁ fluoroalkyl. In certain embodiments, R² is C₁₋₂ trifluoroalkyl.

As defined generally above, R⁴ is hydrogen or C₁₋₄ alkyl, or R⁴ and R⁵ are taken together with the nitrogen atom to which they are attached to form a 3-7 membered saturated heterocyclic ring containing the nitrogen atom and 0 or 1 additional nitrogen atom; wherein the additional nitrogen atom is optionally substituted with C₁₋₄ alkyl. In certain embodiments, R⁴ is hydrogen or C₁₋₄ alkyl. In certain embodiments, R⁴ is hydrogen or methyl. In certain embodiments, R⁴ is hydrogen. In certain embodiments, R⁴ is C₁₋₄ alkyl. In certain embodiments, R⁴ is methyl.

As defined generally above, R⁵ is hydrogen, C₁₋₄ alkyl, C₃₋₆ cycloalkyl, phenyl, or 5-6 membered heteroaryl; wherein the phenyl is substituted with 0 or 1 occurrence of R⁹; or R⁴ and R⁵ are taken together with the nitrogen atom to which they are attached to form a 3-7 membered saturated heterocyclic ring containing the nitrogen atom and 0 or 1 additional nitrogen atom; wherein the additional nitrogen atom is optionally substituted with C₁₋₄ alkyl.

In certain embodiments, R⁵ is hydrogen, C₁₋₄ alkyl, C₃₋₆ cycloalkyl, phenyl, or 5-6 membered heteroaryl; wherein the phenyl is substituted with 0 or 1 occurrence of R⁹. In certain embodiments, R⁵ is hydrogen or C₁₋₄ alkyl. In certain embodiments, R⁵ is hydrogen. In certain embodiments, R⁵ is C₁₋₄ alkyl. In certain embodiments, R⁵ is hydrogen, C₁₋₄ alkyl, C₃₋₆ cycloalkyl, or phenyl.

In certain embodiments, R⁵ is C₃₋₆ cycloalkyl, phenyl, or 5-6 membered heteroaryl; wherein the phenyl is substituted with 0 or 1 occurrence of R⁹. In certain embodiments, R⁵ is C₃₋₆ cycloalkyl, phenyl, or 6-membered heteroaryl; wherein the phenyl is substituted with 0 or 1 occurrence of R⁹. In certain embodiments, R⁵ is C₃₋₆ cycloalkyl, phenyl, or 5-6 membered heteroaryl. In certain embodiments, R⁵ is C₃₋₆ cycloalkyl, phenyl, or 6-membered heteroaryl. In certain embodiments, R⁵ is C₃₋₆ cycloalkyl. In certain embodiments, R⁵ is phenyl substituted with 0 or 1 occurrence of R⁹. In certain embodiments, R⁵ is phenyl. In certain embodiments, R⁵ is 5-6 membered heteroaryl. In certain embodiments, R⁵ is 6-membered heteroaryl.

In certain embodiments, R⁴ and R⁵ are taken together with the nitrogen atom to which they are attached to form a 3-7 membered saturated heterocyclic ring containing the nitrogen atom and 0 or 1 additional nitrogen atom; wherein the additional nitrogen atom is optionally substituted with C₁₋₄ alkyl. In certain embodiments, R⁴ and R⁵ are taken together with the nitrogen atom to which they are attached to form a 3-7 membered heterocyclic ring containing the nitrogen atom and no additional heteroatoms.

As defined generally above, R⁹ is C₁₋₄ alkyl, fluoro, chloro, —OH, or —NH₂. In certain embodiments, R⁹ is C₁₋₄ alkyl, fluoro, or chloro. In certain embodiments, R⁹ is methyl, fluoro, or chloro. In certain embodiments, R⁹ is —OH or —NH₂. In certain embodiments, R⁹ is C₁₋₄ alkyl. In certain embodiments, R⁹ is methyl. In certain embodiments, R⁹ is fluoro or chloro. In certain embodiments, R⁹ is —OH. In certain embodiments, R⁹ is —NH₂.

As defined generally above, X is C₁₋₃ alkylene. In certain embodiments, X is —CH₂CH₂— or —CH₂CH(CH₃)—. In certain embodiments, X is —CH₂CH₂—. In certain embodiments, X is —CH₂CH₂CH₂—. In certain embodiments, X is —CH₂CH(CH₃)—. In certain embodiments, X is —CH₂—.

The description above describes multiple embodiments relating to compounds of Formula I-A. The patent application specifically contemplates all combinations of the embodiments.

Another aspect of the invention provides a compound represented by Formula I-aa:

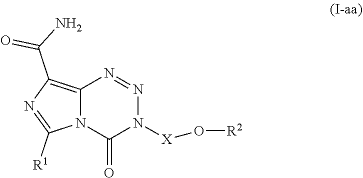

or a pharmaceutically acceptable salt thereof, wherein:
R¹ is hydrogen or methyl;
R² is C₁₋₂ fluoroalkyl; and
X is C₁₋₃ alkylene.

The definitions of variables in Formula I-aa above encompass multiple chemical groups. The application contemplates embodiments where, for example, i) the definition of a variable is a single chemical group selected from those chemical groups set forth above, ii) the definition of a variable is a collection of two or more of the chemical groups selected from those set forth above, and iii) the compound is defined by a combination of variables in which the variables are defined by (i) or (ii).

In certain embodiments, the compound is a compound of Formula I-aa.

As defined generally above, R¹ is hydrogen or methyl. In certain embodiments, R¹ is hydrogen. In certain embodiments, R¹ is methyl. In certain embodiments, R¹ is selected from the groups depicted in compounds I-1 and I-2 in Table 1 below.

As defined generally above, R² is C₁₋₂ fluoroalkyl. In certain embodiments, R² is trifluoromethyl. In certain embodiments, R² is C₁ fluoroalkyl. In certain embodiments, R² is C₁₋₂ trifluoroalkyl. In certain embodiments, R² is selected from the groups depicted in compounds I-1 and I-2 in Table 1 below.

As defined generally above, X is C₁₋₃ alkylene. In certain embodiments, X is —CH₂CH₂—. In certain embodiments, X is —CH₂CH₂CH₂—. In certain embodiments, X is —CH₂—. In certain embodiments, X is selected from the groups depicted in compounds I-1 and I-2 in Table 1 below.

The description above describes multiple embodiments relating to compounds of Formula I-aa. The patent application specifically contemplates all combinations of the embodiments.

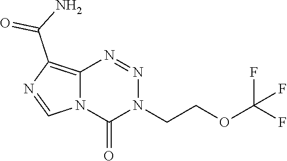 [SMILES: NC(=O)c1ncn2c(=O)n(CCOC(F)(F)F)nnc12]

In certain embodiments, the compound i 0 or a pharmaceutically acceptable salt thereof. In certain embodiments, the compound is

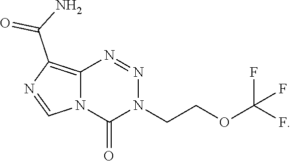 [SMILES: NC(=O)c1ncn2c(=O)n(CCOC(F)(F)F)nnc12]

Another aspect of the invention provides a compound represented by Formula II:

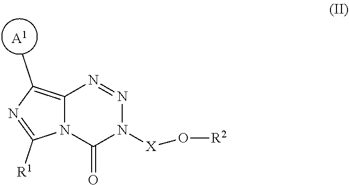

or a pharmaceutically acceptable salt thereof, wherein:
X is C₁₋₃ alkylene or C₁₋₃ deuteroalkylene;
A¹ is a 5-10 membered monocyclic or bicyclic heteroaryl containing 1, 2, 3, or 4 heteroatoms independently selected from oxygen, nitrogen, and sulfur; or A¹ is phenyl; wherein A¹ is substituted with m occurrences of R³ and n occurrences of R⁴;
R¹ is hydrogen or C₁₋₄ alkyl;
R² is C₁₋₄ fluoroalkyl;
R³ represents independently for each occurrence C₁₋₆ alkyl, C₁₋₆ haloalkyl, C₃₋₆ cycloalkyl, halogen, cyano, C₁₋₄ alkoxyl, or C₁₋₄haloalkoxyl;
R⁴ is C₁₋₆ alkyl, C₁₋₆ haloalkyl, cyano, halogen, —(C₁₋₆ alkylene)-R⁵, —(C₁₋₆ alkylene)-N(R⁶)(R⁷), —C(O)—R⁵, —C(O)N(R⁶)(R⁷), —C(O)-(saturated C₁₋₆ aliphatic), —(C₁₋₆ alkylene)-C(O)—R⁵, —(C₁₋₆ alkylene)-C(O)N(R⁶)(R⁷), C₁₋₄ alkoxyl, C₁₋₄haloalkoxyl, or R⁵;
R⁵ is phenyl; C₃₋₇ cycloalkyl; a 3-7 membered saturated heterocyclyl containing 1, 2, or 3 heteroatoms independently selected from oxygen, nitrogen, and sulfur; or a 5-10 membered monocyclic or bicyclic heteroaryl containing 1, 2, or 3 heteroatoms independently selected from oxygen, nitrogen, and sulfur; wherein the phenyl, cycloalkyl, heterocyclyl, and heteroaryl are optionally substituted with 1, 2, or 3 occurrences of R⁸;
R⁶ and R⁷ are independently hydrogen, C₁₋₆ alkyl, C₂₋₆ hydroxylalkyl, —(C₁₋₆ alkylene)-C₃₋₆ cycloalkyl, —(C₂₋₆ alkylene)-N(R⁹)(R¹⁰), or R⁵;
R⁸ represents independently for each occurrence halogen, cyano, saturated C₁₋₆ aliphatic, or C₁₋₄ alkoxyl; or two occurrences of R⁸ are taken together with their intervening atoms to form a ring;
R⁹ and R¹⁰ each represent independently for each occurrence hydrogen or C₁₋₆ alkyl;
m is 0, 1, or 2; and
n is 0 or 1.

The definitions of variables in Formula II above encompass multiple chemical groups. The application contemplates embodiments where, for example, i) the definition of a variable is a single chemical group selected from those chemical groups set forth above, ii) the definition of a variable is a collection of two or more of the chemical groups selected from those set forth above, and iii) the compound is defined by a combination of variables in which the variables are defined by (i) or (ii).

In certain embodiments, the compound is a compound of Formula II.

As defined generally above, X is C₁₋₃ alkylene or C₁₋₃ deuteroalkylene. In certain embodiments, X is —CH₂CH₂— or —CH₂CH(CH₃)—. In certain embodiments, X is —CH₂CH₂—. In certain embodiments, X is —CH₂CH₂CH₂—. In certain embodiments, X is —CH₂CH(CH₃)—. In certain embodiments, X is —CH₂—. In certain embodiments, X is C₁₋₃ deuteroalkylene.

In certain embodiments, X is —CZ₂CZ₂—, wherein each Z is hydrogen or deuterium, provided that the abundance of deuterium in Z is at least 75%. In certain embodiments, the abundance of deuterium in Z is at least 90%. In certain embodiments, the abundance of deuterium in Z is at least 95%. In certain embodiments, X is —CD₂CH₂—. In certain embodiments, X is —CH₂CD₂—. In certain embodiments, X is —CD₂CD₂-.

In certain embodiments, X is selected from the groups depicted in the compounds in Table 2 below.

As defined generally above, A¹ is a 5-10 membered monocyclic or bicyclic heteroaryl containing 1, 2, 3, or 4 heteroatoms independently selected from oxygen, nitrogen, and sulfur; or A¹ is phenyl; wherein A¹ is substituted with m occurrences of R³ and n occurrences of R⁴.

In certain embodiments, A¹ is a 5-10 membered monocyclic or bicyclic heteroaryl containing 1, 2, 3, or 4 heteroatoms independently selected from oxygen, nitrogen, and sulfur, wherein the heteroaryl is substituted with m occurrences of R³ and n occurrences of R⁴. In certain embodiments, A¹ is a 5-6 membered monocyclic heteroaryl containing 1, 2, or 3 heteroatoms independently selected from oxygen, nitrogen, and sulfur, wherein the heteroaryl is substituted with m occurrences of R³ and n occurrences of R⁴. In certain embodiments, A¹ is a 6-membered monocyclic heteroaryl containing 1, 2, or 3 heteroatoms independently selected from oxygen, nitrogen, and sulfur, wherein the heteroaryl is substituted with m occurrences of R³ and n occurrences of R⁴. In certain embodiments, A¹ is pyridyl substituted with m occurrences of R³ and n occurrences of R⁴.

In certain embodiments, A¹ is a 5-membered monocyclic heteroaryl containing 1, 2, or 3 heteroatoms independently selected from oxygen, nitrogen, and sulfur, wherein the heteroaryl is substituted with m occurrences of R³ and n occurrences of R⁴. In certain embodiments, A¹ is a 5-membered monocyclic heteroaryl containing 2 or 3 heteroatoms independently selected from oxygen, nitrogen, and sulfur, wherein at least one of the heteroatoms is nitrogen; wherein the heteroaryl is substituted with m occurrences of R³ and n occurrences of R⁴. In certain embodiments, A¹ is thiazolyl, thiadiazolyl, oxadiazolyl, oxazolyl, or imidazolyl; each of which is substituted with m occurrences of R³ and n occurrences of R⁴. In certain embodiments, A¹ is thiazol-2-yl, 1,2,4-thiadiazol-5-yl, 1,2,4-oxadiazol-5-yl, oxazol-2-yl, or imidazol-2-yl; each of which is substituted with m occurrences of R³ and n occurrences of R⁴.

In certain embodiments, A¹ is a 9-10 membered bicyclic heteroaryl containing 1, 2, or 3 heteroatoms independently selected from oxygen, nitrogen, and sulfur, wherein the heteroaryl is substituted with m occurrences of R³ and n occurrences of R⁴. In certain embodiments, A¹ is a 9-membered bicyclic heteroaryl containing 1, 2, or 3 heteroatoms independently selected from oxygen, nitrogen, and sulfur, wherein the heteroaryl is substituted with m occurrences of R³ and n occurrences of R⁴. In certain embodiments, A¹ is a 9-membered bicyclic heteroaryl containing 2 or 3 heteroatoms independently selected from oxygen, nitrogen, and sulfur, wherein at least one of the heteroatoms is nitrogen; wherein the heteroaryl is substituted with m occurrences of R³ and n occurrences of R⁴. In certain embodiments, A¹ is benzo[d]oxazolyl, oxazolo[4,5-b]pyridinyl, or benzo[d]imidazolyl; each of which is substituted with m occurrences of R³ and n occurrences of R⁴. In certain embodiments, A¹ is benzo[d]oxazol-2-yl, benzo[d]imidazol-2-yl, or benzo[d]thiazol-2-yl; each of which is substituted with m occurrences of R³ and n occurrences of R⁴.

In certain embodiments, A¹ is a 5-10 membered monocyclic or bicyclic heteroaryl containing 1, 2, 3, or 4 heteroatoms independently selected from oxygen, nitrogen, and sulfur. In certain embodiments, A¹ is a 5-6 membered monocyclic heteroaryl containing 1, 2, or 3 heteroatoms independently selected from oxygen, nitrogen, and sulfur. In certain embodiments, A¹ is a 5-membered monocyclic heteroaryl containing 1, 2, or 3 heteroatoms independently selected from oxygen, nitrogen, and sulfur. In certain embodiments, A¹ is thiazolyl, thiadiazolyl, oxadiazolyl, oxazolyl, or imidazolyl. In certain embodiments, A¹ is thiazol-2-yl, 1,2,4-thiadiazol-5-yl, 1,2,4-oxadiazol-5-yl, oxazol-2-yl, or imidazol-2-yl. In certain embodiments, A¹ is a 6-membered monocyclic heteroaryl containing 1, 2, or 3 heteroatoms independently selected from oxygen, nitrogen, and sulfur. In certain embodiments, A¹ is pyridyl.

In certain embodiments, A¹ is a 9-10 membered bicyclic heteroaryl containing 1, 2, or 3 heteroatoms independently selected from oxygen, nitrogen, and sulfur. In certain embodiments, A¹ is a 9-membered bicyclic heteroaryl containing 1, 2, or 3 heteroatoms independently selected from oxygen, nitrogen, and sulfur. In certain embodiments, A¹ is benzo[d]oxazolyl, oxazolo[4,5-b]pyridinyl, or benzo[d]imidazolyl. In certain embodiments, A¹ is benzo[d]oxazol-2-yl, benzo[d]imidazol-2-yl, or benzo[d]thiazol-2-yl.

In certain embodiments, A¹ is phenyl substituted with m occurrences of R³ and n occurrences of R⁴. In certain embodiments, A¹ is phenyl. In certain embodiments, A¹ is selected from the groups depicted in the compounds in Table 2 below.

As defined generally above, R¹ is hydrogen or C₁₋₄ alkyl. In certain embodiments, R¹ is hydrogen or methyl. In certain embodiments, R¹ is hydrogen. In certain embodiments, R¹ is C₁₋₄ alkyl. In certain embodiments, R¹ is methyl. In certain embodiments, R¹ is selected from the groups depicted in the compounds in Table 2 below.

As defined generally above, R² is C₁₋₄ fluoroalkyl. In certain embodiments, R² is C₁₋₂ fluoroalkyl. In certain embodiments, R² is C₁ fluoroalkyl. In certain embodiments, R² is C₁₋₂ trifluoroalkyl. In certain embodiments, the fluoroalkyl contains at least three fluorine atoms. In certain embodiments, R² is trifluoromethyl. In certain embodiments, R² is selected from the groups depicted in the compounds in Table 2 below.

As defined generally above, R³ represents independently for each occurrence C₁₋₆ alkyl, C₁₋₆ haloalkyl, C₃₋₆ cycloalkyl, halogen, cyano, C₁₋₄ alkoxyl, or C₁₋₄ haloalkoxyl. In certain embodiments, R³ represents independently for each occurrence C₁₋₆ alkyl, C₁₋₆ haloalkyl, or C₃₋₆ cycloalkyl. In certain embodiments, R³ represents independently for each occurrence halogen, cyano, C₁₋₄ alkoxyl, or C₁₋₄haloalkoxyl. In certain embodiments, R³ represents independently for each occurrence C₁₋₆ alkyl, C₁₋₆ haloalkyl, or halogen. In certain embodiments, R³ represents independently for each occurrence methyl, ethyl, or halogen.

In certain embodiments, R³ represents independently for each occurrence C₁₋₆ alkyl. In certain embodiments, R³ represents independently for each occurrence methyl or ethyl. In certain embodiments, R³ is methyl. In certain embodiments, R³ is ethyl. In certain embodiments, R³ represents independently for each occurrence C₁₋₆ haloalkyl. In certain embodiments, R³ represents independently for each occurrence C₃₋₆ cycloalkyl. In certain embodiments, R³ represents independently for each occurrence halogen. In certain embodiments, R³ is cyano. In certain embodiments, R³ represents independently for each occurrence C₁₋₄ alkoxyl. In certain embodiments, R³ represents independently for each occurrence C₁₋₄ haloalkoxyl. In certain embodiments, R³ is selected from the groups depicted in the compounds in Table 2 below.

As defined generally above, R⁴ is C₁₋₆ alkyl, C₁₋₆ haloalkyl, cyano, halogen, —(C₁₋₆ alkylene)-R⁵, —(C₁₋₆ alkylene)-N(R⁶)(R⁷), —C(O)—R⁵, —C(O)N(R⁶)(R⁷), —C(O)-(saturated C₁₋₆ aliphatic), —(C₁₋₆ alkylene)-C(O)—R⁵, —(C₁₋₆ alkylene)-C(O)N(R⁶)(R⁷), C₁₋₄ alkoxyl, C₁₋₄ haloalkoxyl, or R⁵.

In certain embodiments, R⁴ is C₁₋₆ alkyl, C₁₋₆ haloalkyl, cyano, or halogen. In certain embodiments, R⁴ is —(C₁₋₆ alkylene)-R⁵, —(C₁₋₆ alkylene)-N(R⁶)(R⁷), —C(O)—R⁵, —C(O)N(R⁶)(R⁷), —C(O)-(saturated C₁₋₆ aliphatic), —(C₁₋₆ alkylene)-C(O)—R⁵, —(C₁₋₆ alkylene)-C(O)N(R⁶)(R⁷), C₁₋₄ alkoxyl, or C₁₋₄ haloalkoxyl. In certain embodiments, R⁴ is —(C₁₋₆ alkylene)-R⁵, —C(O)—R⁵, —(C₁₋₆ alkylene)-C(O)—R⁵, or R⁵. In certain embodiments, R⁴ is —C(O)—R⁵ or —(C₁₋₆ alkylene)-C(O)—R⁵. In certain embodiments, R⁴ is —(C₁₋₆ alkylene)-N(R⁶)(R⁷), —C(O)N(R⁶)(R⁷), or —(C₁₋₆ alkylene)-C(O)N(R⁶)(R⁷). In certain embodiments, R⁴ is —C(O)N(R⁶)(R⁷) or —(C₁₋₆ alkylene)-C(O)N(R⁶)(R⁷). In certain embodiments, R⁴ is C₁₋₆ haloalkyl, cyano, halogen, or C₁₋₄ haloalkoxyl. In certain embodiments, R⁴ is C₁₋₆ alkyl, cyano, or R⁵.

In certain embodiments, R⁴ is C₁₋₆ alkyl. In certain embodiments, R⁴ is methyl. In certain embodiments, R⁴ is C₁₋₆ haloalkyl. In certain embodiments, R⁴ is cyano. In certain embodiments, R⁴ is halogen. In certain embodiments, R⁴ is —(C₁₋₆ alkylene)-R⁵. In certain embodiments, R⁴ is —(C₁₋₆ alkylene)-N(R⁶)(R⁷). In certain embodiments, R⁴ is —C(O)—R⁵. In certain embodiments, R⁴ is —C(O)N(R⁶)(R⁷). In certain embodiments, R⁴ is —C(O)-(saturated C₁₋₆ aliphatic). In certain embodiments, R⁴ is —(C₁₋₆ alkylene)-C(O)—R⁵. In certain embodiments, R⁴ is —(C₁₋₆ alkylene)-C(O)N(R⁶)(R⁷). In certain embodiments, R⁴ is C₁₋₄ alkoxyl. In certain embodiments, R⁴ is C₁₋₄haloalkoxyl. In certain embodiments, R⁴ is R⁵. In certain embodiments, R⁴ is selected from the groups depicted in the compounds in Table 2 below.

As defined generally above, R⁵ is phenyl; C₃₋₇ cycloalkyl; a 3-7 membered saturated heterocyclyl containing 1, 2, or 3 heteroatoms independently selected from oxygen, nitrogen, and sulfur; or a 5-10 membered monocyclic or bicyclic heteroaryl containing 1, 2, or 3 heteroatoms independently selected from oxygen, nitrogen, and sulfur; wherein the phenyl, cycloalkyl, heterocyclyl, and heteroaryl are optionally substituted with 1, 2, or 3 occurrences of R⁸.

In certain embodiments, R⁵ is phenyl or a 5-10 membered monocyclic or bicyclic heteroaryl containing 1, 2, or 3 heteroatoms independently selected from oxygen, nitrogen, and sulfur; wherein the phenyl and heteroaryl are optionally substituted with 1, 2, or 3 occurrences of R⁸. In certain embodiments, R⁵ is phenyl or a 5-10 membered monocyclic or bicyclic heteroaryl containing 1, 2, or 3 heteroatoms independently selected from oxygen, nitrogen, and sulfur. In certain embodiments, R⁵ is phenyl or a 5-6 membered monocyclic heteroaryl containing 1, 2, or 3 heteroatoms independently selected from oxygen, nitrogen, and sulfur; wherein the phenyl and heteroaryl are optionally substituted with 1, 2, or 3 occurrences of R⁸. In certain embodiments, R⁵ is phenyl or a 5-6 membered monocyclic heteroaryl containing 1, 2, or 3 heteroatoms independently selected from oxygen, nitrogen, and sulfur.

In certain embodiments, R⁵ is phenyl optionally substituted with 1, 2, or 3 occurrences of R⁸. In certain embodiments, R⁵ is phenyl, 4-fluorophenyl, 4-cyanophenyl, 3-cyanophenyl, 2-cyanophenyl, 4-methylphenyl, 3-methylphenyl, or 2-methylphenyl. In certain embodiments, R⁵ is phenyl.

In certain embodiments, R⁵ is C₃₋₇ cycloalkyl optionally substituted with 1, 2, or 3 occurrences of R⁸. In certain embodiments, R⁵ is C₃₋₇ cycloalkyl. In certain embodiments, R⁵ is cyclopropyl, cyclobutyl, or cyclopentyl.

In certain embodiments, R⁵ is a 3-7 membered saturated heterocyclyl containing 1, 2, or 3 heteroatoms independently selected from oxygen, nitrogen, and sulfur; wherein the heterocyclyl is optionally substituted with 1, 2, or 3 occurrences of R⁸. In certain embodiments, R³ is azetidinyl, pyrrolidinyl, piperidinyl, morpholinyl, or piperazinyl; each of which is optionally substituted with 1, 2, or 3 occurrences of R⁸. In certain embodiments, R⁵ is a 3-7 membered saturated heterocyclyl containing 1, 2, or 3 heteroatoms independently selected from oxygen, nitrogen, and sulfur.

In certain embodiments, R⁵ is a 5-10 membered monocyclic or bicyclic heteroaryl containing 1, 2, or 3 heteroatoms independently selected from oxygen, nitrogen, and sulfur; wherein the heteroaryl is optionally substituted with 1, 2, or 3 occurrences of R⁸. In certain embodiments, R⁵ is a 5-10 membered monocyclic or bicyclic heteroaryl containing 1, 2, or 3 heteroatoms independently selected from oxygen, nitrogen, and sulfur.

In certain embodiments, R⁵ is a 5-6 membered monocyclic heteroaryl containing 1, 2, or 3 heteroatoms independently selected from oxygen, nitrogen, and sulfur; wherein the heteroaryl is optionally substituted with 1, 2, or 3 occurrences of R⁸. In certain embodiments, R⁵ is a 5-6 membered monocyclic heteroaryl containing 1, 2, or 3 heteroatoms independently selected from oxygen, nitrogen, and sulfur. In certain embodiments, R⁵ is thiazolyl, imidazolinyl, oxazolyl, pyridinyl, pyrimidinyl, or pyrazinyl; each of which is optionally substituted with 1, 2, or 3 occurrences of R⁸. In certain embodiments, R⁵ is thiazolyl, imidazolinyl, oxazolyl, pyridinyl, pyrimidinyl, or pyrazinyl.

In certain embodiments, R⁵ is a 5-membered monocyclic heteroaryl containing 1, 2, or 3 heteroatoms independently selected from oxygen, nitrogen, and sulfur; wherein the heteroaryl is optionally substituted with 1, 2, or 3 occurrences of R⁸. In certain embodiments, R⁵ is a 5-membered monocyclic heteroaryl containing 1, 2, or 3 heteroatoms independently selected from oxygen, nitrogen, and sulfur.

In certain embodiments, R⁵ is a 6-membered monocyclic heteroaryl containing 1, 2, or 3 heteroatoms independently selected from oxygen, nitrogen, and sulfur; wherein the heteroaryl is optionally substituted with 1, 2, or 3 occurrences of R⁸. In certain embodiments, R⁵ is a 6-membered monocyclic heteroaryl containing 1, 2, or 3 heteroatoms independently selected from oxygen, nitrogen, and sulfur.

In certain embodiments, R⁵ is a 8-10 membered bicyclic heteroaryl containing 1, 2, or 3 heteroatoms independently selected from oxygen, nitrogen, and sulfur; wherein the heteroaryl is optionally substituted with 1, 2, or 3 occurrences of R⁸. In certain embodiments, R⁵ is a 8-10 membered bicyclic heteroaryl containing 1, 2, or 3 heteroatoms independently selected from oxygen, nitrogen, and sulfur. In certain embodiments, R⁵ is selected from the groups depicted in the compounds in Table 2 below.

As defined generally above, R⁶ and R⁷ are independently hydrogen, C₁₋₆ alkyl, C₂₋₆ hydroxylalkyl, —(C₁₋₆ alkylene)-C₃₋₆ cycloalkyl, —(C₂₋₆ alkylene)-N(R⁹)(R¹⁰), or R⁵. In certain embodiments, R⁶ and R⁷ are independently hydrogen or C₁₋₆ alkyl. In certain embodiments, R⁶ is hydrogen or C₁₋₆ alkyl, and R⁷ is C₂₋₆ hydroxylalkyl, —(C₁₋₆ alkylene)-C₃₋₆ cycloalkyl, —(C₂₋₆ alkylene)-N(R⁹)(R¹⁰), or R⁵. In certain embodiments, R⁶ is hydrogen or C₁₋₆ alkyl, and R⁷ is C₂₋₆ hydroxylalkyl or —(C₂₋₆ alkylene)-N(R⁹)(R¹⁰). In certain embodiments, R⁶ and R⁷ are selected from the groups depicted in the compounds in Table 2 below.

As defined generally above, R⁸ represents independently for each occurrence halogen, cyano, saturated C₁₋₆ aliphatic, or C₁₋₄ alkoxyl; or two occurrences of R⁸ are taken together with their intervening atoms to form a ring. In certain embodiments, R⁸ represents independently for each occurrence halogen, cyano, saturated C₁₋₆ aliphatic, or C₁₋₄ alkoxyl. In certain embodiments, R⁸ represents independently for each occurrence halogen or cyano. In certain embodiments, two occurrences of R⁸ are taken together with their intervening atoms to form a ring. In certain embodiments, R⁸ is selected from the groups depicted in the compounds in Table 2 below.

As defined generally above, R⁹ and R¹⁰ each represent independently for each occurrence hydrogen or C₁₋₆ alkyl. In certain embodiments, R⁹ and R¹⁰ are hydrogen. In certain embodiments, R⁹ and R¹⁰ each represent independently for each occurrence C₁₋₆ alkyl. In certain embodiments, R⁹ and R¹⁰ are selected from the groups depicted in the compounds in Table 2 below.

As defined generally above, m is 0, 1, or 2. In certain embodiments, m is 0 or 1. In certain embodiments, m is 1 or 2. In certain embodiments, m is 0. In certain embodiments, m is 1. In certain embodiments, m is 2. In certain embodiments, m is selected from the values represented in the compounds in Table 2 below.

As defined generally above, n is 0 or 1. In certain embodiments, n is 0. In certain embodiments, n is 1. In certain embodiments, m is selected from the values represented in the compounds in Table 2 below.

In certain embodiments, the compound of Formula II is a compound of Formula IIa or IIb:

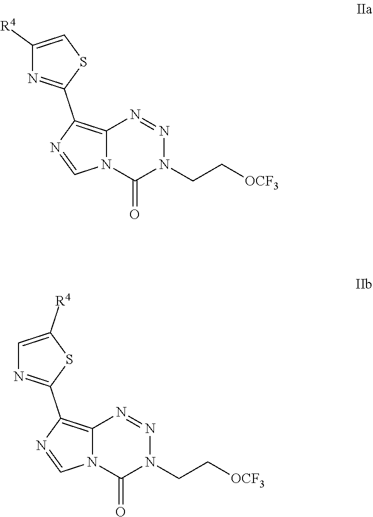

or a pharmaceutically acceptable salt thereof, wherein R⁴ is as defined in embodiments herein. In certain embodiments, the compound is a compound of Formula IIa or IIb.

In certain embodiments, the compound of Formula II is a compound of Formula IIc or IId:

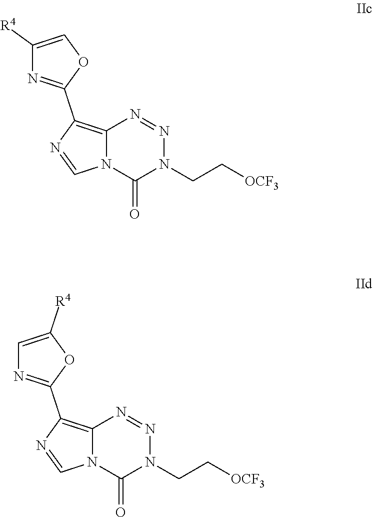

or a pharmaceutically acceptable salt thereof, wherein R⁴ is as defined in embodiments herein. In certain embodiments, the compound is a compound of Formula IIc or IId.

In certain embodiments, the compound of Formula II is a compound of Formula IIe or IIf:

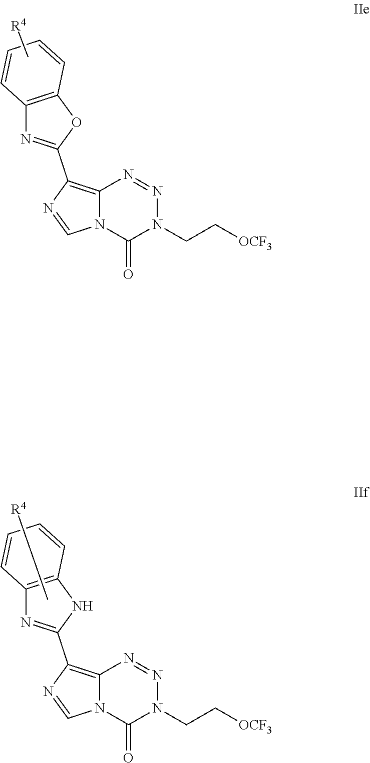

or a pharmaceutically acceptable salt thereof, wherein R⁴ is as defined in embodiments herein. In certain embodiments, the compound is a compound of Formula IIe or IIf.

The description above describes multiple embodiments relating to compounds of Formula II. The patent application specifically contemplates all combinations of the embodiments.

Another aspect of the invention provides a compound represented by Formula IIIa or Formula IIIb:

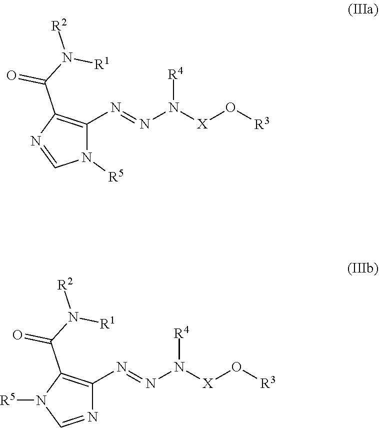

or a pharmaceutically acceptable salt thereof, wherein:
R¹ is hydrogen or C₁₋₄ alkyl;
R² is hydrogen, C₁₋₄ alkyl, C₁₋₄ alkenyl, C₁₋₄ alkynyl, C₁₋₄ haloalkyl, —(C₀₋₄ alkylene)-(C₃₋₇ cycloalkyl), or —(C₁₋₄ alkylene)-OR⁶; or R¹ and R² are taken together with the nitrogen atom to which they are attached to form a 3-7 membered heterocyclic ring containing the nitrogen atom and 0 or 1 additional heteroatom selected from nitrogen, oxygen, and sulfur, wherein the 3-7 membered heterocyclic ring is substituted with 0, 1, 2, or 3 occurrences of R⁷;
R³ is C₁₋₄ fluoroalkyl;
R⁴ is C₁₋₄ alkyl, hydrogen, C₁₋₄ haloalkyl, —(C₀₋₄ alkylene)-(C₃₋₇ cycloalkyl), or —C(O)—(C₁₋₄ alkyl);
R⁵ and R⁶ each represent independently hydrogen or C₁₋₄ alkyl; and
R⁷ represents independently for each occurrence C₁₋₄ alkyl, C₃₋₆ cycloalkyl, or halo; and
X is C₁₋₃ alkylene or C₁₋₃ deuteroalkylene.

The definitions of variables in Formula IIIa and Formula IIIb above encompass multiple chemical groups. The application contemplates embodiments where, for example, i) the definition of a variable is a single chemical group selected from those chemical groups set forth above, ii) the definition of a variable is a collection of two or more of the chemical groups selected from those set forth above, and iii) the compound is defined by a combination of variables in which the variables are defined by (i) or (ii).

In certain embodiments, the compound is a compound of Formula IIIa, or a pharmaceutically acceptable salt thereof. In certain embodiments, the compound is a compound of Formula IIIa. In certain embodiments, the compound is a compound of Formula IIIb, or a pharmaceutically acceptable salt thereof. In certain embodiments, the compound is a compound of Formula IIIb. In certain embodiments, the compound is a compound of Formula IIIa of Formula IIIb.

As defined generally above, R¹ is hydrogen or C₁₋₄ alkyl; or R¹ and R² are taken together with the nitrogen atom to which they are attached to form a 3-7 membered heterocyclic ring containing the nitrogen atom and 0 or 1 additional heteroatom selected from nitrogen, oxygen, and sulfur, wherein the 3-7 membered heterocyclic ring is substituted with 0, 1, 2, or 3 occurrences of R⁷.

In certain embodiments, R¹ is hydrogen or C₁₋₄ alkyl. In certain embodiments, R¹ is hydrogen or methyl. In certain embodiments, R¹ is hydrogen. In certain embodiments, R¹ is C₁₋₄ alkyl. In certain embodiments, R¹ is methyl. In certain embodiments, R¹ is selected from the groups depicted in the compounds in Table 3 below.

As defined generally above, R² is hydrogen, C₁₋₄ alkyl, C₁₋₄ alkenyl, C₁₋₄ alkynyl, C₁₋₄ haloalkyl, —(C₀₋₄ alkylene)-(C₃₋₇ cycloalkyl), or —(C₁₋₄ alkylene)-OR⁶; or R¹ and R² are taken together with the nitrogen atom to which they are attached to form a 3-7 membered heterocyclic ring containing the nitrogen atom and 0 or 1 additional heteroatom selected from nitrogen, oxygen, and sulfur, wherein the 3-7 membered heterocyclic ring is substituted with 0, 1, 2, or 3 occurrences of R⁷.

In certain embodiments, R² is hydrogen, C₁₋₄ alkyl, C₁₋₄ alkenyl, C₁₋₄ alkynyl, C₁₋₄ haloalkyl, —(C₀₋₄ alkylene)-(C₃₋₇ cycloalkyl), or —(C₁₋₄ alkylene)-OR⁶. In certain embodiments, R² is C₁₋₄ alkenyl, C₁₋₄ alkynyl, C₁₋₄ haloalkyl, —(C₀₋₄ alkylene)-(C₃₋₇ cycloalkyl), or —(C₁₋₄ alkylene)-OR⁶.

In certain embodiments, R² is hydrogen, C₁₋₄ alkyl, C₁₋₄ alkenyl, C₁₋₄ alkynyl, or C₁₋₄ haloalkyl. In certain embodiments, R² is hydrogen or C₁₋₄ alkyl. In certain embodiments, R² is hydrogen or methyl. In certain embodiments, R² is C₁₋₄ alkyl, C₁₋₄ alkenyl, C₁₋₄ alkynyl, or C₁₋₄ haloalkyl. In certain embodiments, R² is hydrogen. In certain embodiments, R² is C₁₋₄ alkyl. In certain embodiments, R² is methyl. In certain embodiments, R² is C₁₋₄ alkenyl. In certain embodiments, R² is C₁₋₄ alkynyl. In certain embodiments, R² is C₁₋₄ haloalkyl.

In certain embodiments, R² is —(C₀₋₄ alkylene)-(C₃₋₇ cycloalkyl) or —(C₁₋₄ alkylene)-OR⁶. In certain embodiments, R² is —(C₀₋₄ alkylene)-(C₃₋₇ cycloalkyl). In certain embodiments, R² is —(C₁₋₄ alkylene)-OR⁶. In certain embodiments, R² is selected from the groups depicted in the compounds in Table 3 below.

In certain embodiments, R¹ and R² are taken together with the nitrogen atom to which they are attached to form a 3-7 membered heterocyclic ring containing the nitrogen atom and 0 or 1 additional heteroatom selected from nitrogen, oxygen, and sulfur, wherein the 3-7 membered heterocyclic ring is substituted with 0, 1, 2, or 3 occurrences of R⁷. In certain embodiments, R¹ and R² are taken together with the nitrogen atom to which they are attached to form a 3-7 membered heterocyclic ring containing the nitrogen atom and 0 or 1 additional heteroatom selected from nitrogen, oxygen, and sulfur, wherein the additional nitrogen atom is optionally substituted with C₁₋₄ alkyl. In certain embodiments, R¹ and R² are taken together with the nitrogen atom to which they are attached to form a 3-7 membered heterocyclic ring containing the nitrogen atom and 0 or 1 additional nitrogen atom; wherein the additional nitrogen atom is optionally substituted with C₁₋₄ alkyl. In certain embodiments, R¹ and R² are taken together with the nitrogen atom to which they are attached to form a 3-7 membered heterocyclic ring containing the nitrogen atom and no additional heteroatoms; wherein the 3-7 membered heterocyclic ring is substituted with 0, 1, 2, or 3 occurrences of R⁷.

In certain embodiments, R¹ and R² are taken together with the nitrogen atom to which they are attached to form a 3-7 membered heterocyclic ring containing the nitrogen atom and 0 or 1 additional heteroatom selected from nitrogen, oxygen, and sulfur. In certain embodiments, R¹ and R² are taken together with the nitrogen atom to which they are attached to form a 3-7 membered heterocyclic ring containing the nitrogen atom and 0 or 1 additional nitrogen atom. In certain embodiments, R¹ and R² are taken together with the nitrogen atom to which they are attached to form a 3-7 membered heterocyclic ring containing the nitrogen atom and no additional heteroatoms. In certain embodiments, R¹ and R² are selected from the groups depicted in the compounds in Table 3 below.

As defined generally above, R³ is C₁₋₄ fluoroalkyl. In certain embodiments, R³ is C₁₋₂ fluoroalkyl. In certain embodiments, R³ is C₁ fluoroalkyl. In certain embodiments, the fluoroalkyl contains at least three fluorine atoms. In certain embodiments, R³ is C₁₋₂ trifluoroalkyl. In certain embodiments, R³ is trifluoromethyl. In certain embodiments, R³ is selected from the groups depicted in the compounds in Table 3 below.

As defined generally above, R⁴ is C₁₋₄ alkyl, hydrogen, C₁₋₄haloalkyl, —(C₀₋₄ alkylene)-(C₃₋₇ cycloalkyl), or —C(O)—(C₁₋₄ alkyl). In certain embodiments, R⁴ is C₁₋₄ alkyl, C₁₋₄haloalkyl, —(C₀₋₄ alkylene)-(C₃₋₇ cycloalkyl), or —C(O)—(C₁₋₄ alkyl). In certain embodiments, R⁴ is C₁₋₄ alkyl, hydrogen, C₁₋₄ haloalkyl, —(C₀₋₄ alkylene)-(C₃₋₇ cycloalkyl). In certain embodiments, R⁴ is C₁₋₄ haloalkyl, —(C₀₋₄ alkylene)-(C₃₋₇ cycloalkyl), or —C(O)—(C₁₋₄ alkyl).

In certain embodiments, R⁴ is C₁₋₄ alkyl. In certain embodiments, R⁴ is methyl. In certain embodiments, R⁴ is hydrogen. In certain embodiments, R⁴ is C₁₋₄haloalkyl. In certain embodiments, R⁴ is —(C₀₋₄ alkylene)-(C₃₋₇ cycloalkyl). In certain embodiments, R⁴ is C₃₋₇ cycloalkyl. In certain embodiments, R⁴ is —(C₁₋₄ alkylene)-(C₃₋₇ cycloalkyl). In certain embodiments, R⁴ is —C(O)—(C₁₋₄ alkyl). In certain embodiments, R⁴ is selected from the groups depicted in the compounds in Table 3 below.

As defined generally above, R⁵ and R⁶ each represent independently hydrogen or C₁₋₄ alkyl. In certain embodiments, R⁵ and R⁶ are hydrogen. In certain embodiments, R⁵ and R⁶ each represent independently C₁₋₄ alkyl. In certain embodiments, R⁵ is hydrogen. In certain embodiments, R⁵ is C₁₋₄ alkyl. In certain embodiments, R⁶ is hydrogen. In certain embodiments, R⁶ is C₁₋₄ alkyl. In certain embodiments, R⁵ and R⁶ are selected from the groups depicted in the compounds in Table 3 below.

As defined generally above, R⁷ represents independently for each occurrence C₁₋₄ alkyl, C₃₋₆ cycloalkyl, or halo. In certain embodiments, R⁷ represents independently for each occurrence C₁₋₄ alkyl or C₃₋₆ cycloalkyl. In certain embodiments, R⁷ represents independently for each occurrence C₁₋₄ alkyl or halo. In certain embodiments, R⁷ represents independently for each occurrence C₃₋₆ cycloalkyl or halo.

In certain embodiments, R⁷ represents independently for each occurrence C₁₋₄ alkyl. In certain embodiments, R⁷ represents independently for each occurrence C₃₋₆ cycloalkyl. In certain embodiments, R⁷ represents independently for each occurrence halo. In certain embodiments, R⁷ is selected from the groups depicted in the compounds in Table 3 below.

As defined generally above, X is C₁₋₃ alkylene or C₁₋₃ deuteroalkylene. In certain embodiments, X is —CH₂CH₂— or —CH₂CH(CH₃)—. In certain embodiments, X is —CH₂CH₂—. In certain embodiments, X is —CH₂CH₂CH₂—. In certain embodiments, X is —CH₂CH(CH₃)—. In certain embodiments, X is —CH₂—. In certain embodiments, X is C₁₋₃ deuteroalkylene.

In certain embodiments, X is —CZ₂CZ₂—, wherein each Z is hydrogen or deuterium, provided that the abundance of deuterium in Z is at least 75%. In certain embodiments, the abundance of deuterium in Z is at least 90%. In certain embodiments, the abundance of deuterium in Z is at least 95%. In certain embodiments, X is —CD₂CH₂—. In certain embodiments, X is —CH₂CD₂—. In certain embodiments, X is —CD₂CD₂-.

In certain embodiments, X is selected from the groups depicted in the compounds in Table 3 below.

The description above describes multiple embodiments relating to compounds of Formula IIIa and Formula IIIb. The patent application specifically contemplates all combinations of the embodiments.

Another aspect of the invention provides a compound represented by Formula IV:

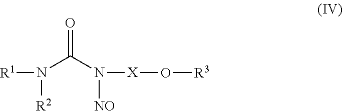

or a pharmaceutically acceptable salt thereof, wherein:
R¹ is hydrogen or C₁₋₄ alkyl;
R² is hydrogen, C₁₋₄ alkyl, C₁₋₄ alkenyl, C₁₋₄ alkynyl, C₁₋₄ haloalkyl, —(C₀₋₄ alkylene)-(C₃₋₇ cycloalkyl), —(C₁₋₄ alkylene)-OR⁵, —(C₁₋₄ alkylene)-N(R⁵)(R⁶), —(C₁₋₄ alkylene)-P(O)(OR⁵)(OR⁶), —(C₀₋₄ alkylene)-phenyl, —(C₀₋₄ alkylene)-(5-10 membered monocyclic or bicyclic heteroaryl containing 1, 2, 3, or 4 heteroatoms independently selected from oxygen, nitrogen, and sulfur), or —(C₀₋₄ alkylene)-(3-10 membered monocyclic or bicyclic, saturated or partially unsaturated heterocyclyl containing 1, 2, or 3 heteroatoms independently selected from oxygen, nitrogen, and sulfur); wherein the cycloalkyl, phenyl, heteroaryl, and heterocyclyl are substituted by n occurrences of R⁴;
or R¹ and R² are taken together with the nitrogen atom to which they are attached to form a 3-7 membered heterocyclic ring containing the nitrogen atom and 0 or 1 additional heteroatom selected from nitrogen, oxygen, and sulfur, wherein the 3-7 membered heterocyclic ring is substituted with 0, 1, 2, or 3 occurrences of R⁴;
R³ is C₁₋₄ fluoroalkyl;
R⁴ represents independently for each occurrence C₁₋₄ alkyl, C₁₋₄ haloalkyl, halo, —(C₀₋₄ alkylene)-OR⁵, or —(C₀₋₄ alkylene)-N(R⁵)(R⁶);
R⁵ and R⁶ each represent independently for each occurrence hydrogen, C₁₋₄ alkyl, or C₁₋₄ haloalkyl;
X is C₁₋₃ alkylene or C₁₋₃ deuteroalkylene; and
n is 0, 1, 2, 3, or 4.

The definitions of variables in Formula IV above encompass multiple chemical groups. The application contemplates embodiments where, for example, i) the definition of a variable is a single chemical group selected from those chemical groups set forth above, ii) the definition of a variable is a collection of two or more of the chemical groups selected from those set forth above, and iii) the compound is defined by a combination of variables in which the variables are defined by (i) or (ii).

In certain embodiments, the compound is a compound of Formula IV.

As defined generally above, R¹ is hydrogen or C₁₋₄ alkyl; or R¹ and R² are taken together with the nitrogen atom to which they are attached to form a 3-7 membered heterocyclic ring containing the nitrogen atom and 0 or 1 additional heteroatom selected from nitrogen, oxygen, and sulfur, wherein the 3-7 membered heterocyclic ring is substituted with 0, 1, 2, or 3 occurrences of R⁴.

In certain embodiments, R¹ is hydrogen or C₁₋₄ alkyl. In certain embodiments, R¹ is hydrogen or methyl. In certain embodiments, R¹ is hydrogen. In certain embodiments, R¹ is C₁₋₄ alkyl. In certain embodiments, R¹ is methyl. In certain embodiments, R¹ is selected from the groups depicted in the compounds in Table 4 below.

As defined generally above, R² is hydrogen, C₁₋₄ alkyl, C₁₋₄ alkenyl, C₁₋₄ alkynyl, C₁₋₄ haloalkyl, —(C₀₋₄ alkylene)-(C₃₋₇ cycloalkyl), —(C₁₋₄ alkylene)-OR⁵, —(C₁₋₄ alkylene)-N(R⁵)(R⁶), —(C₁₋₄ alkylene)-P(O)(OR⁵)(OR⁶), —(C₀₋₄ alkylene)-phenyl, —(C₀₋₄ alkylene)-(5-10 membered monocyclic or bicyclic heteroaryl containing 1, 2, 3, or 4 heteroatoms independently selected from oxygen, nitrogen, and sulfur), or —(C₀₋₄ alkylene)-(3-10 membered monocyclic or bicyclic, saturated or partially unsaturated heterocyclyl containing 1, 2, or 3 heteroatoms independently selected from oxygen, nitrogen, and sulfur); wherein the cycloalkyl, phenyl, heteroaryl, and heterocyclyl are substituted by n occurrences of R⁴; or R¹ and R² are taken together with the nitrogen atom to which they are attached to form a 3-7 membered heterocyclic ring containing the nitrogen atom and 0 or 1 additional heteroatom selected from nitrogen, oxygen, and sulfur, wherein the 3-7 membered heterocyclic ring is substituted with 0, 1, 2, or 3 occurrences of R⁴.

In certain embodiments, R² is hydrogen, C₁₋₄ alkyl, C₁₋₄ alkenyl, C₁₋₄ alkynyl, C₁₋₄ haloalkyl, —(C₀₋₄ alkylene)-(C₃₋₇ cycloalkyl), —(C₁₋₄ alkylene)-OR⁵, —(C₁₋₄ alkylene)-N(R⁵)(R⁶), —(C₁₋₄ alkylene)-P(O)(OR⁵)(OR⁶), —(C₀₋₄ alkylene)-phenyl, —(C₀₋₄ alkylene)-(5-10 membered monocyclic or bicyclic heteroaryl containing 1, 2, 3, or 4 heteroatoms independently selected from oxygen, nitrogen, and sulfur), or —(C₀₋₄ alkylene)-(3-10 membered monocyclic or bicyclic, saturated or partially unsaturated heterocyclyl containing 1, 2, or 3 heteroatoms independently selected from oxygen, nitrogen, and sulfur); wherein the cycloalkyl, phenyl, heteroaryl, and heterocyclyl are substituted by n occurrences of R⁴.

In certain embodiments, R² is hydrogen, C₁₋₄ alkyl, C₁₋₄ alkenyl, C₁₋₄ alkynyl, or C₁₋₄ haloalkyl. In certain embodiments, R² is hydrogen or C₁₋₄ alkyl. In certain embodiments, R² is hydrogen or methyl. In certain embodiments, R² is C₁₋₄ alkyl, C₁₋₄ alkenyl, C₁₋₄ alkynyl, or C₁₋₄ haloalkyl. In certain embodiments, R² is hydrogen. In certain embodiments, R² is C₁₋₄ alkyl. In certain embodiments, R² is methyl. In certain embodiments, R² is C₁₋₄ alkenyl. In certain embodiments, R² is C₁₋₄ alkynyl. In certain embodiments, R² is C₁₋₄ haloalkyl.

In certain embodiments, R² is —(C₁₋₄ alkylene)-OR⁵, —(C₁₋₄ alkylene)-N(R⁵)(R⁶), or —(C₁₋₄ alkylene)-P(O)(OR⁵)(OR⁶). In certain embodiments, R² is —(C₁₋₄ alkylene)-OR⁵. In certain embodiments, R² is —(C₁₋₄ alkylene)-N(R⁵)(R⁶). In certain embodiments, R² is —(C₁₋₄ alkylene)-P(O)(OR⁵)(OR⁶).

In certain embodiments, R² is —(C₀₋₄ alkylene)-(C₃₋₇ cycloalkyl), —(C₀₋₄ alkylene)-phenyl, —(C₀₋₄ alkylene)-(5-10 membered monocyclic or bicyclic heteroaryl containing 1, 2, 3, or 4 heteroatoms independently selected from oxygen, nitrogen, and sulfur), or —(C₀₋₄ alkylene)-(3-10 membered monocyclic or bicyclic, saturated or partially unsaturated heterocyclyl containing 1, 2, or 3 heteroatoms independently selected from oxygen, nitrogen, and sulfur); wherein the cycloalkyl, phenyl, heteroaryl, and heterocyclyl are substituted by n occurrences of R⁴.

In certain embodiments, R² is —(C₀₋₄ alkylene)-(C₃₋₇ cycloalkyl) or —(C₀₋₄ alkylene)-phenyl; wherein the cycloalkyl and phenyl are substituted by n occurrences of R⁴. In certain embodiments, R² is —(C₀₋₄ alkylene)-(C₃₋₇ cycloalkyl); wherein the cycloalkyl is substituted by n occurrences of R⁴. In certain embodiments, R² is —(C₁₋₄ alkylene)-(C₃₋₇ cycloalkyl); wherein the cycloalkyl is substituted by n occurrences of R⁴. In certain embodiments, R² is C₃₋₇ cycloalkyl substituted by n occurrences of R⁴. In certain embodiments, R² is C₅₋₆ cycloalkyl substituted by n occurrences of R⁴. In certain embodiments, R² is —(C₀₋₄ alkylene)-phenyl; wherein the phenyl is substituted by n occurrences of R⁴. In certain embodiments, R² is —(C₁₋₄ alkylene)-phenyl; wherein the phenyl is substituted by n occurrences of R⁴. In certain embodiments, R² is phenyl substituted by n occurrences of R⁴.

In certain embodiments, R² is —(C₀₋₄ alkylene)-(5-10 membered monocyclic or bicyclic heteroaryl containing 1, 2, 3, or 4 heteroatoms independently selected from oxygen, nitrogen, and sulfur) or —(C₀₋₄ alkylene)-(3-10 membered monocyclic or bicyclic, saturated or partially unsaturated heterocyclyl containing 1, 2, or 3 heteroatoms independently selected from oxygen, nitrogen, and sulfur); wherein the heteroaryl and heterocyclyl are substituted by n occurrences of R⁴.

In certain embodiments, R² is —(C₀₋₄ alkylene)-(5-10 membered monocyclic or bicyclic heteroaryl containing 1, 2, 3, or 4 heteroatoms independently selected from oxygen, nitrogen, and sulfur); wherein the heteroaryl is substituted by n occurrences of R⁴.

In certain embodiments, R² is —(C₀₋₄ alkylene)-(5-6 membered monocyclic heteroaryl containing 1, 2, or 3 heteroatoms independently selected from oxygen, nitrogen, and sulfur); wherein the heteroaryl is substituted by n occurrences of R⁴. In certain embodiments, R² is —(C₁₋₄ alkylene)-(5-6 membered monocyclic heteroaryl containing 1, 2, or 3 heteroatoms independently selected from oxygen, nitrogen, and sulfur); wherein the heteroaryl is substituted by n occurrences of R⁴. In certain embodiments, R² is a 5-6 membered monocyclic heteroaryl containing 1, 2, or 3 heteroatoms independently selected from oxygen, nitrogen, and sulfur; wherein the heteroaryl is substituted by n occurrences of R⁴.

In certain embodiments, R² is —(C₀₋₄ alkylene)-(5-membered monocyclic heteroaryl containing 1, 2, or 3 heteroatoms independently selected from oxygen, nitrogen, and sulfur); wherein the heteroaryl is substituted by n occurrences of R⁴. In certain embodiments, R² is —(C₁₋₄ alkylene)-(5-membered monocyclic heteroaryl containing 1, 2, or 3 heteroatoms independently selected from oxygen, nitrogen, and sulfur); wherein the heteroaryl is substituted by n occurrences of R⁴. In certain embodiments, R² is a 5-membered monocyclic heteroaryl containing 1, 2, or 3 heteroatoms independently selected from oxygen, nitrogen, and sulfur; wherein the heteroaryl is substituted by n occurrences of R⁴.

In certain embodiments, R² is —(C₀₋₄ alkylene)-(6-membered monocyclic heteroaryl containing 1 or 2 heteroatoms selected from nitrogen); wherein the heteroaryl is substituted by n occurrences of R⁴. In certain embodiments, R² is —(C₁₋₄ alkylene)-(6-membered monocyclic heteroaryl containing 1 or 2 heteroatoms selected from nitrogen); wherein the heteroaryl is substituted by n occurrences of R⁴. In certain embodiments, R² is a 6-membered monocyclic heteroaryl containing 1 or 2 heteroatoms selected from nitrogen; wherein the heteroaryl is substituted by n occurrences of R⁴.

In certain embodiments, R² is —(C₀₋₄ alkylene)-(8-10 membered bicyclic heteroaryl containing 1, 2, 3, or 4 heteroatoms independently selected from oxygen, nitrogen, and sulfur); wherein the heteroaryl is substituted by n occurrences of R⁴. In certain embodiments, R² is —(C₁₋₄ alkylene)-(8-10 membered bicyclic heteroaryl containing 1, 2, 3, or 4 heteroatoms independently selected from oxygen, nitrogen, and sulfur); wherein the heteroaryl is substituted by n occurrences of R⁴. In certain embodiments, R² is an 8-10 membered bicyclic heteroaryl containing 1, 2, 3, or 4 heteroatoms independently selected from oxygen, nitrogen, and sulfur; wherein the heteroaryl is substituted by n occurrences of R⁴.

In certain embodiments, R² is —(C₀₋₄ alkylene)-(3-10 membered monocyclic or bicyclic, saturated or partially unsaturated heterocyclyl containing 1, 2, or 3 heteroatoms independently selected from oxygen, nitrogen, and sulfur); wherein the heterocyclyl is substituted by n occurrences of R⁴.

In certain embodiments, R² is —(C₀₋₄ alkylene)-(3-7 membered monocyclic, saturated or partially unsaturated heterocyclyl containing 1, 2, or 3 heteroatoms independently selected from oxygen, nitrogen, and sulfur); wherein the heterocyclyl is substituted by n occurrences of R⁴. In certain embodiments, R² is —(C₀₋₄ alkylene)-(5-6 membered monocyclic, saturated heterocyclyl containing 1 or 2 heteroatoms independently selected from oxygen and nitrogen); wherein the heterocyclyl is substituted by n occurrences of R⁴. In certain embodiments, R² is —(C₁₋₄ alkylene)-(5-6 membered monocyclic, saturated heterocyclyl containing 1 or 2 heteroatoms independently selected from oxygen and nitrogen); wherein the heterocyclyl is substituted by n occurrences of R⁴. In certain embodiments, R² is a 5-6 membered monocyclic, saturated heterocyclyl containing 1 or 2 heteroatoms independently selected from oxygen and nitrogen; wherein the heterocyclyl is substituted by n occurrences of R⁴.

In certain embodiments, R² is —(C₀₋₄ alkylene)-(7-10 membered bicyclic, saturated or partially unsaturated heterocyclyl containing 1, 2, or 3 heteroatoms independently selected from oxygen, nitrogen, and sulfur); wherein the heterocyclyl is substituted by n occurrences of R⁴. In certain embodiments, R² is —(C₁₋₄ alkylene)-(7-10 membered bicyclic, saturated or partially unsaturated heterocyclyl containing 1, 2, or 3 heteroatoms independently selected from oxygen, nitrogen, and sulfur); wherein the heterocyclyl is substituted by n occurrences of R⁴. In certain embodiments, R² is a 7-10 membered bicyclic, saturated or partially unsaturated heterocyclyl containing 1, 2, or 3 heteroatoms independently selected from oxygen, nitrogen, and sulfur; wherein the heterocyclyl is substituted by n occurrences of R⁴.

In certain embodiments, R² is selected from the groups depicted in the compounds in Table 4 below.

In certain embodiments, R¹ and R² are taken together with the nitrogen atom to which they are attached to form a 3-7 membered heterocyclic ring containing the nitrogen atom and 0 or 1 additional heteroatom selected from nitrogen, oxygen, and sulfur, wherein the 3-7 membered heterocyclic ring is substituted with 0, 1, 2, or 3 occurrences of R⁴. In certain embodiments, R¹ and R² are taken together with the nitrogen atom to which they are attached to form a 3-7 membered heterocyclic ring containing the nitrogen atom and 0 or 1 additional heteroatom selected from nitrogen, oxygen, and sulfur, wherein the additional nitrogen atom is optionally substituted with C₁₋₄ alkyl. In certain embodiments, R¹ and R² are taken together with the nitrogen atom to which they are attached to form a 3-7 membered heterocyclic ring containing the nitrogen atom and 0 or 1 additional nitrogen atom; wherein the additional nitrogen atom is optionally substituted with C₁₋₄ alkyl. In certain embodiments, R¹ and R² are taken together with the nitrogen atom to which they are attached to form a 3-7 membered heterocyclic ring containing the nitrogen atom and no additional heteroatoms; wherein the 3-7 membered heterocyclic ring is substituted with 0, 1, 2, or 3 occurrences of R⁴.

In certain embodiments, R¹ and R² are taken together with the nitrogen atom to which they are attached to form a 3-7 membered heterocyclic ring containing the nitrogen atom and 0 or 1 additional heteroatom selected from nitrogen, oxygen, and sulfur. In certain embodiments, R¹ and R² are taken together with the nitrogen atom to which they are attached to form a 3-7 membered heterocyclic ring containing the nitrogen atom and 0 or 1 additional nitrogen atom. In certain embodiments, R¹ and R² are taken together with the nitrogen atom to which they are attached to form a 3-7 membered heterocyclic ring containing the nitrogen atom and no additional heteroatoms. In certain embodiments, R¹ and R² are selected from the groups depicted in the compounds in Table 4 below.

As defined generally above, R³ is C₁₋₄ fluoroalkyl. In certain embodiments, R³ is C₁₋₂ fluoroalkyl. In certain embodiments, R³ is C₁ fluoroalkyl. In certain embodiments, the fluoroalkyl contains at least three fluorine atoms. In certain embodiments, R³ is C₁₋₂ trifluoroalkyl. In certain embodiments, R³ is trifluoromethyl. In certain embodiments, R³ is selected from the groups depicted in the compounds in Table 4 below.

As defined generally above, R⁴ represents independently for each occurrence C₁₋₄ alkyl, C₁₋₄haloalkyl, halo, —(C₀₋₄ alkylene)-OR⁵, or —(C₀₋₄ alkylene)-N(R⁵)(R⁶). In certain embodiments, R⁴ represents independently for each occurrence C₁₋₄ alkyl, C₁₋₄haloalkyl, or halo. In certain embodiments, R⁴ represents independently for each occurrence —(C₀₋₄ alkylene)-OR⁵ or —(C₀₋₄ alkylene)-N(R⁵)(R⁶). In certain embodiments, R⁴ represents independently for each occurrence methyl, fluoro, —OH, —CH₂—OH, —NH₂.

In certain embodiments, R⁴ represents independently for each occurrence C₁₋₄ alkyl. In certain embodiments, R⁴ represents independently for each occurrence C₁₋₄ haloalkyl. In certain embodiments, R⁴ represents independently for each occurrence halo. In certain embodiments, R⁴ represents independently for each occurrence —(C₀₋₄ alkylene)-OR⁵. In certain embodiments, R⁴ represents independently for each occurrence —(C₀₋₄ alkylene)-N(R⁵)(R⁶). In certain embodiments, R⁴ is selected from the groups depicted in the compounds in Table 4 below.

As defined generally above, R⁵ and R⁶ each represent independently for each occurrence hydrogen, C₁₋₄ alkyl, or C₁₋₄ haloalkyl. In certain embodiments, R⁵ and R⁶ are hydrogen. In certain embodiments, R⁵ and R⁶ each represent independently for each occurrence C₁₋₄ alkyl. In certain embodiments, R⁵ and R⁶ each represent independently for each occurrence C₁₋₄ haloalkyl. In certain embodiments, R⁵ and R⁶ are selected from the groups depicted in the compounds in Table 4 below.

As defined generally above, X is C₁₋₃ alkylene or C₁₋₃ deuteroalkylene. In certain embodiments, X is —CH₂CH₂— or —CH₂CH(CH₃)—. In certain embodiments, X is —CH₂CH₂—. In certain embodiments, X is —CH₂CH₂CH₂—. In certain embodiments, X is —CH₂CH(CH₃)—. In certain embodiments, X is —CH₂—. In certain embodiments, X is C₁₋₃ deuteroalkylene.

In certain embodiments, X is —CZ₂CZ₂—, wherein each Z is hydrogen or deuterium, provided that the abundance of deuterium in Z is at least 75%. In certain embodiments, the abundance of deuterium in Z is at least 90%. In certain embodiments, the abundance of deuterium in Z is at least 95%. In certain embodiments, X is —CD₂CH₂—. In certain embodiments, X is —CH₂CD₂—. In certain embodiments, X is —CD₂CD₂-.

In certain embodiments, X is selected from the groups depicted in the compounds in Table 4 below.

As defined generally above, n is 0, 1, 2, 3, or 4. In certain embodiments, n is 0, 1, or 2. In certain embodiments, n is 0. In certain embodiments, n is 1. In certain embodiments, n is 2. In certain embodiments, n is 3. In certain embodiments, n is 4. In certain embodiments, n is selected from the values represented in the compounds in Table 4 below.

The description above describes multiple embodiments relating to compounds of Formula IV. The patent application specifically contemplates all combinations of the embodiments.

Another aspect of the invention provides a compound represented by Formula V:

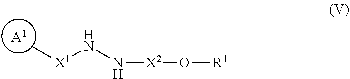

or a pharmaceutically acceptable salt thereof, wherein:
A¹ is phenyl, pyridinyl, thiazolyl, dihydroisoquinolinyl, or quinazolinonyl; each of which is substituted with one occurrence of R²;
R¹ is C₁₋₄ fluoroalkyl;
R² is —C(O)N(R³)(R⁴); —CO₂R³; C₁₋₄ alkyl; C₁₋₄haloalkyl; —C(O)—(C₁₋₄ alkyl); a 5- or 6-membered monocyclic heteroaryl containing 1, 2, 3, or 4 heteroatoms independently selected from oxygen, nitrogen, and sulfur; or a 3- to 7-membered monocyclic saturated heterocyclyl containing 1 or 2 heteroatoms independently selected from oxygen, nitrogen, and sulfur; wherein R² is optionally substituted with one occurrence of —N(R⁶)(R⁷), unless R² is —C(O)N(R³)(R⁴);
R³ is hydrogen or C₁₋₄ alkyl;
R⁴ is hydrogen, C₁₋₄ alkyl, C₁₋₄ alkenyl, C₁₋₄ alkynyl, C₁₋₄ haloalkyl, —(C₀₋₄ alkylene)-(C₃₋₇ cycloalkyl), or —(C₁₋₄ alkylene)-OR⁶ optionally substituted with one occurrence of —C(O)N(R⁶)(R⁷); or R³ and R⁴ are taken together with the nitrogen atom to which they are attached to form a 3-7 membered heterocyclic ring containing the nitrogen atom and 0 or 1 additional heteroatom selected from nitrogen, oxygen, and sulfur, wherein the 3-7 membered heterocyclic ring is substituted with 0, 1, 2, or 3 occurrences of R⁶;
R⁶ and R⁷ each represent independently for each occurrence hydrogen or C₁₋₄ alkyl;
R⁸ represents independently for each occurrence C₁₋₄ alkyl, C₃₋₆ cycloalkyl, or halo; and
X¹ and X² each represent independently C₁₋₃ alkylene.

The definitions of variables in Formula V above encompass multiple chemical groups. The application contemplates embodiments where, for example, i) the definition of a variable is a single chemical group selected from those chemical groups set forth above, ii) the definition of a variable is a collection of two or more of the chemical groups selected from those set forth above, and iii) the compound is defined by a combination of variables in which the variables are defined by (i) or (ii).

In certain embodiments, the compound is a compound of Formula V.

As defined generally above, A¹ is phenyl, pyridinyl, thiazolyl, dihydroisoquinolinyl, or quinazolinonyl; each of which is substituted with one occurrence of R². In certain embodiments, A¹ is phenyl or pyridinyl, each of which is substituted with one occurrence of R². In certain embodiments, A¹ is pyridinyl, thiazolyl, dihydroisoquinolinyl, or quinazolinonyl; each of which is substituted with one occurrence of R².

In certain embodiments, A¹ is phenyl substituted with one occurrence of R². In certain embodiments, A¹ is pyridinyl substituted with one occurrence of R². In certain embodiments, A¹ is thiazolyl substituted with one occurrence of R². In certain embodiments, A¹ is dihydroisoquinolinyl substituted with one occurrence of R². In certain embodiments, A¹ is quinazolinonyl substituted with one occurrence of R².

In certain embodiments, A¹ is

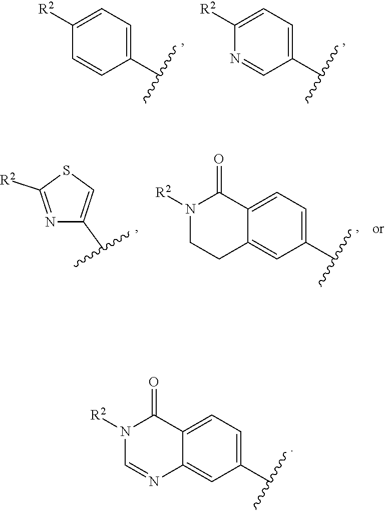

In certain embodiments, A¹ is

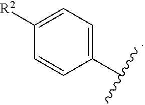

In certain embodiments, A¹ is

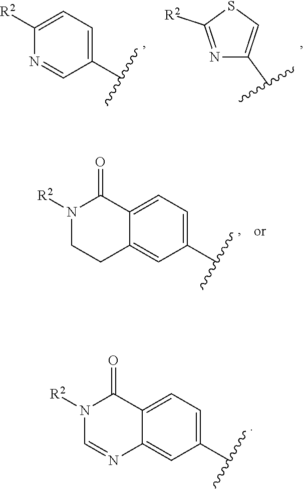

In certain embodiments, A¹ is

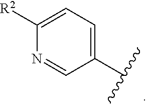

In certain embodiments, A¹ is

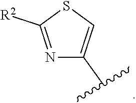

In certain embodiments, A¹ is

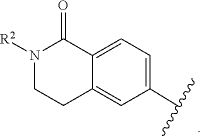

In certain embodiments, A¹ is

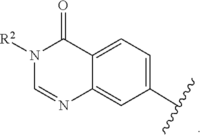

In certain embodiments, A¹ is

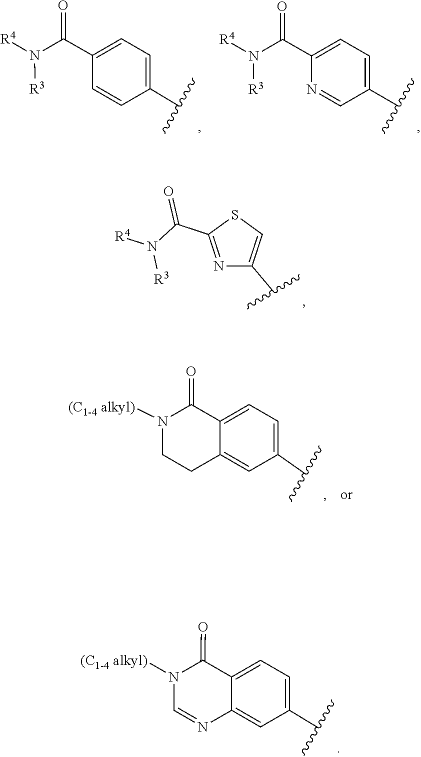

In certain embodiments, A¹ is

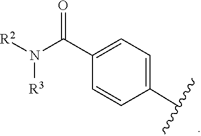

In certain embodiments, A¹ is

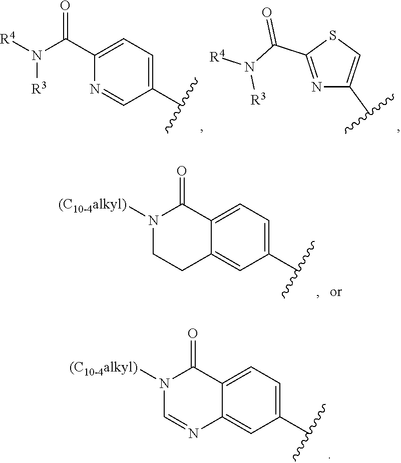

In certain embodiments, A¹ is

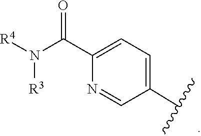

In certain embodiments, A¹ is

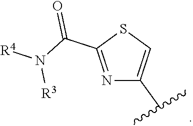

In certain embodiments, A¹ is

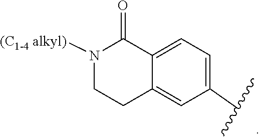

In certain embodiments, A¹ is

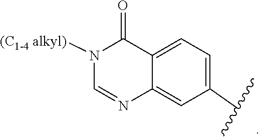

In certain embodiments, R¹ is selected from the groups depicted in the compounds in Table 5 below.

As defined generally above, R¹ is C₁₋₄ fluoroalkyl. In certain embodiments, R¹ is C₁₋₂ fluoroalkyl. In certain embodiments, R¹ is C₁ fluoroalkyl. In certain embodiments, the fluoroalkyl contains at least three fluorine atoms. In certain embodiments, R¹ is C₁₋₂ trifluoroalkyl. In certain embodiments, R¹ is trifluoromethyl. In certain embodiments, R¹ is selected from the groups depicted in the compounds in Table 5 below.

As defined generally above, R² is —C(O)N(R³)(R⁴); —CO₂R³; C₁₋₄ alkyl, C₁₋₄ haloalkyl; —C(O)—(C₁₋₄ alkyl); a 5- or 6-membered monocyclic heteroaryl containing 1, 2, 3, or 4 heteroatoms independently selected from oxygen, nitrogen, and sulfur; or a 3- to 7-membered monocyclic saturated heterocyclyl containing 1 or 2 heteroatoms independently selected from oxygen, nitrogen, and sulfur; wherein R² is optionally substituted with one occurrence of —N(R⁶)(R⁷), unless R² is —C(O)N(R³)(R⁴). In certain embodiments, R² is —C(O)N(R³)(R⁴).

In certain embodiments, R² is —CO₂R³, C₁₋₄ alkyl, C₁₋₄haloalkyl, —C(O)—(C₁₋₄ alkyl), or a 3- to 7-membered monocyclic saturated heterocyclyl containing 1 or 2 heteroatoms independently selected from oxygen, nitrogen, and sulfur; wherein R² is optionally substituted with one occurrence of —N(R⁶)(R⁷). In certain embodiments, R² is —CO₂R³, C₁₋₄ haloalkyl, —C(O)—(C₁₋₄ alkyl), or a 3- to 7-membered monocyclic saturated heterocyclyl containing 1 or 2 heteroatoms independently selected from oxygen, nitrogen, and sulfur; wherein R² is optionally substituted with one occurrence of —N(R⁶)(R⁷). In certain embodiments, R² is C₁₋₄ alkyl or C₁₋₄ haloalkyl, each of which is optionally substituted with one occurrence of —N(R⁶)(R⁷). In certain embodiments, R² is C₁₋₄ alkyl optionally substituted with one occurrence of —N(R⁶)(R⁷). In certain embodiments, R² is C₁₋₄ haloalkyl optionally substituted with one occurrence of —N(R⁶)(R⁷). In certain embodiments, R² is —CO₂R³ optionally substituted with one occurrence of —N(R⁶)(R⁷). In certain embodiments, R² is —C(O)—(C₁₋₄ alkyl) optionally substituted with one occurrence of —N(R⁶)(R⁷). In certain embodiments, R² is a 3- to 7-membered monocyclic saturated heterocyclyl containing 1 or 2 heteroatoms independently selected from oxygen, nitrogen, and sulfur; wherein R² is optionally substituted with one occurrence of —N(R⁶)(R⁷).

In certain embodiments, R² is —CO₂R³, C₁₋₄ alkyl, C₁₋₄haloalkyl, —C(O)—(C₁₋₄ alkyl), or a 3- to 7-membered monocyclic saturated heterocyclyl containing 1 or 2 heteroatoms independently selected from oxygen, nitrogen, and sulfur; wherein R² is substituted with one occurrence of —N(R⁶)(R⁷). In certain embodiments, R² is —CO₂R³, C₁₋₄haloalkyl, —C(O)—(C₁₋₄ alkyl), or a 3- to 7-membered monocyclic saturated heterocyclyl containing 1 or 2 heteroatoms independently selected from oxygen, nitrogen, and sulfur; wherein R² is substituted with one occurrence of —N(R⁶)(R⁷). In certain embodiments, R² is C₁₋₄ alkyl or C₁₋₄ haloalkyl, each of which is substituted with one occurrence of —N(R⁶)(R⁷). In certain embodiments, R² is C₁₋₄ alkyl substituted with one occurrence of —N(R⁶)(R⁷). In certain embodiments, R² is C₁₋₄haloalkyl substituted with one occurrence of —N(R⁶)(R⁷). In certain embodiments, R² is —CO₂R³ substituted with one occurrence of —N(R⁶)(R⁷). In certain embodiments, R² is —C(O)—(C₁₋₄ alkyl) substituted with one occurrence of —N(R⁶)(R⁷). In certain embodiments, R² is a 3- to 7-membered monocyclic saturated heterocyclyl containing 1 or 2 heteroatoms independently selected from oxygen, nitrogen, and sulfur; wherein R² is substituted with one occurrence of —N(R⁶)(R⁷).

In certain embodiments, R² is —CO₂R³, C₁₋₄ alkyl, C₁₋₄haloalkyl, —C(O)—(C₁₋₄ alkyl), or a 3- to 7-membered monocyclic saturated heterocyclyl containing 1 or 2 heteroatoms independently selected from oxygen, nitrogen, and sulfur. In certain embodiments, R² is —CO₂R³, C₁₋₄ haloalkyl, —C(O)—(C₁₋₄ alkyl), or a 3- to 7-membered monocyclic saturated heterocyclyl containing 1 or 2 heteroatoms independently selected from oxygen, nitrogen, and sulfur. In certain embodiments, R² is C₁₋₄ alkyl or C₁₋₄haloalkyl. In certain embodiments, R² is C₁₋₄ alkyl. In certain embodiments, R² is C₁₋₄haloalkyl. In certain embodiments, R² is —CO₂R³. In certain embodiments, R² is —C(O)—(C₁₋₄ alkyl). In certain embodiments, R² is a 3- to 7-membered monocyclic saturated heterocyclyl containing 1 or 2 heteroatoms independently selected from oxygen, nitrogen, and sulfur.

In certain embodiments, R² is a 5- or 6-membered monocyclic heteroaryl containing 1, 2, 3, or 4 heteroatoms independently selected from oxygen, nitrogen, and sulfur; wherein R² is optionally substituted with one occurrence of —N(R⁶)(R⁷). In certain embodiments, R² is a 5-membered monocyclic heteroaryl containing 1, 2, or 3 heteroatoms independently selected from oxygen, nitrogen, and sulfur; wherein R² is optionally substituted with one occurrence of —N(R⁶)(R⁷). In certain embodiments, R² is a 6-membered monocyclic heteroaryl containing 1, 2, or 3 heteroatoms which are nitrogen; wherein R² is optionally substituted with one occurrence of —N(R⁶)(R⁷).

In certain embodiments, R² is a 5- or 6-membered monocyclic heteroaryl containing 1, 2, 3, or 4 heteroatoms independently selected from oxygen, nitrogen, and sulfur. In certain embodiments, R² is a 5-membered monocyclic heteroaryl containing 1, 2, or 3 heteroatoms independently selected from oxygen, nitrogen, and sulfur. In certain embodiments, R² is imidazolyl, oxazolyl, or thiazolyl. In certain embodiments, R² is imidazol-2-yl, oxazol-2-yl, or thiazol-2-yl. In certain embodiments, R² is a 6-membered monocyclic heteroaryl containing 1, 2, or 3 heteroatoms which are nitrogen. In certain embodiments, R² is selected from the groups depicted in the compounds in Table 5 below.

As defined generally above, R³ is hydrogen or C₁₋₄ alkyl; or R³ and R⁴ are taken together with the nitrogen atom to which they are attached to form a 3-7 membered heterocyclic ring containing the nitrogen atom and 0 or 1 additional heteroatom selected from nitrogen, oxygen, and sulfur, wherein the 3-7 membered heterocyclic ring is substituted with 0, 1, 2, or 3 occurrences of R⁸.

In certain embodiments, R³ is hydrogen or C₁₋₄ alkyl. In certain embodiments, R³ is hydrogen or methyl. In certain embodiments, R³ is hydrogen. In certain embodiments, R³ is C₁₋₄ alkyl. In certain embodiments, R³ is methyl. In certain embodiments, R³ is selected from the groups depicted in the compounds in Table 5 below.

As defined generally above, R⁴ is hydrogen, C₁₋₄ alkyl, C₁₋₄ alkenyl, C₁₋₄ alkynyl, C₁₋₄ haloalkyl, or —(C₁₋₄ alkylene)-OR⁶ optionally substituted with one occurrence of —C(O)N(R⁶)(R⁷); or R³ and R⁴ are taken together with the nitrogen atom to which they are attached to form a 3-7 membered heterocyclic ring containing the nitrogen atom and 0 or 1 additional heteroatom selected from nitrogen, oxygen, and sulfur, wherein the 3-7 membered heterocyclic ring is substituted with 0, 1, 2, or 3 occurrences of R⁸.

In certain embodiments, R⁴ is hydrogen, C₁₋₄ alkyl, C₁₋₄ alkenyl, C₁₋₄ alkynyl, C₁₋₄ haloalkyl, or —(C₁₋₄ alkylene)-OR⁶ optionally substituted with one occurrence of —C(O)N(R⁶)(R⁷). In certain embodiments, R⁴ is hydrogen, C₁₋₄ alkyl, C₁₋₄ alkenyl, C₁₋₄ alkynyl, or C₁₋₄haloalkyl. In certain embodiments, R⁴ is hydrogen or C₁₋₄ alkyl. In certain embodiments, R⁴ is hydrogen or methyl. In certain embodiments, R⁴ is C₁₋₄ alkyl, C₁₋₄alkenyl, C₁₋₄ alkynyl, or C₁₋₄ haloalkyl. In certain embodiments, R⁴ is hydrogen. In certain embodiments, R⁴ is C₁₋₄ alkyl. In certain embodiments, R⁴ is methyl. In certain embodiments, R⁴ is C₁₋₄ alkenyl. In certain embodiments, R⁴ is C₁₋₄ alkynyl. In certain embodiments, R⁴ is C₁₋₄ haloalkyl.

In certain embodiments, R⁴ is —(C₁₋₄ alkylene)-OR⁶ optionally substituted with one occurrence of —C(O)N(R⁶)(R⁷). In certain embodiments, R⁴ is —(C₁₋₄ alkylene)-OR⁶. In certain embodiments, R⁴ is —(C₁₋₄ alkylene)-OR⁶ substituted with one occurrence of —C(O)N(R⁶)(R⁷). In certain embodiments, R⁴ is —(C₁₋₄ alkylene)-OH. In certain embodiments, R⁴ is —(C₁₋₄ alkylene)-OH substituted with one occurrence of —C(O)NH₂. In certain embodiments, R⁴ is selected from the groups depicted in the compounds in Table 5 below.

In certain embodiments, R³ and R⁴ are taken together with the nitrogen atom to which they are attached to form a 3-7 membered heterocyclic ring containing the nitrogen atom and 0 or 1 additional heteroatom selected from nitrogen, oxygen, and sulfur, wherein the 3-7 membered heterocyclic ring is substituted with 0, 1, 2, or 3 occurrences of R⁸. In certain embodiments, R³ and R⁴ are taken together with the nitrogen atom to which they are attached to form a 3-7 membered heterocyclic ring containing the nitrogen atom and 0 or 1 additional heteroatom selected from nitrogen, oxygen, and sulfur, wherein the additional nitrogen atom is optionally substituted with C₁₋₄ alkyl. In certain embodiments, R³ and R⁴ are taken together with the nitrogen atom to which they are attached to form a 3-7 membered heterocyclic ring containing the nitrogen atom and 0 or 1 additional nitrogen atom; wherein the additional nitrogen atom is optionally substituted with C₁₋₄ alkyl. In certain embodiments, R³ and R⁴ are taken together with the nitrogen atom to which they are attached to form a 3-7 membered heterocyclic ring containing the nitrogen atom and no additional heteroatoms; wherein the 3-7 membered heterocyclic ring is substituted with 0, 1, 2, or 3 occurrences of R⁸.

In certain embodiments, R³ and R⁴ are taken together with the nitrogen atom to which they are attached to form a 3-7 membered heterocyclic ring containing the nitrogen atom and 0 or 1 additional heteroatom selected from nitrogen, oxygen, and sulfur. In certain embodiments, R³ and R⁴ are taken together with the nitrogen atom to which they are attached to form a 3-7 membered heterocyclic ring containing the nitrogen atom and 0 or 1 additional nitrogen atom. In certain embodiments, R³ and R⁴ are taken together with the nitrogen atom to which they are attached to form a 3-7 membered heterocyclic ring containing the nitrogen atom and no additional heteroatoms. In certain embodiments, R³ and R⁴ are selected from the groups depicted in the compounds in Table 5 below.

As defined generally above, R⁶ and R⁷ each represent independently for each occurrence hydrogen or C₁₋₄ alkyl. In certain embodiments, R⁶ and R⁷ are hydrogen. In certain embodiments, R⁶ and R⁷ each represent independently for each occurrence C₁₋₄ alkyl. In certain embodiments, R⁶ and R⁷ are methyl. In certain embodiments, R⁶ is hydrogen. In certain embodiments, R⁶ is C₁₋₄ alkyl. In certain embodiments, R⁶ is methyl. In certain embodiments, R⁷ is hydrogen. In certain embodiments, R⁷ is C₁₋₄ alkyl. In certain embodiments, R⁷ is methyl. In certain embodiments, R⁶ and R⁷ are selected from the groups depicted in the compounds in Table 5 below.

As defined generally above, R⁸ represents independently for each occurrence C₁₋₄ alkyl, C₃₋₆ cycloalkyl, or halo. In certain embodiments, R⁸ represents independently for each occurrence C₁₋₄ alkyl or C₃₋₆ cycloalkyl. In certain embodiments, R⁸ represents independently for each occurrence C₁₋₄ alkyl or halo. In certain embodiments, R⁸ represents independently for each occurrence C₃₋₆ cycloalkyl or halo.

In certain embodiments, R⁸ represents independently for each occurrence C₁₋₄ alkyl. In certain embodiments, R⁸ represents independently for each occurrence C₃₋₆ cycloalkyl. In certain embodiments, R⁸ represents independently for each occurrence halo. In certain embodiments, R⁸ is selected from the groups depicted in the compounds in Table 5 below.

As defined generally above, X¹ and X² each represent independently C₁₋₃ alkylene C₁₋₃ deuteroalkylene. In certain embodiments, X¹ is C₁₋₃ alkylene. In certain embodiments, X¹ is —CH₂CH₂— or —CH₂CH(CH₃)—. In certain embodiments, X¹ is —CH₂CH₂—. In certain embodiments, X¹ is —CH₂CH₂CH₂—. In certain embodiments, X¹ is —CH₂CH(CH₃)—. In certain embodiments, X¹ is —CH₂—. In certain embodiments, X¹ is C₁₋₃ deuteroalkylene.

In certain embodiments, X¹ is —CZ₂CZ₂—, wherein each Z is hydrogen or deuterium, provided that the abundance of deuterium in Z is at least 75%. In certain embodiments, the abundance of deuterium in Z is at least 90%. In certain embodiments, the abundance of deuterium in Z is at least 95%. In certain embodiments, X is —CD₂CH₂—. In certain embodiments, X¹ is —CH₂CD₂—. In certain embodiments, X¹ is —CD₂CD₂-.

In certain embodiments, X¹ is selected from the groups depicted in the compounds in Table 5 below.

In certain embodiments, X² is C₁₋₃ alkylene. In certain embodiments, X² is —CH₂CH₂-or —CH₂CH(CH₃)—. In certain embodiments, X² is —CH₂CH₂—. In certain embodiments, X² is —CH₂CH₂CH₂—. In certain embodiments, X² is —CH₂CH(CH₃)—. In certain embodiments, X² is —CH₂—.

In certain embodiments, X² is C₁₋₃ deuteroalkylene. In certain embodiments, X² is —CZ₂CZ₂—, wherein each Z is hydrogen or deuterium, provided that the abundance of deuterium in Z is at least 75%. In certain embodiments, the abundance of deuterium in Z is at least 90%. In certain embodiments, the abundance of deuterium in Z is at least 95%. In certain embodiments, X is —CD₂CH₂—. In certain embodiments, X² is —CH₂CD₂—. In certain embodiments, X² is —CD₂CD₂-.

In certain embodiments, X² is selected from the groups depicted in the compounds in Table 5 below.

The description above describes multiple embodiments relating to compounds of Formula V. The patent application specifically contemplates all combinations of the embodiments.

### Exemplary Specific Compounds

In certain embodiments, the compound is a compound in Table 1 or a pharmaceutically acceptable salt thereof. In certain embodiments, the compound is a compound in Table 1. In certain embodiments, the compound is one of compound I-1, I-3 to I-12, or I-24 to I-40 in Table 1, or a pharmaceutically acceptable salt thereof. In certain embodiments, the compound is one of compound I-1, I-3 to I-12, or I-24 to I-40 in Table 1. In certain embodiments, the compound is a compound in Table 1 or a pharmaceutically acceptable salt thereof, wherein the compound has an entry in bioactivity Table 6. In certain embodiments, the compound is a compound in Table 1, wherein the compound has an entry in bioactivity Table 6. In certain embodiments, the compound is compound I-1 or I-2 in Table 1, or a pharmaceutically acceptable salt thereof. In certain embodiments, the compound is compound I-1 or I-2 in Table 1.

TABLE 1
Exemplary Compounds

| Compound | |
|---|---|
| No. | Chemical Structure |
| I-1 | 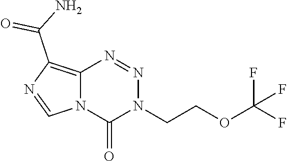 [SMILES: NC(=O)c1ncn2c(=O)n(CCOC(F)(F)F)nnc12] |
| I-2 | 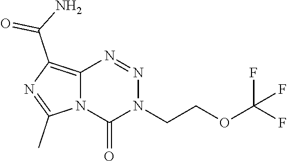 [SMILES: Cc1nc(C(N)=O)c2nnn(CCOC(F)(F)F)c(=O)n12] |
| I-3 | 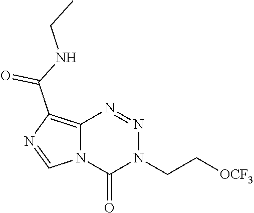 [SMILES: CCNC(=O)c1ncn2c(=O)n(CCOC(F)(F)F)nnc12] |
| I-4 | 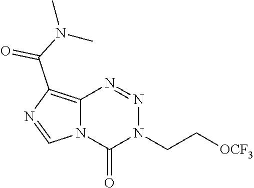 [SMILES: CN(C)C(=O)c1ncn2c(=O)n(CCOC(F)(F)F)nnc12] |
| I-5 | 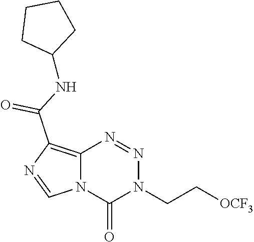 [SMILES: O=C(NC1CCCC1)c1ncn2c(=O)n(CCOC(F)(F)F)nnc12] |
| I-6 | 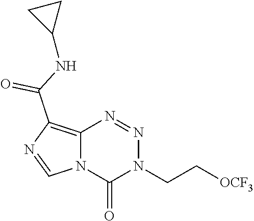 [SMILES: O=C(NC1CC1)c1ncn2c(=O)n(CCOC(F)(F)F)nnc12] |
| I-7 | 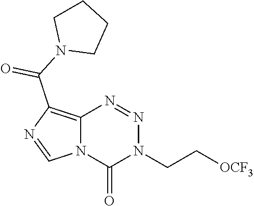 [SMILES: O=C(c1ncn2c(=O)n(CCOC(F)(F)F)nnc12)N1CCCC1] |
| I-8 | 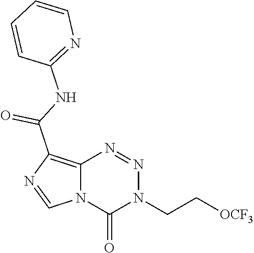 [SMILES: O=C(Nc1ccccn1)c1ncn2c(=O)n(CCOC(F)(F)F)nnc12] |
| I-9 | 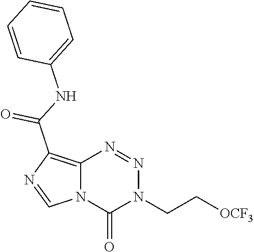 [SMILES: O=C(Nc1ccccc1)c1ncn2c(=O)n(CCOC(F)(F)F)nnc12] |
| I-10 | 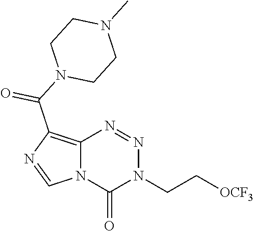 [SMILES: CN1CCN(C(=O)c2ncn3c(=O)n(CCOC(F)(F)F)nnc23)CC1] |
| I-11 | 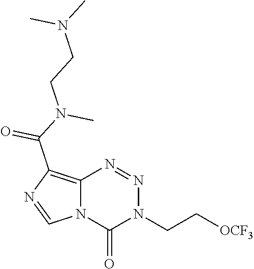 [SMILES: CN(C)CCN(C)C(=O)c1ncn2c(=O)n(CCOC(F)(F)F)nnc12] |
| I-12 | 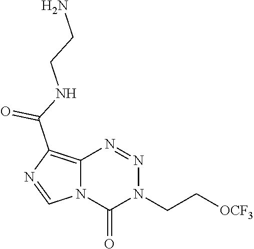 [SMILES: NCCNC(=O)c1ncn2c(=O)n(CCOC(F)(F)F)nnc12] |
| I-13 | 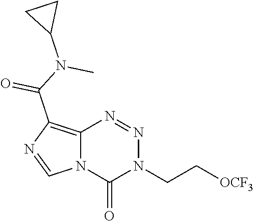 [SMILES: CN(C(=O)c1ncn2c(=O)n(CCOC(F)(F)F)nnc12)C1CC1] |
| I-14 | 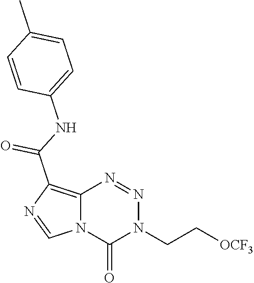 [SMILES: Cc1ccc(NC(=O)c2ncn3c(=O)n(CCOC(F)(F)F)nnc23)cc1] |
| I-15 | 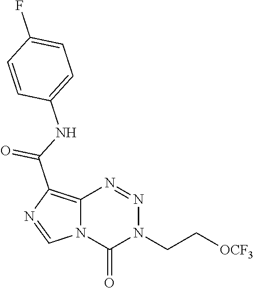 [SMILES: O=C(Nc1ccc(F)cc1)c1ncn2c(=O)n(CCOC(F)(F)F)nnc12] |
| I-16 | 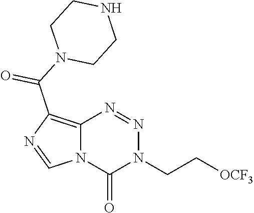 [SMILES: O=C(c1ncn2c(=O)n(CCOC(F)(F)F)nnc12)N1CCNCC1] |
| I-17 | 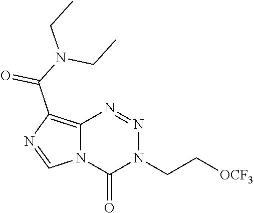 [SMILES: CCN(CC)C(=O)c1ncn2c(=O)n(CCOC(F)(F)F)nnc12] |
| I-18 | 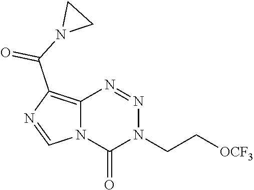 [SMILES: O=C(c1ncn2c(=O)n(CCOC(F)(F)F)nnc12)N1CC1] |
| I-19 | 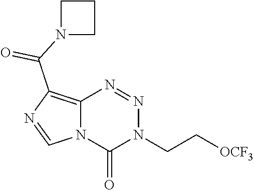 [SMILES: O=C(c1ncn2c(=O)n(CCOC(F)(F)F)nnc12)N1CCC1] |
| I-20 | 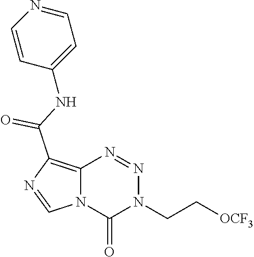 [SMILES: O=C(Nc1ccncc1)c1ncn2c(=O)n(CCOC(F)(F)F)nnc12] |
| I-21 | 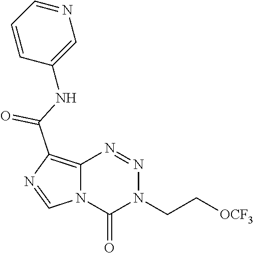 [SMILES: O=C(Nc1cccnc1)c1ncn2c(=O)n(CCOC(F)(F)F)nnc12] |
| I-22 | 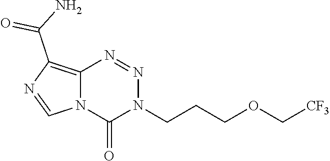 [SMILES: NC(=O)c1ncn2c(=O)n(CCCOCC(F)(F)F)nnc12] |
| I-23 | 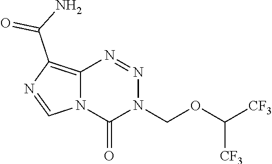 [SMILES: NC(=O)c1ncn2c(=O)n(COC(C(F)(F)F)C(F)(F)F)nnc12] |
| I-24 | 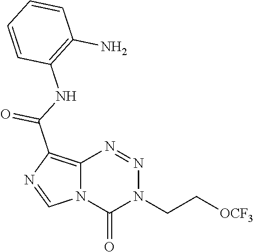 [SMILES: Nc1ccccc1NC(=O)c1ncn2c(=O)n(CCOC(F)(F)F)nnc12] |
| I-25 | 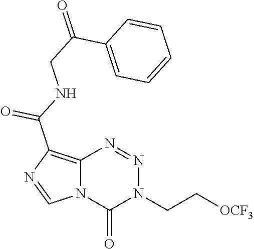 [SMILES: O=C(CNC(=O)c1ncn2c(=O)n(CCOC(F)(F)F)nnc12)c1ccccc1] |
| I-26 | 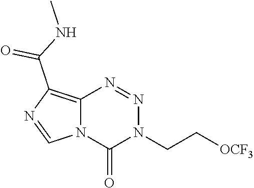 [SMILES: CNC(=O)c1ncn2c(=O)n(CCOC(F)(F)F)nnc12] |
| I-27 | 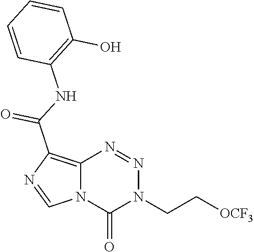 [SMILES: O=C(Nc1ccccc1O)c1ncn2c(=O)n(CCOC(F)(F)F)nnc12] |
| I-28 | 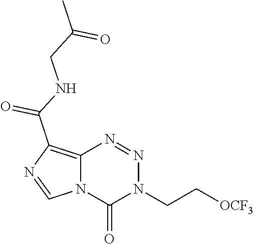 [SMILES: CC(=O)CNC(=O)c1ncn2c(=O)n(CCOC(F)(F)F)nnc12] |
| I-29 | 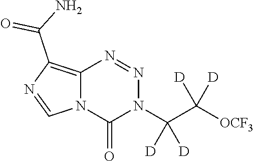 [SMILES: [2H]C([2H])(OC(F)(F)F)C([2H])([2H])n1nnc2c(C(N)=O)ncn2c1=O] |
| I-30 | 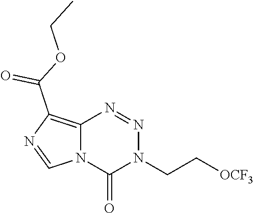 [SMILES: CCOC(=O)c1ncn2c(=O)n(CCOC(F)(F)F)nnc12] |
| I-31 | 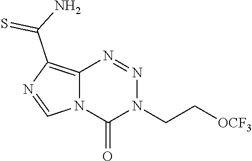 [SMILES: NC(=S)c1ncn2c(=O)n(CCOC(F)(F)F)nnc12] |
| I-32 | 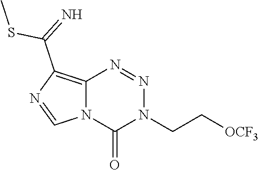 [SMILES: CSC(=N)c1ncn2c(=O)n(CCOC(F)(F)F)nnc12] |
| I-33 | 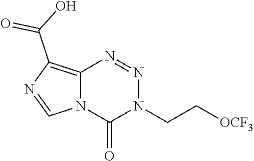 [SMILES: O=C(O)c1ncn2c(=O)n(CCOC(F)(F)F)nnc12] |
| I-34 | 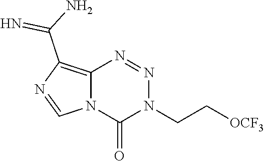 [SMILES: N=C(N)c1ncn2c(=O)n(CCOC(F)(F)F)nnc12] |
| I-35 | 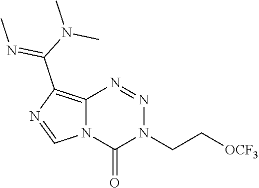 [SMILES: C/N=C(/c1ncn2c(=O)n(CCOC(F)(F)F)nnc12)N(C)C] |
| I-36 | 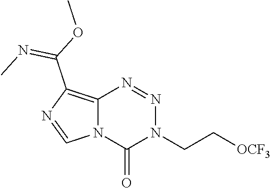 [SMILES: C/N=C(/OC)c1ncn2c(=O)n(CCOC(F)(F)F)nnc12] |
| I-37 | 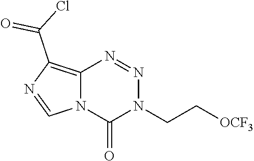 [SMILES: O=C(Cl)c1ncn2c(=O)n(CCOC(F)(F)F)nnc12] |
| I-38 | 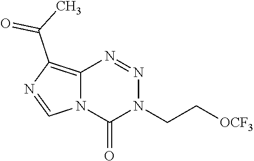 [SMILES: CC(=O)c1ncn2c(=O)n(CCOC(F)(F)F)nnc12] |
| I-39 | 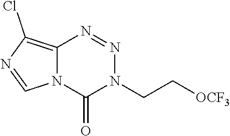 [SMILES: O=c1n(CCOC(F)(F)F)nnc2c(Cl)ncn12] |
| I-40 | 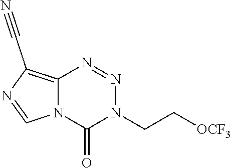 [SMILES: N#Cc1ncn2c(=O)n(CCOC(F)(F)F)nnc12] |
| I-41 | 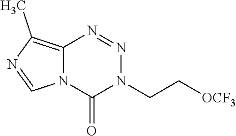 [SMILES: Cc1ncn2c(=O)n(CCOC(F)(F)F)nnc12] |
| I-42 | 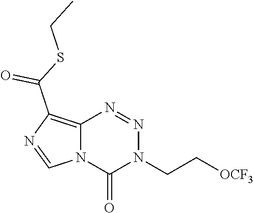 [SMILES: CCSC(=O)c1ncn2c(=O)n(CCOC(F)(F)F)nnc12] |
| I-43 | 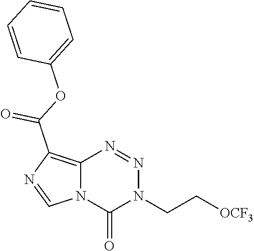 [SMILES: O=C(Oc1ccccc1)c1ncn2c(=O)n(CCOC(F)(F)F)nnc12] |
| I-44 | 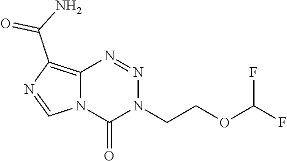 [SMILES: NC(=O)c1ncn2c(=O)n(CCOC(F)F)nnc12] |

In certain embodiments, the compound is a compound in Table 2 or a pharmaceutically acceptable salt thereof. In certain embodiments, the compound is a compound in Table 2. In certain embodiments, the compound is one of compound II-1 to II-6 in Table 2, or a pharmaceutically acceptable salt thereof. In certain embodiments, the compound is one of compound II-1 to II-6 in Table 2.

TABLE 2
Exemplary Compounds

| Com- | |
|---|---|
| pound | |
| No. | Chemical Structure |
| II-1 | 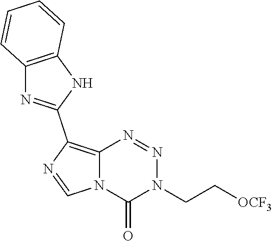 [SMILES: O=c1n(CCOC(F)(F)F)nnc2c(-c3nc4ccccc4[nH]3)ncn12] |
| II-2 | 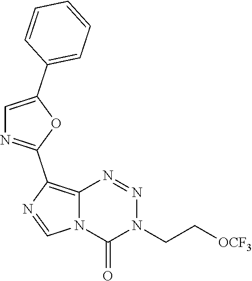 [SMILES: O=c1n(CCOC(F)(F)F)nnc2c(-c3ncc(-c4ccccc4)o3)ncn12] |
| II-3 | 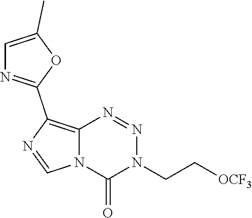 [SMILES: Cc1cnc(-c2ncn3c(=O)n(CCOC(F)(F)F)nnc23)o1] |
| II-4 | 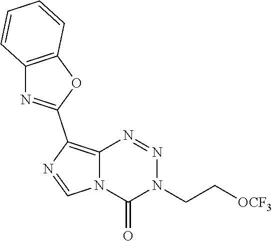 [SMILES: O=c1n(CCOC(F)(F)F)nnc2c(-c3nc4ccccc4o3)ncn12] |
| II-5 | 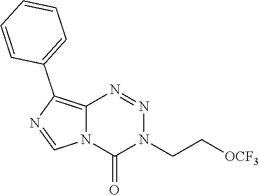 [SMILES: O=c1n(CCOC(F)(F)F)nnc2c(-c3ccccc3)ncn12] |
| II-6 | 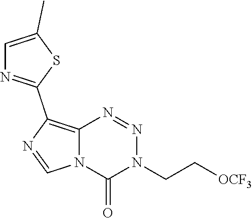 [SMILES: Cc1cnc(-c2ncn3c(=O)n(CCOC(F)(F)F)nnc23)s1] |
| II-7 | 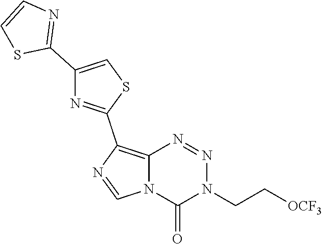 [SMILES: O=c1n(CCOC(F)(F)F)nnc2c(-c3nc(-c4nccs4)cs3)ncn12] |
| II-8 | 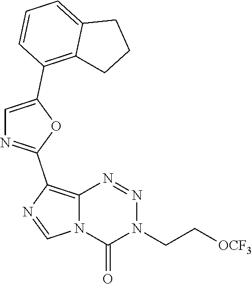 [SMILES: O=c1n(CCOC(F)(F)F)nnc2c(-c3ncc(-c4cccc5c4CCC5)o3)ncn12] |
| II-9 | 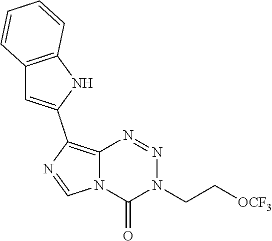 [SMILES: O=c1n(CCOC(F)(F)F)nnc2c(-c3cc4ccccc4[nH]3)ncn12] |
| II-10 | 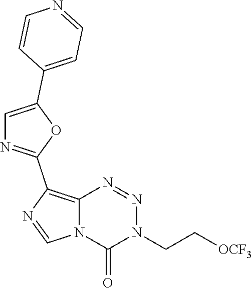 [SMILES: O=c1n(CCOC(F)(F)F)nnc2c(-c3ncc(-c4ccncc4)o3)ncn12] |
| II-11 | 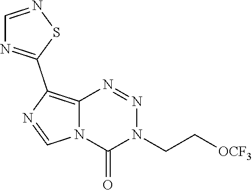 [SMILES: O=c1n(CCOC(F)(F)F)nnc2c(-c3ncns3)ncn12] |
| II-12 | 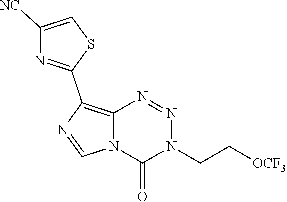 [SMILES: N#Cc1csc(-c2ncn3c(=O)n(CCOC(F)(F)F)nnc23)n1] |
| II-13 | 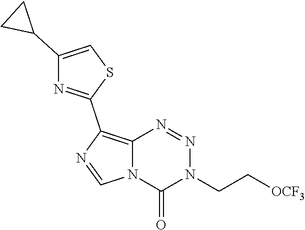 [SMILES: O=c1n(CCOC(F)(F)F)nnc2c(-c3nc(C4CC4)cs3)ncn12] |
| II-14 | 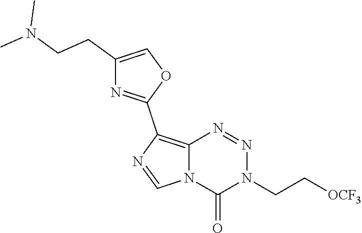 [SMILES: CN(C)CCc1coc(-c2ncn3c(=O)n(CCOC(F)(F)F)nnc23)n1] |
| II-15 | 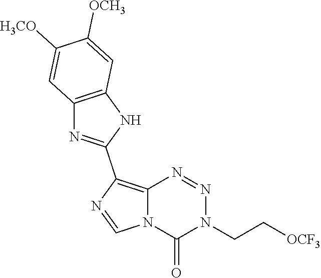 [SMILES: COc1cc2nc(-c3ncn4c(=O)n(CCOC(F)(F)F)nnc34)[nH]c2cc1OC] |
| II-16 | 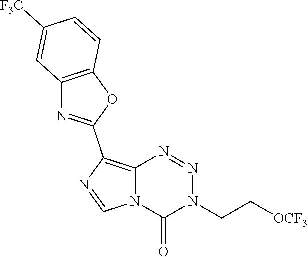 [SMILES: O=c1n(CCOC(F)(F)F)nnc2c(-c3nc4cc(C(F)(F)F)ccc4o3)ncn12] |
| II-17 | 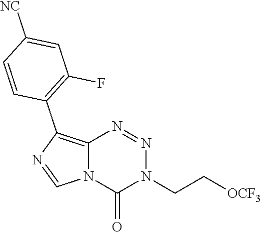 [SMILES: N#Cc1ccc(-c2ncn3c(=O)n(CCOC(F)(F)F)nnc23)c(F)c1] |
| II-18 | 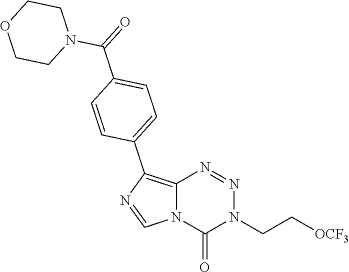 [SMILES: O=C(c1ccc(-c2ncn3c(=O)n(CCOC(F)(F)F)nnc23)cc1)N1CCOCC1] |

In certain embodiments, the compound is a compound in Table 3 or a pharmaceutically acceptable salt thereof. In certain embodiments, the compound is a compound in Table 3.

TABLE 3
Exemplary Compounds

| Compound No. | Chemical Structure |
|---|---|
| III-1 | 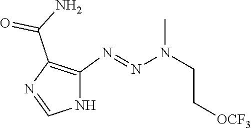 [SMILES: CN(CCOC(F)(F)F)/N=N/c1[nH]cnc1C(N)=O] |
| III-2 | 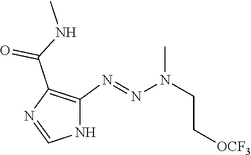 [SMILES: CNC(=O)c1nc[nH]c1/N=N/N(C)CCOC(F)(F)F] |
| III-3 | 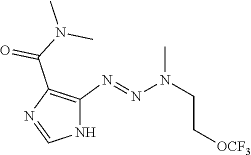 [SMILES: CN(CCOC(F)(F)F)/N=N/c1[nH]cnc1C(=O)N(C)C] |
| III-4 | 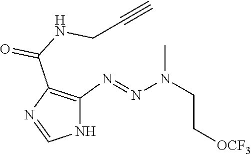 [SMILES: C#CCNC(=O)c1nc[nH]c1/N=N/N(C)CCOC(F)(F)F] |
| III-5 | 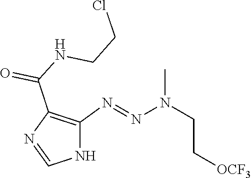 [SMILES: CN(CCOC(F)(F)F)/N=N/c1[nH]cnc1C(=O)NCCCl] |
| III-6 | 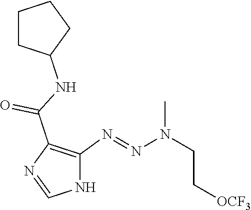 [SMILES: CN(CCOC(F)(F)F)/N=N/c1[nH]cnc1C(=O)NC1CCCC1] |
| III-7 | 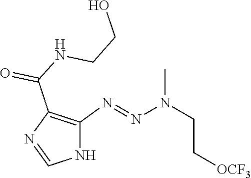 [SMILES: CN(CCOC(F)(F)F)/N=N/c1[nH]cnc1C(=O)NCCO] |
| III-8 | 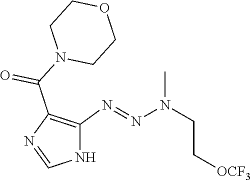 [SMILES: CN(CCOC(F)(F)F)/N=N/c1[nH]cnc1C(=O)N1CCOCC1] |
| III-9 | 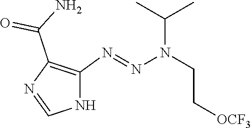 [SMILES: CC(C)N(CCOC(F)(F)F)/N=N/c1[nH]cnc1C(N)=O] |
| III-10 | 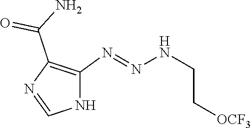 [SMILES: NC(=O)c1nc[nH]c1/N=N/NCCOC(F)(F)F] |
| III-11 | 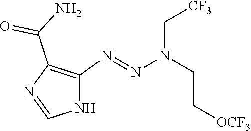 [SMILES: NC(=O)c1nc[nH]c1/N=N/N(CCOC(F)(F)F)CC(F)(F)F] |
| III-12 | 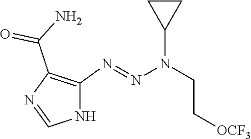 [SMILES: NC(=O)c1nc[nH]c1/N=N/N(CCOC(F)(F)F)C1CC1] |
| III-13 | 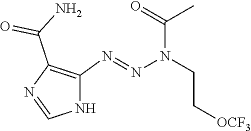 [SMILES: CC(=O)N(CCOC(F)(F)F)/N=N/c1[nH]cnc1C(N)=O] |
| III-14 | 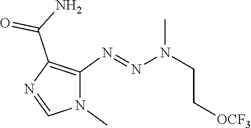 [SMILES: CN(CCOC(F)(F)F)/N=N/c1c(C(N)=O)ncn1C] |
| III-15 | 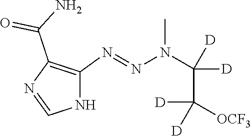 [SMILES: [2H]C([2H])(OC(F)(F)F)C([2H])([2H])N(C)/N=N/c1[nH]cnc1C(N)=O] |
| III-16 | 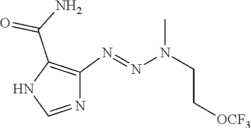 [SMILES: CN(CCOC(F)(F)F)/N=N/c1nc[nH]c1C(N)=O] |
| III-17 | 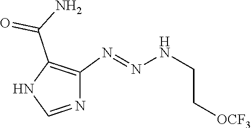 [SMILES: NC(=O)c1[nH]cnc1/N=N/NCCOC(F)(F)F] |
| III-18 | 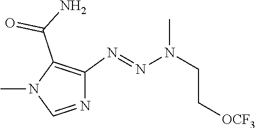 [SMILES: CN(CCOC(F)(F)F)/N=N/c1ncn(C)c1C(N)=O] |
| III-19 | 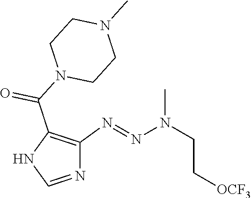 [SMILES: CN1CCN(C(=O)c2[nH]cnc2/N=N/N(C)CCOC(F)(F)F)CC1] |
| III-20 | 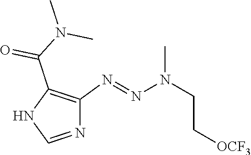 [SMILES: CN(CCOC(F)(F)F)/N=N/c1nc[nH]c1C(=O)N(C)C] |
| III-21 | 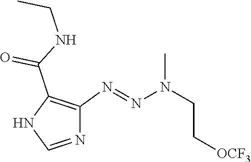 [SMILES: CCNC(=O)c1[nH]cnc1/N=N/N(C)CCOC(F)(F)F] |

In certain embodiments, the compound is a compound in Table 4 or a pharmaceutically acceptable salt thereof. In certain embodiments, the compound is a compound in Table 4. In certain embodiments, the compound is compound IV-1 or IV-2 in Table 4, or a pharmaceutically acceptable salt thereof. In certain embodiments, the compound is compound IV-1 or IV-2 in Table 4.

TABLE 4
Exemplary Compounds

| Compound | |
|---|---|
| No. | Chemical Structure |
| IV-1 | 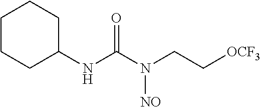 |
| IV-2 | 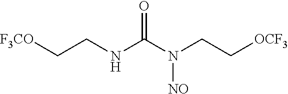 |
| IV-3 | 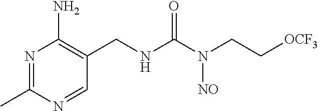 |
| IV-4 | 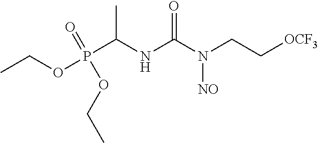 |
| IV-5 | 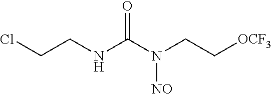 |
| IV-6 | 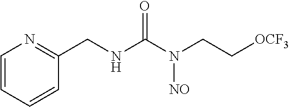 |
| IV-7 | 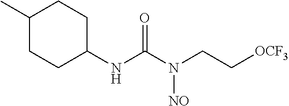 |
| IV-8 | 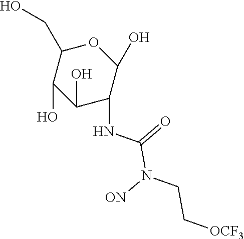 |
| IV-9 | 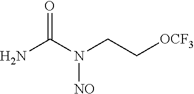 |
| IV-10 | 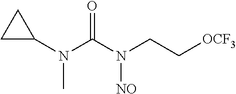 |
| IV-11 | 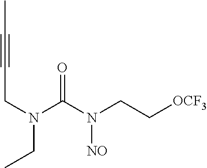 |
| IV-12 | 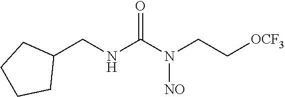 |
| IV-13 | 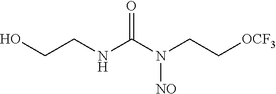 |
| IV-14 | 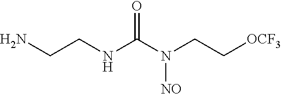 |
| IV-15 | 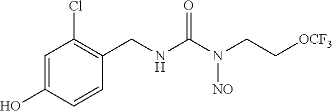 |
| IV-16 | 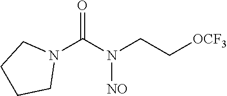 |
| IV-17 | 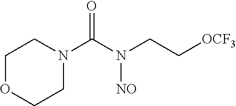 |
| IV-18 | 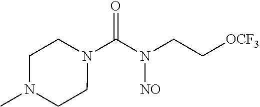 |

In certain embodiments, the compound is a compound in Table 5 or a pharmaceutically acceptable salt thereof. In certain embodiments, the compound is a compound in Table 5. In certain embodiments, the compound is compound V-1 in Table 5, or a pharmaceutically acceptable salt thereof. In certain embodiments, the compound is compound V-1 in Table 5.

TABLE 5
Exemplary Compounds

| Compound No. | Chemical Structure |
|---|---|
| V-1 | 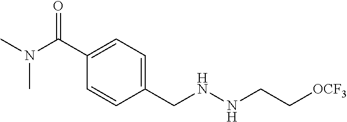 [SMILES: CN(C)C(=O)c1ccc(CNNCCOC(F)(F)F)cc1] |
| V-2 | 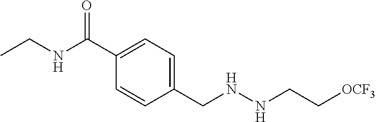 [SMILES: CCNC(=O)c1ccc(CNNCCOC(F)(F)F)cc1] |
| V-3 | 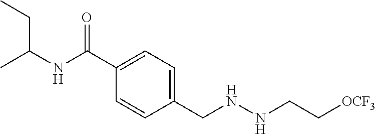 [SMILES: CCC(C)NC(=O)c1ccc(CNNCCOC(F)(F)F)cc1] |
| V-4 | 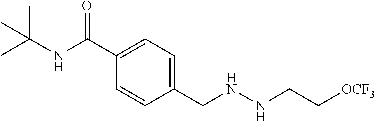 [SMILES: CC(C)(C)NC(=O)c1ccc(CNNCCOC(F)(F)F)cc1] |
| V-5 | 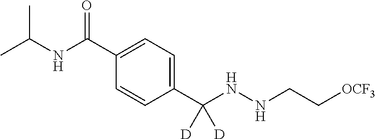 [SMILES: [2H]C([2H])(NNCCOC(F)(F)F)c1ccc(C(=O)NC(C)C)cc1] |
| V-6 | 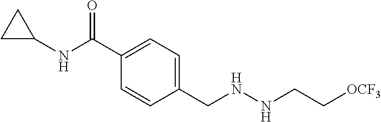 [SMILES: O=C(NC1CC1)c1ccc(CNNCCOC(F)(F)F)cc1] |
| V-7 | 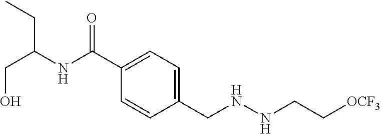 [SMILES: CCC(CO)NC(=O)c1ccc(CNNCCOC(F)(F)F)cc1] |
| V-8 | 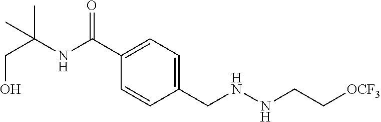 [SMILES: CC(C)(CO)NC(=O)c1ccc(CNNCCOC(F)(F)F)cc1] |
| V-9 | 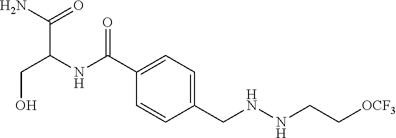 [SMILES: NC(=O)C(CO)NC(=O)c1ccc(CNNCCOC(F)(F)F)cc1] |
| V-10 | 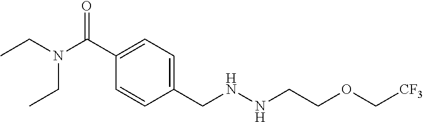 [SMILES: CCN(CC)C(=O)c1ccc(CNNCCOCC(F)(F)F)cc1] |
| V-11 | 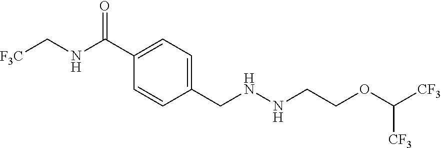 [SMILES: O=C(NCC(F)(F)F)c1ccc(CNNCCOC(C(F)(F)F)C(F)(F)F)cc1] |
| V-12 | 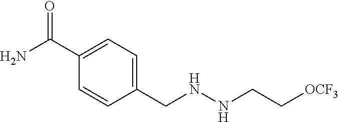 [SMILES: NC(=O)c1ccc(CNNCCOC(F)(F)F)cc1] |
| V-13 | 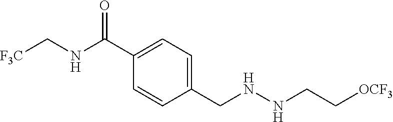 [SMILES: O=C(NCC(F)(F)F)c1ccc(CNNCCOC(F)(F)F)cc1] |
| V-14 | 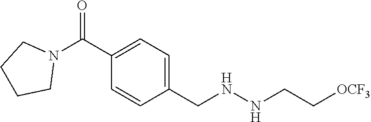 [SMILES: O=C(c1ccc(CNNCCOC(F)(F)F)cc1)N1CCCC1] |
| V-15 | 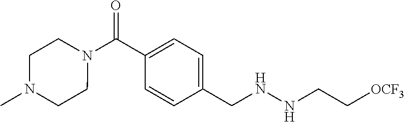 [SMILES: CN1CCN(C(=O)c2ccc(CNNCCOC(F)(F)F)cc2)CC1] |
| V-16 | 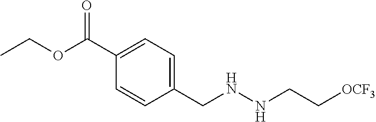 [SMILES: CCOC(=O)c1ccc(CNNCCOC(F)(F)F)cc1] |
| V-17 | 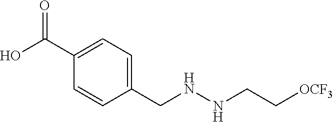 [SMILES: O=C(O)c1ccc(CNNCCOC(F)(F)F)cc1] |
| V-18 | 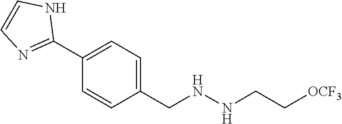 [SMILES: FC(F)(F)OCCNNCc1ccc(-c2ncc[nH]2)cc1] |
| V-19 | 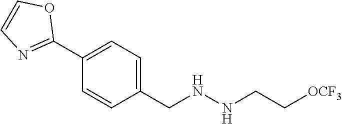 [SMILES: FC(F)(F)OCCNNCc1ccc(-c2ncco2)cc1] |
| V-20 | 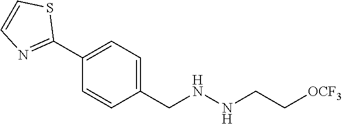 [SMILES: FC(F)(F)OCCNNCc1ccc(-c2nccs2)cc1] |
| V-21 | 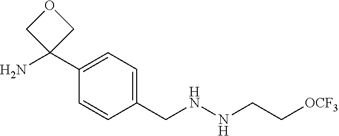 [SMILES: NC1(c2ccc(CNNCCOC(F)(F)F)cc2)COC1] |
| V-22 | 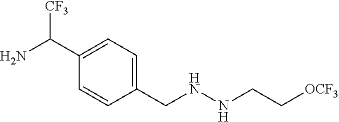 [SMILES: NC(c1ccc(CNNCCOC(F)(F)F)cc1)C(F)(F)F] |
| V-23 | 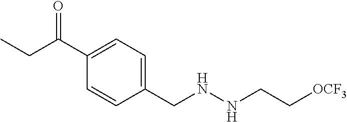 [SMILES: CCC(=O)c1ccc(CNNCCOC(F)(F)F)cc1] |
| V-24 | 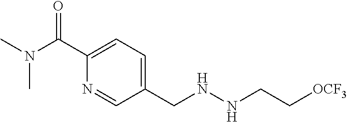 [SMILES: CN(C)C(=O)c1ccc(CNNCCOC(F)(F)F)cn1] |
| V-25 | 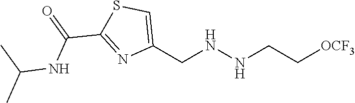 [SMILES: CC(C)NC(=O)c1nc(CNNCCOC(F)(F)F)cs1] |
| V-26 | 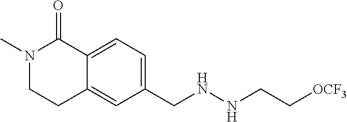 [SMILES: CN1CCc2cc(CNNCCOC(F)(F)F)ccc2C1=O] |
| V-27 | 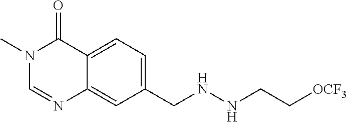 [SMILES: Cn1cnc2cc(CNNCCOC(F)(F)F)ccc2c1=O] |

### Synthetic Methods

Methods for preparing compounds described herein are illustrated in the following synthetic Schemes. The Schemes are given for the purpose of illustrating the invention, and not intended to limit the scope or spirit of the invention. Starting materials shown in the Schemes can be obtained from commercial sources or can be prepared based on procedures described in the literature.

Scheme 1 illustrates a general method for preparing (trifluoromethoxy)ethyl-imidazo[5,1-d]tetrazine carboxamide B. Reaction of 2-(trifluoromethoxy)ethan-1-amine with diphosgene provides 1-isocyanato-2-(trifluoromethoxy)ethane. Reaction of imidazolyl A with 1-isocyanato-2-(trifluoromethoxy)ethane provides (trifluoromethoxy)ethyl-imidazo[5,1-d]tetrazine carboxamide B.

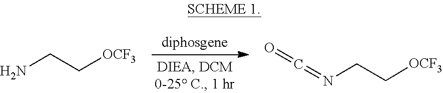 [SMILES: NCCOC(F)(F)F ; O=C=NCCOC(F)(F)F]

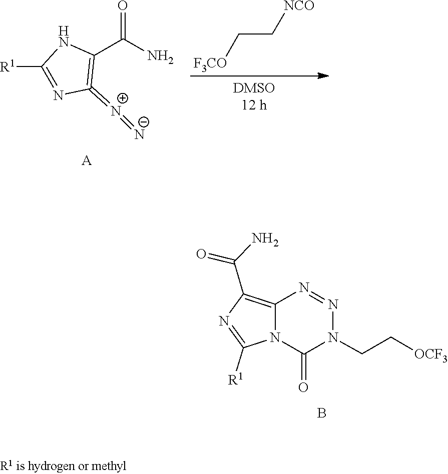

Scheme 2 illustrates a general method for preparing (fluoroalkoxy)alkylene dihydroimidazo[5,1-d]tetrazine carboxamides D, such as those defined by Formula I wherein R³ is —C(O)N(R⁴)(R⁵) and certain of the compounds depicted in Table 1. Hydrolysis of carboxamide A (prepared by analogy to the procedure depicted in Scheme 1 above), using NaNO₂ in TFA and water, for example, affords carboxylic acid B. Amide coupling of carboxylic acid B and amine C using known amide coupling reagents, such as T₃P or HATU, and a base, such as Et₃N or NMM, in a polar aprotic solvent, such as DMF, affords (fluoroalkoxy)alkylene dihydroimidazo[5,1-d]tetrazine carboxamides D. Variables R¹, R², R⁴, R⁵, and X may be, for example, as defined above in connection with Formula I.

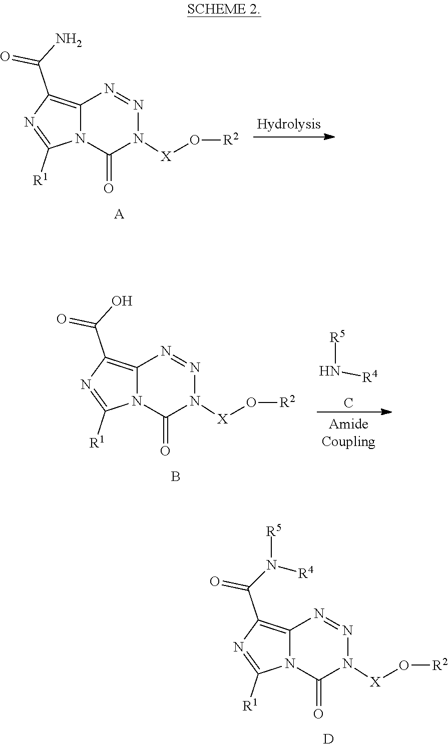

Additional dihydroimidazo[5,1-d]tetrazine compounds of Formula I (such as those defined by Formula I wherein R³ is —CO₂R⁵, —C(O)SR⁴, —C(S)N(R⁴)(R⁵), —C(═NR)OR⁴, —C(═NR⁷)SR⁴, —C(═NR⁷)N(R⁴)(R⁵), —C(O)-(halo), —C(O)—(C₁₋₄ alkyl), —CN, or halo, and certain of the compounds depicted in Table 1) may be prepared by functional group transformations of compounds A, B, and D depicted in Scheme 2, as described in detail for certain compounds in the Examples (for example, Examples 14, 16-20, and 28). Additionally, the acid chloride of carboxylic acid B from Scheme 2 (prepared, for example, by treating with a reagent such as SOCl₂) may be condensed with various nucleophiles, such as alcohols, phenols, and thiols, to provide compounds of Formula I wherein R³ is —CO₂R⁵ or —C(O)SR⁴. Literature references are provided below which describe additional functional group transformations.

Scheme 3 illustrates a general method for preparing (fluoroalkoxy)alkylene dihydroimidazo[5,1-d]tetrazines D, such as those defined by Formula I wherein R³ is C₁₋₄ alkyl, those defined by Formula II wherein A¹ is phenyl, and certain of the compounds depicted in Tables 1 and 2. Diazotization of amino-imidazole A using, for example, NaNO₂ in acid, such as hydrochloric acid, provides diazo-imidazole B. Condensation of diazo-imidazole B with isocyanate C in a solvent, such as DMSO, affords (fluoroalkoxy)alkylene dihydroimidazo[5,1-d]tetrazine D. Variables R¹, R², and X may be, for example, as defined above in connection with Formula I or Formula II.

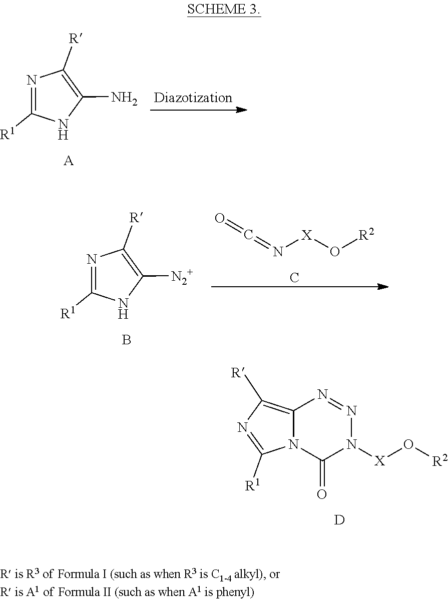

Heteroaryl compounds such as those defined by Formula II and those depicted in Table 2 may be prepared by coupling and cyclocondensation of compounds A, B, and D depicted in Scheme 2, and the thioamide analogue of carboxamide A (prepared, for example, by treating carboxamide A with Lawesson's reagent). Exemplary strategies and procedures for heteroaryl synthesis can be found, for example, in Svec, R. L., et al. “Tunable Stability of Imidazotetrazines Leads to a Potent Compound for Glioblastoma,” ACS Chemical Biology, 2018, 13, p. 3206-3216, and WO 2020/033880. Schemes 3 and 4 illustrate exemplary general methods for heteroaryl synthesis.

Scheme 4 illustrates exemplary general methods for preparing (fluoroalkoxy)alkylene dihydroimidazo[5,1-d]tetrazine monocyclic heteroaryls B, E, and H, such as those defined by Formula II wherein A¹ is oxazolyl, thiazolyl, oxadiazolyl, thiadiazolyl, and certain of the compounds depicted in Table 2. Cyclocondesation using a dehydration reagent, such as POCl₃, of carboxamide A (prepared as described for carboxamide B in Scheme 2) affords oxazole B. Tandem coupling and cyclocondensation of thioamide C (prepared, for example, by treating carboxamide A in Scheme 2 with Lawesson's reagent) with carbonyl-containing compound D (wherein LG is a leaving group, such as bromo) provides thiazole E. Tandem coupling and cyclocondensation of carboxamide or thioamide F (compound A in Scheme 2, or compound C in Scheme 3, respectively) with N,N-dimethylformamide dimethyl acetal G, using procedures such as those described in Lin, Y. et al. “New Synthesis of 1,2,4-Thiadiazoles,” J. Org. Chem., 1980, 45, p. 3750-3753, affords 1,2,4-oxadiazole or 1,2,4-thiadiazole H. Variables R¹, R², R³, R⁴ and X may be, for example, as defined above in connection with Formula II.

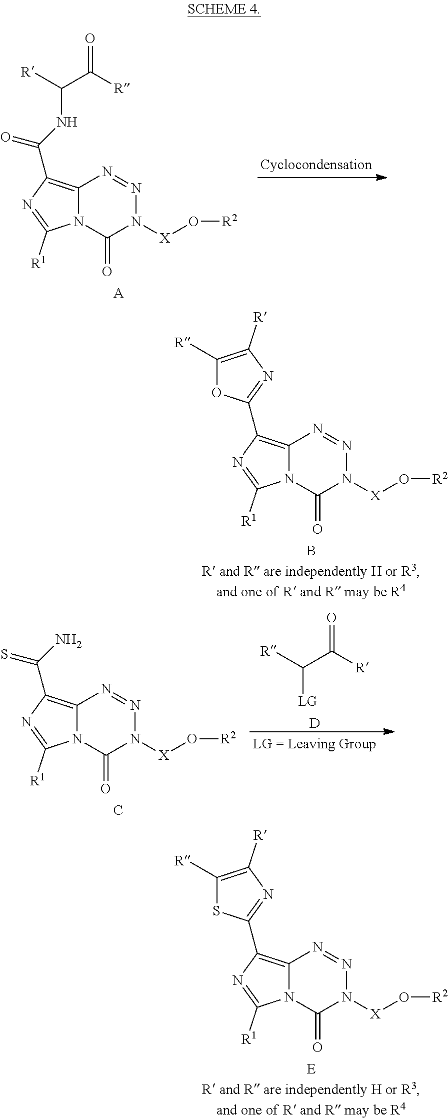

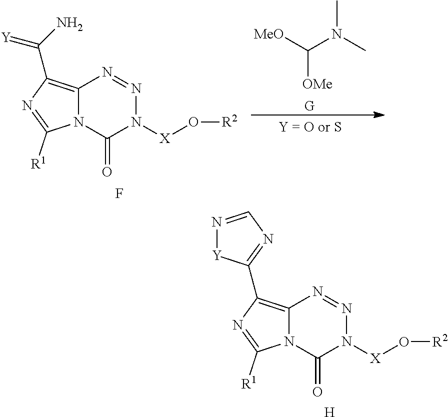

Scheme 5 illustrates exemplary general methods for preparing (fluoroalkoxy)alkylene dihydroimidazo[5,1-d]tetrazine bicyclic heteroaryls D, such as those defined by Formula II wherein A¹ is benzimidazolyl, benzoxazolyl, and benzthiazolyl, and certain of the compounds depicted in Table 2. Coupling carboxylic acid A (compound B in Scheme 2) with aniline B using, for example, HBTU or HATU, provides carboxamide C. Cyclocondensation of carboxamide C using, for example, POCl₃ or PPh₃ and DIAD, provides benzimidazole, benzoxazole, or benzthiazole D. Variables R¹, R², R³, R⁴, X, m, and n may be, for example, as defined above in connection with Formula II.

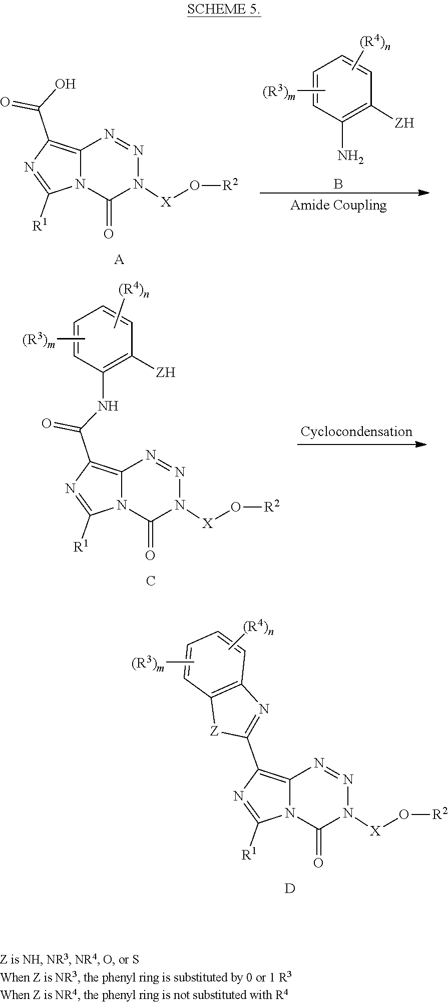

Scheme 6 illustrates exemplary general methods for preparing (fluoroalkoxy)alkylene imidazotriazenes D and G, such as those defined by Formula IIIa and IIb and those depicted in Table 3. Diazotization of amino-imidazoles A or E using, for example, NaNO₂ in acid, such as hydrochloric acid, provides diazo-imidazoles B or F. Condensation of diazo-imidazoles B or F with amine C in the presence of a base, such as Et₃N or DIPEA, affords (fluoroalkoxy)alkylene triazenyl-imidazole-carboxamides D or G. Variables R¹, R², R³, R⁴, R⁵, and X may be, for example, as defined above in connection with Formula IIa and Formula IIb.

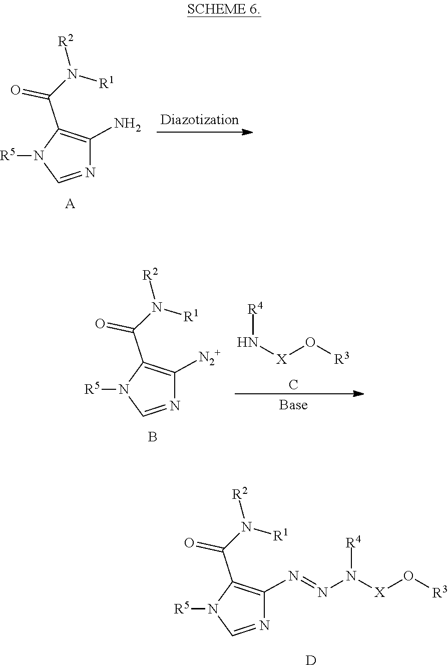

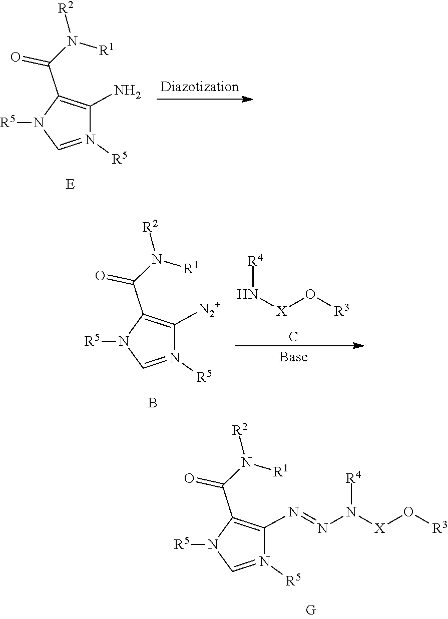

Scheme 7 illustrates a general method for preparing (fluoroalkoxy)alkylene nitrosoureas D, such as those defined by Formula IV and those depicted in Table 4. Condensation of isocyanate A with (fluoroalkoxy)alkylamine B in the presence of a base, such as Et₃N or DIPEA, in a solvent, such as Et₂O or THF, affords urea C, where R¹ is hydrogen. Preparation of urea C where R¹ is C₁₋₄ alkyl can be accomplished by alkylation, using an alkylating agent, such as an alkyl halide, and a base, such as Et₃N. Nitrosylation of urea C, using NaNO₂ in formic acid, for example, provides nitrosourea D. Variables R¹, R², R³, and X may be, for example, as defined above in connection with Formula IV.

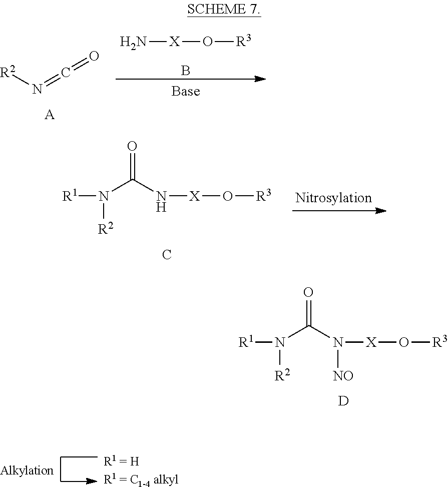

Scheme 8 illustrates exemplary general methods for preparing (fluoroalkoxy)alkylene hydrazines F, such as those defined by Formula V and those depicted in Table 5. Protection of the nitrogen atoms of hydrazino-alcohol A using, for example, Boc₂O and a solvent, such as dioxane (when protecting group PG is Boc), provides protected alcohol B. Fluoroalkylation of alcohol B using, for example, TMSCF₃, AgOTf, Selectfluor, and 2-fluoropyridine in a solvent such as EtOAc (when R¹ is CF₃), provides fluoroalkoxy protected hydrazine C. Alkylation of hydrazine C with alkylating agent D (where LG is a leaving group, such as bromide) using, for example, a base, such as NaH, and a solvent, such as DMF or THF, affords protected disubstituted hydrazine E. Deprotection of hydrazine E using, for example, an acid, such as hydrochloric acid, in a solvent, such as EtOAc (when PG is Boc) affords (fluoroalkoxy)alkylene hydrazine F. The R² substituent on ring A¹ may be contained in its final form in alkylating agent D (for example, when R² is —C(O)N(R³)(R⁴), C₁₋₄ alkyl, C₁₋₄ haloalkyl, or —C(O)—(C₁₋₄ alkyl)), or the R² substituent on ring A¹ may be elaborated to its final form either before or after the deprotection step (for example, by converting a carboxamide or carboxylic acid substituent on A¹ to a heteroaryl R² group, by analogy to the functional group transformations described for Scheme 3). Variables A¹, R¹, X¹, and X² may be, for example, as defined above in connection with Formula V.

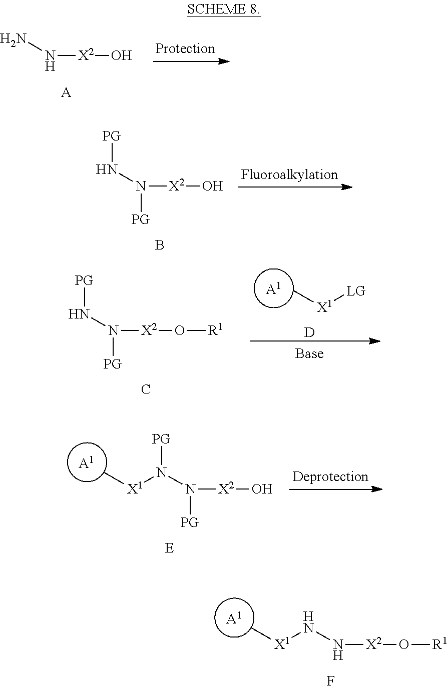

In the Schemes, it is understood by one skilled in the art of organic synthesis that the functionality present on various portions of the molecule should be compatible with the reagents and reactions proposed. Substituents not compatible with the reaction conditions will be apparent to one skilled in the art, and alternate methods are therefore indicated (for example, use of protecting groups or alternative reactions). Protecting group chemistry and strategy is well known in the art, for example, as described in detail in “Protecting Groups in Organic Synthesis”, T. W. Greene and P. G. M. Wuts, 3^{rd} edition, John Wiley & Sons, 1999, the entire contents of which are hereby incorporated by reference.

The modular synthetic route illustrated in Schemes 1-8 can be readily modified to provide additional compounds by conducting functional group transformations on the intermediate and/or final compounds. Such functional group transformations are well known in the art, as described in, for example, Comprehensive Organic Synthesis (B. M. Trost & I. Fleming, eds., 1991-1992); Organic Synthesis, 3rd Ed. (Michael B. Smith, Wavefunction, Inc., Irvine: 2010); Modern Methods of Organic Synthesis, 4th Ed. (William Carruthers and lain Coldham, Cambridge University Press, Cambridge: 2004); March's Advanced Organic Chemistry: Reactions, Mechanisms, and Structure, 8th Ed., (Michael B. Smith, John Wiley & Sons, New York: 2020); and Comprehensive Organic Transformations: A Guide to Functional Group Preparations, 3rd Ed. (Richard C. Larock, ed., John Wiley & Sons, New York: 2018).

### II. Therapeutic Applications

Another aspect of the invention provides a method of treating cancer, wherein the method comprises administering to a subject in need thereof a therapeutically effective amount of a compound described herein, such as a compound of Formula I, I-A, I-aa, II, III, IV, or V, to treat the cancer. In certain embodiments, the particular compound of Formula I, I-A, I-aa, II, III, IV, or V is a compound defined by one of the embodiments described in Section I, above.

In certain embodiments, the invention provides a method of treating cancer, wherein the method comprises administering to a subject in need thereof a therapeutically effective amount of a compound described herein, such as a compound of Formula I, to treat the cancer. In certain embodiments, the particular compound of Formula I is a compound defined by one of the embodiments described in Section I, above. In certain embodiments, the invention provides a method of treating cancer, wherein the method comprises administering to a subject in need thereof a therapeutically effective amount of a compound described herein, such as a compound of Formula I-A, to treat the cancer. In certain embodiments, the particular compound of Formula I-A is a compound defined by one of the embodiments described in Section I, above. In certain embodiments, the invention provides a method of treating cancer, wherein the method comprises administering to a subject in need thereof a therapeutically effective amount of a compound described herein, such as a compound of Formula I-aa, to treat the cancer. In certain embodiments, the particular compound of Formula I-aa is a compound defined by one of the embodiments described in Section I, above. In certain embodiments, the invention provides a method of treating cancer, wherein the method comprises administering to a subject in need thereof a therapeutically effective amount of a compound described herein, such as a compound of Formula II, to treat the cancer. In certain embodiments, the particular compound of Formula II is a compound defined by one of the embodiments described in Section I, above. In certain embodiments, the invention provides a method of treating cancer, wherein the method comprises administering to a subject in need thereof a therapeutically effective amount of a compound described herein, such as a compound of Formula III, to treat the cancer. In certain embodiments, the particular compound of Formula III is a compound defined by one of the embodiments described in Section I, above. In certain embodiments, the invention provides a method of treating cancer, wherein the method comprises administering to a subject in need thereof a therapeutically effective amount of a compound described herein, such as a compound of Formula IV, to treat the cancer. In certain embodiments, the particular compound of Formula IV is a compound defined by one of the embodiments described in Section I, above. In certain embodiments, the invention provides a method of treating cancer, wherein the method comprises administering to a subject in need thereof a therapeutically effective amount of a compound described herein, such as a compound of Formula V, to treat the cancer. In certain embodiments, the particular compound of Formula V is a compound defined by one of the embodiments described in Section I, above.

Another aspect of the invention provides a method of producing a DNA lesion in a subject, wherein the method comprises administering to a subject an effective amount of a compound described herein, such as a compound of Formula I, I-A, I-aa, II, III, IV, or V, to produce a DNA lesion in the subject. In certain embodiments, the subject has cancer. In certain embodiments, the compound is a compound of Formula I. In certain embodiments, the particular compound of Formula I is a compound defined by one of the embodiments described in Section I, above. In certain embodiments, the compound is a compound of Formula I-A. In certain embodiments, the particular compound of Formula I-A is a compound defined by one of the embodiments described in Section I, above. In certain embodiments, the compound is a compound of Formula I-aa. In certain embodiments, the particular compound of Formula I-aa is a compound defined by one of the embodiments described in Section I, above. In certain embodiments, the compound is a compound of Formula II. In certain embodiments, the particular compound of Formula II is a compound defined by one of the embodiments described in Section I, above. In certain embodiments, the compound is a compound of Formula III. In certain embodiments, the particular compound of Formula III is a compound defined by one of the embodiments described in Section I, above. In certain embodiments, the compound is a compound of Formula IV. In certain embodiments, the particular compound of Formula IV is a compound defined by one of the embodiments described in Section I, above. In certain embodiments, the compound is a compound of Formula V. In certain embodiments, the particular compound of Formula V is a compound defined by one of the embodiments described in Section I, above. Without being bound by a particular theory, it is understood that compounds of Formula I, I-A, I-aa, II, III, IV, or V herein generate a reactive alkylating agent in vivo that reacts with DNA in a subject to product a DNA lesion. The DNA lesion can be alkylated DNA.

### Further Features

Methods described herein may be further defined according to additional features, such as the identity of the cancer and/or the subject.

In certain embodiments, the cancer is ovarian cancer, uterine cancer, endometrial cancer, cervical cancer, prostate cancer, testicular cancer, breast cancer, brain cancer, lung cancer, oral cancer, esophageal cancer, head and neck cancer, stomach cancer, colon cancer, rectal cancer, skin cancer, sebaceous gland carcinoma, bile duct cancer, gallbladder cancer, liver cancer, pancreatic cancer, bladder cancer, urinary tract cancer, kidney cancer, eye cancer, thyroid cancer, lymphoma, leukemia, urothelial cancer, colorectal cancer, or glioblastoma multiforme.

In certain embodiments, the cancer is a breast invasive carcinoma, colon adenocarcinoma, head and neck cancer, lung adenocarcinoma, rectal adenocarcinoma, acute myeloid leukemia, glioblastoma multiforme, brain lower grade glioma, colorectal cancer, or metastatic melanoma. In certain embodiments, the cancer is a melanoma. In certain embodiments, the cancer is a glioblastoma multiforme.

In certain embodiments, the disorder is a cancer selected from the group consisting of ovarian cancer, uterine cancer, endometrial cancer, cervical cancer, prostate cancer, testicular cancer, breast cancer, brain cancer, lung cancer, oral cancer, esophageal cancer, head and neck cancer, stomach cancer, colon cancer, rectal cancer, skin cancer, sebaceous gland carcinoma, bile duct cancer, gallbladder cancer, liver cancer, pancreatic cancer, bladder cancer, urinary tract cancer, kidney cancer, eye cancer, thyroid cancer, lymphoma, and leukemia.

In certain embodiments, the cancer is a solid tumor. In certain embodiments, the cancer is a sarcoma or carcinoma. In certain embodiments, the cancer is ovarian cancer, uterine cancer, endometrial cancer, cervical cancer, prostate cancer, testicular cancer, breast cancer, brain cancer, lung cancer, oral cancer, esophageal cancer, head and neck cancer, stomach cancer, colon cancer, rectal cancer, skin cancer, sebaceous gland carcinoma, bile duct cancer, gallbladder cancer, liver cancer, pancreatic cancer, bladder cancer, urinary tract cancer, kidney cancer, eye cancer, thyroid cancer, lymphoma, or leukemia.

In certain embodiments, the cancer is prostate cancer, breast cancer, lung cancer, liver cancer, bladder cancer, urinary tract cancer, or eye cancer. In certain embodiments, the cancer is prostate cancer. In certain embodiments, the cancer is breast cancer. In certain embodiments, the cancer is lung cancer. In certain embodiments, the cancer is liver cancer. In certain embodiments, the cancer is bladder cancer. In certain embodiments, the cancer is urinary tract cancer. In certain embodiments, the cancer is eye cancer.

In certain embodiments, the cancer is squamous-cell carcinoma, basal cell carcinoma, adenocarcinoma, hepatocellular carcinomas, and renal cell carcinomas, cancer of the bladder, bowel, breast, cervix, colon, esophagus, head, kidney, liver, lung, neck, ovary, pancreas, prostate, and stomach; leukemias; benign and malignant lymphomas (e.g., Burkitt's lymphoma and Non-Hodgkin's lymphoma); benign and malignant melanomas; myeloproliferative diseases; sarcomas, including Ewing's sarcoma, hemangiosarcoma, Kaposi's sarcoma, liposarcoma, myosarcomas, peripheral neuroepithelioma, synovial sarcoma, gliomas, astrocytomas, oligodendrogliomas, ependymomas, gliobastomas, neuroblastomas, ganglioneuromas, gangliogliomas, medulloblastomas, pineal cell tumors, meningiomas, meningeal sarcomas, neurofibromas, and Schwannomas; bowel cancer, breast cancer, prostate cancer, cervical cancer, uterine cancer, lung cancer, ovarian cancer, testicular cancer, thyroid cancer, astrocytoma, esophageal cancer, pancreatic cancer, stomach cancer, liver cancer, colon cancer, melanoma; carcinosarcoma, Hodgkin's disease, Wilms' tumor and teratocarcinomas.

In certain embodiments, the cancer is a neuroblastoma, craniopharyngioma, glioma, glioblastoma, schwannoma, astrocytoma, oligodendroglioma, medulloblastoma, pinealoma, hemangioblastoma, retinoblastoma, ependymoma, chordoma, meningioma, medullary carcinoma, small cell lung carcinoma, papillary adenocarcinoma, papillary carcinoma, mesothelioma, nasopharyngeal carcinoma, acoustic neuroma, oral cancer, esophageal cancer, head and neck cancer, stomach cancer, colon cancer, rectal cancer, skin cancer, melanoma, sweat gland carcinoma, sebaceous gland carcinoma, squamous cell carcinoma, basal cell carcinoma, bile duct cancer, gallbladder cancer, liver cancer, hepatocellular carcinoma, pancreatic cancer, bladder carcinoma, renal cell carcinoma, kidney cancer, Wilms' tumor, thyroid cancer, parathyroid tumor, synovioma, soft tissue sarcoma (e.g., rhabdomyosarcoma (RMS)), Kaposi sarcoma, synovial sarcoma, osteosarcoma, Ewing's sarcoma, malignant rhabdoid tumor, leiomyosarcoma, liposarcoma, lymphangioendothelio-sarcoma, lymphangiosarcoma, myxosarcoma, osteogenic sarcoma, fibrosarcoma, chondrosarcoma, or endotheliosarcoma.

In certain embodiments, the cancer is a lymphoma. In certain embodiments, the cancer is Burkitt's lymphoma, diffuse large B-cell lymphoma (DLBCL), follicular lymphoma, non-Hodgkin's lymphoma, lymphoid malignancies of T-cell or B-cell origin, peripheral T-cell lymphoma, adult T-cell leukemia-lymphoma, or Waldenstrom's macroglobulinemia.

In certain embodiments, the cancer is a leukemia. In certain embodiments, the cancer is acute leukemia, lymphoblastic leukemia, acute lymphoblastic leukemia, myelogenous leukemia, acute myelogenous leukemia, acute T-cell leukemia, chronic leukemia, chronic lymphocytic leukemia, chronic myelocytic leukemia, chronic myelogenous leukemia, polycythemia vera, multiple myeloma, or erythroleukemia.

In certain embodiments, the cancer is a myelodysplastic and/or myeloproliferative syndrome. In certain embodiments, the cancer is a myelodysplastic syndrome. In certain embodiments, the cancer is a myeloproliferative syndrome.

In certain embodiments, the cancer is a cancer or related myeloproliferative disorder selected from histiocytosis, essential thrombocythemia, myelofibrosis, heavy chain disease, and other malignancies and hyperproliferative disorders of the bladder, breast, colon, lung, ovaries, pancreas, prostate, skin and uterus.

In certain embodiments, the cancer is a B-cell non-Hodgkin's lymphoma, advanced solid tumor, soft tissue sarcoma, INI1-deficient cancer, BAP1-deficient cancer, follicular lymphoma, relapsed/refractory follicular lymphoma, diffuse large B-cell lymphoma, relapsed/refractory diffuse large B-cell lymphoma, non-Hodgkin's lymphoma, pediatric non-Hodgkin's lymphoma, pediatric non-Hodgkin's lymphoma with EZH2, SMARCB1, or SMARCA4 mutation, histiocytic disorder, pediatric histiocytic disorder, pediatric histiocytic disorder with EZH2, SMARCB1, or SMARCA4 mutation, solid tumor with EZH2, SMARCB1, or SMARCA4 mutation, resistant prostate cancer, relapsed/refractory small-cell lung carcinoma, B-cell lymphoma, relapsed/refractory B-cell lymphoma, adult T-cell leukemia-lymphoma, or advanced diffuse large B-cell lymphoma.

In certain embodiments, the cancer is a malignant rhabdoid tumor, atypical teratoid rhabdoid tumor, epithelioid sarcoma, renal medullary carcinoma, pancreatic undifferentiated rhabdoid carcinoma, schwannoma, epithelioid malignant peripheral nerve sheath tumor, or diffuse intrinsic glioma.

In certain embodiments, the cancer is retinoblastoma multiforme, metastatic castration-resistant prostate cancer, prostate small cell neuroendocrine carcinoma, small-cell lung cancer, triple-negative breast cancer, hepatocellular carcinoma, bladder cancer, or urinary tract cancer.

In certain embodiments, the cancer is fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, epithelial carcinoma, glioma, astrocytoma, medulloblastoma, and hemangioblastoma. In certain embodiments, the cancer is a neuroblastoma, meningioma, hemangiopericytoma, multiple brain metastase, glioblastoma multiforms, glioblastoma, brain stem glioma, poor prognosis malignant brain tumor, malignant glioma, anaplastic astrocytoma, anaplastic oligodendroglioma, neuroendocrine tumor, rectal adeno carcinoma, Dukes C & D colorectal cancer, unresectable colorectal carcinoma, metastatic hepatocellular carcinoma, Kaposi's sarcoma, karotype acute myeloblastic leukemia, Hodgkin's lymphoma, non-Hodgkin's lymphoma, cutaneous T-Cell lymphoma, cutaneous B-Cell lymphoma, diffuse large B-Cell lymphoma, low grade follicular lymphoma, metastatic melanoma, localized melanoma, malignant mesothelioma, malignant pleural effusion mesothelioma syndrome, peritoneal carcinoma, papillary serous carcinoma, gynecologic sarcoma, soft tissue sarcoma, scelroderma, cutaneous vasculitis, Langerhans cell histiocytosis, leiomyosarcoma, fibrodysplasia ossificans progressive, hormone refractory prostate cancer, resected high-risk soft tissue sarcoma, unrescectable hepatocellular carcinoma, Waidenstrom's macroglobulinemia, smoldering myeloma, indolent myeloma, fallopian tube cancer, androgen independent prostate cancer, androgen dependent stage IV non-metastatic prostate cancer, hormone-insensitive prostate cancer, chemotherapy-insensitive prostate cancer, papillary thyroid carcinoma, follicular thyroid carcinoma, medullary thyroid carcinoma, or leiomyoma.

In certain embodiments, the cancer is a metastatic cancer. In certain embodiments, the cancer is a relapsed and/or refractory cancer.

In certain embodiments, the cancer is ovarian cancer, uterine cancer, gestational trophoblastic disease, endometrial cancer, cervical cancer, embryonal carcinoma, choriocarcinoma, prostate cancer (including hormone insensitive and castrate resistant prostate cancers), testicular tumors (including germ cell testicular cancer/seminoma), cystadenocarcinoma, breast cancer (including estrogen-receptor positive breast cancer), brain tumors (including neuroblastoma, craniopharyngioma, glioma, glioblastoma, schwannoma, astrocytoma, oligodendroglioma, medulloblastoma, and pinealoma), hemangioblastoma, retinoblastoma, ependymoma, chordoma, meningioma, medullary carcinoma, lung cancer (including small cell lung carcinoma, papillary adenocarcinomas, and papillary carcinoma), mesothelioma, nasopharyngeal carcinoma, acoustic neuroma, oral cancer, esophageal cancer, head and neck cancer, stomach cancer, colon cancer, rectal cancer, skin cancer, melanoma, sweat gland carcinoma, sebaceous gland carcinoma, squamous cell carcinoma, basal cell carcinoma, bile duct cancer, gallbladder cancer, liver cancer, hepatocellular carcinoma, pancreatic cancer, bladder carcinoma, renal cell carcinoma, kidney cancer, Wilms' tumor, thyroid cancer, parathyroid tumor, synovioma, soft tissue sarcoma (e.g., rhabdomyosarcoma (RMS)), Kaposi sarcoma, synovial sarcoma, osteosarcoma, Ewing's sarcoma, malignant rhabdoid tumor, leiomyosarcoma, liposarcoma, lymphangioendothelio-sarcoma, lymphangiosarcoma, myxosarcoma, osteogenic sarcoma, fibrosarcoma, chondrosarcoma, or endotheliosarcoma.

In certain embodiments, the cancer is MGMT deficient. “MGMT deficient” (or MGMT⁻) cancers means cancers that have more than one standard deviation lower abundance of the mRNA transcript for the MGMT gene or more than one standard deviation lower abundance of the associated functional protein itself normalized to the relevant healthy control tissue. This deficiency can occur through promoter methylation, mutations in the gene, or through other methods resulting in downregulation of the gene. MGMT expression levels in various cancer cell lines have been determined. Exemplary MGMT expression data is provided, for example, in Tables 7-11 in Examples 36 and 37, below.

In certain embodiments, the cancer is MMR deficient. “MMR deficient” (or MMR⁻) cancers means cancers that have more than one standard deviation lower abundance of the mRNA transcript for any of the MMR genes (MSH2, MSH6, MLH1, MLH3, PMS2, PMS1) or more than one standard deviation lower abundance of the respective functional protein(s) normalized to the relevant healthy control tissue. Alternatively, cancers that exhibit the microsatellite instability high phenotype (MSI-H) are also considered to be MMR deficient. See, for example, Li et al.—Microsatellite instability: a review of what the oncologist should know—Cancer Cell International, Article Number 16 (2020).

In certain embodiments, the cancer is resistant to treatment using temozolomide.

### Subjects

In certain embodiments, the subject is a human. In certain embodiments, the subject is an adult human. In certain embodiments, the subject is a pediatric human.

### Medical Uses

Another aspect of the invention provides for the use of a compound described herein (such as a compound of Formula I, I-A, I-aa, II, III, IV, or V, or other compounds in Section I) in the manufacture of a medicament. In certain embodiments, the medicament is for treating a disorder described herein, such as cancer.

Another aspect of the invention provides for the use of a compound described herein (such as a compound of Formula I, I-A, I-aa, II, III, IV, or V, or other compounds in Section I) for treating a medical disorder, such as a medical disorder described herein, such as cancer.

### Biological Activity Testing

Compounds may be evaluated for ability to kill cancer cells according to assay procedures described in the literature for evaluating ability of a test compound to kill the cancer cell. Additionally, compounds may be evaluated for ability to kill cancer cells according to assay procedures described below:

Cytotoxicity of the compounds may be measured in short-term cell viability assays against four isogenic LN229 glioblastoma cell lines engineered to be proficient or deficient in MGMT and/or MMR activity by using short hairpin RNAs (shRNAs) targeting MSH2. This approach allows for determination of the relationship between MGMT status, MMR status, and compound activity. LN229 cells are maintained in Dulbecco's Modified Eagle Medium (DMEM) supplemented with 10% fetal bovine serum, 1% Penicillin/Streptomycin, and 0.1% Fungin. DAY 0: Cells are plated into sterile 96-well plates (Corning Costar 96-well) at a concentration of 500 cells/well using a Multichannel pipettor. Then, assay plates are incubated overnight in a 37° C. 5% CO₂ incubator. DAY 1: Compounds are prepared as 50 mM stocks in dimethyl sulfoxide (DMSO) and stored protected from light at room temperature (RT) until use. Prior to compound addition, a two-fold serial dilution of compound stock solutions is performed in DMSO from 30 mM to 0.117 mM in a 96-well Master plate. Vehicle control wells contain DMSO. 97 μL of media is added to each well of a new 96-well plate (Daughter plate) and 3 μL of drug solution from Master plate is added to corresponding wells in the Daughter plate resulting in a 3× plate. Three replicate dilution curves of each compound are run on each assay plate. The final concentration of compounds ranges from 300 μM to 1.171 μM (9-point, 2-fold dilution dose response curve), and the final DMSO concentration is 1%.

Assay plates are incubated for 120 hours at 37° C. in a humidified 5% CO₂ incubator. DAY 5: Following incubation, cells are fixed with 4% paraformaldehyde and stained with Hoechst dye for nuclei visualization. Fixation, staining and washing is performed by using a Thermo Scientific Multidrop Combi. Images are acquired on a BioTek Cytation 5 Cell Imaging Multimode Reader and quantified using Cell Profiler image analysis software. Raw cell count data for test compounds are normalized to percent viability relative to the DMSO vehicle control.

### Generation of 2-(Trifluoromethoxy)ethane-1-diazonium and DNA Adducts

Without wishing to be bound by theory, another aspect pertains to using compounds described herein to generate 2-(trifluoromethoxy)ethane-1-diazonium in vivo, which is a potent alkylating agent that can react with DNA to result in a 2-(trifluoromethoxy)ethane-1-DNA adduct. 2-(Trifluoromethoxy)ethane-1-diazonium has the following chemical structure:

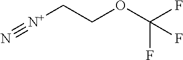

The generation of a 2-(trifluoromethoxy)ethane-1-DNA adduct ultimately results in death of a cell containing the 2-(trifluoromethoxy)ethane-1-DNA adduct, for those cells having a low amount of the DNA repair protein O⁶-methylguanine-DNA-methyltransferase (MGMT). Numerous types of cancer cells have a low amount of MGMT, and death of the cancer results following administration of the compound containing a 3-(2-(trifluoromethoxy)ethyl) imidazo[5,1-d][1,2,3,5]tetrazin-4(3H)-one scaffold. Healthy cells have sufficient amount of MGMT; and, therefore, healthy cells do not succumb (i.e., die) to the effects of a compound containing a 3-(2-(trifluoromethoxy)ethyl) imidazo[5,1-d][1,2,3,5]tetrazin-4(3H)-one scaffold.

Accordingly, without wishing to be bound by theory, compounds containing a 3-(2-(trifluoromethoxy)ethyl)imidazo[5,1-d][1,2,3,5]tetrazin-4(3H)-one scaffold described herein undergo conversion in vivo to generate 2-(trifluoromethoxy)ethane-1-diazonium. The 2-(trifluoromethoxy)ethane-1-diazonium reacts with DNA in a cell to result in a 2-(trifluoromethoxy)ethane-1-DNA adduct. Cancer cells with a low amount of MGMT die as a result of 2-(trifluoromethoxy)ethane-1-DNA adduct.

Accordingly, one aspect provides a method of treating a MGMT-deficient cancer in a patient, comprising exposing a MGMT-deficient cancer cell in the patient in need thereof to 2-(C₁₋₄ fluoroalkoxy)ethane-1-diazonium, to thereby treat the MGMT-deficient cancer.

Another aspect provides a method of treating a MGMT-deficient cancer in a patient, comprising exposing a MGMT-deficient cancer cell in the patient in need thereof to 2-(trifluoromethoxy)ethane-1-diazonium, to thereby treat the MGMT-deficient cancer.

Another aspect provides a method of treating a MGMT-deficient cancer in a patient, comprising forming 2-(trifluoromethoxy)ethane-1-diazonium in a MGMT-deficient cancer cell in the patient in need of treatment, to thereby treat the MGMT-deficient cancer.

Another aspect provides a method of treating a MGMT-deficient cancer in a patient, comprising forming a 2-(C₁₋₄ fluoroalkoxy)ethane-1-DNA adduct in a MGMT-deficient cancer cell in the patient in need thereof, to thereby treat the MGMT-deficient cancer.

Another aspect provides a method of treating a MGMT-deficient cancer in a patient, comprising forming a 2-(trifluoromethoxy)ethane-1-DNA adduct in a MGMT-deficient cancer cell in the patient in need thereof, to thereby treat the MGMT-deficient cancer.

Another aspect provides a method of treating a MGMT-deficient cancer in a patient, comprising exposing DNA to 2-(trifluoromethoxy)ethane-1-diazonium to thereby form a 2-(trifluoromethoxy)ethane-1-DNA adduct in a MGMT-deficient cancer cell in the patient in need thereof, to thereby treat the MGMT-deficient cancer.

Another aspect provides a method, comprising exposing DNA to 2-(trifluoromethoxy)ethane-1-diazonium in a cancer cell to thereby form a 2-(trifluoromethoxy)ethane-1-DNA adduct in the cancer cell.

Another aspect provides a method of treating a MGMT-deficient cancer in a patient, comprising administering to the patient in need thereof a compound comprising a 2-(trifluoromethoxy)ethanyl group, to thereby treat the patient, wherein said compound undergoes conversion in vivo to 2-(trifluoromethoxy)ethane-1-diazonium.

Another aspect provides a method of forming a 2-(trifluoromethoxy)ethane-1-DNA adduct, comprising exposing DNA to a compound comprising a 2-(trifluoromethoxy)ethanyl group to thereby form a 2-(trifluoromethoxy)ethane-1-DNA adduct, wherein said compound undergoes conversion in vivo to 2-(trifluoromethoxy)ethane-1-diazonium.

Another aspect provides a method of forming a 2-(trifluoromethoxy)ethane-1-DNA adduct, comprising exposing DNA to 2-(trifluoromethoxy)ethane-1-diazonium to thereby form a 2-(trifluoromethoxy)ethane-1-DNA adduct. In certain embodiments, the method comprises exposing DNA in a cancer patient to 2-(trifluoromethoxy)ethane-1-diazonium. In certain embodiments, the cancer patient has a cancer that is MGMT-deficient.

Another aspect provides a DNA adduct, comprising DNA covalently bonded to one or more occurrences of

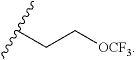 [SMILES: *CCOC(F)(F)F]

In certain embodiments, the DNA adduct comprises DNA covalently bonded to 1 to 10 occurrences of

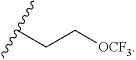 [SMILES: *CCOC(F)(F)F]

In certain embodiments, the DNA adduct comprises DNA covalently bonded to 1 to 5 occurrences of

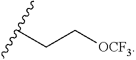 [SMILES: *CCOC(F)(F)F]

In certain embodiments, the DNA adduct comprises DNA covalently bonded to 1 to 2 occurrences of

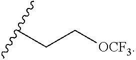 [SMILES: *CCOC(F)(F)F]

In certain embodiments, the DNA adduct comprises DNA covalently bonded to 1 occurrence of

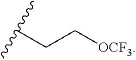 [SMILES: *CCOC(F)(F)F]

In certain embodiments, the DNA adduct comprises the following group

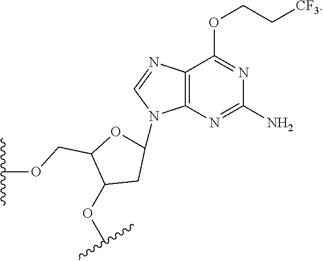 [SMILES: *OCC1OC(n2cnc3c(OCCC(F)(F)F)nc(N)nc32)CC1O*]

Another aspect provides a method of treating a MGMT-deficient cancer in a patient, comprising administering to the patient in need thereof a compound comprising a 2-(trifluoromethoxy)ethanyl group, to thereby treat the patient, wherein said compound undergoes conversion in vivo to 2-(trifluoromethoxy)ethane-1-diazonium. In certain embodiments, said compound is a small organic compound having a molecule weight less than 2000 g/mol. In certain embodiments, said compound is a small organic compound having a molecule weight less than 1000 g/mol. In certain embodiments, said compound is a small organic compound having a molecule weight less than 900, 800, 700, 600, 500, 400, or 300 g/mol.

### III. Combination Therapy

Another aspect of the invention provides for combination therapy. Compounds described herein (such as a compound of Formula I, I-A, I-aa, II, III, IV, or V, or other compounds in Section I) or their pharmaceutically acceptable salts may be used in combination with additional therapeutic agents to treat medical disorders, such as an autoimmune disorder or a cancer.

In some embodiments, the present invention provides a method of treating a disclosed disease or condition comprising administering to a patient in need thereof an effective amount of a compound disclosed herein or a pharmaceutically acceptable salt thereof and co-administering simultaneously or sequentially an effective amount of one or more additional therapeutic agents, such as those described herein. In some embodiments, the method includes co-administering one additional therapeutic agent. In some embodiments, the method includes co-administering two additional therapeutic agents. In some embodiments, the combination of the disclosed compound and the additional therapeutic agent or agents acts synergistically.

One or more other therapeutic agent may be administered separately from a compound or composition of the invention, as part of a multiple dosage regimen. Alternatively, one or more other therapeutic agents may be part of a single dosage form, mixed together with a compound of this invention in a single composition. If administered as a multiple dosage regime, one or more other therapeutic agent and a compound or composition of the invention may be administered simultaneously, sequentially or within a period of time from one another, for example within 1, 2, 3, 4, 5, 6, 7, 8, 9, 10, 11, 12, 13, 14, 15, 16, 17, 18, 19, 20, 21, 22, 23, or 24 hours from one another. In some embodiments, one or more other therapeutic agent and a compound or composition of the invention are administered as a multiple dosage regimen more than 24 hours apart.

### Anti-Cancer Agents

Exemplary therapeutic agents that may be used as part of a combination therapy in treating cancer, include, for example, mitomycin, tretinoin, ribomustin, gemcitabine, vincristine, etoposide, cladribine, mitobronitol, methotrexate, doxorubicin, carboquone, pentostatin, nitracrine, zinostatin, cetrorelix, letrozole, raltitrexed, daunorubicin, fadrozole, fotemustine, thymalfasin, sobuzoxane, nedaplatin, cytarabine, bicalutamide, vinorelbine, vesnarinone, aminoglutethimide, amsacrine, proglumide, elliptinium acetate, ketanserin, doxifluridine, etretinate, isotretinoin, streptozocin, nimustine, vindesine, flutamide, drogenil, butocin, carmofur, razoxane, sizofilan, carboplatin, mitolactol, tegafur, ifosfamide, prednimustine, picibanil, levamisole, teniposide, improsulfan, enocitabine, lisuride, oxymetholone, tamoxifen, progesterone, mepitiostane, epitiostanol, formestane, interferon-alpha, interferon-2 alpha, interferon-beta, interferon-gamma, colony stimulating factor-1, colony stimulating factor-2, denileukin diftitox, interleukin-2, and leutinizing hormone releasing factor.

Radiation therapy may also be used as part of a combination therapy.

An additional class of agents that may be used as part of a combination therapy in treating cancer is immune checkpoint inhibitors (also referred to as immune checkpoint blockers). Immune checkpoint inhibitors are a class of therapeutic agents that have the effect of blocking immune checkpoints. See, for example, Pardoll in Nature Reviews Cancer (2012) vol. 12, pages 252-264. Exemplary immune checkpoint inhibitors include agents that inhibit one or more of (i) cytotoxic T-lymphocyte-associated antigen 4 (CTLA4), (ii) programmed cell death protein 1 (PD1), (iii) PDL1, (iv) LAB3, (v) B7-H3, (vi) B7-H4, and (vii) TIM3. The CTLA4 inhibitor ipilumumab has been approved by the United States Food and Drug Administration for treating melanoma. In certain embodiments, the immune checkpoint inhibitor comprises pembrolizumab.

Yet other agents that may be used as part of a combination therapy in treating cancer are monoclonal antibody agents that target non-checkpoint targets (e.g., herceptin) and non-cytotoxic agents (e.g., tyrosine-kinase inhibitors).

Accordingly, another aspect of the invention provides a method of treating cancer in a patient, where the method comprises administering to the patient in need thereof (i) a therapeutically effective amount of a compound described herein and (ii) a second anti-cancer agent, in order to treat the cancer, where the second therapeutic agent may be one of the additional therapeutic agents described above (e.g., mitomycin, tretinoin, ribomustin, gemcitabine, an immune checkpoint inhibitor, or a monoclonal antibody agent that targets non-checkpoint targets) or one of the following:
an inhibitor selected from an ALK Inhibitor, an ATR Inhibitor, an A2A Antagonist, a Base Excision Repair Inhibitor, a Bcr-Abl Tyrosine Kinase Inhibitor, a Bruton's Tyrosine Kinase Inhibitor, a CDC7 Inhibitor, a CHK1 Inhibitor, a Cyclin-Dependent Kinase Inhibitor, a DNA-PK Inhibitor, an Inhibitor of both DNA-PK and mTOR, a DNMT1 Inhibitor, a DNMT1 Inhibitor plus 2-chloro-deoxyadenosine, an HDAC Inhibitor, a Hedgehog Signaling Pathway Inhibitor, an IDO Inhibitor, a JAK Inhibitor, a mTOR Inhibitor, a MEK Inhibitor, a MELK Inhibitor, a MTH1 Inhibitor, a PARP Inhibitor, a Phosphoinositide 3-Kinase Inhibitor, an Inhibitor of both PARP1 and DHODH, a Proteasome Inhibitor, a Topoisomerase-II Inhibitor, a Tyrosine Kinase Inhibitor, a VEGFR Inhibitor, a WEE1 Inhibitor, and a ubiquitin-specific protease 1 (USP1) inhibitor;
an agonist of OX40, CD137, CD40, GITR, CD27, HVEM, TNFRSF25, or ICOS;
a therapeutic antibody targeting one of the following: CD20, CD30, CD33, CD52, EpCAM, CEA, gpA33, a mucin, TAG-72, CAIX, PSMA, a folate-binding protein, a ganglioside, Le, VEGF, VEGFR, VEGFR2, integrin αVβ3, integrin α5β1, EGFR, ERBB2, ERBB3, MET, IGF1R, EPHA3, TRAILR1, TRAILR2, RANKL, FAP, tenascin, CD19, KIR, NKG2A, CD47, CEACAM1, c-MET, VISTA, CD73, CD38, BAFF, interleukin-1 beta, B4GALNT1, interleukin-6, and interleukin-6 receptor;
a cytokine selected from IL-12, IL-15, GM-CSF, and G-CSF;
a therapeutic agent selected from sipuleucel-T, aldesleukin (a human recombinant interleukin-2 product having the chemical name des-alanyl-1, serine-125 human interleukin-2), dabrafenib (a kinase inhibitor having the chemical name N-{3-[5-(2-aminopyrimidin-4-yl)-2-tert-butyl-1,3-thiazol-4-yl]-2-fluorophenyl}-2,6-difluorobenzenesulfonamide), vemurafenib (a kinase inhibitor having the chemical name propane-1-sulfonic acid {3-[5-(4-chlorophenyl)-1H-pyrrolo[2,3-b]pyridine-3-carbonyl]-2,4-difluoro-phenyl}-amide), and 2-chloro-deoxyadenosine; or
a placental growth factor, an antibody-drug conjugate, an oncolytic virus, or an anti-cancer vaccine.

In certain embodiments, the second anti-cancer agent is an ALK Inhibitor. In certain embodiments, the second anti-cancer agent is an ALK Inhibitor comprising ceritinib or crizotinib. In certain embodiments, the second anti-cancer agent is an ATR Inhibitor. In certain embodiments, the second anti-cancer agent is an ATR Inhibitor comprising AZD6738 or VX-970. In certain embodiments, the second anti-cancer agent is an A2A Antagonist. In certain embodiments, the second anti-cancer agent is a Base Excision Repair Inhibitor comprising methoxyamine. In certain embodiments, the second anti-cancer agent is a Base Excision Repair Inhibitor, such as methoxyamine. In certain embodiments, the second anti-cancer agent is a Bcr-Abl Tyrosine Kinase Inhibitor. In certain embodiments, the second anti-cancer agent is a Bcr-Abl Tyrosine Kinase Inhibitor comprising dasatinib or nilotinib. In certain embodiments, the second anti-cancer agent is a Bruton's Tyrosine Kinase Inhibitor. In certain embodiments, the second anti-cancer agent is a Bruton's Tyrosine Kinase Inhibitor comprising ibrutinib. In certain embodiments, the second anti-cancer agent is a CDC7 Inhibitor. In certain embodiments, the second anti-cancer agent is a CDC7 Inhibitor comprising RXDX-103 or AS-141.

In certain embodiments, the second anti-cancer agent is a CHK1 Inhibitor. In certain embodiments, the second anti-cancer agent is a CHK1 Inhibitor comprising MK-8776, ARRY-575, or SAR-020106. In certain embodiments, the second anti-cancer agent is a Cyclin-Dependent Kinase Inhibitor. In certain embodiments, the second anti-cancer agent is a Cyclin-Dependent Kinase Inhibitor comprising palbociclib. In certain embodiments, the second anti-cancer agent is a DNA-PK Inhibitor. In certain embodiments, the second anti-cancer agent is a DNA-PK Inhibitor comprising MSC2490484A. In certain embodiments, the second anti-cancer agent is Inhibitor of both DNA-PK and mTOR. In certain embodiments, the second anti-cancer agent comprises CC-115.

In certain embodiments, the second anti-cancer agent is a DNMT1 Inhibitor. In certain embodiments, the second anti-cancer agent is a DNMT1 Inhibitor comprising decitabine, RX-3117, guadecitabine, NUC-8000, or azacytidine. In certain embodiments, the second anti-cancer agent comprises a DNMT1 Inhibitor and 2-chloro-deoxyadenosine. In certain embodiments, the second anti-cancer agent comprises ASTX-727.

In certain embodiments, the second anti-cancer agent is a HDAC Inhibitor. In certain embodiments, the second anti-cancer agent is a HDAC Inhibitor comprising OBP-801, CHR-3996, etinostate, resminostate, pracinostat, CG-200745, panobinostat, romidepsin, mocetinostat, belinostat, AR-42, ricolinostat, KA-3000, or ACY-241.

In certain embodiments, the second anti-cancer agent is a Hedgehog Signaling Pathway Inhibitor. In certain embodiments, the second anti-cancer agent is a Hedgehog Signaling Pathway Inhibitor comprising sonidegib or vismodegib. In certain embodiments, the second anti-cancer agent is an IDO Inhibitor. In certain embodiments, the second anti-cancer agent is an IDO Inhibitor comprising INCB024360. In certain embodiments, the second anti-cancer agent is a JAK Inhibitor. In certain embodiments, the second anti-cancer agent is a JAK Inhibitor comprising ruxolitinib or tofacitinib. In certain embodiments, the second anti-cancer agent is a mTOR Inhibitor. In certain embodiments, the second anti-cancer agent is a mTOR Inhibitor comprising everolimus or temsirolimus. In certain embodiments, the second anti-cancer agent is a MEK Inhibitor. In certain embodiments, the second anti-cancer agent is a MEK Inhibitor comprising cobimetinib or trametinib. In certain embodiments, the second anti-cancer agent is a MELK Inhibitor. In certain embodiments, the second anti-cancer agent is a MELK Inhibitor comprising ARN-7016, APTO-500, or OTS-167. In certain embodiments, the second anti-cancer agent is a MTH1 Inhibitor. In certain embodiments, the second anti-cancer agent is a MTH1 Inhibitor comprising (S)-crizotinib, TH287, or TH588.

In certain embodiments, the second anti-cancer agent is a PARP Inhibitor. In certain embodiments, the second anti-cancer agent is a PARP Inhibitor comprising MP-124, olaparib, BGB-290, talazoparib, veliparib, niraparib, E7449, rucaparb, or ABT-767. In certain embodiments, the second anti-cancer agent is a Phosphoinositide 3-Kinase Inhibitor. In certain embodiments, the second anti-cancer agent is a Phosphoinositide 3-Kinase Inhibitor comprising idelalisib. In certain embodiments, the second anti-cancer agent is an inhibitor of both PARP1 and DHODH (i.e., an agent that inhibits both poly ADP ribose polymerase 1 and dihydroorotate dehydrogenase).

In certain embodiments, the second anti-cancer agent is a Proteasome Inhibitor. In certain embodiments, the second anti-cancer agent is a Proteasome Inhibitor comprising bortezomib or carfilzomib. In certain embodiments, the second anti-cancer agent is a Topoisomerase-II Inhibitor. In certain embodiments, the second anti-cancer agent is a Topoisomerase-II Inhibitor comprising vosaroxin.

In certain embodiments, the second anti-cancer agent is a Tyrosine Kinase Inhibitor. In certain embodiments, the second anti-cancer agent is a Tyrosine Kinase Inhibitor comprising bosutinib, cabozantinib, imatinib or ponatinib. In certain embodiments, the second anti-cancer agent is a VEGFR Inhibitor. In certain embodiments, the second anti-cancer agent is a VEGFR Inhibitor comprising regorafenib. In certain embodiments, the second anti-cancer agent is a WEE1 Inhibitor. In certain embodiments, the second anti-cancer agent is a WEE1 Inhibitor comprising AZD1775. In certain embodiments, the second anti-cancer agent is a USP1 inhibitor comprising TNG-348 or KSQ-4279.

In certain embodiments, the second anti-cancer agent is an agonist of OX40, CD137, CD40, GITR, CD27, HVEM, TNFRSF25, or ICOS. In certain embodiments, the second anti-cancer agent is a therapeutic antibody selected from the group consisting of rituximab, ibritumomab tiuxetan, tositumomab, obinutuzumab, ofatumumab, brentuximab vedotin, gemtuzumab ozogamicin, alemtuzumab, IGN101, adecatumumab, labetuzumab, huA33, pemtumomab, oregovomab, minetumomab, cG250, J591, Mov18, farletuzumab, 3F8, ch14.18, KW-2871, hu3S193, lgN311, bevacizumab, IM-2C6, pazopanib, sorafenib, axitinib, CDP791, lenvatinib, ramucirumab, etaracizumab, volociximab, cetuximab, panitumumab, nimotuzumab, 806, afatinib, erlotinib, gefitinib, osimertinib, vandetanib, trastuzumab, pertuzumab, MM-121, AMG 102, METMAB, SCH 900105, AVE1642, IMC-A12, MK-0646, R1507, CP 751871, KB004, IIIA-4, mapatumumab, HGS-ETR2, CS-1008, denosumab, sibrotuzumab, F19, 81C6, MEDI551, lirilumab, MEDI9447, daratumumab, belimumab, canakinumab, dinutuximab, siltuximab, and tocilizumab.

In certain embodiments, the second anti-cancer agent is a placental growth factor. In certain embodiments, the second anti-cancer agent is a placental growth factor comprising ziv-aflibercept. In certain embodiments, the second anti-cancer agent is an antibody-drug conjugate. In certain embodiments, the second anti-cancer agent is an antibody-drug conjugate selected from the group consisting of brentoxumab vedotin and trastuzumab emtransine. In certain embodiments, the second anti-cancer agent is an antibody-drug conjugate selected from the group consisting of brentoxumab vedotin, trastuzumab emtansine, and trastuzumab deruxtecan.

In certain embodiments, the second anti-cancer agent is an oncolytic virus. In certain embodiments, the second anti-cancer agent is the oncolytic virus talimogene laherparepvec. In certain embodiments, the second anti-cancer agent is an anti-cancer vaccine. In certain embodiments, the second anti-cancer agent is an anti-cancer vaccine selected from the group consisting of a GM-CSF tumor vaccine, a STING/GM-CSF tumor vaccine, and NY-ESO-1. In certain embodiments, the second anti-cancer agent is a cytokine selected from IL-12, IL-15, GM-CSF, and G-CSF.

In certain embodiments, the second anti-cancer agent is a therapeutic agent selected from sipuleucel-T, aldesleukin (a human recombinant interleukin-2 product having the chemical name des-alanyl-1, serine-125 human interleukin-2), dabrafenib (a kinase inhibitor having the chemical name N-{3-[5-(2-aminopyrimidin-4-yl)-2-tert-butyl-1,3-thiazol-4-yl]-2-fluorophenyl}-2,6-difluorobenzenesulfonamide), vemurafenib (a kinase inhibitor having the chemical name propane-1-sulfonic acid {3-[5-(4-chlorophenyl)-1H-pyrrolo[2,3-b]pyridine-3-carbonyl]-2,4-difluoro-phenyl}-amide), and 2-chloro-deoxyadenosine.

### Additional Considerations

The doses and dosage regimen of the active ingredients used in the combination therapy may be determined by an attending clinician. In certain embodiments, the compound described herein (such as a compound of Formula I, I-A, I-aa, II, III, IV, or V, or other compounds in Section I) and the additional therapeutic agent(s) are administered in doses commonly employed when such agents are used as monotherapy for treating the disorder. In other embodiments, the compound described herein (such as a compound of Formula I, I-A, I-aa, II, III, IV, or V, or other compounds in Section I) and the additional therapeutic agent(s) are administered in doses lower than the doses commonly employed when such agents are used as monotherapy for treating the disorder. In certain embodiments, the compound described herein (such as a compound of Formula I, I-A, I-aa, II, III, IV, or V, or other compounds in Section I) and the additional therapeutic agent(s) are present in the same composition, which is suitable for oral administration.

In certain embodiments, the compound described herein (such as a compound of Formula I, I-A, I-aa, II, III, IV, or V, or other compounds in Section I) and the additional therapeutic agent(s) may act additively or synergistically. A synergistic combination may allow the use of lower dosages of one or more agents and/or less frequent administration of one or more agents of a combination therapy. A lower dosage or less frequent administration of one or more agents may lower toxicity of the therapy without reducing the efficacy of the therapy.

Another aspect of this invention is a kit comprising a therapeutically effective amount of the compound described herein (such as a compound of Formula I, I-A, I-aa, II, III, IV, or V, or other compounds in Section I), a pharmaceutically acceptable carrier, vehicle or diluent, and optionally at least one additional therapeutic agent listed above.

### IV. Pharmaceutical Compositions and Dosing Considerations

As indicated above, the invention provides pharmaceutical compositions, which comprise a therapeutically-effective amount of one or more of the compounds described above, formulated together with one or more pharmaceutically acceptable carriers (additives) and/or diluents. The pharmaceutical compositions may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally. In certain embodiments, the invention provides a pharmaceutical composition comprising a compound described herein (such as a compound of Formula I, I-A, I-aa, II, III, IV, or V, or other compounds in Section I) and a pharmaceutically acceptable carrier.

The phrase “therapeutically effective amount” as used herein means that amount of a compound, material, or composition comprising a compound of the present invention which is effective for producing some desired therapeutic effect in at least a sub-population of cells in an animal at a reasonable benefit/risk ratio applicable to any medical treatment.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 0.1 percent to about ninety-nine percent of active ingredient, preferably from about 5 percent to about 70 percent, most preferably from about 10 percent to about 30 percent.

In certain embodiments, a formulation of the present invention comprises an excipient selected from the group consisting of cyclodextrins, celluloses, liposomes, micelle forming agents, e.g., bile acids, and polymeric carriers, e.g., polyesters and polyanhydrides; and a compound of the present invention. In certain embodiments, an aforementioned formulation renders orally bioavailable a compound of the present invention.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules, trouches and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the compounds of the present invention are administered as pharmaceuticals, to humans and animals, they can be given per se or as a pharmaceutical composition containing, for example, 0.1 to 99% (more preferably, 10 to 30%) of active ingredient in combination with a pharmaceutically acceptable carrier.

The preparations of the present invention may be given orally, parenterally, topically, or rectally. They are of course given in forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Oral administrations are preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically-acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above. Preferably, the compounds are administered at about 0.01 mg/kg to about 200 mg/kg, more preferably at about 0.1 mg/kg to about 100 mg/kg, even more preferably at about 0.5 mg/kg to about 50 mg/kg. When the compounds described herein are co-administered with another agent (e.g., as sensitizing agents), the effective amount may be less than when the agent is used alone.

If desired, the effective daily dose of the active compound may be administered as two, three, four, five, six or more sub-doses administered separately at appropriate intervals throughout the day, optionally, in unit dosage forms. Preferred dosing is one administration per day.

The invention further provides a unit dosage form (such as a tablet or capsule) comprising a compound described herein in a therapeutically effective amount for the treatment of a medical disorder described herein.

### EXAMPLES

The invention now being generally described, will be more readily understood by reference to the following examples, which are included merely for purposes of illustration of certain aspects and embodiments of the present invention, and is not intended to limit the invention.

### Example 1—Preparation of 4-Oxo-3-(2-(trifluoromethoxy)ethyl)-3,4-dihydroimidazo[5,1-d][1,2,3,5]tetrazine-8-carboxamide (I-1)

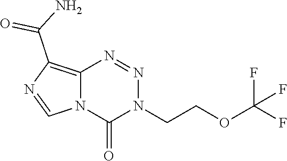 [SMILES: NC(=O)c1ncn2c(=O)n(CCOC(F)(F)F)nnc12]

### Part I—Synthesis of 1-Isocyanato-2-(trifluoromethoxy)ethane

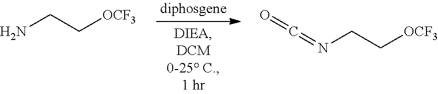 [SMILES: NCCOC(F)(F)F ; O=C=NCCOC(F)(F)F]

A mixture of 2-(trifluoromethoxy)ethanamine (1.50 g, 9.06 mmol, 1.00 eq, HCl salt) and DIEA (2.46 g, 19.0 mmol, 3.31 mL, 2.10 eq) in DCM (15.0 mL) was added dropwise via syringe pump over 10 min to a solution of diphosgene (1.13 g, 5.71 mmol, 689 μL, 0.63 eq) in DCM (15.0 mL) at 0° C. After addition was completed, the cooling bath was removed, and the reaction mixture was allowed to warm to 25° C. and stirred for 1 hr. The reaction mixture was transferred to a separatory funnel. The organic layer was washed sequentially with 1 N aqueous hydrochloric acid solution (30 mL, precooled to 0° C.) and saturated aqueous sodium chloride solution (30 mL, precooled to 0° C.). The washed organic layer was dried over magnesium sulfate. The dried solution was filtered, and the filtrate was concentrated at ˜10° C. to 15° C. Compound 1-isocyanato-2-(trifluoromethoxy)ethane (1.40 g, 9.03 mmol, 99.6% yield) was obtained as a yellow oil, and used in the next step directly without further purification.

### Part II—Synthesis of 4-Oxo-3-(2-(trifluoromethoxy)ethyl)-3,4-dihydroimidazo[5,1-d][1,2,3,5]tetrazine-8-carboxamide

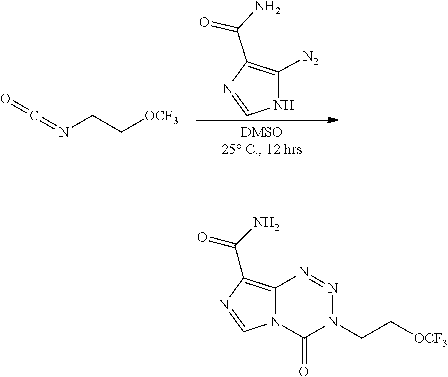

To a solution of 4-carbamoyl-1H-imidazole-5-diazonium (500 mg, 3.62 mmol, 1.00 eq) in DMSO (3.00 mL) was added 1-isocyanato-2-(trifluoromethoxy)ethane (1.40 g, 9.03 mmol, 2.50 eq), and then the resulting mixture was stirred at 25° C. for 12 hrs. Then, the reaction mixture was filtered. The filtrate was purified by prep-HPLC (column: Phenomenex luna C18 250*50 mm*10 μm; Mobile phase: [H₂O (0.1% TFA)-ACN]; gradient: 10%-40% B over 10.0 min), to afford the title compound as a pink solid (804.9 mg, 2.75 mmol, 76.1% yield). ¹H NMR (400 MHz, DMSO-d₆) δ 8.88 (s, 1H), 7.84 (s, 1H), 7.71 (s, 1H), 4.67-4.62 (m, 2H), 4.51-4.46 (m, 2H). MS (ESI): mass calcd. for C₈H₇F₃N₆O₃ 292.05, m/z found 293.0 [M+H]⁺.

### Example 2—Preparation of 4-Oxo-3-(2-(trifluoromethoxy)ethyl)-3,4-dihydroimidazo[5,1-d][1,2,3,5]tetrazine-8-carboxylic Acid (I-33)

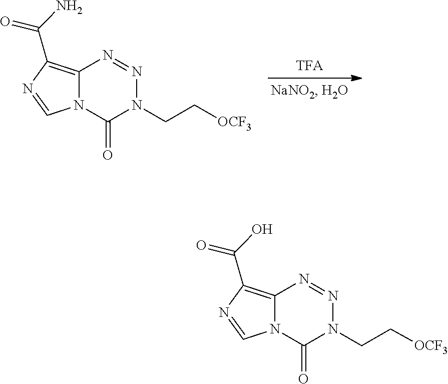 [SMILES: NC(=O)c1ncn2c(=O)n(CCOC(F)(F)F)nnc12 ; O=C(O)c1ncn2c(=O)n(CCOC(F)(F)F)nnc12]

A solution of 4-oxo-3-[2-(trifluoromethoxy)ethyl]imidazo[5,1-d][1,2,3,5]tetrazine-8-carboxamide (1.2 g, 4.11 mmol, 1 eq) in TFA (8.57 g, 75.1 mmol, 5.58 mL, 18.3 eq) was added NaNO₂ (652 mg, 9.45 mmol, 2.3 eq) in H₂O (3 mL) at 0° C. The mixture was stirred at 35° C. for 12 h. Desired solid product precipitated from the reaction mixture, and it was isolated by filtration to give 4-oxo-3-[2-(trifluoromethoxy)ethyl]imidazo[5,1-d][1,2,3,5]tetrazine-8-carboxylic acid (0.66 g, 2.25 mmol, 54.8% yield) as a yellow solid.

### Example 3—Preparation of N-Ethyl-4-oxo-3-(2-(trifluoromethoxy)ethyl)-3,4-dihydroimidazo[5,1-d][1,2,3,5]tetrazine-8-carboxamide (I-3)

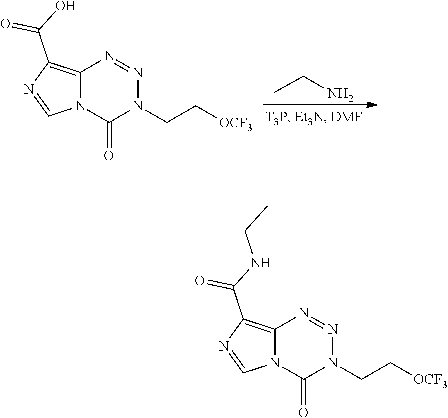

To a mixture of 4-oxo-3-[2-(trifluoromethoxy)ethyl]imidazo[5,1-d][1,2,3,5]tetrazine-8-carboxylic acid (100 mg, 341 μmol, 1.0 eq) and ethylamine-HCl (27.8 mg, 341 μmol, 1.0 eq) in DMF (3 mL) was added triethylamine (138 mg, 1.36 mmol, 190 μL, 4.0 eq) and T₃P (propylphosphonic anhydride solution, 492 mg, 682 μmol, 50% purity, 2.0 eq) at 0° C., and then the mixture was stirred at 25° C. for 0.5 hour. LCMS showed the reaction was completed. The mixture was filtered, and the filtrate was purified by prep-HPLC (column: Phenomenex Luna C18 75*30 mm*3 m; mobile phase: [H₂O (0.1% TFA)-ACN]; gradient: 10%-40% B over 8.0 min) to afford the title compound as a purple solid (52 mg, 156 μmol, 45.8% yield, 96.16% purity). ¹H NMR (400 MHz, DMSO-d₆) δ 8.90 (s, 1H), 8.56 (br t, J=6.0 Hz, 1H), 4.73-4.63 (m, 2H), 4.57-4.41 (m, 2H), 3.33-3.28 (m, 2H), 1.14 (t, J=7.2 Hz, 3H). LC/MS [M+H]⁺ 321.08 (calculated); LC/MS [M+H]⁺ 321.08 (observed).

### Example 4—Preparation of 4-Oxo-N-phenyl-3-(2-(trifluoromethoxy)ethyl)-3,4-dihydroimidazo[5,1-d][1,2,3,5]tetrazine-8-carboxamide (I-9)

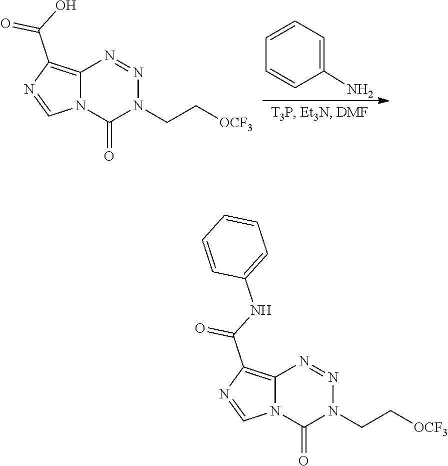 [SMILES: O=C(O)c1ncn2c(=O)n(CCOC(F)(F)F)nnc12 ; Nc1ccccc1 ; O=C(Nc1ccccc1)c1ncn2c(=O)n(CCOC(F)(F)F)nnc12]

To a solution of 4-oxo-3-[2-(trifluoromethoxy)ethyl]imidazo[5,1-d][1,2,3,5]tetrazine-8-carboxylic acid (0.1 g, 341 μmol, 1 eq) and aniline (63.5 mg, 682 μmol, 2 eq) in DMF (1 mL) was added triethylamine (103 mg, 1.02 mmol, 142 μL, 3 eq) and T₃P (propylphosphonic anhydride solution, 245 mg, 682 μmol, 2 eq). The mixture was stirred at 20° C. for 1 hr. LCMS showed the reaction was completed. The reaction mixture was quenched by addition of TFA (0.1 mL) at 0° C. Then the mixture was filtered, and the filtrate was purified by prep-HPLC (column: Phenomenex Luna C18 75*30 mm*3 m; mobile phase: [H₂O (0.1% TFA)-ACN]; gradient: 25%-65% B over 8.0 min) to afford the title compound as a pink solid (19.6 mg, 53.2 mol, 15.6% yield). ¹H NMR (400 MHz, DMSO-d₆) δ 10.41 (s, 1H), 9.04 (s, 1H), 7.91-7.83 (m, 2H), 7.42-7.32 (m, 2H), 7.17-7.08 (m, 1H), 4.70-4.64 (m, 2H), 4.54-4.49 (m, 2H). LC/MS [M+H]⁺ 369.1 (calculated); LC/MS [M+H]⁺ 369.1 (observed).

### Example 5—Preparation of N-(2-(Dimethylamino)ethyl)-N-methyl-4-oxo-3-(2-(trifluoromethoxy)ethyl)-3,4-dihydroimidazo[5,1-d][1,2,3,5]tetrazine-8-carboxamide (I-11)

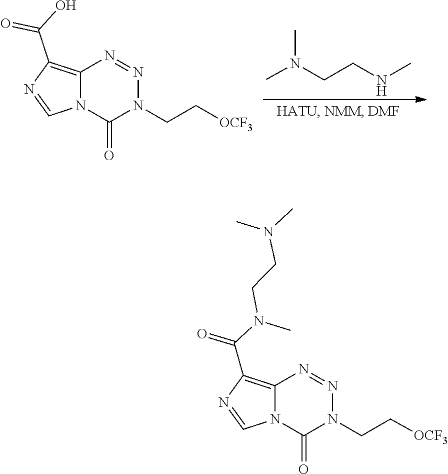

To a solution of 4-oxo-3-[2-(trifluoromethoxy)ethyl]imidazo[5,1-d][1,2,3,5]tetrazine-8-carboxylic acid (0.1 g, 341 μmol, 1 eq) in DMF (1 mL) were added HATU (130 mg, 341 μmol, 1 eq), N-methylmorpholine (34.5 mg, 341 μmol, 1 eq), and N1,N1,N2-trimethylethane-1,2-diamine (34.7 mg, 341 μmol, 1 eq), and then the mixture was stirred at 20° C. for 1 hr. LCMS showed the reaction was completed. The reaction mixture was quenched by addition of TFA (0.1 mL) at 0° C. Then the mixture was filtered, and the filtrate was purified by prep-HPLC (column: Phenomenex Luna C18 75*30 mm*3 m; mobile phase: [H₂O (0.1% TFA)-ACN]; gradient: 1%-35% B over 8.0 min) to provide the title compound as a pink oil (0.083 g, 168.92 mol, 49.52% yield, TFA). ¹H NMR (400 MHz, DMSO-d₆) δ 8.84 (s, 1H), 4.74-4.62 (m, 2H), 4.57-4.50 (m, 2H), 3.92-3.85 (m, 2H), 3.48-3.40 (m, 2H), 3.17 (s, 3H), 2.85 (s, 6H). LC/MS [M+H]378.14 (calculated); LC/MS [M+H]⁺ 377.8 (observed).

### Example 6—Preparation of 8-(4-Methylpiperazine-1-carbonyl)-3-(2-(trifluoromethoxy) ethyl)imidazo[5,1-d][1,2,3,5]tetrazin-4(3H)-one (I-10)

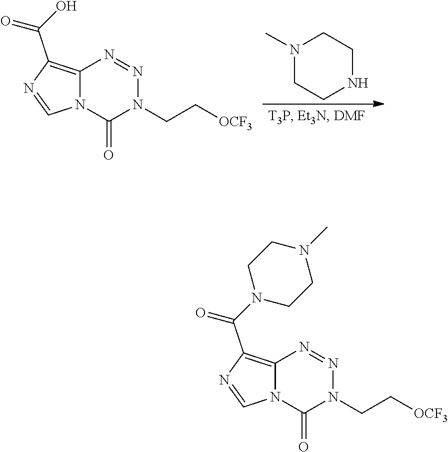

To a solution of 4-oxo-3-[2-(trifluoromethoxy)ethyl]imidazo[5,1-d][1,2,3,5]tetrazine-8-carboxylic acid (100 mg, 343 μmol, 1 eq) and 1-methylpiperazine (68.8 mg, 686 μmol, 2 eq) in DMF (1 mL) was added Et₃N (104 mg, 1.03 mmol, 143 μL, 3 eq), followed by T₃P (propylphosphonic anhydride solution, 495 mg, 686 μmol, 50% purity, 2 eq) at 0° C., and then the mixture was stirred at 20° C. for 1 hr. LCMS showed the starting material was consumed and desired MS was observed. The mixture was filtered, and the filtrate was purified by prep-HPLC (column: Phenomenex Gemini-NX 150*30 mm*5 m; mobile phase: [H₂O (0.1% TFA)-ACN]; gradient: 5%-35% B over 20.0 min) to afford the title compound as a purple oil (18.3 mg, 45.35 mol, 13.20% yield). ¹H NMR (400 MHz, DMSO-d₆) δ 8.96 (s, 1H), 4.68-4.62 (m, 2H), 4.53-4.42 (m, 2H), 3.66-3.43 (m, 4H), 3.30-3.05 (m, 4H), 2.85 (s, 3H). LC/MS [M+H]⁺ 376.1 (calculated); LC/MS [M+H]⁺ 376.1 (observed).

### Example 7—Preparation of 8-(Pyrrolidine-1-carbonyl)-3-(2-(trifluoromethoxy) ethyl)imidazo[5,1-d][1,2,3,5]tetrazin-4(3H)-one (I-7)

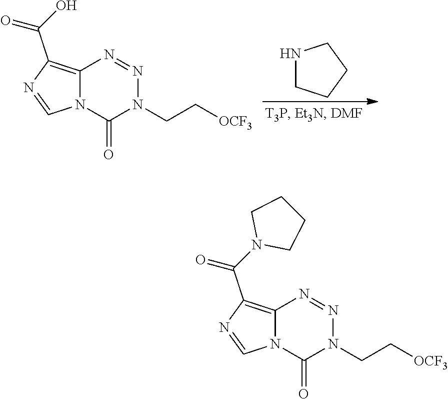 [SMILES: O=C(O)c1ncn2c(=O)n(CCOC(F)(F)F)nnc12 ; C1CCNC1 ; O=C(c1ncn2c(=O)n(CCOC(F)(F)F)nnc12)N1CCCC1]

To a solution of 4-oxo-3-[2-(trifluoromethoxy)ethyl]imidazo[5,1-d][1,2,3,5]tetrazine-8-carboxylic acid (0.11 g, 375 μmol, 1 eq) and pyrrolidine (53.4 mg, 750 μmol, 2 eq) in DMF (1 mL) were added Et₃N (114 mg, 1.13 mmol, 157 μL, 3 eq) and T₃P (propylphosphonic anhydride solution, 540 mg, 750 μmol, 50% purity, 2 eq) at 0° C., and then the mixture was stirred at 25° C. for 1 hr. LCMS showed the starting material was consumed completely and the desired MS was observed. The reaction mixture was filtered, and the filtrate was purified by prep-HPLC (column: Phenomenex Luna C18 75*30 mm*3 m; mobile phase: [H₂O (0.1% TFA)-ACN]; gradient: 20%-60% B over 8.0 min) to afford the title compound as a white solid (16.3 mg, 46.3 μmol, 12.3% yield). ¹H NMR (400 MHz, DMSO-d₆) δ 8.88 (s, 1H), 4.68-4.57 (m, 2H), 4.54-4.44 (m, 2H), 3.64 (br t, J=6.8 Hz, 2H), 3.54 (br t, J=6.8 Hz, 2H), 1.92-1.83 (m, 4H). LC/MS [M+H]347.1 (calculated); LC/MS [M+H]⁺ 347.1 (observed).

### Example 8—Preparation of N,N-Dimethyl-4-oxo-3-(2-(trifluoromethoxy)ethyl)-3,4-dihydroimidazo[5,1-d][1,2,3,5]tetrazine-8-carboxamide (I-4)

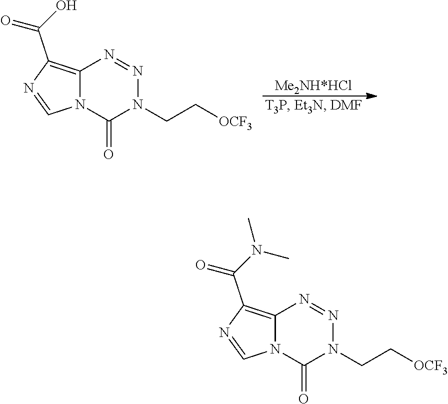 [SMILES: O=C(O)c1ncn2c(=O)n(CCOC(F)(F)F)nnc12 ; CN(C)C(=O)c1ncn2c(=O)n(CCOC(F)(F)F)nnc12]

To a solution of 4-oxo-3-[2-(trifluoromethoxy)ethyl]imidazo[5,1-d][1,2,3,5]tetrazine-8-carboxylic acid (100 mg, 341 μmol, 1 eq) and N,N-dimethylamine hydrochloride (55.6 mg, 682 μmol, 2 eq) in DMF (1 mL) were added triethylamine (172 mg, 1.71 mmol, 237 μL, 5 eq), and T₃P (propylphosphonic anhydride solution, 491 mg, 682 μmol, 50% purity, 2 eq) at 0° C., and then the mixture was stirred at 25° C. for 1 hr. LCMS showed the reaction was completed. The reaction mixture was filtered, and the filtrate was purified by prep-HPLC (column: Phenomenex Luna C18 80*30 mm*3 m; mobile phase: [H₂O (0.1% TFA)-ACN]; gradient: 10%-40% B over 8.0 min) to afford the title compound as a purple solid (76 mg, 230.5 μmol, 67.6% yield). ¹H NMR (400 MHz, DMSO-d₆) δ 8.88 (s, 1H), 4.62 (t, J=5.2 Hz, 2H), 4.48 (t, J=5.2 Hz, 2H), 3.06 (s, 6H). LC/MS [M+H]⁺ 321.1 (calculated); LC/MS [M+H]⁺ 321.2 (observed).

### Example 9—Preparation of N-Cyclopropyl-4-oxo-3-(2-(trifluoromethoxy)ethyl)-3,4-dihydroimidazo[5,1-d][1,2,3,5]tetrazine-8-carboxamide (I-6)

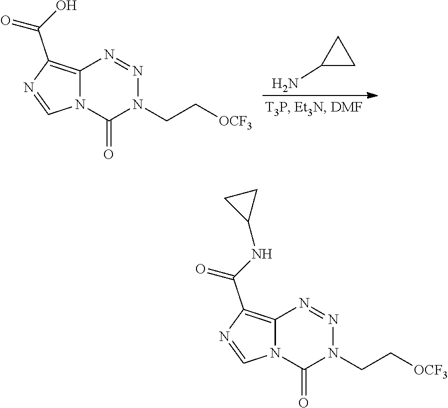

To a mixture of 4-oxo-3-[2-(trifluoromethoxy)ethyl]imidazo[5,1-d][1,2,3,5]tetrazine-8-carboxylic acid (80.0 mg, 273 μmol, 1.0 eq) and cyclopropanamine (31.2 mg, 546 μmol, 2.0 eq) in DMF (3 mL) were added Et₃N (110 mg, 1.09 mmol, 152 μL, 4.0 eq) and T₃P (propylphosphonic anhydride solution, 393 mg, 546 μmol, 50% purity, 2.0 eq) at 0° C., and then the mixture was stirred at 25° C. for 0.5 hour. LCMS showed the reaction was completed. The mixture was filtered, and the filtrate was purified by prep-HPLC (column: Phenomenex Gemini-NX 80*40 mm*3 m; mobile phase: [H₂O (0.1% TFA)-ACN]; gradient: 10%-40% B over 20.0 min) to afford the title compound as a purple solid (10.1 mg, 29.7 μmol, 10.8% yield). ¹H NMR (400 MHz, DMSO-d₆) δ 8.89 (s, 1H), 8.55 (br d, J=4.4 Hz, 1H), 4.69-4.59 (m, 2H), 4.55-4.44 (m, 2H), 2.96-2.86 (m, 1H), 0.76-0.62 (m, 4H). LC/MS [M+H]⁺ 333.2 (calculated); LC/MS [M+H]⁺ 333.2 (observed).

### Example 10—Preparation of Hydrochloride Salt of N-(2-Aminoethyl)-4-oxo-3-(2-(trifluoromethoxy)ethyl)-3,4-dihydroimidazo[5,1-d][1,2,3,5]tetrazine-8-carboxamide (I-12)

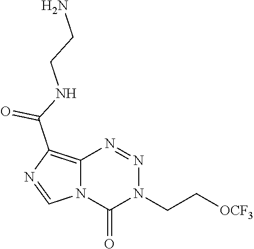 [SMILES: NCCNC(=O)c1ncn2c(=O)n(CCOC(F)(F)F)nnc12]

### Part I—Synthesis of tert-Butyl (2-(4-Oxo-3-(2-(trifluoromethoxy)ethyl)-3,4-dihydroimidazo[5,1-d][1,2,3,5]tetrazine-8-carboxamido)ethyl)carbamate

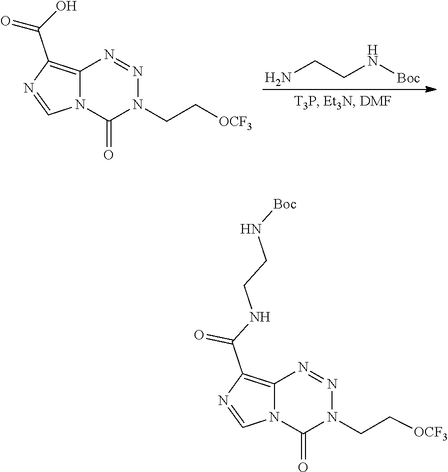 [SMILES: O=C(O)c1ncn2c(=O)n(CCOC(F)(F)F)nnc12 ; CC(C)(C)OC(=O)NCCN ; CC(C)(C)OC(=O)NCCNC(=O)c1ncn2c(=O)n(CCOC(F)(F)F)nnc12]

To a solution of 4-oxo-3-[2-(trifluoromethoxy)ethyl]imidazo[5,1-d][1,2,3,5]tetrazine-8-carboxylic acid (150 mg, 512 μmol, 1.00 eq) in DMF (3.00 mL) were added tert-butyl N-(2-aminoethyl)carbamate (123 mg, 767 μmol, 121 μL, 1.50 eq), Et₃N (155 mg, 1.54 mmol, 214 μL, 3.00 eq) and T₃P (propylphosphonic anhydride solution, 737 mg, 1.02 mmol, 50% purity, 2.00 eq) at 0° C., and then the mixture was stirred at 25° C. for 1 hr. LCMS showed the reaction was completed. The reaction mixture was diluted with H₂O (16.0 mL), and extracted with EtOAc (10.0 mL×3). The combined organic layers were washed with brine (15.0 mL), dried over Na₂SO₄, filtered, and concentrated under reduced pressure to give a residue. The residue was triturated with MTBE (5.00 mL) at 15° C. for 5 min to give the title compound as a white solid (143 mg, 328 μmol, 64.2% yield).

### Part II—Synthesis of Hydrochloride Salt of N-(2-Aminoethyl)-4-oxo-3-(2-(trifluoromethoxy)ethyl)-3,4-dihydroimidazo[5,1-d][1,2,3,5]tetrazine-8-carboxamide (I-12)

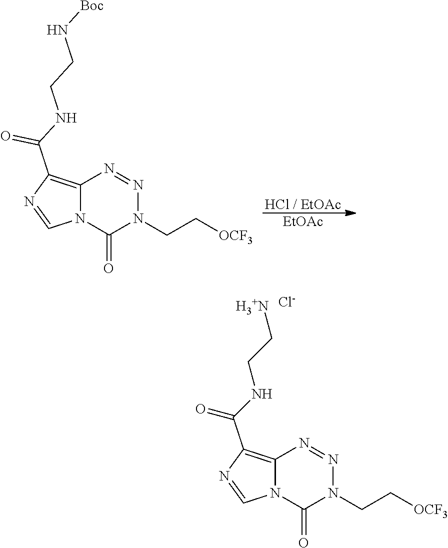

To a solution of tert-butyl N-[2-[[4-oxo-3-[2-(trifluoromethoxy)ethyl]imidazo[5,1-d][1,2,3,5]tetrazine-8-carbonyl]amino]ethyl]carbamate (143 mg, 328 μmol, 1.00 eq) in EtOAc (3.00 mL) was added HCl/EtOAc (4.00 μM, 5.00 mL), and then the mixture was stirred at 25° C. for 20 min. LCMS showed the reaction was completed. The reaction mixture was concentrated under reduced pressure. The residue was triturated with EtOAc (5 mL) at 15° C. for 5 min to afford the title compound as a white solid (67.8 mg, 182.3 μmol, 55.5% yield). ¹H NMR (400 MHz, DMSO-d₆) δ 8.96 (s, 1H), 8.77 (t, J=5.6 Hz, 1H), 7.96 (br s, 3H), 4.75-4.60 (m, 2H), 4.57-4.44 (m, 2H), 3.59-3.54 (m, 2H), 3.06-2.93 (m, 2H). LC/MS [M+H]⁺ 336.1 (calculated); LC/MS [M]⁺336.1 (observed).

### Example 11—Preparation of N-Cyclopentyl-4-oxo-3-(2-(trifluoromethoxy)ethyl)-3,4-dihydroimidazo[5,1-d][1,2,3,5]tetrazine-8-carboxamide (I-5)

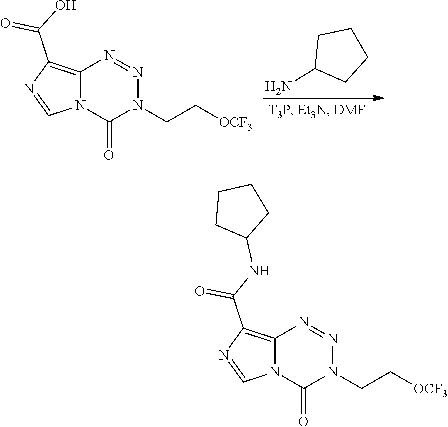 [SMILES: O=C(O)c1ncn2c(=O)n(CCOC(F)(F)F)nnc12 ; NC1CCCC1 ; O=C(NC1CCCC1)c1ncn2c(=O)n(CCOC(F)(F)F)nnc12]

To a solution of 4-oxo-3-[2-(trifluoromethoxy)ethyl]imidazo[5,1-d][1,2,3,5]tetrazine-8-carboxylic acid (90 mg, 307 μmol, 1 eq) and cyclopentanamine (39.2 mg, 460 μmol, 1.5 eq) in DMF (1 mL) was added triethylamine (93.2 mg, 921 μmol, 128 μL, 3 eq), followed by T₃P (propylphosphonic anhydride solution, 442 mg, 614 μmol, 50% purity, 2 eq) at 0° C., and then the mixture was stirred at 20° C. for 1 hr. LCMS showed the starting material was consumed, and the desired MS was observed. The mixture was filtered, and the filtrate was purified by prep-HPLC (column: Phenomenex luna C18 100*40 mm*3 m; mobile phase: [H₂O (0.1% TFA)-ACN]; gradient: 20%-55% B over 8.0 min) to afford the title compound as a white solid (49.6 mg, 132.9 μmol, 43.3% yield). ¹H NMR (400 MHz, DMSO-d₆) δ 8.90 (s, 1H) 8.31 (d, J=7.6 Hz, 1H), 4.68-4.63 (m, 2H) 4.53-4.47 (m, 2H), 4.32-4.23 (m, 1H), 1.93-1.86 (m, 2H), 1.73-1.66 (m, 2H) 1.64-1.51 (m, 4H). LC/MS [M+H]⁺ 361.1 (calculated); LC/MS [M+H]⁺ 361.1 (observed).

### Example 12—Preparation of 4-Oxo-N-(2-pyridyl)-3-[2-(trifluoromethoxy)ethyl]imidazo[5,1-d][1,2,3,5]tetrazine-8-carboxamide (I-8)

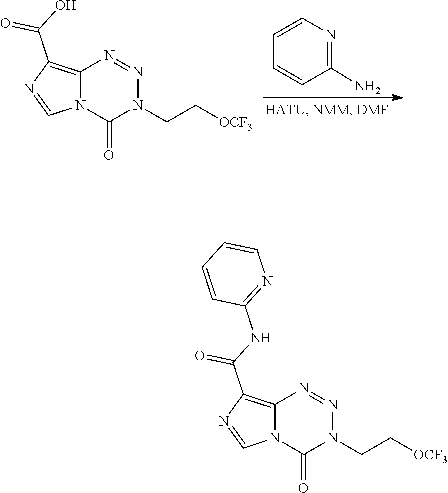 [SMILES: O=C(O)c1ncn2c(=O)n(CCOC(F)(F)F)nnc12 ; Nc1ccccn1 ; O=C(Nc1ccccn1)c1ncn2c(=O)n(CCOC(F)(F)F)nnc12]

To a solution of 4-oxo-3-[2-(trifluoromethoxy)ethyl]imidazo[5,1-d][1,2,3,5]tetrazine-8-carboxylic acid (0.1 g, 341 μmol, 1 eq) in DMF (2 mL) were added HATU (156 mg, 409 μmol, 1.2 e q), N-methylmorpholine (69.0 mg, 682 μmol, 75.0 μL, 2 eq) and pyridin-2-amine (64.2 mg, 682 μmol, 2 eq), and then the mixture was stirred at 20° C. for 2 hr. LCMS showed the reaction was completed. The reaction mixture was filtered, and the filtrate was purified by prep-HPLC (column: Xselect CSH C18 100*30 mm*5 m; mobile phase: [H₂O (0.04% HCl)-ACN]; gradient: 20%-50% B over 15.0 min) to afford the title compound as a white solid (34 mg, 92.1 μmol, 27.0% yield). ¹H NMR (400 MHz, DMSO-d₆) δ 10.38 (s, 1H), 9.04 (s, 1H), 8.52-8.37 (m, 1H), 8.25 (d, J=8.4 Hz, 1H), 8.02 (br t, J=7.2 Hz, 1H), 7.35-7.25 (m, 1H), 4.77-4.64 (m, 2H), 4.57-4.47 (m, 2H). LC/MS [M+H]⁺ 370.08 (calculated); LC/MS [M+H]⁺ 370.2 (observed).

### Example 13—Preparation of 4-Oxo-3-(2-(trifluoromethoxy)ethyl-1,1,2,2-d4)-3,4-dihydroimidazo[5,1-d][1,2,3,5]tetrazine-8-carboxamide (I-29)

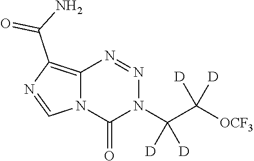 [SMILES: [2H]C([2H])(OC(F)(F)F)C([2H])([2H])n1nnc2c(C(N)=O)ncn2c1=O]

The title compound was prepared. ¹H NMR (400 MHz, DMSO-d₆) δ 8.88 (s, 1H), 7.85 (s, 1H), 7.71 (s, 1H). LC/MS [M+H]⁺ 297.1 (calculated); LC/MS [M+H]⁺ 297.1 (observed).

### Example 14—Preparation of 4-Oxo-3-(2-(trifluoromethoxy)ethyl)-3,4-dihydroimidazo[5,1-d][1,2,3,5]tetrazine-8-carbothioamide (I-31)

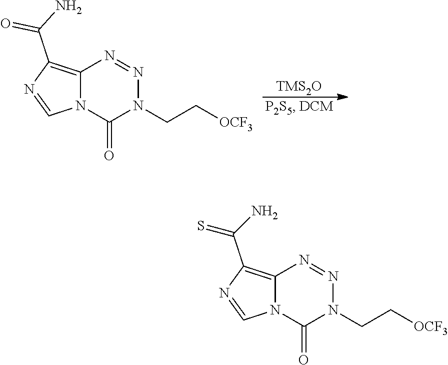 [SMILES: NC(=O)c1ncn2c(=O)n(CCOC(F)(F)F)nnc12 ; NC(=S)c1ncn2c(=O)n(CCOC(F)(F)F)nnc12]

To a solution of 4-oxo-3-[2-(trifluoromethoxy)ethyl]imidazo[5,1-d][1,2,3,5]tetrazine-8-carboxamide (0.5 g, 1.71 mmol, 1 eq) in DCM (5 mL) was added P₂S₅ (304 mg, 1.37 mmol, 146 μL, 0.8 eq) and trimethyl(trimethylsilyloxy)silane (611 mg, 3.76 mmol, 800 μL, 2.2 eq). The mixture was stirred at 45° C. for 16 hrs. The reaction mixture was concentrated under reduced pressure to obtain a solid, which was triturated with MTBE (20 mL) at 25° C. for 10 min to afford the title compound as a yellow solid. (0.48 g, 1.56 mmol, 91.0% yield). ¹H NMR (400 MHz, DMSO-d₆) δ 9.98 (br s, 1H), 9.51 (br s, 1H), 8.86 (s, 1H), 4.67-4.63 (m, 2H), 4.53-4.49 (m, 2H). LC/MS [M+H]⁺ 309.0 (calculated); LC/MS [M+H]⁺ 309.0 (observed).

### Example 15—Preparation of 8-(5-Methylthiazol-2-yl)-3-(2-(trifluoromethoxy) ethyl)imidazo[5,1-d][1,2,3,5]tetrazin-4(3H)-one (II-6)

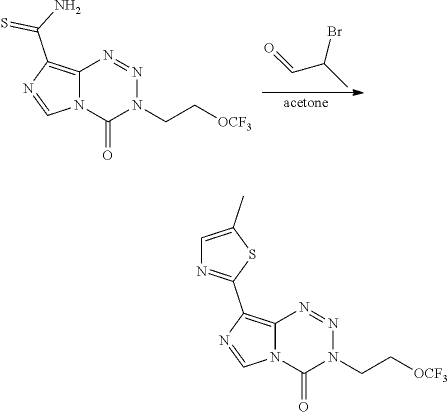

To a solution of 4-oxo-3-(2-(trifluoromethoxy)ethyl)-3,4-dihydroimidazo[5,1-d][1,2,3,5]tetrazine-8-carbothioamide (0.38 g, 1.23 mmol, 1 eq) in acetone (10 mL) was added 2-bromopropanal (507 mg, 3.70 mmol, 3 eq), and then the mixture was stirred at 70° C. for 16 hrs. The mixture was filtered and concentrated in vacuum. The residue was purified by prep-HPLC (column: Phenomenex luna C18 100*40 mm*5 um; mobile phase: [H₂O (0.2% FA)-ACN]; gradient: 30%-60% B over 8.0 min) to afford the title compound as a yellow solid (50.9 mg, 144 μmol, 11.71% yield). ¹H NMR (400 MHz, DMSO-d₆) δ 8.91 (s, 1H), 7.76 (d, J=1.2 Hz, 1H), 4.62 (t, J=5.2 Hz, 2H), 4.49 (t, J=5.2 Hz, 2H), 2.54 (s, 3H). LC/MS [M+H]⁺ 347.1 (calculated); LC/MS [M+H]⁺ 347.1 (observed).

### Example 16—Preparation of Methyl 4-Oxo-3-(2-(trifluoromethoxy)ethyl)-3,4-dihydroimidazo[5,1-d][1,2,3,5]tetrazine-8-carboximidothioate (I-32)

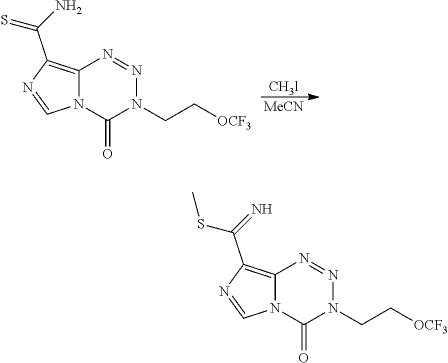 [SMILES: NC(=S)c1ncn2c(=O)n(CCOC(F)(F)F)nnc12 ; CSC(=N)c1ncn2c(=O)n(CCOC(F)(F)F)nnc12]

To a solution of 4-oxo-3-[2-(trifluoromethoxy)ethyl]imidazo[5,1-d][1,2,3,5]tetrazine-8-carbothioamide (0.48 g, 1.56 mmol, 1 eq) in MeCN (6 mL) was added CH₃I (2.21 g, 15.57 mmol, 10 eq). The mixture was stirred at 25° C. for 16 hr. The reaction mixture was filtered and then concentrated under reduced pressure. The crude product was triturated with MTBE (10 mL) at 25° C. for 10 min to afford the title compound as a yellow solid (0.5 g, 1.55 mmol, 99.6% yield). ¹H NMR (400 MHz, DMSO-d₆) δ 9.22 (s, 1H) 4.72 (t, J=4.8 Hz, 2H) 4.51 (t, J=5.0 Hz, 2H) 2.84 (s, 3H).

### Example 17—Preparation of 4-Oxo-3-(2-(trifluoromethoxy)ethyl)-3,4-dihydroimidazo[5,1-d][1,2,3,5]tetrazine-8-carboximidamide (I-34)

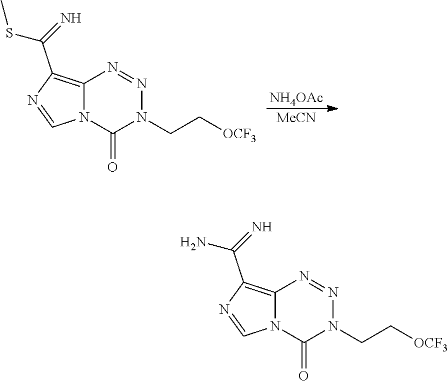 [SMILES: CSC(=N)c1ncn2c(=O)n(CCOC(F)(F)F)nnc12 ; N=C(N)c1ncn2c(=O)n(CCOC(F)(F)F)nnc12]

To a solution of methyl 4-oxo-3-[2-(trifluoromethoxy)ethyl]imidazo[5,1-d][1,2,3,5]tetrazine-8-carboximidothioate (0.22 g, 683 μmol, 1 eq) in MeCN (5 mL) was added ammonium acetate (158 mg, 2.05 mmol, 3 eq). The mixture was stirred at 40° C. for 2 hr. The reaction mixture was concentrated under reduced pressure to afford a crude solid. The crude product was purified by prep-HPLC (column: Phenomenex Luna C18 75*30 mm*3 um; mobile phase: [H₂O (0.1% TFA)-ACN]; gradient: 1%-30% B over 8.0 min) to afford the title compound as a white solid (17.6 mg, 60.44 μmol, 8.85% yield). ¹H NMR (400 MHz, DMSO-d₆) δ 9.25 (d, J=12.88 Hz, 3H) 9.22 (s, 1H) 4.75-4.70 (m, 2H) 4.53 (t, J=4.88 Hz, 2H). LC/MS [M+H]⁺ 292.1 (calculated); LC/MS [M+H]⁺ 292.1 (observed).

### Example 18—Preparation of 4-Oxo-3-(2-(trifluoromethoxy)ethyl)-3,4-dihydroimidazo[5,1-d][1,2,3,5]tetrazine-8-carbonitrile (I-40)

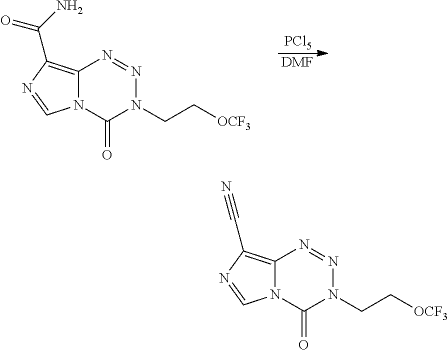

To a solution of 4-oxo-3-[2-(trifluoromethoxy)ethyl]imidazo[5,1-d][1,2,3,5]tetrazine-8-carboxamide (1 g, 3.42 mmol, 1 eq) in DMF (10 mL) was added PCl₅ (713 mg, 3.42 mmol, 1 eq) at 0° C. The mixture was warmed to 25° C. and stirred for 10 hr. The reaction mixture was filtered and concentrated under reduced pressure. The residue was purified by prep-HPLC (column: Phenomenex luna C18 250*50 mm*15 um; mobile phase: [H₂O (0.1% TFA)-ACN]; gradient: 30%-60% B over 10.0 min) to afford the title compound as a black oil (485 mg, 1.77 mmol, 51.7% yield). ¹H NMR (400 MHz, DMSO-d₆) δ 9.12 (s, 1H) 4.71 (t, J=5.00 Hz, 2H) 4.56-4.45 (m, 2H). LC/MS [M+H]⁺ 275.0 (calculated); LC/MS [M+H]⁺ 275.1 (observed).

### Example 19—Preparation of Ethyl 4-Oxo-3-(2-(trifluoromethoxy)ethyl)-3,4-dihydroimidazo[5,1-d][1,2,3,5]tetrazine-8-carboxylate (I-30)

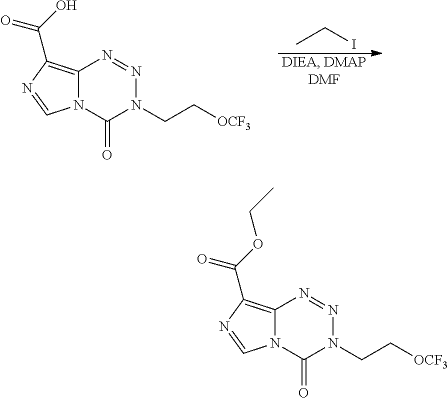

To a solution of 4-oxo-3-(2-(trifluoromethoxy)ethyl)-3,4-dihydroimidazo[5,1-d][1,2,3,5]tetrazine-8-carboxylic acid (80 mg, 273 μmol, 1 eq) in DMF (1 mL) was added iodoethane (63.8 mg, 409 μmol, 1.5 eq), N,N-diisopropylethylamine (70.5 mg, 546 μmol, 2 eq) and 4-dimethylaminopyridine (6.67 mg, 54.6 μmol, 0.2 eq). The mixture was stirred at 25° C. for 16 hr. The mixture was filtered, and the filtrate was purified by prep-HPLC (column: Phenomenex Luna C18 75*30 mm*3 um; mobile phase: [H₂O (0.1% TFA)-ACN]; gradient: 25%-55% B over 8.0 min) to afford the title compound as a white solid (69 mg, 215 μmol, 78.7% yield). ¹H NMR (400 MHz, DMSO-d₆) δ 8.91 (s, 1H), 4.69-4.64 (m, 2H), 4.53-4.48 (m, 2H), 4.45-4.37 (m, 2H), 1.35 (t, J=7.2 Hz, 3H). LC/MS [M+H]⁺ 322.1 (calculated); LC/MS [M+H]⁺ 322.1 (observed).

### Example 20—Preparation of 8-Chloro-3-(2-(trifluoromethoxy)ethyl)imidazo[5,1-d][1,2,3,5]tetrazin-4(3H)-one (I-39)

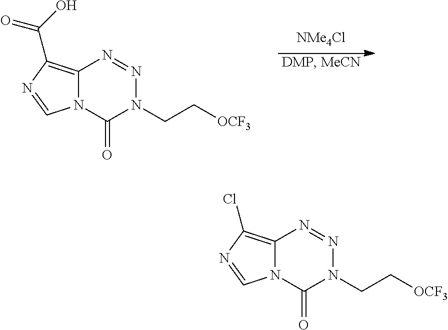

To a solution of 4-oxo-3-(2-(trifluoromethoxy)ethyl)-3,4-dihydroimidazo[5,1-d][1,2,3,5]tetrazine-8-carboxylic acid (100 mg, 341 μmol, 1 eq) in acetone (2 mL) was added Dess-Martin periodinane (579 mg, 1.36 mmol, 4 eq) and tetramethylammonium chloride (97.2 mg, 887 μmol, 2.6 eq) under N₂. The mixture was stirred at 60° C. for 4 hr. The reaction mixture was filtered and purified by prep-HPLC (column: Phenomenex Luna C18 75*30 mm*3 um; mobile phase: [H₂O (0.1% TFA)-ACN]; gradient: 25%-65% B over 8.0 min) to afford the title compound as a white solid (13.2 mg, 46.6 μmol, 13.7% yield). ¹H NMR (400 MHz, DMSO-d₆) δ 8.90 (s, 1H), 4.60 (t, J=5.2 Hz, 2H), 4.47 (t, J=5.6 Hz, 2H). LC/MS [M+H]⁺ 284.0 (calculated); LC/MS [M+H]⁺ 284.2 (observed).

### Example 21—Preparation of 8-Acetyl-3-(2-(trifluoromethoxy)ethyl)imidazo[5,1-d][1,2,3,5]tetrazin-4(3H)-one (I-38)

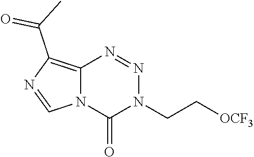 [SMILES: CC(=O)c1ncn2c(=O)n(CCOC(F)(F)F)nnc12]

The title compound was prepared. ¹H NMR (400 MHz, DMSO-d₆) δ 8.92 (s, 1H), 4.67 (t, J=5.2 Hz, 2H), 4.53-4.48 (m, 2H), 2.70 (s, 3H). LC/MS [M+H]⁺ 292.1 (calculated); LC/MS [M+H]⁺ 292.1 (observed).

### Example 22—Preparation of N-(2-Aminophenyl)-4-oxo-3-(2-(trifluoromethoxy)ethyl)-3,4-dihydroimidazo[5,1-d][1,2,3,5]tetrazine-8-carboxamide (I-24)

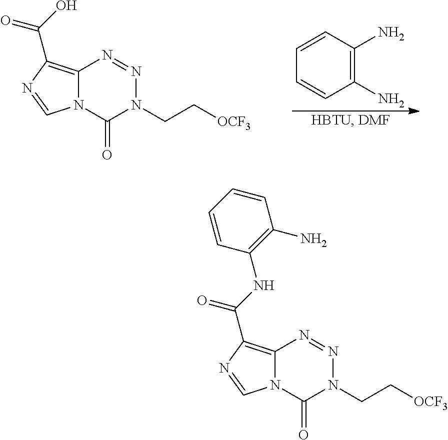

To a solution of HBTU (427 mg, 1.13 mmol, 1.1 eq) and 4-oxo-3-[2-(trifluoromethoxy)ethyl]imidazo[5,1-d][1,2,3,5]tetrazine-8-carboxylic acid (300 mg, 1.02 mmol, 1 eq) in DMF (5 mL) was added a solution of benzene-1,2-diamine (166 mg, 1.54 mmol, 1.5 eq) in DMF (0.5 mL). The mixture was stirred at 20° C. for 12 hrs. The reaction mixture was quenched by addition H₂O (20 mL) at 25° C., and extracted with EtOAc (20 mL×3). The combined organic layers were washed with brine (15 mL), dried over Na₂SO₄, filtered, and concentrated under reduced pressure. The residue was purified by flash silica gel chromatography (ISCO®; 12 g SepaFlash® Silica Flash Column, Eluent of 0˜50% ethyl acetate/petroleum ether gradient @90 mL/min) to afford the title compound as a yellow solid (300 mg, 783 μmol, 76.49% yield. ¹H NMR (400 MHz, DMSO-d₆) δ 9.79 (s, 1H), 9.02 (s, 1H), 7.43 (dd, J=7.75, 1.13 Hz, 1H), 7.05-6.94 (m, 1H), 6.84 (dd, J=8.00, 1.25 Hz, 1H), 6.74-6.56 (m, 1H), 5.06-4.80 (m, 2H), 4.74-4.64 (m, 2H), 4.58-4.46 (m, 2H).

### Example 23—Alternative Preparation of 8-(1H-Benzo[d]imidazol-2-yl)-3-(2-(trifluoromethoxy) ethyl)imidazo[5,1-d][1,2,3,5]tetrazin-4(3H)-one (II-1)

The title compound was prepared according to the following procedures.

### Step 1: Preparation of N-(2-aminophenyl)-4-oxo-3-(2-(trifluoromethoxy)ethyl)-3,4-dihydroimidazo[5,1-d][1,2,3,5]tetrazine-8-carboxamide

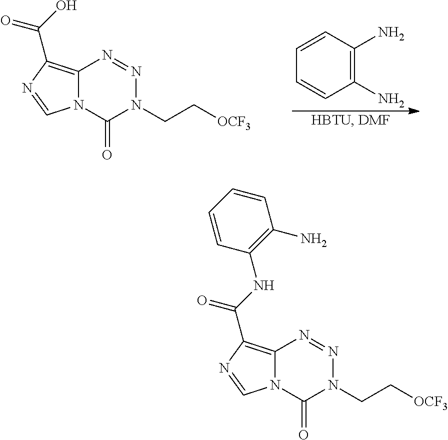

To a solution of HBTU (427 mg, 1.13 mmol, 1.1 eq) and 4-oxo-3-[2-(trifluoromethoxy)ethyl]imidazo[5,1-d][1,2,3,5]tetrazine-8-carboxylic acid (300 mg, 1.02 mmol, 1 eq) in DMF (5 mL) was added a solution of benzene-1,2-diamine (166 mg, 1.54 mmol, 1.5 eq) in DMF (0.5 mL). The mixture was stirred at 20° C. for 12 hrs. The reaction mixture was quenched by addition H₂O 20 mL at 25° C., and extracted with EtOAc (20 mL×3). The combined organic layers were washed with brined (15 mL), dried over Na₂SO₄, filtered and concentrated under reduced pressure. The residue was purified by flash silica gel chromatography (ISCO®; 12 g SepaFlash® Silica Flash Column, Eluent of 0˜50% Ethyl acetate/Petroleum ether gradient @90 mL/min to afford the titled compound as a yellow solid (300 mg, 783 μmol, 76.49% yield. ¹H NMR (400 MHz, DMSO-d₆) δ 9.79 (s, 1H), 9.02 (s, 1H), 7.43 (dd, J=7.75, 1.13 Hz, 1H), 7.05-6.94 (m, 1H), 6.84 (dd, J=8.00, 1.25 Hz, 1H), 6.74-6.56 (m, 1H), 5.06-4.80 (m, 2H), 4.74-4.64 (m, 2H), 4.58-4.46 (m, 2H).

### Step 2: Preparation of 8-(1H-benzo[d]imidazol-2-yl)-3-(2-(trifluoromethoxy)ethyl) imidazo[5,1-d][1,2,3,5]tetrazin-4(3H)-one

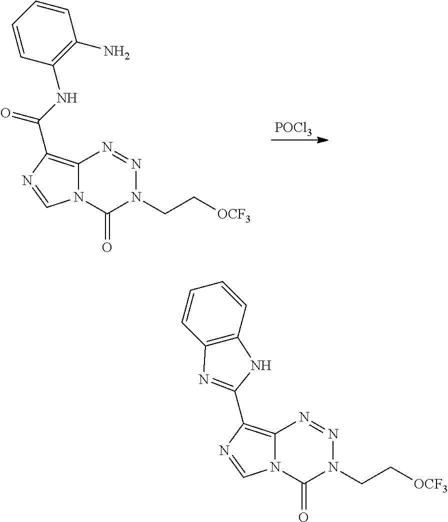 [SMILES: Nc1ccccc1NC(=O)c1ncn2c(=O)n(CCOC(F)(F)F)nnc12 ; "POCl3" ; O=c1n(CCOC(F)(F)F)nnc2c(-c3nc4ccccc4[nH]3)ncn12]

A solution of N-(2-aminophenyl)-4-oxo-3-[2-(trifluoromethoxy) ethyl]imidazo[5,1-d][1,2,3,5]tetrazine-8-carboxamide (270 mg, 704 μmol, 1 eq) in POCl₃ (5 mL) was stirred at 80° C. for 2 hr. The reaction mixture was concentrated under reduced pressure to give a residue. The residue was purified by prep-HPLC (TFA condition; column: Phenomenex Luna C18 75*30 mm*3 um; mobile phase: [H₂O (0.1% TFA)-ACN]; gradient: 15%-40% B over 8.0 min) to afford the titled compound as a yellow soild (161.1 mg, 422 μmol, 59.85% yield, 95.59% purity). H NMR (400 MHz, DMSO-d6) δ 9.14 (s, 1H), 7.83-7.64 (m, 2H), 7.40-7.35 (m, 2H), 4.72-4.66 (m, 2H), 4.57-4.51 (m, 2H). MS (ESI): mass calcd. For C₁₋₄H₁₀F₃N₇O₂ 365.08, m/z found 366.1 [M+H]⁺.

### Example 24—Preparation of N-(2-Hydroxyphenyl)-4-oxo-3-(2-(trifluoromethoxy)ethyl)-3,4-dihydroimidazo[5,1-d][1,2,3,5]tetrazine-8-carboxamide (I-27)

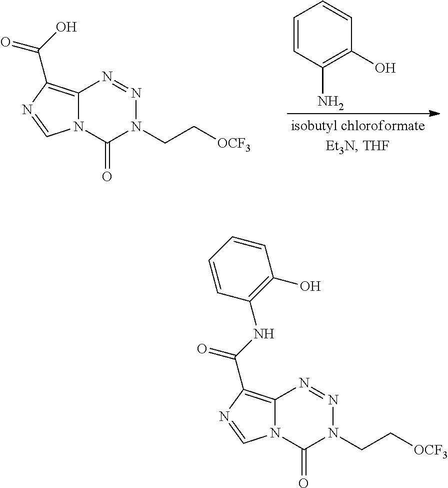 [SMILES: O=C(O)c1ncn2c(=O)n(CCOC(F)(F)F)nnc12 ; Nc1ccccc1O ; O=C(Nc1ccccc1O)c1ncn2c(=O)n(CCOC(F)(F)F)nnc12]

To a solution of 4-oxo-3-[2-(trifluoromethoxy)ethyl]imidazo[5,1-d][1,2,3,5]tetrazine-8-carboxylic acid (0.3 g, 1.02 mmol, 1 eq) was added isobutyl chloroformate (147 mg, 1.07 mmol, 141 μL, 1.05 eq) followed by Et₃N (109 mg, 1.07 mmol, 150 μL, 1.05 eq) in THF (10 mL), and then the mixture was stirred for 2 hours at 20° C. The precipitate was removed by vacuum filtration, and then 2-aminophenol (117 mg, 1.07 mmol, 1.05 eq) was added to the filtrate. The resulting mixture was stirred at 20° C. under nitrogen for 12 hrs. The reaction mixture was filtered and to concentrated to dryness. The crude product was triturated with EtOAc (10 mL) at 0° C. for 5 min to afford the title compound as a yellow solid (0.3 g, 781 μmol, 76.3% yield).

### Example 25—Preparation of 8-(benzo[d]oxazol-2-yl)-3-(2-(trifluoromethoxy)ethyl) imidazo[5,1-d][1,2,3,5]tetrazin-4(3H)-one (II-4)

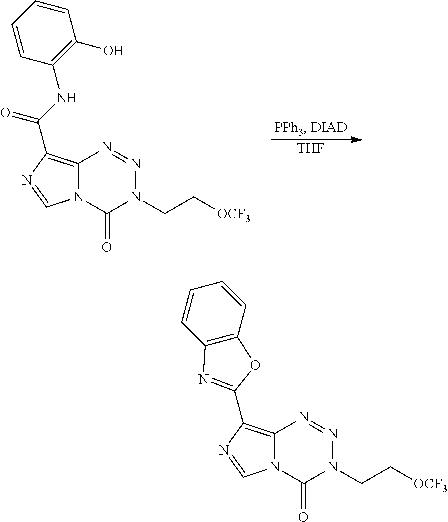

To a solution of N-(2-hydroxyphenyl)-4-oxo-3-[2-(trifluoromethoxy)ethyl]imidazo [5,1-d][1,2,3,5]tetrazine-8-carboxamide (0.2 g, 520 μmol, 1 eq) in THE (10 mL) was added PPh₃ (683 mg, 2.60 mmol, 5 eq) and DIAD (526 mg, 2.60 mmol, 504 μL, 5 eq). The mixture was stirred at 20° C. for 12 hrs. The reaction mixture was filtered and concentrated. The crude product was triturated with MTBE (10 mL) and EtOAc (2 mL) at 20° C. for 5 min to afford the title compound as a light green solid (43.1 mg, 117.7 μmol, 22.6% yield). ¹H NMR (400 MHz, DMSO-d₆) δ 9.15 (s, 1H), 7.99-7.86 (m, 2H), 7.55-7.467 (m, 2H), 4.75-4.67 (m, 2H), 4.57-4.48 (m, 2H). LC/MS [M+H]⁺ 367.1 (calculated); LC/MS [M+H]⁺ 367.2 (observed).

### Example 26—Preparation of 4-Oxo-N-(2-oxo-2-phenylethyl)-3-(2-(trifluoromethoxy) ethyl)-3,4-dihydroimidazo[5,1-d][1,2,3,5]tetrazine-8-carboxamide (I-25)

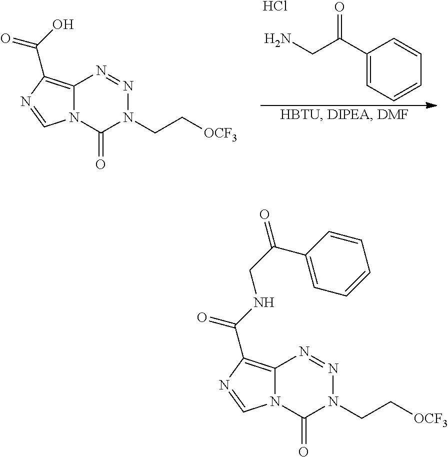 [SMILES: Cl ; O=C(O)c1ncn2c(=O)n(CCOC(F)(F)F)nnc12 ; NCC(=O)c1ccccc1 ; "HBTU, DIPEA, DMF" ; O=C(CNC(=O)c1ncn2c(=O)n(CCOC(F)(F)F)nnc12)c1ccccc1]

To a solution of 4-oxo-3-[2-(trifluoromethoxy)ethyl]imidazo[5,1-d][1,2,3,5]tetrazine-8-carboxylic acid (0.2 g, 682 μmol, 1 eq) in DMF (2 mL) was added HBTU (271.66 mg, 716.33 mol, 1.05 eq), and then the mixture was stirred for 20 min at 25° C. Then 2-amino-1-phenyl-ethanone (128.79 mg, 750.45 μmol, 1.1 eq, HCl) was added, followed by DIPEA (352.69 mg, 2.73 mmol, 475.32 μL, 4 eq), and the reaction mixture was stirred at 25° C. for 2 hr. The mixture was poured into H₂O (10 mL) and extracted with EtOAc (5 mL×3). The combined organic layer was dried over Na₂SO₄ and concentrated. The residue was purified by flash silica gel chromatography (ISCO®; 4 g SepaFlash® Silica Flash Column, eluent of 0˜30% ethyl acetate/petroleum ether gradient @75 mL/min) to afford the title compound as a yellow oil (156 mg, 380 μmol, 55.7% yield).

### Example 27—Preparation of 8-(5-Phenyloxazol-2-yl)-3-(2-(trifluoromethoxy) ethyl)imidazo[5,1-d][1,2,3,5]tetrazin-4(3H)-one (II-2)

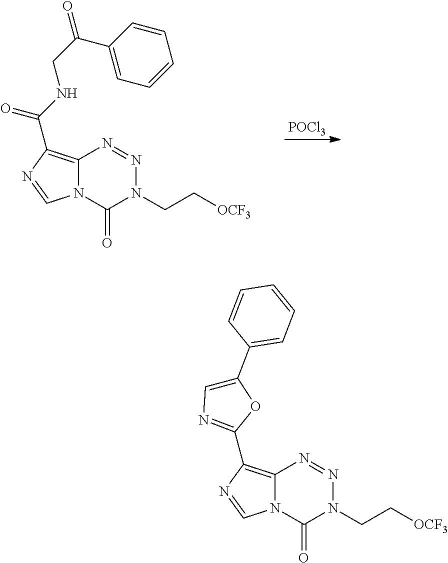 [SMILES: O=C(CNC(=O)c1ncn2c(=O)n(CCOC(F)(F)F)nnc12)c1ccccc1 ; "POCl3" ; O=c1n(CCOC(F)(F)F)nnc2c(-c3ncc(-c4ccccc4)o3)ncn12]

A mixture of 4-oxo-N-phenacyl-3-[2-(trifluoromethoxy)ethyl]imidazo[5,1-d][1,2,3,5]tetrazine-8-carboxamide (0.13 g, 317 μmol, 1 eq) in POCl₃ (1 mL) was stirred at 80° C. for 0.5 hrs. After completion, the reaction mixture was concentrated, quenched with H₂O (2 mL), and then extracted with EtOAc (2 mL×3). The combined organic layer was dried over Na₂SO₄ and then concentrated. The residue was purified by prep-HPLC (column: Phenomenex Luna C18 75*30 mm*3 m; mobile phase: [H₂O (0.1% TFA)-ACN]; gradient: 35%-65% B over 8.0 min) to provide the title compound as a yellow solid (21.7 mg, 55.3 μmol, 17.5% yield). ¹H NMR (400 MHz, DMSO-d₆) δ 9.01 (s, 1H), 8.01 (s, 1H), 7.85 (d, J=7.38 Hz, 2H), 7.55 (t, J=7.69 Hz, 2H), 7.48-7.40 (m, 1H), 4.67 (t, J=4.8 Hz, 2H), 4.53 (t, J=4.8 Hz, 2H). MS (ESI): mass calc'd. 392.08, m/z found 393.1 [M+H]⁺.

### Example 28—Preparation of 4-Oxo-3-(2-(trifluoromethoxy)ethyl)-3,4-dihydroimidazo[5,1-d][1,2,3,5]tetrazine-8-carbonyl Chloride (I-37)

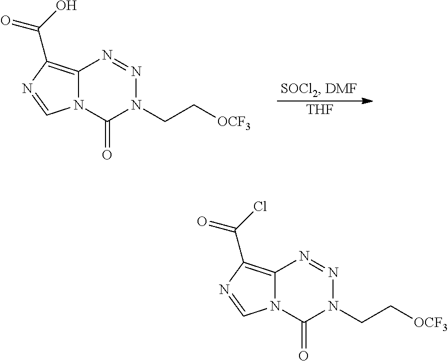

To a mixture of 4-oxo-3-(2-(trifluoromethoxy)ethyl)-3,4-dihydroimidazo[5,1-d][1,2,3,5]tetrazine-8-carboxylic acid (150 mg, 512 μmol, 1 eq) and DMF (3.74 mg, 0.1 eq) in THF (5 mL) was added SOCl₂ (73.1 mg, 614 μmol, 1.2 eq) at 25° C. under N₂. The reaction mixture was heated to 70° C. and stirred for 2 hours. The reaction mixture was concentrated in vacuum to afford the title compound as a yellow oil (0.15 g, crude).

### Example 29—Preparation of 4-Oxo-N-(2-oxopropyl)-3-(2-(trifluoromethoxy)ethyl)-3,4-dihydroimidazo[5,1-d][1,2,3,5]tetrazine-8-carboxamide (I-28)

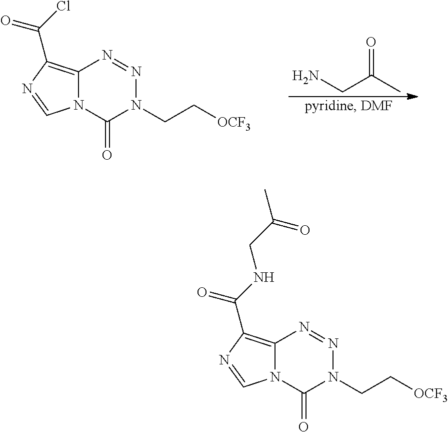

To a mixture of 4-oxo-3-(2-(trifluoromethoxy)ethyl)-3,4-dihydroimidazo[5,1-d][1,2,3,5]tetrazine-8-carbonyl chloride (0.15 g, 481 μmol, 1 eq) and 1-aminopropan-2-one (52.7 mg, 481 μmol, 1 eq, HCl) in DMF (3 mL) was added pyridine (76.1 mg, 963 μmol, 2 eq) at 25° C. under N₂, and the mixture was stirred for 12 hours. The mixture was quenched with H₂O (4 mL), and the aqueous phase was extracted with ethyl acetate (5 mL×3). The combined organic phase was washed with brine (5 mL), dried with anhydrous Na₂SO₄, filtered, and concentrated in vacuum. The residue was purified by flash silica gel chromatography (Biotage®; 4 g SepaFlash® Silica Flash Column, Eluent of 0˜100% Ethyl acetate/Petroleum ether gradient @50 mL/min) to provide the title compound as a yellow solid (0.15 g, 431 μmol, 89.5% yield). ¹H NMR (400 MHz, DMSO-d₆) δ 8.92 (s, 1H), 8.63 (t, J=5.6 Hz, 1H), 4.64 (t, J=5.2 Hz, 2H), 4.49 (t, J=5.2 Hz, 2H), 4.16 (d, J=5.6 Hz, 2H), 2.14 (s, 3H).

### Example 30—Preparation of 8-(5-Methyloxazol-2-yl)-3-(2-(trifluoromethoxy)ethyl) imidazo[5,1-d][1,2,3,5]tetrazin-4(3H)-one (II-3)

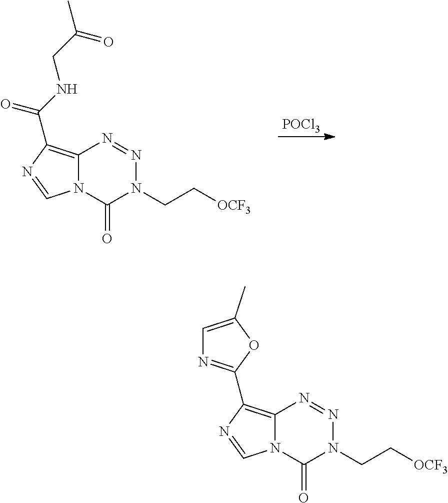 [SMILES: CC(=O)CNC(=O)c1ncn2c(=O)n(CCOC(F)(F)F)nnc12 ; "POCl3" ; Cc1cnc(-c2ncn3c(=O)n(CCOC(F)(F)F)nnc23)o1]

A solution of 4-oxo-N-(2-oxopropyl)-3-(2-(trifluoromethoxy)ethyl)-3,4-dihydroimidazo[5,1-d][1,2,3,5]tetrazine-8-carboxamide (0.1 g, 287 μmol, 1 eq) in POCl₃ (3 mL) was stirred at 60° C. for 12 hours. The reaction mixture was concentrated in vacuum. The residue was poured into ice-water (w/w=1/1; 30 mL) and stirred for 5 min. The aqueous phase was extracted with DCM (15 mL×3). The combined organic phase was washed with brine (15 mL), dried with anhydrous Na₂SO₄, filtered, and concentrated in vacuum. The residue was purified by prep-HPLC (column: Phenomenex luna C18 100×40 mm×5 um; mobile phase: [H₂O (0.2% FA)-ACN]; gradient: 30%-65% B over 8.0 min) to afford the title compound as a yellow solid (21.0 mg, 62.6 μmol, 21.8% yield). ¹H NMR (400 MHz, DMSO-d₆) δ 8.94 (s, 1H), 7.14 (s, 1H), 4.64 (t, J=4.8 Hz, 2H), 4.50 (t, J=5.2 Hz, 2H), 2.44 (s, 3H). LC/MS [M+H]⁺ 331.1 (calculated); LC/MS [M+H]⁺ 331.1 (observed).

### Example 31—Preparation of 8-Phenyl-3-(2-(trifluoromethoxy)ethyl)imidazo[5,1-d][1,2,3,5]tetrazin-4(3H)-one (II-5)

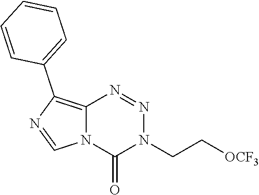 [SMILES: O=c1n(CCOC(F)(F)F)nnc2c(-c3ccccc3)ncn12]

### Part I—Synthesis of 5-Nitro-4-phenyl-1H-imidazole

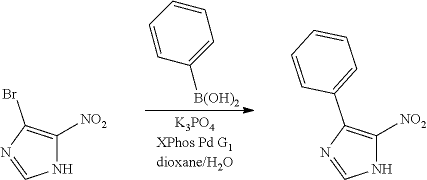 [SMILES: O=[N+]([O-])c1[nH]cnc1Br ; OB(O)c1ccccc1 ; O=[N+]([O-])c1[nH]cnc1-c1ccccc1]

A mixture of 4-bromo-5-nitro-1H-imidazole (5 g, 26.1 mmol, 1 eq), phenylboronic acid (6.35 g, 52.1 mmol, 2 eq), [2-(2-aminoethyl)phenyl]-chloro-palladium; dicyclohexyl-[2-(2,4,6-triisopropylphenyl)phenyl]phosphane (1.92 g, 2.60 mmol, 0.1 eq), K₃PO₄ (16.59 g, 78.14 mmol, 3 eq) in dioxane (50 mL) and H₂O (50 mL) was degassed and purged with N₂ for 3 times then stirred at 110° C. for 16 hrs under N₂ atmosphere. The reaction mixture was extracted with EtOAc (30 mL×3). The combined organic layers were washed with brine (30 mL), dried over Na₂SO₄, filtered, and concentrated under reduced pressure to give a residue. The residue was purified by flash silica gel chromatography (ISCO®; 20 g SepaFlash® Silica Flash Column, Eluent of 0˜50% Ethyl acetate/Petroleum ether gradient @66 mL/min) to provide the title compound as a yellow solid (3.2 g, 16.9 mmol, 65.0% yield). H NMR (400 MHz, DMSO-d₆) δ 7.90 (s, 1H), 7.70-7.63 (m, 2H), 7.56-7.46 (m, 3H).

### Part II—Synthesis of 4-Phenyl-1H-imidazol-5-amine

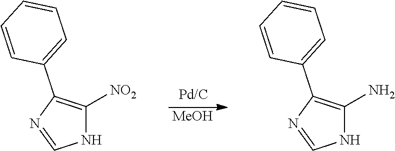 [SMILES: "MeOH" ; O=[N+]([O-])c1[nH]cnc1-c1ccccc1 ; Nc1[nH]cnc1-c1ccccc1]

To a solution of 5-nitro-4-phenyl-1H-imidazole (3.2 g, 16.9 mmol, 1 eq) in MeOH (30 mL) was added Pd/C (1.80 g, 1.69 mmol, 10% purity, 0.1 eq). The suspension was degassed under vacuum and purged with H₂ several times. The reaction mixture was stirred at 20° C. for 2 hrs under H₂ (15 psi). The reaction mixture was filtered, and the filtrate was concentrated. The residue was purified by flash silica gel chromatography (ISCO®; 80 g SepaFlash® Silica Flash Column, Eluent of 0˜100% Ethyl acetate/Petroleum ether gradient @80 mL/min) to afford the title compound as a red-brown oil (1.2 g, 7.54 mmol, 44.6% yield). ¹H NMR (400 MHz, DMSO-d₆) δ 11.76 (s, 1H), 7.54 (br s, 2H), 7.35-7.30 (m, 3H), 7.05 (t, J=7.4 Hz, 1H), 4.61 (br s, 2 h).

### Part III—Synthesis 4-Phenyl-1H-imidazole-5-diazonium

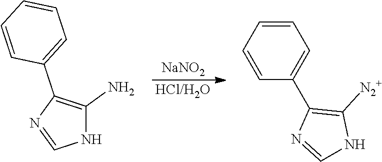

To a solution of 4-phenyl-1H-imidazol-5-amine (0.27 g, 1.70 mmol, 1 eq) in HCl (2 μM, 1.70 mL, 2 eq) was added NaNO₂ (176 mg, 2.54 mmol, 1.5 eq) in H₂O (0.9 mL) dropwise at 0° C. and stirred for 1 hour. The reaction mixture was filtered, and the filtrate was freeze-dried to afford the crude pink solid which was used without further purification (0.29 g, 1.69 mmol, 99.9% yield).

### Part IV—Synthesis of 8-Phenyl-3-(2-(trifluoromethoxy)ethyl)imidazo[5,1-d][1,2,3,5]tetrazin-4(3H)-one (II-5)

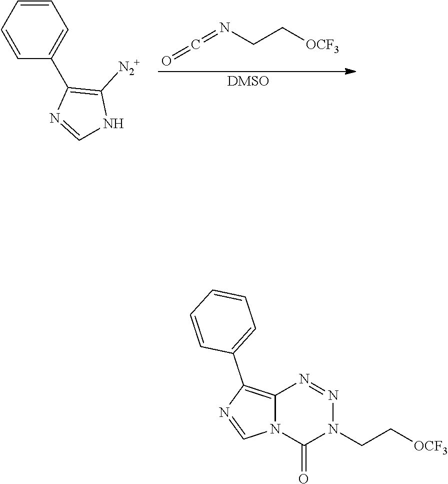

A mixture of 4-phenyl-1H-imidazole-5-diazonium (276 mg, 1.61 mmol, 1 eq) and 1-isocyanato-2-(trifluoromethoxy)ethane (0.25 g, 1.61 mmol, 1 eq) in DMSO (5 mL) was degassed and purged with N₂ three times, and then the mixture was stirred at 25° C. for 2 hrs. The reaction mixture was filtered, and the filtrate was concentrated. The residue was purified by prep-HPLC (column: Phenomenex Luna C18 75*30 mm*3 μm; mobile phase: [H₂O (0.1% TFA)-ACN]; gradient: 40%-75% B over 8.0 min) to afford the title compound as a brown solid (24.5 mg, 75.3 μmol, 4.67% yield). ¹H NMR (400 MHz, DMSO-d₆) δ 8.90 (s, 1H), 8.31 (d, J=7.4 Hz, 2H), 7.58-7.53 (m, 2H), 7.49-7.43 (m, 1H), 4.64-4.60 (m, 2H), 4.54-4.49 (m, 2H). LC/MS [M+H]⁺ 326.1 (calculated); LC/MS [M+H]⁺ 326.1 (observed).

### Example 32—Preparation of 3-Cyclohexyl-1-nitroso-1-(2-(trifluoromethoxy)ethyl)urea (IV-1)

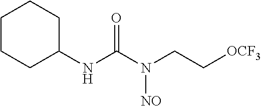

### Part I—Synthesis of 1-Cyclohexyl-3-(2-(trifluoromethoxy)ethyl)urea

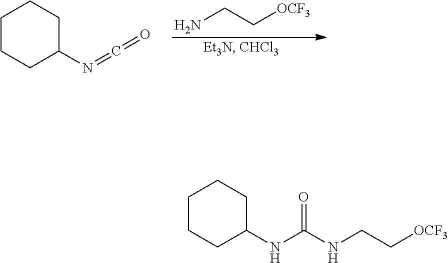

To a solution of cyclohexyl isocyanate (0.3 g, 2.40 mmol, 306 μL, 1 eq) and 2-(trifluoromethoxy)ethanamine (396.77 mg, 2.40 mmol, 1 eq, HCl) in CHCl₃ (30 mL) was added dropwise Et₃N (242.53 mg, 2.40 mmol, 333.60 μL, 1 eq) at 0° C. After stirring at 0° C. for 2 hr, the reaction mixture was warmed to 25° C. and stirred for another 2 hr. The mixture was concentrated to remove CHCl₃, and the residue was washed with H₂O (5 mL) to remove hydrochloride salt. The remaining solid was filtered and the filtrate was redissolved in CHCl₃ (5 mL), dried over Na₂SO₄ and re-concentrated. The crude product was triturated with MTBE (5 mL×2) at 0° C. to obtain the title compound as white solid (0.6 g). ¹H NMR (400 MHz, CDCl₃) δ 4.73-4.58 (m, 1H), 4.34 (s, 1H) 3.97 (t, J=5.00 Hz, 2H) 3.43 (d, J=4.00 Hz, 3H) 1.90-1.82 (m, 2H), 1.63-1.58 (m, 2H), 1.57-1.47 (m, 1H), 1.22-1.36 (m, 2H), 1.15-0.98 (m, 3H).

### Part II—Synthesis of 3-Cyclohexyl-1-nitroso-1-(2-(trifluoromethoxy)ethyl)urea (IV-1)

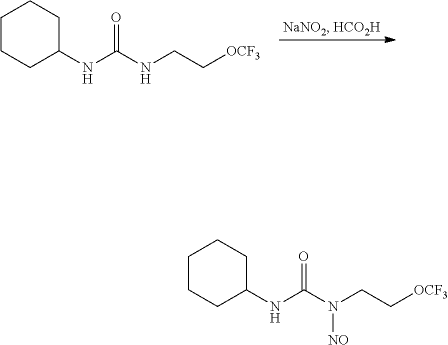

To a solution of 1-cyclohexyl-3-[2-(trifluoromethoxy)ethyl]urea (0.4 g, 1.57 mmol, 1 eq) in HCOOH (2 mL) was added NaNO₂ (379.94 mg, 5.51 mmol, 3.5 eq) in portions at 0° C., and after addition the reaction mixture was stirred at 0° C. for 2 hr. The reaction was quenched with H₂O (10 mL), and the aqueous was extracted with CHCl₃ (5 mL×2). The combined organic layer was dried over Na₂SO₄ and concentrated to provide the title compound as a yellow oil (0.15 g, 34%). ¹H NMR (400 MHz, CDCl₃) δ 6.77-6.66 (m, 1H), 4.13-4.05 (m, 2H), 3.94-3.88 (m, 2H), 3.86-3.77 (m, 1H), 2.05-1.95 (m, 2H), 1.75-1.65 (m, 2H), 1.63-1.53 (m, 1H), 1.41-1.29 (m, 2H), 1.29-1.12 (m, 3H).

### Example 33—Preparation of 1-Nitroso-1,3-bis(2-(trifluoromethoxy)ethyl)urea (IV-2)

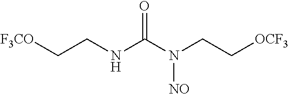

### Part I—Synthesis of 1,3-Bis(2-(trifluoromethoxy)ethyl)urea

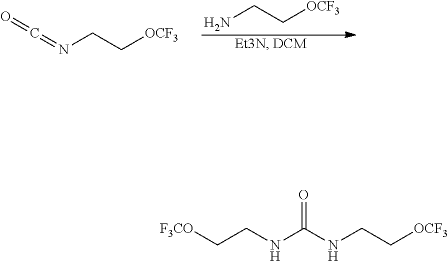

To a solution of 1-isocyanato-2-(trifluoromethoxy)ethane (0.9 g, 5.80 mmol, 1 eq) and 2-(trifluoromethoxy)ethanamine (960 mg, 5.80 mmol, 1 eq, HCl) in CHCl₃ (10 mL) was added dropwise Et₃N (587 mg, 5.80 mmol, 808 μL, 1 eq) at 0° C. After stirring at 0° C. for 2 hrs, the mixture was warmed up to 25° C. and stirred for another 2 hrs. The mixture was concentrated and the residue was purified by flash silica gel chromatography (ISCO®; 2 g SepaFlash® Silica Flash Column, Eluent of 0˜X % Ethyl acetate/Petroleum ether gradient @70 mL/min) to afford the title compound as a white solid (0.72 g, 2.53 mmol, 43.7% yield). ¹H NMR (400 MHz, CDCl₃) δ 4.93 (br s, 2H), 3.97 (t, J=5.2 Hz, 4H), 3.44 (br t, J=4.8 Hz, 4H).

### Part II—Synthesis of 1-Nitroso-1,3-bis(2-(trifluoromethoxy)ethyl)urea (IV-21)

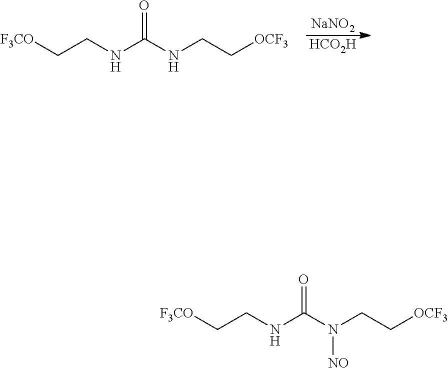

To a solution of 1,3-bis[2-(trifluoromethoxy)ethyl]urea (0.36 g, 1.27 mmol, 1 eq) in formic acid (2 mL) was added NaNO₂ (306 mg, 4.43 mmol, 3.5 eq) in portions at 0° C. After addition, the reaction mixture was stirred at 0° C. for 2 hrs. Water (10 mL) was added, and the aqueous was extracted with CHCl₃ (5 mL×2). The combined organic layer was dried over Na₂SO₄ then concentrated to afford the title compound as yellow oil (0.3 g, crude). ¹H NMR (400 MHz, CDCl₃) δ 7.28 (br s, 1H), 4.21 (q, J=5.3 Hz, 4H) 4.04-3.99 (m, 2H) 3.84 (q, J=5.4 Hz, 2H).

### Example 34—Preparation of N,N-Dimethyl-4-((2-(2-(trifluoromethoxy)ethyl) hydrazinyl)methyl)benzamide (V-1)

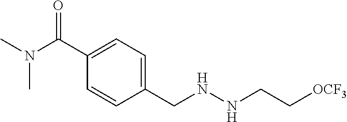 [SMILES: CN(C)C(=O)c1ccc(CNNCCOC(F)(F)F)cc1]

### Part I—Synthesis of di-tert-Butyl 1-(2-Hydroxyethyl)hydrazine-1,2-dicarboxylate

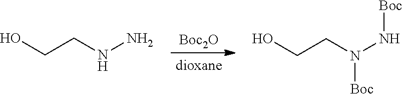 [SMILES: "Boc2O" ; NNCCO ; "dioxane" ; CC(C)(C)OC(=O)NN(CCO)C(=O)OC(C)(C)C]

To a solution of 2-hydrazinoethanol (5 g, 65.7 mmol, 4.46 mL, 1 eq) in dioxane (100 mL) was added Boc₂O (31.55 g, 144.55 mmol, 33.21 mL, 2.2 eq) at 0° C. The mixture was stirred at 25° C. for 16 hrs. The reaction mixture was poured into water (30 mL), and the resulting mixture was extracted with EtOAc (30 mL×3). The organic layer was washed with brine (20 mL), dried over anhydrous Na₂SO₄, filtered, and concentrated under reduced pressure. The residue was purified by flash silica gel chromatography (ISCO®; 80 g SepaFlash® Silica Flash Column, Eluent of 0˜15% Ethyl acetate/Petroleum ether gradient @80 mL/min) to afford the title compound as a white solid (11 g, 39.8 mmol, 60.6% yield). ¹H NMR (400 MHz, CDCl₃) δ 6.40 (s, 1H), 3.71 (br s, 2H), 3.58 (br s, 2H), 1.52-1.45 (br s, 18H).

### Part II—Synthesis of di-tert-Butyl 1-(2-(Trifluoromethoxy)ethyl)hydrazine-1,2-dicarboxylate

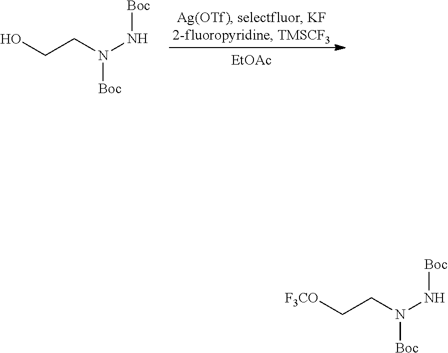 [SMILES: "EtOAc" ; CC(C)(C)OC(=O)NN(CCO)C(=O)OC(C)(C)C ; "Ag(OTf), selectfluor, KF2-fluoropyridine, TMSCF3" ; CC(C)(C)OC(=O)NN(CCOC(F)(F)F)C(=O)OC(C)(C)C]

To a solution of KF (4.20 g, 72.4 mmol, 4 eq), AgOTf (13.95 g, 54.28 mmol, 3 eq), Selectfluor (9.62 g, 27.14 mmol, 1.5 eq), and di-tert-butyl 1-(2-hydroxyethyl)hydrazine-1,2-dicarboxylate (5 g, 18.09 mmol, 1 eq) in EtOAc (100 mL), were added 2-fluoropyridine (5.27 g, 54.3 mmol, 4.66 mL, 3 eq) and TMSCF₃ (7.72 g, 54.28 mmol, 3 eq) at 30° C. The resulting mixture was stirred at 30° C. for 16 hrs under N₂ atmosphere. The reaction mixture was poured into water (30 mL), and the resulting mixture was then extracted with EtOAc (3×30 mL). The organic layer was washed with brine (20 mL), dried over anhydrous Na₂SO₄, filtered, and concentrated under reduced pressure. The residue was purified by flash silica gel chromatography (ISCO®; 80 g SepaFlash® Silica Flash Column, Eluent of 0˜10% Ethyl acetate/Petroleum ether gradient @80 mL/min) to afford the title compound as a white solid (0.69 g, 2.00 mmol, 11.1% yield). ¹H NMR (400 MHz, CDCl₃) δ 6.33 (br s, 1H), 4.05-4.00 (m, 2H), 3.69 (br s, 2H), 1.52-1.41 (br s, 18H).

### Part III—Synthesis of di-tert-Butyl 1-(4-(Dimethylcarbamoyl)benzyl)-2-(2-(trifluoromethoxy)ethyl)hydrazine-1,2-dicarboxylate

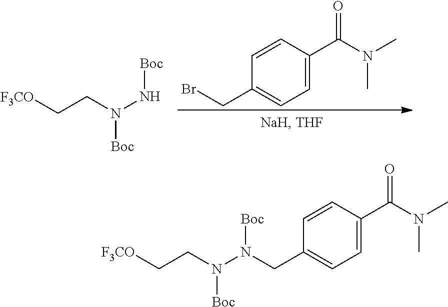 [SMILES: CC(C)(C)OC(=O)NN(CCOC(F)(F)F)C(=O)OC(C)(C)C ; CN(C)C(=O)c1ccc(CBr)cc1 ; CN(C)C(=O)c1ccc(CN(C(=O)OC(C)(C)C)N(CCOC(F)(F)F)C(=O)OC(C)(C)C)cc1]

To a solution of di-tert-butyl 1-(2-(trifluoromethoxy)ethyl)hydrazine-1,2-dicarboxylate (0.1 g, 290 μmol, 1 eq) in DMF (2 mL) was added NaH (14.0 mg, 348 μmol, 60% purity, 1.2 eq) at 0° C. After stirring for 5 mins at 0° C., 4-(bromomethyl)-N,N-dimethyl-benzamide (105.5 mg, 435.6 μmol, 1.5 eq) was added, and the resulting mixture was stirred for 30 mins at 25° C. The reaction mixture was quenched with H₂O (1 mL) and then extracted with EtOAc (5 mL×3). The organic layer was washed with brine (5 mL), dried over anhydrous Na₂SO₄, filtered, and concentrated under reduced pressure. The residue was purified by flash silica gel chromatography (ISCO®; 4 g SepaFlash® Silica Flash Column, Eluent of 0˜20% Ethyl acetate/Petroleum ether gradient @45 mL/min) to afford the title compound as a white oil (0.07 g, 138 μmol, 47.7% yield).

### Part IV—Synthesis of N,N-Dimethyl-4-((2-(2-(trifluoromethoxy)ethyl)hydrazinyl)methyl) benzamide (V-1)

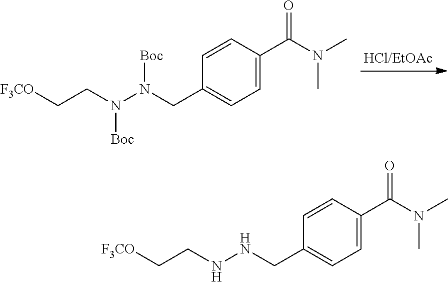 [SMILES: CN(C)C(=O)c1ccc(CN(C(=O)OC(C)(C)C)N(CCOC(F)(F)F)C(=O)OC(C)(C)C)cc1 ; CN(C)C(=O)c1ccc(CNNCCOC(F)(F)F)cc1]

A solution of di-tert-butyl 1-(4-(dimethylcarbamoyl)benzyl)-2-(2-(trifluoromethoxy) ethyl)hydrazine-1,2-dicarboxylate (0.07 g, 138 μmol, 1 eq) in HCl/EtOAc (3 mL) was stirred at 25° C. for 16 hrs. The mixture was filtered and concentrated under reduced pressure. The residue was purified by prep-HPLC (column: Phenomenex Luna C18 75*30 mm*3 um; mobile phase: [H₂O (0.1% TFA)-ACN]; gradient: 1%-30% B over 8.0 min) to afford the title compound as a yellow oil (14 mg, 46 μmol, 33% yield)). ¹H NMR (400 MHz, D₂O) δ 7.48-7.37 (m, 4H), 4.18-4.13 (m, 2H), 4.10 (s, 2H), 3.21-3.17 (m, 2H), 3.03 (s, 3H), 2.92 (s, 3H). LC/MS [M+H]⁺ 306.1 (calculated); LC/MS [M+H]⁺ 306.1 (observed).

### Example 35—General Procedures for Preparation of Nitrosourea Compounds

General procedures that may be used to prepare nitrosourea compounds are provided below.

### Step 1: Preparation of 1-(2-(trifluoromethoxy)ethyl)urea

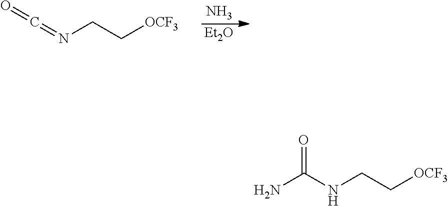

A solution of 1-isocyanato-2-(trifluoromethoxy)ethane (1.0 equivalent) in diethyl ether is added dropwise to a solution of ammonia in THE (1.0 equivalent) and the reaction is stirred at room temperature for 6 to 12 h. After, the reaction is concentrated to dryness on a rotary evaporator to afford the title compound.

### Step 2: Preparation of 1-nitroso-1-(2-(trifluoromethoxy)ethyl)urea

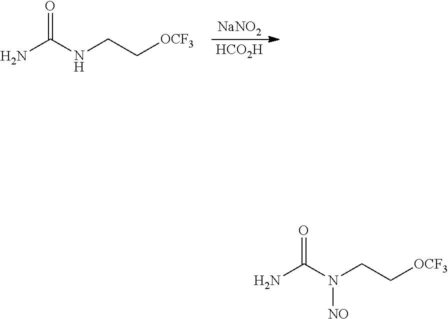

To a solution of 1-(2-(trifluoromethoxy)ethyl)urea (1 eq) in HCOOH (0.3 μM) is added NaNO₂ (3.5 eq) in portions at 0° C., after addition the reaction mixture is stirred at 0° C. for 2 hr. The reaction is quenched with H₂O (10 vol), and the aqueous is extracted with CHCl₃ (5 vol×2), the combined organic layer is dried over Na₂SO₄ and concentrated to provide the title compound.

### Example 36—Additional General Procedures for Preparation of Nitrosourea Compounds

The reaction schemes below outline additional exemplary ways that nitrosourea compounds may be prepared, based in part on general procedures described in Example 35.

Scheme 1 is general procedure for preparing 3-((4-amino-2-methylpyrimidin-5-yl)methyl)-1-nitroso-1-(2-(trifluoromethoxy)ethyl)urea.

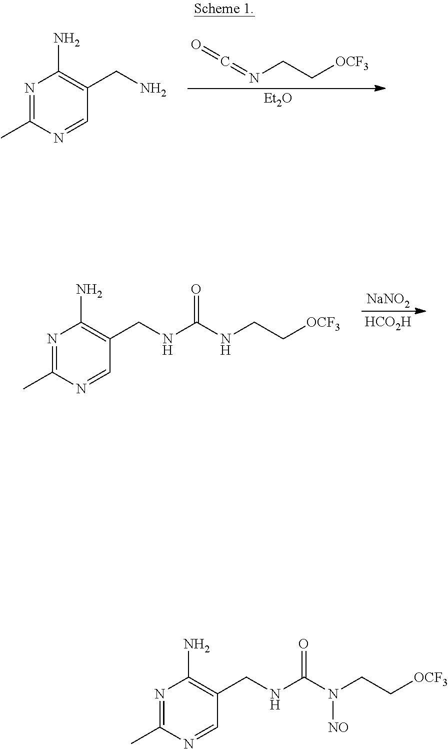

Scheme 2 is general procedure for preparing 3-(4-methylcyclohexyl)-1-nitroso-1-(2-(trifluoromethoxy)ethyl)urea.

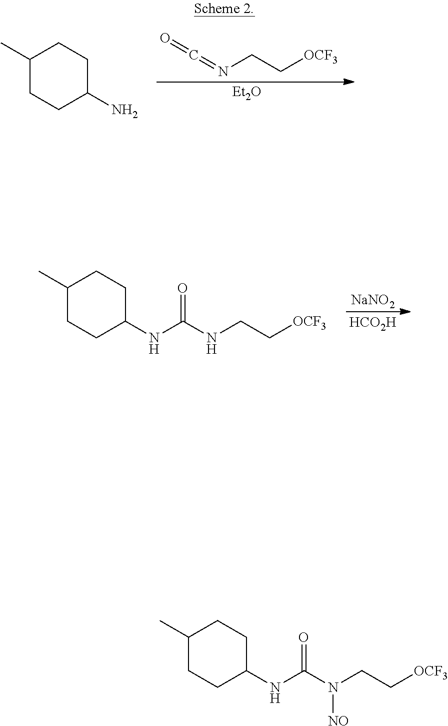

Scheme 3 is general procedure for preparing diethyl (1-(3-nitroso-3-(2-(trifluoromethoxy)ethyl)ureido)ethyl)phosphonate.

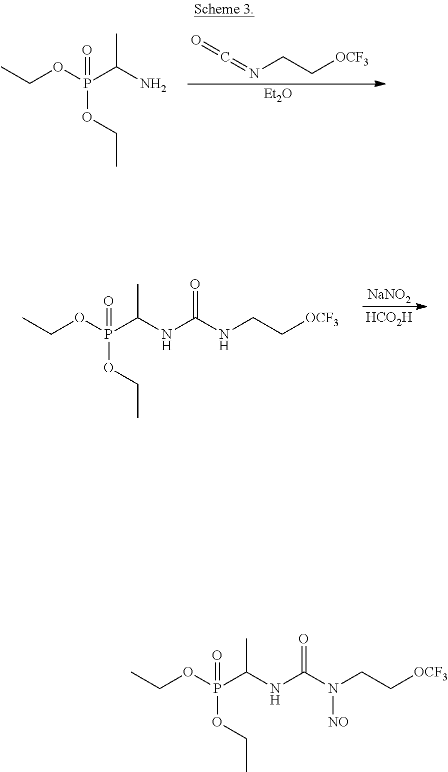

### Example 37—General Procedure for Preparation of N-cyclopropyl-4-((2-(2-(trifluoromethoxy)ethyl)hydrazineyl)methyl)benzamide

The following general procedures may be used to prepare the title compound.

### Step 1: Preparation of 4-((1,2-bis(tert-butoxycarbonyl)-2-(2-(trifluoromethoxy)ethyl) hydrazineyl)methyl)benzoic acid

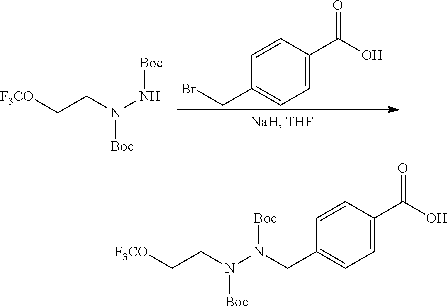 [SMILES: CC(C)(C)OC(=O)NN(CCOC(F)(F)F)C(=O)OC(C)(C)C ; O=C(O)c1ccc(CBr)cc1 ; CC(C)(C)OC(=O)N(CCOC(F)(F)F)N(Cc1ccc(C(=O)O)cc1)C(=O)OC(C)(C)C]

To a solution of di-tert-butyl 1-(2-(trifluoromethoxy)ethyl)hydrazine-1,2-dicarboxylate (1 eq) in DMF (0.2 μM) is added NaH (60% purity, 1.2 eq) at 0° C. The reaction mixture is stirred for 5 mins at 0° C. then 4-(bromomethyl)benzoic acid (1.5 eq) is added and the resulting mixture is stirred for 30 mins at 25° C. The reaction mixture is quenched with H₂O (1 vol), and then extracted with EtOAc (5 vol×3). The organic layer is washed with brine (5 vol), dried over anhydrous Na₂SO₄, filtered, and concentrated under reduced pressure. The residue is purified by flash silica gel chromatography to afford the title compound.

### Step 2: Preparation of 4-((2-(2-(trifluoromethoxy)ethyl)hydrazineyl)methyl)benzoic acid

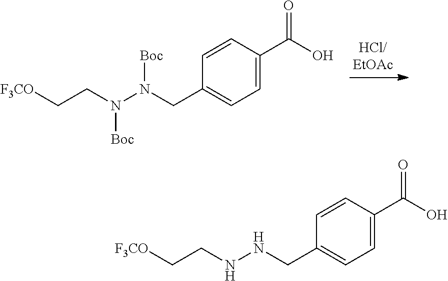 [SMILES: CC(C)(C)OC(=O)N(CCOC(F)(F)F)N(Cc1ccc(C(=O)O)cc1)C(=O)OC(C)(C)C ; O=C(O)c1ccc(CNNCCOC(F)(F)F)cc1]

A solution of 4-((1,2-bis(tert-butoxycarbonyl)-2-(2-(trifluoromethoxy)ethyl) hydrazineyl)methyl)benzoic acid (1 eq) in HCl/EtOAc (0.3 μM) is stirred at 25° C. for 16 hrs. The mixture is filtered and concentrated under reduced pressure. The residue is purified by prep-HPLC to afford the titled compound.

### Step 3: Preparation of N-cyclopropyl-4-((2-(2-(trifluoromethoxy)ethyl)hydrazineyl) methyl)benzamide

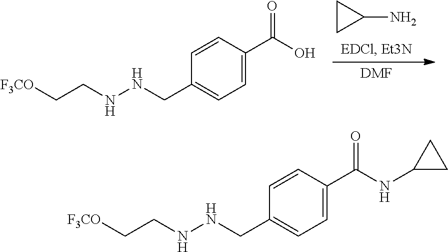

A solution of 4-((2-(2-(trifluoromethoxy)ethyl)hydrazineyl)methyl)benzoic acid (1 eq) in DMF (0.1 μM) is added EDCI (1.2 eq) and triethylamine (2.5 eq). N-cyclopropylamine (1 eq) is added and the reaction is stirred at room temperature for 24 h. The solvent is removed by rotary evaporation and the crude residue is purified (e.g., by prep-HPLC) to afford the title compound.

### Example 38—Additional General Procedures for Preparation of Procarbazine Compounds

The reaction schemes below outline additional exemplary ways that nitrosourea compounds may be prepared, based in part on general procedures described in Example 35.

Scheme 1 is general procedure for preparing N-(sec-butyl)-4-((2-(2-(trifluoromethoxy)ethyl)hydrazineyl)methyl)benzamide.

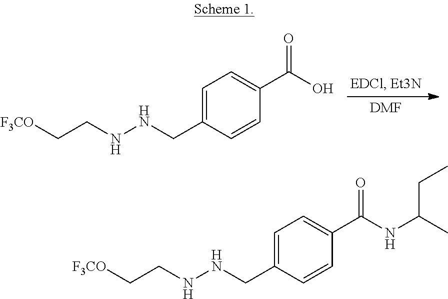

Scheme 2 is general procedure for preparing N-(1-hydroxybutan-2-yl)-4-((2-(2-(trifluoromethoxy)ethyl)hydrazineyl)methyl)benzamide.

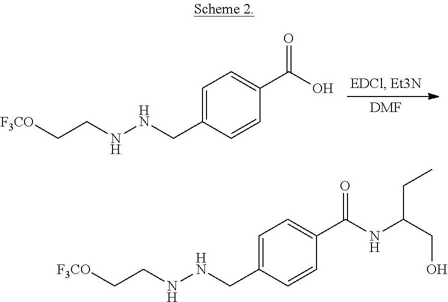

Scheme 3 is general procedure for preparing N-(1-hydroxy-2-methylpropan-2-yl)-4-((2-(2-(trifluoromethoxy)ethyl)hydrazineyl)methyl)benzamide.

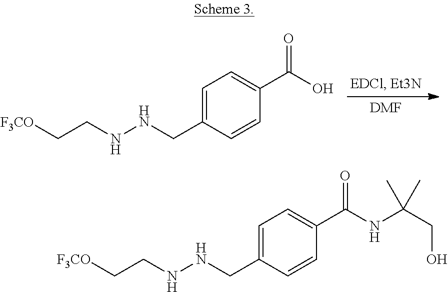

Scheme 4 is general procedure for preparing N-ethyl-4-((2-(2-(trifluoromethoxy)ethyl)hydrazineyl)methyl)benzamide.

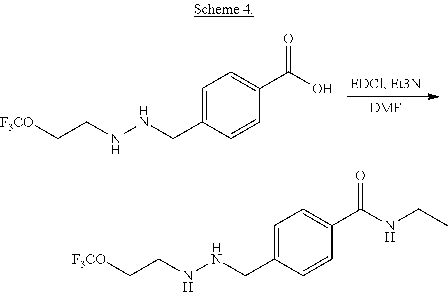

Scheme 5 is general procedure for preparing N-(tert-butyl)-4-((2-(2-(trifluoromethoxy)ethyl)hydrazineyl)methyl)benzamide.

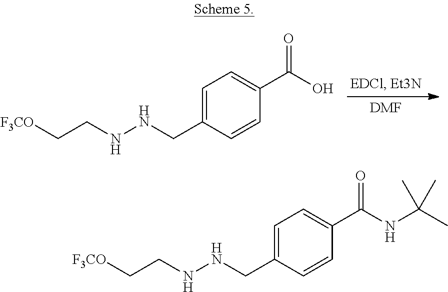

Scheme 6 is general procedure for preparing N-(1-amino-3-hydroxy-1-oxopropan-2-yl)-4-((2-(2-(trifluoromethoxy)ethyl)hydrazineyl)methyl)benzamide.

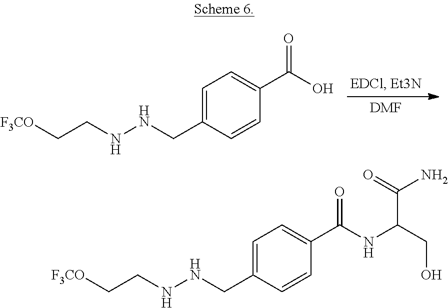

### Example 39—General Procedure for Preparation of Phenyl 4-oxo-3-(2-(trifluoromethoxy)ethyl)-3,4-dihydroimidazo[5,1-d][1,2,3,5]tetrazine-8-carboxylate

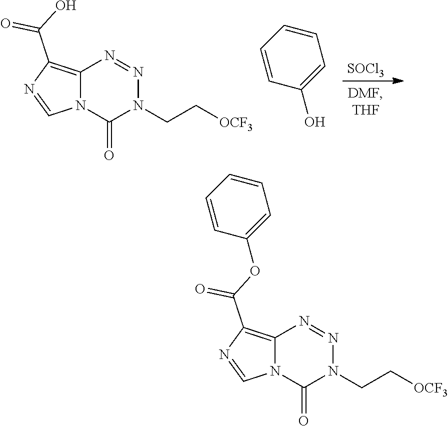

The following general procedure may be used to prepare the title compound.

To a solution of 4-oxo-3-(2-(trifluoromethoxy)ethyl)-3,4-dihydroimidazo[5,1-d][1,2,3,5]tetrazine-8-carboxylic acid (1 eq.) in THE (0.2 μM) is added DMF (0.05 eq.) and the reaction is cooled in an ice bath. Then, SOCl₂ (1.1 eq.) is added and the reaction is stirred at 0° C. for 1 h. Then, phenol (1.5 eq.) is added and the reaction is warmed to room temperature and stirred for 24 h. The reaction is concentrated to dryness on a rotary evaporator and the residue is purified (e.g., by prep-HPLC) to afford the title compound.

### Example 40—General Procedure for Preparation of S-ethyl 4-oxo-3-(2-(trifluoromethoxy)ethyl)-3,4-dihydroimidazo[5,1-d][1,2,3,5]tetrazine-8-carbothioate

The reaction scheme below outlines an exemplary synthetic route for making the title compound.

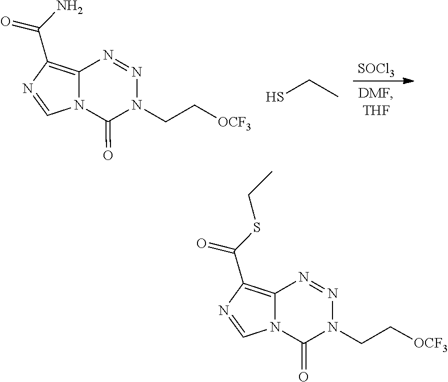

### Example 41—General Procedure for Preparation of 8-Methyl-3-(2-(trifluoromethoxy)ethyl)imidazo[5,1-d][1,2,3,5]tetrazin-4(3H)-one

The reaction scheme below outlines an exemplary synthetic route for making the title compound.

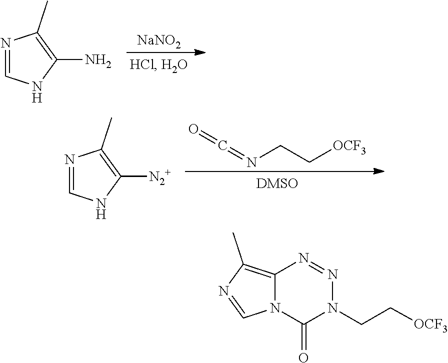

### Example 42—General Procedure for Preparation of (E)-5-(3-methyl-3-(2-(trifluoromethoxy)ethyl)triaz-1-en-1-yl)-1H-imidazole-4-carboxamide

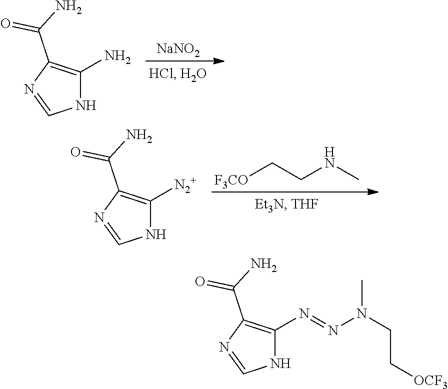

The following general procedures may be used to prepare the title compound.

### Step 1: Preparation of 4-carbamoyl-1H-imidazole-5-diazonium

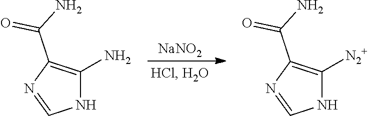

To a stirred solution of 5-amino-1H-imidazole-4-carboxamide. HCl salt (1 eq) in 1 μM HCl (0.2 μM) is added NaNO₂ solution (0.5 μM, 1.1 eq) at 0° C. and stirred for 10 min. After the reaction is completed the precipitated solid is filtered, washed with cold water (2 vol), heptane (3 vol) and dried under vacuum to afford the title compound.

### Step 2: Preparation of (E)-5-(3-methyl-3-(2-(trifluoromethoxy)ethyl)triaz-1-en-1-yl)-1H-imidazole-4-carboxamide

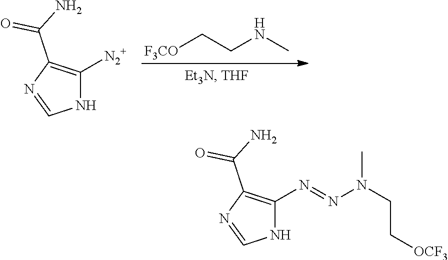

To a stirred solution of 4-carbamoyl-1H-imidazole-5-diazonium salt (1 eq) in THE (0.2 μM) is added N-methyl-2-(trifluoromethoxy)ethan-1-amine hydrochloride (1.09 eq) and Et₃N (1.09 eq) at room temperature and stirred for 6 h. After the reaction is complete, precipitated solids are filtered, washed with ethyl acetate (2×5 vol) and dried under vacuum to afford the title compound.

### Example 43—Anti-Cancer Biological Activity Assay

Exemplary compounds were evaluated for anti-cancer activity in the assay described herein below.

### Part I—Experimental Procedure

Isogenic LN229 gliobastoma cells were maintained in Dulbecco's Modified Eagle Medium (DMEM) supplemented with 10% fetal bovine serum, 1% Penicillin/Streptomycin, and 0.1% Fungin. DAY 0: Cells were plated into sterile 96-well plates (Corning Costar 96-well) at a concentration of 500 cells/well using a Multichannel pipettor. Then, assay plates were incubated overnight in a 37° C. 5% CO₂ incubator. DAY 1: Compounds were prepared as 50 mM stocks in dimethyl sulfoxide (DMSO) and stored protected from light at room temperature (RT) until use. Prior to compound addition, a two-fold serial dilution of compound stock solutions was performed in DMSO from 30 mM to 0.117 mM in a 96-well Master plate. Vehicle control wells contained DMSO. 97 μL of media was added to each well of a new 96-well plate (Daughter plate) and 3 μL of drug solution from Master plate was added to corresponding wells in the Daughter plate resulting in a 3× plate. Three replicate dilution curves of each compound were run on each assay plate. The final concentration of compounds ranged from 300 μM to 1.171 μM (9-point, 2-fold dilution dose response curve), and the final DMSO concentration was 1%.

Assay plates were incubated for 120 hours at 37° C. in a humidified 5% CO₂ incubator. DAY 5: Following incubation, cells were fixed with 4% paraformaldehyde and stained with Hoechst dye for nuclei visualization. Fixation, staining and washing was performed by using a Thermo Scientific Multidrop Combi. Images were acquired on a BioTek Cytation 5 Cell Imaging Multimode Reader and quantified using Cell Profiler image analysis software. Raw cell count data for test compounds were normalized to percent viability relative to the DMSO vehicle control.

### Part II—Results

Inhibition data for the compounds tested in the assay are provided in the table below. The symbol “++++” indicates an IC₅₀ less than or equal to 20 μM. The symbol “+++” indicates an IC₅₀ in the range of greater than 20 μM to 50 μM. The symbol “++” indicates an IC₅₀ in the range of greater than 50 μM to 100 μM. The symbol “+” indicates an IC₅₀ greater than 100 μM. “MGMT+” means that cells expressed a normal amount of MGMT. “MMR+” means that cells expressed a normal amount of MMR.

TABLE 6

| | MGMT+ | MGMT+ | MGMT− | MGMT− |
|---|---|---|---|---|
| Compound | MMR+ | MMR− | MMR+ | MMR− |
| No. | Activity | Activity | Activity | Activity |
| I-1 | + | + | ++++ | ++++ |
| I-3 | + | + | ++ | ++ |
| I-4 | ++ | +++ | +++ | +++ |
| I-5 | + | + | ++ | +++ |
| I-6 | +++ | ++++ | ++++ | +++ |
| I-7 | +++ | ++ | +++ | +++ |
| I-8 | + | + | ++ | +++ |
| I-9 | ++ | + | +++ | ++++ |
| I-10 | ++ | ++ | +++ | +++ |
| I-11 | ++ | ++ | ++++ | ++++ |
| I-12 | + | + | ++ | +++ |
| I-22 | + | + | + | + |
| I-23 | + | + | + | + |
| I-26 | + | + | +++ | ++++ |
| I-29 | + | + | +++ | ++++ |
| I-30 | + | + | + | ++++ |
| I-31 | + | + | +++ | ++++ |
| I-34 | ++ | ++ | ++++ | ++++ |
| I-39 | ++++ | +++ | ++++ | ++++ |
| I-44 | + | + | + | + |
| II-1 | ++++ | ++ | ++++ | + |
| II-2 | ++++ | +++ | ++ | +++ |
| II-3 | +++ | +++ | +++ | +++ |
| II-4 | ++++ | ++++ | +++ | ++++ |
| II-5 | ++++ | ++++ | ++++ | +++ |
| II-6 | ++++ | ++++ | +++ | ++++ |
| IV-1 | +++ | +++ | +++ | +++ |
| IV-2 | + | + | ++ | +++ |

### Example 44—Cell Panel Anti-Cancer Biological Activity Assay by Nuclear Dye

Compound I-1 was evaluated for anti-cancer activity against a panel of cancer cell lines in the assay described herein below.

### Part I—Experimental Procedure

Cells were grown in RPMI1640, 10% FBS, 2 mM L-alanyl-L-glutamine, 1 mM Na pyruvate, or a special medium. Cells were seeded into 384-well plates and incubated in a humidified atmosphere of 5% CO₂ at 37° C. Compound was added the day following cell seeding. At the same time, a time zero untreated cell plate was generated. After a 5-day incubation period, cells were fixed and stained to allow fluorescence imaging of nuclei.

Compounds were serially diluted in 2-fold steps from the highest test concentration specified, and assayed over 10 concentrations with a maximum assay concentration of 0.1% DMSO. Automated fluorescence microscopy was carried out using a Molecular Devices ImageXpress Micro XL high-content imager, and images were collected with a 4× objective. 16-bit TIFF images were acquired and analyzed with MetaXpress 5.1.0.41 software.

### Data Analysis

Cell proliferation was measured by the fluorescence intensity of an incorporated nuclear dye. The output is referred to as the relative cell count, where the measured nuclear intensity is transformed to percent of control (POC) using the following formula:

$$POC = \frac{{I}_{x}}{{I}_{0}} \times 100$$

where Iₓ is the nuclear intensity at concentration x, and I₀ is the average nuclear intensity of the untreated vehicle wells.

Cellular response parameters were calculated using nonlinear regression to a sigmoidal single-site dose response model:

$$y = A + \frac{B - A}{1 + {( C / x )}^{D}}$$

where y is a response measured at concentration x, A and B are the lower and upper limits of the response, C is the concentration at the response midpoint (EC₅₀), and D is the Hill Slope.

Time zero non-treated plates were used to determine the number of doublings during the assay period, using the formula:

$$Doublings = {log}_{2} ( \frac{N}{{N}_{T 0}} )$$

where Nis the cell number in untreated wells at the assay end point and N_{T0} is the cell number at the time of compound addition.

Cell count IC₅₀ is the test compound concentration at 50% of maximal possible response. EC₅₀ is the test compound concentration at the curve inflection point or half the effective response (parameter C of the fitted curve solution). GI₅₀ is the concentration needed to reduce the observed growth by half (midway between the curve maximum and the time zero value). Activity area is an estimate of the integrated area above the curve. Activity area values range from 0-10, where a value of zero indicates no inhibition of proliferation at all concentrations, and a value of 10 indicates complete inhibition of proliferation at all concentrations. In rare instances, values <0 or >10 may be observed. In these instances, values <0 should be considered as equivalent to 0, whereas values >10 should be considered equivalent to 10.

Curve-fitting, calculations, and report generation were performed using a custom data reduction engine and MathIQ-based software (AIM).

### Part II—Results

Data for compound I-1 and the cell lines tested in this assay are provided in Table 7 below. The symbol “++++” indicates an EC₅₀ or IC₅₀ less than or equal to 30 μM. The symbol “+++” indicates an EC₅₀ or IC₅₀ in the range of greater than 30 μM to 70 μM. The symbol “++” indicates an EC₅₀ or IC₅₀ in the range of greater than 70 μM to 150 μM. The symbol “+” indicates an EC₅₀ or IC₅₀ greater than 150 μM.

Also presented in Table 7 below is relative MGMT mRNA expression (on a Log 2 scale) in the cancer cell lines, where the average for the gene was 6.53. The symbol “#” indicates low expression, with a Log 2 value in the range of greater than 2.00 to 4.00. The symbol “##” indicates moderate expression, with a Log 2 value in the range of greater than 4.00 to 6.00. The symbol “###” indicates high expression, with a Log 2 value in the range of greater than 6.00 to 10.00.

TABLE 7

| | | | | MGMT |
|---|---|---|---|---|
| | | | | mRNA |
| | | | Lineage and/or Primary | Expres- |
| Cell Line | EC₅₀ | IC₅₀ | Disease | sion |
| SW1783 | ++ | ++ | Glioma (astrocytoma) | ## |
| SCC-4 | ++++ | + | Head & neck (squamous cell | ### |
| | | | carcinoma, tongue) | |
| NCI-H69 | ++++ | ++++ | Lung (SCLC) | # |
| MT-3 | ++++ | ++++ | Colon | # |
| SU-DHL-8 | ++++ | ++++ | Lymphoma (large cell) | # |
| SW48 | ++++ | ++++ | Colon | # |
| CRO-AP2 | ++++ | ++++ | Lymphoma | # |
| ST486 | ++++ | ++++ | Lymphoma (Burkitt's) | # |
| SR | ++++ | ++++ | Lymphoma (large cell | # |
| | | | immunoblastic) | |
| DMS53 | ++++ | ++++ | Lung (SCLC) | # |
| DB | ++++ | +++ | Lymphoma (B-cell) | ### |
| DK-MG | ++++ | +++ | Glioma | # |
| CHP-212 | ++++ | ++++ | Neuroblastoma | ## |
| BC-1 | ++++ | ++++ | Lymphoma (B-cell) | # |
| CCF-STTG1 | ++++ | +++ | Glioma (astrocytoma) | # |
| SJRH30 | ++++ | ++++ | Sarcoma (rhabdomyosarcoma) | # |
| SaOS2 | ++++ | ++++ | Osteosarcoma | ## |
| TUR | ++++ | ++++ | Lymphoma (histiocytic) | # |
| BCP-1 | ++++ | ++++ | Lymphoma (B-cell) | # |
| Hs 294T | ++++ | ++++ | Melanoma | # |
| C32TG | ++++ | ++++ | Melanoma | # |
| DBTRG-05MG | ++++ | ++++ | Glioma (glioblastoma | ## |
| | | | multiforme) | |
| 639-V | ++++ | ++++ | Bladder (ureter) | ## |
| NCI-H661 | ++++ | + | Lung (NSCLC) | ## |
| SU-DHL-10 | ++++ | +++ | Lymphoma (large cell) | ### |
| A101D | ++++ | ++++ | Melanoma | # |
| NCI-H446 | +++ | +++ | Lung (SCLC) | # |
| M059J | +++ | +++ | Glioma (glioblastoma) | # |
| OE33 | +++ | +++ | Head & neck (esophageal | # |
| | | | carcinoma) | |
| 5637 | +++ | +++ | Bladder | # |
| A427 | +++ | +++ | Lung (NSCLC) | # |
| SNB-19 | +++ | +++ | Glioma (astrocytoma) | # |
| OE21 | +++ | +++ | Head & neck (squamous cell | # |
| | | | carcinoma, esophagus) | |
| Colo 320DM | +++ | +++ | Colon | # |
| WM-266-4 | +++ | +++ | Melanoma | # |
| SH-4 | +++ | +++ | Melanoma | ## |
| T24 | +++ | + | Bladder | ### |
| 647-V | +++ | +++ | Bladder | ## |
| HOS | +++ | +++ | Osteosarcoma | ## |
| C32 | +++ | ++ | Melanoma | # |
| BFTC-905 | +++ | +++ | Bladder | # |
| U-87 MG | +++ | ++ | Glioma (glioblastoma) | # |
| SHP-77 | +++ | ++ | Lung (SCLC) | # |
| Jiyoye | +++ | +++ | Lymphoma (Burkitt's) | # |
| A172 | +++ | ++ | Glioma (glioblastoma) | # |
| SK-LMS-1 | +++ | +++ | Sarcoma (leiomyosarcoma) | # |
| SW620 | ++ | ++ | Colon | ## |
| SCC-9 | ++ | ++ | Head & neck (squamous cell | # |
| | | | carcinoma, tongue) | |
| VA-ES-BJ | ++ | + | Sarcoma (epitheloid) | ### |
| A-253 | ++ | ++ | Head & neck (epidermoid | # |
| | | | carcinoma, salivary gland) | |
| SW1088 | ++ | ++ | Glioma (astrocytoma) | # |
| KHOS-240S | + | + | Osteosarcoma | ## |
| MeWo | + | + | Melanoma | ### |
| SK-N-AS | + | + | Neuroblastoma | ### |
| NCI-H747 | + | + | Colon (cecum) | ### |
| NCI-H441 | + | + | Lung (NSCLC) | ### |

### Example 45—Cell Panel Anti-Cancer Biological Activity Assay by PRISM Assay

Compound I-1 was evaluated for anti-cancer activity against a panel of cancer cell lines in the PRISM assay described herein below.

### Part I—Experimental Procedure

The multiplexed cell-viability platform, PRISM (profiling relative inhibition simultaneously in mixtures), enabled multiplexed screening of Compound I-1 across 900 barcoded cell lines representing more than 45 lineages. Details of the assay technique are described in Yu, C., et al. “High-throughput identification of genotype-specific cancer vulnerabilities in mixtures of barcoded tumor cell lines,” Nat. Biotechnol. 2016, Vol. 34, p. 419-423; and Corsello, S. M., et al. “Discovering the anti-cancer potential of non-oncology drugs by systematic viability profiling,” Nat. Cancer, 2020, Vol. 1, p. 235-248; each of which are hereby incorporated by reference in their entirety.

Briefly, unique oligonucleotide barcodes are stably transduced into individual cancer cell lines. Following barcode transduction, individual cell lines are pooled together in groups of 20-25 based on growth rate similarity, then thawed into 384-well assay-ready plates, and treated with test compound. After 5 days of incubation, isolated mRNA is used to quantify transcribed barcode abundance of each individual cancer cell line to calculate relative viability (Log 2 fold change, “LFC”).

Compound I-1 was screened in the PRISM assay at 8-point dose (3-fold dilution) with a 5-day treatment with 885 cancer cell lines passing QC. Two PRISM cell line collections were used in the assay: PR500 (including only adherent cell lines) and PR300+(including adherent and suspension cell lines). Benchmark compounds (included in the validation compounds) were also tested at doses to ensure high data quality. All compounds were run in triplicate, and each plate contained positive (Bortezomib, 20 μM) and negative (DMSO) controls.

### Part II—Results

Compound I-1 was tested at a concentration of 66.7 μM. Presented in Table 8 are those cell lines where compound I-1 had the most significant anti-cancer affect, resulting a change in the abundance of the cells characterized by a Log 2 fold change value in the range of from −3.5 to −2.0. Presented in Table 9 are those cell lines where compound I-1 had a significant anti-cancer effect, resulting a change in the abundance of the cells characterized by a Log 2 fold change value that was in the range of greater than −2.0 to −1.0. Presented in Table 10 are those cell lines where compound I-1 had an anti-cancer effect that resulted a change in the abundance of the cells characterized in the range of greater than −1.0 to −0.5. Presented in Table 11 are those cell lines where compound I-1 had an anti-cancer effect that resulted a change in the abundance of the cells characterized in the range greater than −0.5 to −0.001. Presented in Table 12 are those cell lines where compound I-1 did not appear to provide an anti-cancer effect at the concentrations tested under the conditions of this experiment.

Tables 8-11 below also provide the Log 2 expression of MGMT in the cell line. The symbol “#” indicates low expression, with a Log 2 value less than or equal to 2.00. The symbol “##” indicates moderate expression, with a Log 2 value in the range of greater than 2.00 to 4.00. The symbol “###” indicates high expression, with a Log 2 value in the range of greater than 4.00 to 7.50.

TABLE 8

| | | | MGMT |
|---|---|---|---|
| Cell Line | Lineage | Primary Disease | Expression |
| SNU398 | Liver | Hepatocellular Carcinoma | # |
| MS751 | Cervix | Cervical Squamous Cell Carcinoma | # |
| PA1 | Ovary/Fallopian Tube | Ovarian Germ Cell Tumor | # |
| COV362 | Ovary/Fallopian Tube | Ovarian Epithelial Tumor | # |
| COGAR359 | CNS/Brain | Embryonal Tumor | # |
| BOKU | Cervix | Cervical Squamous Cell Carcinoma | # |
| PK45H | Pancreas | Pancreatic Adenocarcinoma | # |
| 769P | Kidney | Renal Cell Carcinoma | # |
| MCF7 | Breast | Invasive Breast Carcinoma | # |
| HEC6 | Uterus | Endometrial Carcinoma | # |
| HEC151 | Uterus | Endometrial Carcinoma | # |
| SNU1544 | Bowel | Colorectal Adenocarcinoma | # |
| OCILY18 | Lymphoid | Mature B-Cell Neoplasms | # |
| SNU449 | Liver | Hepatocellular Carcinoma | # |

TABLE 9

| | | | MGMT |
|---|---|---|---|
| Cell Line | Lineage | Primary Disease | Expression |
| PANC0504 | Pancreas | Pancreatic Adenocarcinoma | # |
| SIMA | Peripheral Nervous System | Neuroblastoma | # |
| JHOC5 | Ovary/Fallopian Tube | Ovarian Epithelial Tumor | ### |
| SBC5 | Lung | Lung Neuroendocrine Tumor | # |
| ONCODG1 | Ovary/Fallopian Tube | Ovarian Epithelial Tumor | # |
| HEC265 | Uterus | Endometrial Carcinoma | # |
| KYSE450 | Esophagus/Stomach | Esophageal Squamous Cell | # |
| | | Carcinoma | |
| OVCAR8 | Ovary/Fallopian Tube | Ovarian Epithelial Tumor | # |
| UMUC1 | Bladder/Urinary Tract | Bladder Urothelial Carcinoma | # |
| BICR56 | Head and Neck | Head and Neck Squamous Cell | ### |
| | | Carcinoma | |
| TC205 | Bone | Ewing Sarcoma | # |
| SNU410 | Pancreas | Pancreatic Adenocarcinoma | # |
| SKM1 | Myeloid | Acute Myeloid Leukemia | # |
| TUHR4TKB | Kidney | Renal Cell Carcinoma | # |
| WSUDLCL2 | Lymphoid | Mature B-Cell Neoplasms | # |
| CAL54 | Kidney | Renal Cell Carcinoma | # |
| NCIH28 | Pleura | Pleural Mesothelioma | # |
| JHOS2 | Ovary/Fallopian Tube | Ovarian Epithelial Tumor | # |
| NCIH82 | Lung | Lung Neuroendocrine Tumor | ### |
| NCIH2452 | Pleura | Pleural Mesothelioma | # |
| TGBCITKB | Biliary Tract | Intracholecystic Papillary Neoplasm | ## |
| SNUC2A | Bowel | Colorectal Adenocarcinoma | # |
| ONS76 | CNS/Brain | Embryonal Tumor | # |
| CAKI1 | Kidney | Renal Cell Carcinoma | # |
| G415 | Biliary Tract | Intracholecystic Papillary Neoplasm | # |
| NCIH841 | Lung | Lung Neuroendocrine Tumor | # |
| S117 | Soft Tissue | Sarcoma, NOS | ### |
| LU99 | Lung | Non-Small Cell Lung Cancer | # |
| KURAMOCHI | Ovary/Fallopian Tube | Ovarian Epithelial Tumor | # |
| HCC1937 | Breast | Invasive Breast Carcinoma | # |
| SKES1 | Bone | Ewing Sarcoma | # |
| H376 | Head and Neck | Head and Neck Squamous Cell | ### |
| | | Carcinoma | |
| TOV21G | Ovary/Fallopian Tube | Ovarian Epithelial Tumor | # |
| TCCPAN2 | Pancreas | Pancreatic Adenocarcinoma | # |
| HPAFII | Pancreas | Pancreatic Adenocarcinoma | # |
| IM95 | Esophagus/Stomach | Esophagogastric Adenocarcinoma | # |
| EBC1 | Lung | Non-Small Cell Lung Cancer | ### |
| OC316 | Ovary/Fallopian Tube | Ovarian Epithelial Tumor | # |
| JMSU1 | Bladder/Urinary Tract | Bladder Urothelial Carcinoma | # |
| IHH4 | Thyroid | Well-Differentiated Thyroid Cancer | # |
| YKG1 | CNS/Brain | Diffuse Glioma | ## |
| ZR751 | Breast | Invasive Breast Carcinoma | # |
| VAL | Lymphoid | Mature B-Cell Neoplasms | # |
| NCIH441 | Lung | Non-Small Cell Lung Cancer | # |
| HEC59 | Uterus | Endometrial Carcinoma | # |
| H413 | Head and Neck | Head and Neck Squamous Cell | # |
| | | Carcinoma | |
| 8MGBA | CNS/Brain | Diffuse Glioma | ### |
| OSC20 | Head and Neck | Head and Neck Squamous Cell | # |
| | | Carcinoma | |
| 95T1000 | Soft Tissue | Liposarcoma | # |
| SNU886 | Liver | Hepatocellular Carcinoma | ### |
| SNU638 | Esophagus/Stomach | Esophagogastric Adenocarcinoma | # |
| 253J | Bladder/Urinary Tract | Bladder Urothelial Carcinoma | ## |
| GP5D | Bowel | Colorectal Adenocarcinoma | # |
| HUPT4 | Pancreas | Pancreatic Adenocarcinoma | # |
| MFE296 | Uterus | Endometrial Carcinoma | # |
| HCC1195 | Lung | Non-Small Cell Lung Cancer | # |
| DND41 | Lymphoid | T-Lymphoblastic | # |
| | | Leukemia/Lymphoma | |
| NIHOVCAR3 | Ovary/Fallopian Tube | Ovarian Epithelial Tumor | # |
| SNU601 | Esophagus/Stomach | Esophagogastric Adenocarcinoma | ## |
| HCC15 | Lung | Non-Small Cell Lung Cancer | # |
| RERFLCAD1 | Lung | Non-Small Cell Lung Cancer | # |
| MESSA | Uterus | Uterine Sarcoma/Mesenchymal | # |
| MDAMB436 | Breast | Invasive Breast Carcinoma | # |
| MDAPCA2B | Prostate | Prostate Adenocarcinoma | # |
| CAL120 | Breast | Invasive Breast Carcinoma | ## |
| CAMA1 | Breast | Invasive Breast Carcinoma | ## |
| CAL62 | Thyroid | Anaplastic Thyroid Cancer | # |
| CALU1 | Lung | Non-Small Cell Lung Cancer | ## |
| A673 | Bone | Ewing Sarcoma | # |
| SKOV3 | Ovary/Fallopian Tube | Ovarian Epithelial Tumor | # |
| TT2609C02 | Thyroid | Well-Differentiated Thyroid Cancer | ## |
| ICC10 | Biliary Tract | Intraductal Papillary Neoplasm of | # |
| | | the Bile Duct | |
| NOZ | Biliary Tract | Intracholecystic Papillary Neoplasm | # |
| U2973 | Lymphoid | Mature B-Cell Neoplasms | ## |
| HS294T | Skin | Melanoma | ### |
| NCIH1944 | Lung | Non-Small Cell Lung Cancer | ### |
| MHHES1 | Bone | Ewing Sarcoma | # |
| HS766T | Pancreas | Pancreatic Adenocarcinoma | # |
| SW626 | Bowel | Colorectal Adenocarcinoma | # |
| C32 | Skin | Melanoma | ### |

TABLE 10

| | | | MGMT |
|---|---|---|---|
| Cell Line | Lineage | Primary Disease | Expression |
| OSRC2 | Kidney | Renal Cell Carcinoma | # |
| OV17R | Ovary/Fallopian Tube | Ovarian Epithelial Tumor | # |
| SNGM | Uterus | Endometrial Carcinoma | # |
| 22RV1 | Prostate | Prostate Adenocarcinoma | ### |
| JVM3 | Lymphoid | Mature B-Cell Neoplasms | ### |
| TCCSUP | Bladder/Urinary Tract | Bladder Urothelial Carcinoma | ### |
| UPCISCC111 | Head and Neck | Head and Neck Squamous Cell | ## |
| | | Carcinoma | |
| RCHACV | Lymphoid | B-Lymphoblastic | # |
| | | Leukemia/Lymphoma | |
| SNU1079 | Biliary Tract | Intraductal Papillary Neoplasm | # |
| | | of the Bile Duct | |
| TM87 | Soft Tissue | Rhabdoid Cancer | # |
| NCIH2004RT | Kidney | Rhabdoid Cancer | # |
| CAL78 | Bone | Chondrosarcoma | ### |
| CAPAN2 | Pancreas | Pancreatic Adenocarcinoma | # |
| JHH1 | Liver | Hepatocellular Carcinoma | # |
| NCIH1437 | Lung | Non-Small Cell Lung Cancer | # |
| ESS1 | Uterus | Uterine Sarcoma/Mesenchymal | # |
| PACADD119 | Pancreas | Pancreatic Adenocarcinoma | # |
| CL34 | Bowel | Colorectal Adenocarcinoma | # |
| ACHN | Kidney | Renal Cell Carcinoma | # |
| KMRC20 | Kidney | Renal Cell Carcinoma | # |
| RI1 | Lymphoid | Mature B-Cell Neoplasms | ## |
| OUMS23 | Bowel | Colorectal Adenocarcinoma | # |
| HCC95 | Lung | Non-Small Cell Lung Cancer | # |
| EFM19 | Breast | Invasive Breast Carcinoma | ### |
| MEL285 | Eye | Ocular Melanoma | # |
| HEC1B | Uterus | Endometrial Carcinoma | # |
| KMS12BM | Lymphoid | Mature B-Cell Neoplasms | # |
| EFE184 | Uterus | Endometrial Carcinoma | ## |
| RBE | Biliary Tract | Intraductal Papillary Neoplasm | # |
| | | of the Bile Duct | |
| RPMI8402 | Lymphoid | T-Lymphoblastic | # |
| | | Leukemia/Lymphoma | |
| EFO27 | Ovary/Fallopian Tube | Ovarian Epithelial Tumor | # |
| NCIH1435 | Lung | Non-Small Cell Lung Cancer | ### |
| SUPB15 | Lymphoid | B-Lymphoblastic | # |
| | | Leukemia/Lymphoma | |
| CH157MN | CNS/Brain | Meningothelial Tumor | ### |
| JHUEM2 | Uterus | Endometrial Carcinoma | # |
| NB4 | Myeloid | Acute Myeloid Leukemia | ## |
| LNCAPCLON | Prostate | Prostate Adenocarcinoma | # |
| EFGC | | | |
| SUDHL4 | Lymphoid | Mature B-Cell Neoplasms | # |
| GDM1 | Myeloid | Acute Myeloid Leukemia | ### |
| RCC10RGB | Kidney | Renal Cell Carcinoma | # |
| KHM1B | Lymphoid | Mature B-Cell Neoplasms | ### |
| CORL23 | Lung | Non-Small Cell Lung Cancer | ### |
| SKMEL2 | Skin | Melanoma | ### |
| OCIC4P | Ovary/Fallopian Tube | Ovarian Epithelial Tumor | ## |
| MALME3M | Skin | Melanoma | ### |
| SUIT2 | Pancreas | Pancreatic Adenocarcinoma | # |
| FLO1 | Esophagus/Stomach | Esophagogastric | ### |
| | | Adenocarcinoma | |
| SF295 | CNS/Brain | Diffuse Glioma | ### |
| SNU1041 | Head and Neck | Head and Neck Squamous Cell | ### |
| | | Carcinoma | |
| QGP1 | Pancreas | Pancreatic Neuroendocrine | ### |
| | | Tumor | |
| BT16 | CNS/Brain | Embryonal Tumor | # |
| MKN45 | Esophagus/Stomach | Esophagogastric | # |
| | | Adenocarcinoma | |
| NCCIT | Testis | Non-Seminomatous Germ Cell | ## |
| | | Tumor | |
| JEG3 | Uterus | Gestational Trophoblastic | # |
| | | Disease | |
| CCLFPEDS0003T | Soft Tissue | Undifferentiated Pleomorphic | # |
| | | Sarcoma/Malignant Fibrous | |
| | | Histiocytoma/High-Grade | |
| | | Spindle Cell Sarcoma | |
| OAW42 | Ovary/Fallopian Tube | Ovarian Epithelial Tumor | # |
| SKMES1 | Lung | Non-Small Cell Lung Cancer | ## |
| LS411N | Bowel | Colorectal Adenocarcinoma | ## |
| RH28 | Soft Tissue | Rhabdomyosarcoma | # |
| NCIH747 | Bowel | Colorectal Adenocarcinoma | # |
| JM1 | Lymphoid | B-Lymphoblastic | ### |
| | | Leukemia/Lymphoma | |
| COLO680N | Esophagus/Stomach | Esophageal Squamous Cell | ## |
| | | Carcinoma | |
| COGE352 | Bone | Ewing Sarcoma | # |
| HARA | Lung | Non-Small Cell Lung Cancer | # |
| SCLC21H | Lung | Lung Neuroendocrine Tumor | ### |
| C33A | Cervix | Cervical Squamous Cell | ## |
| | | Carcinoma | |
| AGS | Esophagus/Stomach | Esophagogastric | # |
| | | Adenocarcinoma | |
| OVK18 | Ovary/Fallopian Tube | Ovarian Epithelial Tumor | # |
| U87MG | CNS/Brain | Diffuse Glioma | ### |
| SNU423 | Liver | Hepatocellular Carcinoma | ### |
| JEKO1 | Lymphoid | Mature B-Cell Neoplasms | # |
| EWS502 | Bone | Ewing Sarcoma | ## |
| NCIH460 | Lung | Non-Small Cell Lung Cancer | ### |
| MKN74 | Esophagus/Stomach | Esophagogastric | ## |
| | | Adenocarcinoma | |
| JAR | Uterus | Gestational Trophoblastic | ### |
| | | Disease | |
| OZ | Biliary Tract | Intraductal Papillary Neoplasm | # |
| | | of the Bile Duct | |
| RERFLCMS | Lung | Non-Small Cell Lung Cancer | ## |
| RCK8 | Lymphoid | Mature B-Cell Neoplasms | ### |
| KYSE140 | Esophagus/Stomach | Esophageal Squamous Cell | ### |
| | | Carcinoma | |
| OCIC5X | Ovary/Fallopian Tube | Ovarian Epithelial Tumor | # |
| BT549 | Breast | Invasive Breast Carcinoma | ## |
| GAMG | CNS/Brain | Diffuse Glioma | ### |
| JURKAT | Lymphoid | T-Lymphoblastic | # |
| | | Leukemia/Lymphoma | |
| TEN | Uterus | Endometrial Carcinoma | # |
| CAL72 | Bone | Osteosarcoma | ### |
| MKN1 | Esophagus/Stomach | Esophagogastric | ## |
| | | Adenocarcinoma | |
| HCC1143 | Breast | Invasive Breast Carcinoma | ## |
| LN18 | CNS/Brain | Diffuse Glioma | ## |
| SUM159PT | Breast | Invasive Breast Carcinoma | ### |
| CAL29 | Bladder/Urinary Tract | Bladder Urothelial Carcinoma | ## |
| CBAGPN | Bone | Ewing Sarcoma | # |
| OVKATE | Ovary/Fallopian Tube | Ovarian Epithelial Tumor | ## |
| TE6 | Esophagus/Stomach | Esophageal Squamous Cell | ## |
| | | Carcinoma | |
| ACCMESO1 | Pleura | Pleural Mesothelioma | ## |
| KMRC3 | Kidney | Renal Cell Carcinoma | # |
| NCIH1581 | Lung | Non-Small Cell Lung Cancer | ## |
| HS939T | Skin | Melanoma | ### |
| SW756 | Cervix | Cervical Squamous Cell | ### |
| | | Carcinoma | |
| TF1 | Myeloid | Acute Myeloid Leukemia | ### |
| HCC515 | Lung | Non-Small Cell Lung Cancer | # |
| NCIH1876 | Lung | Lung Neuroendocrine Tumor | ### |
| TE1 | Esophagus/Stomach | Esophageal Squamous Cell | ## |
| | | Carcinoma | |
| ISTMES2 | Pleura | Pleural Mesothelioma | ## |
| NCIH2009 | Lung | Non-Small Cell Lung Cancer | ### |
| OE19 | Esophagus/Stomach | Esophageal Squamous Cell | ## |
| | | Carcinoma | |
| MEL290 | Eye | Ocular Melanoma | # |
| LN229 | CNS/Brain | Diffuse Glioma | ### |
| PANC1005 | Pancreas | Pancreatic Adenocarcinoma | # |
| A101D | Skin | Melanoma | ### |
| MM127 | Skin | Melanoma | ### |
| LS1034 | Bowel | Colorectal Adenocarcinoma | ### |
| OC314 | Ovary/Fallopian Tube | Ovarian Epithelial Tumor | # |
| SCC25 | Head and Neck | Head and Neck Squamous Cell | ### |
| | | Carcinoma | |
| HT55 | Bowel | Colorectal Adenocarcinoma | ## |
| YD8 | Head and Neck | Head and Neck Squamous Cell | # |
| | | Carcinoma | |
| TTC442 | Soft Tissue | Rhabdomyosarcoma | # |
| CAL148 | Breast | Invasive Breast Carcinoma | # |
| TALL1 | Lymphoid | Mature T and NK Neoplasms | ## |
| SW1353 | Bone | Chondrosarcoma | ### |
| PANC0813 | Pancreas | Pancreatic Adenocarcinoma | ### |
| NUDHL1 | Lymphoid | Mature B-Cell Neoplasms | ## |
| IALM | Lung | Non-Small Cell Lung Cancer | ## |
| KYSE270 | Esophagus/Stomach | Esophageal Squamous Cell | ## |
| | | Carcinoma | |
| HT1376 | Bladder/Urinary Tract | Bladder Urothelial Carcinoma | ## |
| SNU245 | Biliary Tract | Intraductal Papillary Neoplasm | # |
| | | of the Bile Duct | |
| SW1783 | CNS/Brain | Diffuse Glioma | ### |
| KP3 | Pancreas | Adenosquamous Carcinoma of | # |
| | | the Pancreas | |
| HA1E | Kidney | Non-Cancerous | ### |
| RKN | Soft Tissue | Leiomyosarcoma | # |
| SW1710 | Bladder/Urinary Tract | Bladder Urothelial Carcinoma | ## |
| NALM6 | Lymphoid | B-Lymphoblastic | ## |
| | | Leukemia/Lymphoma | |
| UMUC7 | Bladder/Urinary Tract | Bladder Urothelial Carcinoma | ### |
| BICR31 | Head and Neck | Head and Neck Squamous Cell | ### |
| | | Carcinoma | |
| RERFLCAI | Lung | Non-Small Cell Lung Cancer | ## |
| LOUNH91 | Lung | Non-Small Cell Lung Cancer | ## |
| CCLP1 | Biliary Tract | Intraductal Papillary Neoplasm | ### |
| | | of the Bile Duct | |
| DAOY | CNS/Brain | Embryonal Tumor | ### |
| BLUE1 | Lymphoid | Mature B-Cell Neoplasms | ## |
| BCPAP | Thyroid | Poorly Differentiated Thyroid | ### |
| | | Cancer | |
| NCIH647 | Lung | Non-Small Cell Lung Cancer | ### |

TABLE 11

| | | | MGMT |
|---|---|---|---|
| Cell Line | Lineage | Primary Disease | Expression |
| KYSE510 | Esophagus/Stomach | Esophageal Squamous Cell | ### |
| | | Carcinoma | |
| LS180 | Bowel | Colorectal Adenocarcinoma | ## |
| HOS | Bone | Osteosarcoma | ### |
| ASPC1 | Pancreas | Pancreatic Adenocarcinoma | # |
| HEP3B217 | Liver | Hepatocellular Carcinoma | ## |
| KLE | Uterus | Endometrial Carcinoma | # |
| CW9019 | Soft Tissue | Rhabdomyosarcoma | ### |
| TT1TKB | Bowel | Colorectal Adenocarcinoma | ## |
| SHMAC5 | Prostate | Prostate Adenocarcinoma | ## |
| SW837 | Bowel | Colorectal Adenocarcinoma | # |
| NP5 | CNS/Brain | Diffuse Glioma | ### |
| MDAMB468 | Breast | Invasive Breast Carcinoma | ## |
| SW579 | Head and Neck | Head and Neck Squamous Cell | ### |
| | | Carcinoma | |
| NUGC3 | Esophagus/Stomach | Esophagogastric Adenocarcinoma | ## |
| C80 | Bowel | Colorectal Adenocarcinoma | # |
| HCA1 | Cervix | Cervical Adenocarcinoma | ### |
| KALS1 | CNS/Brain | Diffuse Glioma | ## |
| SJSA1 | Bone | Osteosarcoma | ### |
| HCC4006 | Lung | Non-Small Cell Lung Cancer | ## |
| KELLY | Peripheral Nervous | Neuroblastoma | ## |
| | System | | |
| SNU213 | Pancreas | Pancreatic Adenocarcinoma | ## |
| TGBC52TKB | Ampulla of Vater | Ampullary Carcinoma | # |
| 2313287 | Esophagus/Stomach | Esophagogastric Adenocarcinoma | # |
| 253JBV | Bladder/Urinary Tract | Bladder Urothelial Carcinoma | ### |
| SW780 | Bladder/Urinary Tract | Bladder Urothelial Carcinoma | ## |
| MDAMB175VII | Breast | Invasive Breast Carcinoma | # |
| BECKER | CNS/Brain | Diffuse Glioma | ### |
| NCIH358 | Lung | Non-Small Cell Lung Cancer | ## |
| RH4 | Soft Tissue | Rhabdomyosarcoma | ### |
| HUH1 | Liver | Hepatocellular Carcinoma | # |
| GSU | Esophagus/Stomach | Esophagogastric Adenocarcinoma | ### |
| A1207 | CNS/Brain | Diffuse Glioma | ### |
| HEC1 | Uterus | Endometrial Carcinoma | # |
| NP2 | CNS/Brain | Diffuse Glioma | ### |
| NCIH661 | Lung | Non-Small Cell Lung Cancer | ## |
| SW1573 | Lung | Non-Small Cell Lung Cancer | ### |
| UMUC5 | Bladder/Urinary Tract | Bladder Squamous Cell Carcinoma | ## |
| G361 | Skin | Melanoma | ## |
| G292CLONEA141B1 | Bone | Osteosarcoma | # |
| GB1 | CNS/Brain | Diffuse Glioma | ### |
| OCIM1 | Myeloid | Acute Myeloid Leukemia | ### |
| NCO2 | Myeloid | Myeloproliferative Neoplasms | ### |
| P12ICHIKAWA | Lymphoid | T-Lymphoblastic | ## |
| | | Leukemia/Lymphoma | |
| BXPC3 | Pancreas | Pancreatic Adenocarcinoma | # |
| SNU489 | CNS/Brain | Diffuse Glioma | ### |
| 647V | Bladder/Urinary Tract | Bladder Urothelial Carcinoma | ### |
| TE617T | Soft Tissue | Rhabdomyosarcoma | # |
| KMRC1 | Kidney | Renal Cell Carcinoma | # |
| CAL51 | Breast | Invasive Breast Carcinoma | # |
| CW2 | Bowel | Colorectal Adenocarcinoma | ### |
| SNU61 | Bowel | Colorectal Adenocarcinoma | # |
| DETROIT562 | Head and Neck | Head and Neck Squamous Cell | ## |
| | | Carcinoma | |
| OCILY3 | Lymphoid | Mature B-Cell Neoplasms | ### |
| U251MG | CNS/Brain | Diffuse Glioma | ### |
| LCLC103H | Lung | Non-Small Cell Lung Cancer | ### |
| HSC5 | Skin | Cutaneous Squamous Cell | ### |
| | | Carcinoma | |
| HCC1419 | Breast | Invasive Breast Carcinoma | ## |
| A2058 | Skin | Melanoma | ### |
| T3M10 | Lung | Non-Small Cell Lung Cancer | ## |
| NCIH1693 | Lung | Non-Small Cell Lung Cancer | # |
| SW1271 | Lung | Lung Neuroendocrine Tumor | # |
| HT | Lymphoid | Mature B-Cell Neoplasms | ## |
| SNU407 | Bowel | Colorectal Adenocarcinoma | # |
| SNU840 | Ovary/Fallopian Tube | Ovarian Epithelial Tumor | ### |
| MPP89 | Pleura | Pleural Mesothelioma | ## |
| MHHNB11 | Peripheral Nervous | Neuroblastoma | # |
| | System | | |
| SNU182 | Liver | Hepatocellular Carcinoma | # |
| NCIH1648 | Lung | Non-Small Cell Lung Cancer | ## |
| J82 | Bladder/Urinary Tract | Bladder Urothelial Carcinoma | ## |
| IGR37 | Skin | Melanoma | ## |
| OCUG1 | Biliary Tract | Intracholecystic Papillary Neoplasm | ## |
| MDAMB231 | Breast | Invasive Breast Carcinoma | ### |
| HT1080 | Soft Tissue | Fibrosarcoma | ### |
| COLO668 | Lung | Lung Neuroendocrine Tumor | ## |
| MB1 | Thyroid | Anaplastic Thyroid Cancer | # |
| RDES | Bone | Ewing Sarcoma | # |
| NCIH157DM | Lung | Non-Small Cell Lung Cancer | ### |
| MOLM14 | Myeloid | Acute Myeloid Leukemia | ### |
| PATU8988T | Pancreas | Pancreatic Adenocarcinoma | # |
| SISO | Cervix | Cervical Adenocarcinoma | ### |
| UPCISCC072 | Head and Neck | Head and Neck Squamous Cell | ## |
| | | Carcinoma | |
| NCIH727 | Lung | Lung Neuroendocrine Tumor | ### |
| CORL105 | Lung | Non-Small Cell Lung Cancer | ## |
| SKNO1 | Myeloid | Acute Myeloid Leukemia | ## |
| NCIH1355 | Lung | Non-Small Cell Lung Cancer | ## |
| GOS3 | CNS/Brain | Diffuse Glioma | ### |
| HCC38 | Breast | Invasive Breast Carcinoma | ## |
| NCIH1573 | Lung | Non-Small Cell Lung Cancer | ## |
| PC14 | Lung | Non-Small Cell Lung Cancer | ## |
| SG231 | Biliary Tract | Intraductal Papillary Neoplasm of | ### |
| | | the Bile Duct | |
| HEC251 | Uterus | Endometrial Carcinoma | ### |
| SEM | Lymphoid | B-Lymphoblastic | ### |
| | | Leukemia/Lymphoma | |
| KP2 | Pancreas | Pancreatic Adenocarcinoma | ## |
| KMH2 | Lymphoid | Hodgkin Lymphoma | ## |
| RMUGS | Ovary/Fallopian Tube | Ovarian Epithelial Tumor | ## |
| BICR16 | Head and Neck | Head and Neck Squamous Cell | ## |
| | | Carcinoma | |
| RERFLCKJ | Lung | Non-Small Cell Lung Cancer | ## |
| KMCH1 | Liver | Hepatocellular Carcinoma plus | # |
| | | Intrahepatic Cholangiocarcinoma | |
| MFE280 | Uterus | Endometrial Carcinoma | ## |
| PC3 | Prostate | Prostate Adenocarcinoma | ### |
| MC116 | Lymphoid | Non-Hodgkin Lymphoma | ## |
| NCIH446 | Lung | Lung Neuroendocrine Tumor | ### |
| T47D | Breast | Invasive Breast Carcinoma | ## |
| KKU100 | Biliary Tract | Intraductal Papillary Neoplasm of | ### |
| | | the Bile Duct | |
| NCIH520 | Lung | Non-Small Cell Lung Cancer | ### |
| TFK1 | Biliary Tract | Intraductal Papillary Neoplasm of | ### |
| | | the Bile Duct | |
| SW13 | Adrenal Gland | Adrenocortical Carcinoma | # |
| EM2 | Myeloid | Myeloproliferative Neoplasms | ### |
| CALU6 | Lung | Non-Small Cell Lung Cancer | ### |
| WM88 | Skin | Melanoma | ### |
| YAPC | Pancreas | Pancreatic Adenocarcinoma | # |
| NCIH838 | Lung | Non-Small Cell Lung Cancer | ## |
| PACADD161 | Pancreas | Pancreatic Adenocarcinoma | ## |
| SNU878 | Liver | Hepatocellular Carcinoma | ## |
| TE4 | Esophagus/Stomach | Esophageal Squamous Cell | ### |
| | | Carcinoma | |
| SNU466 | CNS/Brain | Diffuse Glioma | ## |
| HKGZCC | Biliary Tract | Intraductal Papillary Neoplasm of | ### |
| | | the Bile Duct | |
| KNS42 | CNS/Brain | Diffuse Glioma | ### |
| PK8 | Pancreas | Pancreatic Adenocarcinoma | ## |
| MCC13 | Skin | Merkel Cell Carcinoma | # |
| LOXIMVI | Skin | Melanoma | ### |
| 1777NRPMET | Testis | Non-Seminomatous Germ Cell | ## |
| | | Tumor | |
| NCIH1650 | Lung | Non-Small Cell Lung Cancer | ## |
| OCIAML5 | Myeloid | Acute Myeloid Leukemia | ## |
| MOLM16 | Myeloid | Acute Myeloid Leukemia | ## |
| SNU1105 | CNS/Brain | Diffuse Glioma | ### |
| CI1 | Lymphoid | Non-Hodgkin Lymphoma | ### |
| HUPT3 | Pancreas | Pancreatic Adenocarcinoma | ## |
| UMUC13 | Bladder/Urinary Tract | Bladder Urothelial Carcinoma | ### |
| TGBC18TKB | Ampulla of Vater | Ampullary Carcinoma | # |
| UPCISCC116 | Head and Neck | Head and Neck Squamous Cell | ### |
| | | Carcinoma | |
| SNU46 | Head and Neck | Head and Neck Squamous Cell | # |
| | | Carcinoma | |
| NP3 | CNS/Brain | Diffuse Glioma | ## |
| KNS62 | Lung | Non-Small Cell Lung Cancer | ## |
| NB1 | Peripheral Nervous | Neuroblastoma | ## |
| | System | | |
| A2780 | Ovary/Fallopian Tube | Ovarian Epithelial Tumor | ### |
| TE10 | Esophagus/Stomach | Esophageal Squamous Cell | # |
| | | Carcinoma | |
| SW156 | Kidney | Renal Cell Carcinoma | # |
| RPMI7951 | Skin | Melanoma | ### |
| HCC78 | Lung | Non-Small Cell Lung Cancer | # |
| BFTC909 | Kidney | Renal Cell Carcinoma | ## |
| FTC133 | Thyroid | Well-Differentiated Thyroid Cancer | ## |
| SSP25 | Biliary Tract | Intraductal Papillary Neoplasm of | ### |
| | | the Bile Duct | |
| RH36 | Soft Tissue | Rhabdomyosarcoma | ## |
| SNU1077 | Uterus | Endometrial Carcinoma | ## |
| CFPAC1 | Pancreas | Pancreatic Adenocarcinoma | ### |
| H103 | Head and Neck | Head and Neck Squamous Cell | ### |
| | | Carcinoma | |
| RHJT | Soft Tissue | Rhabdomyosarcoma | ## |
| NCIH226 | Pleura | Pleural Mesothelioma | ### |
| BHY | Head and Neck | Head and Neck Squamous Cell | ### |
| | | Carcinoma | |
| CAL12T | Lung | Non-Small Cell Lung Cancer | ### |
| HEC1A | Uterus | Endometrial Carcinoma | # |
| SKHEP1 | Liver | Hepatocellular Carcinoma | ## |
| DOTC24510 | Cervix | Mixed Cervical Carcinoma | ### |
| BICR78 | Head and Neck | Head and Neck Squamous Cell | ### |
| | | Carcinoma | |
| CHLA06ATRT | CNS/Brain | Embryonal Tumor | # |
| OVISE | Ovary/Fallopian Tube | Ovarian Epithelial Tumor | ## |
| SKGI | Cervix | Cervical Squamous Cell Carcinoma | ## |
| SKGT4 | Esophagus/Stomach | Esophagogastric Adenocarcinoma | ## |
| BHT101 | Thyroid | Anaplastic Thyroid Cancer | ### |
| SNU1066 | Head and Neck | Head and Neck Squamous Cell | ## |
| | | Carcinoma | |
| NCIH1838 | Lung | Non-Small Cell Lung Cancer | ### |
| LN464 | CNS/Brain | Diffuse Glioma | ### |
| PACADD137 | Pancreas | Pancreatic Adenocarcinoma | ### |
| HSKTC | Ovary/Fallopian Tube | Ovarian Cancer, Other | ## |
| MONOMAC1 | Myeloid | Acute Myeloid Leukemia | ### |
| COLO800 | Skin | Melanoma | ### |
| KP4 | Pancreas | Pancreatic Adenocarcinoma | ## |
| RERFGC1B | Esophagus/Stomach | Esophagogastric Adenocarcinoma | # |
| CCLFPEDS0001T | Kidney | Renal Cell Carcinoma | # |
| LI7 | Liver | Hepatocellular Carcinoma | ### |
| VAESBJ | Soft Tissue | Epithelioid Sarcoma | # |
| SCABER | Bladder/Urinary Tract | Bladder Squamous Cell Carcinoma | ### |
| KKU055 | Biliary Tract | Intraductal Papillary Neoplasm of | # |
| | | the Bile Duct | |
| EMTOKA | Uterus | Endometrial Carcinoma | # |
| JHH5 | Liver | Hepatocellular Carcinoma | ## |
| GI1 | CNS/Brain | Diffuse Glioma | ### |
| ES2 | Ovary/Fallopian Tube | Ovarian Epithelial Tumor | ### |
| HDQP1 | Breast | Invasive Breast Carcinoma | ### |
| PEA1 | Ovary/Fallopian Tube | Ovarian Epithelial Tumor | # |
| OV7 | Ovary/Fallopian Tube | Ovarian Epithelial Tumor | ## |
| NCIH2347 | Lung | Non-Small Cell Lung Cancer | ## |
| NCIH23 | Lung | Non-Small Cell Lung Cancer | ### |
| NZOV9 | Ovary/Fallopian Tube | Ovarian Epithelial Tumor | ### |
| UMUC11 | Bladder/Urinary Tract | Bladder Urothelial Carcinoma | ### |
| KARPAS1718 | Lymphoid | Mature B-Cell Neoplasms | ### |
| HPAC | Pancreas | Pancreatic Adenocarcinoma | ## |
| YH13 | CNS/Brain | Diffuse Glioma | ## |
| BEN | Lung | Non-Small Cell Lung Cancer | ### |
| HEL9217 | Myeloid | Acute Myeloid Leukemia | ### |
| JIMT1 | Breast | Invasive Breast Carcinoma | # |
| KU1919 | Bladder/Urinary Tract | Bladder Urothelial Carcinoma | ### |
| MM1S | Lymphoid | Mature B-Cell Neoplasms | ### |
| NCIH1048 | Lung | Lung Neuroendocrine Tumor | ## |
| JHH7 | Liver | Hepatocellular Carcinoma | # |
| KE37 | Lymphoid | Mature T and NK Neoplasms | ## |
| NCIH1915 | Lung | Non-Small Cell Lung Cancer | ### |
| UMRC7 | Kidney | Renal Cell Carcinoma | ## |
| HEPG2 | Liver | Hepatoblastoma | ## |
| LPS141 | Soft Tissue | Liposarcoma | ### |
| WM2664 | Skin | Melanoma | ### |
| HCC56 | Bowel | Colorectal Adenocarcinoma | # |
| LUDLU1 | Lung | Non-Small Cell Lung Cancer | ### |
| EB1 | Lymphoid | Mature B-Cell Neoplasms | ## |
| GP2D | Bowel | Colorectal Adenocarcinoma | # |
| KARPAS299 | Lymphoid | Mature T and NK Neoplasms | ### |
| SKMEL28 | Skin | Melanoma | ### |
| TT | Thyroid | Medullary Thyroid Cancer | ### |
| MDAMB361 | Breast | Invasive Breast Carcinoma | ### |
| SNU1 | Esophagus/Stomach | Esophagogastric Adenocarcinoma | # |
| SKMEL30 | Skin | Melanoma | ### |
| SAOS2 | Bone | Osteosarcoma | ### |
| BC3C | Bladder/Urinary Tract | Bladder Urothelial Carcinoma | ## |
| NCIH1092 | Lung | Lung Neuroendocrine Tumor | ### |
| KNS60 | CNS/Brain | Diffuse Glioma | ### |
| MG63 | Bone | Osteosarcoma | ### |
| WM793 | Skin | Melanoma | ### |
| MOLP2 | Lymphoid | Mature B-Cell Neoplasms | ### |
| SNU201 | CNS/Brain | Diffuse Glioma | ### |
| ECGI10 | Esophagus/Stomach | Esophageal Squamous Cell | ### |
| | | Carcinoma | |
| SKMEL24 | Skin | Melanoma | ### |
| NCIH1299 | Lung | Non-Small Cell Lung Cancer | ## |
| TGW | Peripheral Nervous | Neuroblastoma | ## |
| | System | | |
| OACM51 | Esophagus/Stomach | Esophagogastric Adenocarcinoma | ### |
| FTC238 | Thyroid | Well-Differentiated Thyroid Cancer | ### |
| HS611T | Lymphoid | Hodgkin Lymphoma | ### |
| SKMEL3 | Skin | Melanoma | ### |
| NCIH650 | Lung | Non-Small Cell Lung Cancer | ## |
| T24 | Bladder/Urinary Tract | Bladder Urothelial Carcinoma | ## |
| CHLA15 | Peripheral Nervous | Neuroblastoma | ## |
| | System | | |
| 94T778 | Soft Tissue | Liposarcoma | ### |
| SNU668 | Esophagus/Stomach | Esophagogastric Adenocarcinoma | # |
| MOLT3 | Lymphoid | Mature T and NK Neoplasms | ### |
| SUDHL8 | Lymphoid | Mature B-Cell Neoplasms | ### |
| U2904 | Lymphoid | Mature B-Cell Neoplasms | ### |
| CL11 | Bowel | Colorectal Adenocarcinoma | ## |
| NCIH2052 | Pleura | Pleural Mesothelioma | ### |
| TTC1240 | Kidney | Rhabdoid Cancer | ## |
| CAOV3 | Ovary/Fallopian Tube | Ovarian Epithelial Tumor | ## |
| AU565 | Breast | Invasive Breast Carcinoma | ### |
| SNU1196 | Biliary Tract | Intraductal Papillary Neoplasm of | ## |
| | | the Bile Duct | |
| KE39 | Esophagus/Stomach | Esophagogastric Adenocarcinoma | ## |
| HCC1438 | Lung | Non-Small Cell Lung Cancer | ## |
| IGR1 | Skin | Melanoma | ### |
| KMBC2 | Bladder/Urinary Tract | Bladder Urothelial Carcinoma | ## |
| KMS28BM | Lymphoid | Mature B-Cell Neoplasms | ## |
| CCFSTTG1 | CNS/Brain | Diffuse Glioma | ### |
| MOLT16 | Lymphoid | T-Lymphoblastic | ### |
| | | Leukemia/Lymphoma | |
| SUSA | Testis | Non-Seminomatous Germ Cell | ### |
| | | Tumor | |
| JHH4 | Liver | Hepatocellular Carcinoma | ### |
| HTMMT | Uterus | Endometrial Carcinoma | # |
| KYM1 | Soft Tissue | Rhabdomyosarcoma | ## |
| HCT116 | Bowel | Colorectal Adenocarcinoma | ### |
| OCIAML2 | Myeloid | Acute Myeloid Leukemia | ### |
| NCIH1341 | Lung | Lung Neuroendocrine Tumor | ## |
| HCC827 | Lung | Non-Small Cell Lung Cancer | ### |
| HUH6 | Liver | Hepatoblastoma | # |
| SKNDZ | Peripheral Nervous | Neuroblastoma | ### |
| | System | | |
| SKNFI | Peripheral Nervous | Neuroblastoma | ## |
| | System | | |
| MEG01 | Myeloid | Myeloproliferative Neoplasms | ### |
| U118MG | CNS/Brain | Diffuse Glioma | ### |
| KOPN8 | Lymphoid | B-Lymphoblastic | # |
| | | Leukemia/Lymphoma | |
| NCIH2126 | Lung | Non-Small Cell Lung Cancer | ### |
| LN382 | CNS/Brain | Diffuse Glioma | ### |
| SW900 | Lung | Non-Small Cell Lung Cancer | ## |
| HLF | Liver | Hepatocellular Carcinoma | ## |
| LAMA84 | Myeloid | Myeloproliferative Neoplasms | ### |
| HL60 | Myeloid | Acute Myeloid Leukemia | ## |
| TE9 | Esophagus/Stomach | Esophageal Squamous Cell | ### |
| | | Carcinoma | |
| SHP77 | Lung | Lung Neuroendocrine Tumor | ### |
| SKNBE2 | Peripheral Nervous | Neuroblastoma | ## |
| | System | | |
| CASKI | Cervix | Cervical Squamous Cell Carcinoma | ### |
| NCIH1568 | Lung | Non-Small Cell Lung Cancer | ### |
| PLCPRF5 | Liver | Hepatocellular Carcinoma | ## |
| HSC3 | Head and Neck | Head and Neck Squamous Cell | ### |
| | | Carcinoma | |
| BT474 | Breast | Invasive Breast Carcinoma | ### |
| H357 | Head and Neck | Head and Neck Squamous Cell | ### |
| | | Carcinoma | |
| SW1990 | Pancreas | Pancreatic Adenocarcinoma | ## |
| LXF289 | Lung | Non-Small Cell Lung Cancer | ### |
| HEC108 | Uterus | Endometrial Carcinoma | ## |
| C4I | Cervix | Cervical Squamous Cell Carcinoma | ## |
| NCIH2228 | Lung | Non-Small Cell Lung Cancer | ## |
| SNU216 | Esophagus/Stomach | Esophagogastric Adenocarcinoma | ## |
| DBTRG05MG | CNS/Brain | Diffuse Glioma | ### |
| SUM149PT | Breast | Invasive Breast Carcinoma | ## |
| HT29 | Bowel | Colorectal Adenocarcinoma | ## |
| TE11 | Esophagus/Stomach | Esophageal Squamous Cell | ### |
| | | Carcinoma | |
| SNU1214 | Head and Neck | Head and Neck Squamous Cell | ## |
| | | Carcinoma | |
| TE14 | Esophagus/Stomach | Esophageal Squamous Cell | ### |
| | | Carcinoma | |
| NCIH2023 | Lung | Non-Small Cell Lung Cancer | ## |
| MEWO | Skin | Melanoma | ## |
| HSC1 | Skin | Cutaneous Squamous Cell | ### |
| | | Carcinoma | |
| SUDHL1 | Lymphoid | Mature T and NK Neoplasms | ### |
| CAL27 | Head and Neck | Head and Neck Squamous Cell | ### |
| | | Carcinoma | |
| HOTHC | Thyroid | Anaplastic Thyroid Cancer | ### |
| 921 | Eye | Ocular Melanoma | ## |
| SKPNDW | Bone | Ewing Sarcoma | ### |
| HH | Lymphoid | Non-Hodgkin Lymphoma | ### |
| COGN278 | Peripheral Nervous | Neuroblastoma | # |
| | System | | |
| LMSU | Esophagus/Stomach | Esophagogastric Adenocarcinoma | ### |
| RO82W1 | Thyroid | Well-Differentiated Thyroid Cancer | ### |
| SNUC4 | Bowel | Colorectal Adenocarcinoma | ## |
| NOMO1 | Myeloid | Acute Myeloid Leukemia | ### |
| PK59 | Pancreas | Pancreatic Adenocarcinoma | ### |
| DKMG | CNS/Brain | Diffuse Glioma | ### |
| ICC137 | Biliary Tract | Intraductal Papillary Neoplasm of | ## |
| | | the Bile Duct | |
| CCK81 | Bowel | Colorectal Adenocarcinoma | # |
| PFSK1 | CNS/Brain | Embryonal Tumor | ### |
| MOLM13 | Myeloid | Acute Myeloid Leukemia | ### |
| RL952 | Uterus | Endometrial Carcinoma | ## |
| PANC0203 | Pancreas | Pancreatic Adenocarcinoma | ### |
| SW480 | Bowel | Colorectal Adenocarcinoma | ### |
| SNU719 | Esophagus/Stomach | Esophagogastric Adenocarcinoma | ## |
| ANGMCSS | CNS/Brain | Diffuse Glioma | ## |
| A427 | Lung | Non-Small Cell Lung Cancer | ### |
| MMNK1 | Biliary Tract | Non-Cancerous | ### |
| DANG | Pancreas | Pancreatic Adenocarcinoma | ## |
| KD | Soft Tissue | Rhabdoid Cancer | ### |
| A172 | CNS/Brain | Diffuse Glioma | ### |
| TANOUE | Lymphoid | B-Lymphoblastic | ### |
| | | Leukemia/Lymphoma | |
| TOV112D | Ovary/Fallopian Tube | Ovarian Epithelial Tumor | ## |
| SKMEL5 | Skin | Melanoma | ### |
| OV56 | Ovary/Fallopian Tube | Ovarian Epithelial Tumor | ## |
| SNU308 | Biliary Tract | Intracholecystic Papillary Neoplasm | ### |
| PSN1 | Pancreas | Pancreatic Adenocarcinoma | # |
| TASK1 | CNS/Brain | Embryonal Tumor | # |
| BICR22 | Head and Neck | Head and Neck Squamous Cell | ### |
| | | Carcinoma | |
| PF382 | Lymphoid | T-Lymphoblastic | ## |
| | | Leukemia/Lymphoma | |
| JJN3 | Lymphoid | Mature B-Cell Neoplasms | ### |
| SNU2372 | Breast | Invasive Breast Carcinoma | ### |
| WM983B | Skin | Melanoma | ### |
| CJM | Skin | Melanoma | ### |
| SNU478 | Ampulla of Vater | Ampullary Carcinoma | ## |
| HUH7 | Liver | Hepatocellular Carcinoma | ## |
| UW228 | CNS/Brain | Embryonal Tumor | ### |
| NCIH1623 | Lung | Non-Small Cell Lung Cancer | ### |
| U343 | CNS/Brain | Diffuse Glioma | ### |
| PANC0403 | Pancreas | Pancreatic Adenocarcinoma | ## |
| HT115 | Bowel | Colorectal Adenocarcinoma | # |
| CCSW1 | Biliary Tract | Intraductal Papillary Neoplasm of | ## |
| | | the Bile Duct | |
| A253 | Head and Neck | Head and Neck Squamous Cell | ### |
| | | Carcinoma | |
| HUCCT1 | Biliary Tract | Intraductal Papillary Neoplasm of | ### |
| | | the Bile Duct | |

TABLE 12

| | | | MGMT |
|---|---|---|---|
| Cell Line | Lineage | Primary Disease | Expression |
| RD | Soft Tissue | Rhabdomyosarcoma | ## |
| NCIH1563 | Lung | Non-Small Cell Lung Cancer | ## |
| MONOMAC6 | Myeloid | Acute Myeloid Leukemia | ### |
| ABC1 | Lung | Non-Small Cell Lung Cancer | ## |
| SW1088 | CNS/Brain | Diffuse Glioma | ### |
| COLO679 | Skin | Melanoma | ### |
| SKNMC | Bone | Ewing Sarcoma | ## |
| VMCUB1 | Bladder/Urinary Tract | Bladder Urothelial Carcinoma | ### |
| SW948 | Bowel | Colorectal Adenocarcinoma | ## |
| CHAGOK1 | Lung | Non-Small Cell Lung Cancer | ### |
| 143B | Bone | Osteosarcoma | ### |
| U937 | Myeloid | Acute Myeloid Leukemia | ### |
| HGC27 | Esophagus/Stomach | Esophagogastric | ## |
| | | Adenocarcinoma | |
| GCT | Soft Tissue | Undifferentiated Pleomorphic | ### |
| | | Sarcoma/Malignant Fibrous | |
| | | Histiocytoma/High-Grade | |
| | | Spindle Cell Sarcoma | |
| PECAPJ49 | Head and Neck | Head and Neck Squamous Cell | ### |
| | | Carcinoma | |
| NCIH524 | Lung | Lung Neuroendocrine Tumor | ### |
| ICC4 | Biliary Tract | Intraductal Papillary Neoplasm | ### |
| | | of the Bile Duct | |
| TYKNU | Ovary/Fallopian Tube | Ovarian Epithelial Tumor | ### |
| RERFLCAD2 | Lung | Non-Small Cell Lung Cancer | ## |
| DU4475 | Breast | Invasive Breast Carcinoma | ### |
| NCIH196 | Lung | Lung Neuroendocrine Tumor | ## |
| MSTO211H | Pleura | Pleural Mesothelioma | ## |
| HCC1806 | Breast | Breast Ductal Carcinoma In Situ | ## |
| TM31 | CNS/Brain | Diffuse Glioma | ### |
| YD15 | Head and Neck | Head and Neck Squamous Cell | ### |
| | | Carcinoma | |
| MCAS | Ovary/Fallopian Tube | Ovarian Epithelial Tumor | ### |
| PATU8988S | Pancreas | Pancreatic Adenocarcinoma | ## |
| SKUT1 | Uterus | Uterine Sarcoma/Mesenchymal | ## |
| EGI1 | Biliary Tract | Intraductal Papillary Neoplasm | ### |
| | | of the Bile Duct | |
| ICC15 | Biliary Tract | Intraductal Papillary Neoplasm | ### |
| | | of the Bile Duct | |
| PATU8902 | Pancreas | Pancreatic Adenocarcinoma | ### |
| RMSYM | Soft Tissue | Rhabdomyosarcoma | ### |
| TDOTT | Esophagus/Stomach | Esophageal Squamous Cell | ### |
| | | Carcinoma | |
| JURLMK1 | Myeloid | Myeloproliferative Neoplasms | ## |
| NCIH2196 | Lung | Lung Neuroendocrine Tumor | ### |
| NCIH2030 | Lung | Non-Small Cell Lung Cancer | ### |
| HS944T | Skin | Melanoma | ### |
| PECAPJ15 | Head and Neck | Head and Neck Squamous Cell | ### |
| | | Carcinoma | |
| H4 | CNS/Brain | Diffuse Glioma | ### |
| LK2 | Lung | Non-Small Cell Lung Cancer | ### |
| IGROV1 | Ovary/Fallopian Tube | Ovarian Epithelial Tumor | ### |
| K029AX | Skin | Melanoma | ### |
| OCILY132 | Lymphoid | Mature B-Cell Neoplasms | ## |
| LOVO | Bowel | Colorectal Adenocarcinoma | # |
| KYSE30 | Esophagus/Stomach | Esophageal Squamous Cell | ### |
| | | Carcinoma | |
| CMLT1 | Myeloid | Myeloproliferative Neoplasms | ## |
| U266B1 | Lymphoid | Mature B-Cell Neoplasms | ### |
| COLO783 | Skin | Melanoma | ## |
| NCIH1373 | Lung | Non-Small Cell Lung Cancer | ### |
| KOSC2 | Head and Neck | Head and Neck Squamous Cell | ### |
| | | Carcinoma | |
| KYSE410 | Esophagus/Stomach | Esophageal Squamous Cell | ### |
| | | Carcinoma | |
| RCM1 | Bowel | Colorectal Adenocarcinoma | ### |
| HUT78 | Lymphoid | Mature T and NK Neoplasms | ## |
| KYSE70 | Esophagus/Stomach | Esophageal Squamous Cell | ### |
| | | Carcinoma | |
| 5637 | Bladder/Urinary Tract | Bladder Urothelial Carcinoma | ### |
| KYO1 | Myeloid | Myeloproliferative Neoplasms | ### |
| DEL | Lymphoid | Mature T and NK Neoplasms | ### |
| PANC0327 | Pancreas | Pancreatic Adenocarcinoma | ### |
| U178 | CNS/Brain | Diffuse Glioma | ### |
| HMC18 | Breast | Invasive Breast Carcinoma | ## |
| HT1197 | Bladder/Urinary Tract | Bladder Urothelial Carcinoma | ### |
| HS852T | Skin | Melanoma | ### |
| NCIH2172 | Lung | Non-Small Cell Lung Cancer | ### |
| BFTC905 | Bladder/Urinary Tract | Bladder Urothelial Carcinoma | ### |
| L33 | Pancreas | Adenosquamous Carcinoma of | ### |
| | | the Pancreas | |
| DL | Soft Tissue | Rhabdoid Cancer | ## |
| OE33 | Esophagus/Stomach | Esophagogastric | ### |
| | | Adenocarcinoma | |
| A3KAW | Lymphoid | Mature B-Cell Neoplasms | ### |
| SW954 | Vulva/Vagina | Squamous Cell Carcinoma of | ### |
| | | the Vulva/Vagina | |
| A704 | Kidney | Renal Cell Carcinoma | ## |
| HCSC1 | Cervix | Cervical Adenocarcinoma | ### |
| K562 | Myeloid | Myeloproliferative Neoplasms | ### |
| HEYA8 | Ovary/Fallopian Tube | Ovarian Epithelial Tumor | ### |
| GB2 | Biliary Tract | Intracholecystic Papillary | # |
| | | Neoplasm | |
| LC1SQSF | Lung | Non-Small Cell Lung Cancer | ### |
| NCIH2170 | Lung | Non-Small Cell Lung Cancer | ### |
| BV173 | Myeloid | Myeloproliferative Neoplasms | ## |
| SKLMS1 | Soft Tissue | Leiomyosarcoma | ### |
| GSS | Esophagus/Stomach | Esophagogastric | ### |
| | | Adenocarcinoma | |
| UMUC10 | Bladder/Urinary Tract | Bladder Urothelial Carcinoma | ### |
| SNU899 | Head and Neck | Head and Neck Squamous Cell | ## |
| | | Carcinoma | |
| VMRCRCW | Kidney | Renal Cell Carcinoma | ## |
| AMO1 | Lymphoid | Mature B-Cell Neoplasms | ### |
| KMS27 | Lymphoid | Mature B-Cell Neoplasms | ### |
| HUH28 | Biliary Tract | Intraductal Papillary Neoplasm | ### |
| | | of the Bile Duct | |
| NCIH2122 | Lung | Non-Small Cell Lung Cancer | ### |
| SUPT11 | Lymphoid | Mature T and NK Neoplasms | ## |
| HKA1 | Skin | Cutaneous Squamous Cell | ## |
| | | Carcinoma | |
| HSC2 | Head and Neck | Head and Neck Squamous Cell | ### |
| | | Carcinoma | |
| KYSE150 | Esophagus/Stomach | Esophageal Squamous Cell | ### |
| | | Carcinoma | |
| ONDA9 | CNS/Brain | Diffuse Glioma | ## |
| EFO21 | Ovary/Fallopian Tube | Ovarian Epithelial Tumor | ## |
| MON | Soft Tissue | Rhabdoid Cancer | ### |
| SW620 | Bowel | Colorectal Adenocarcinoma | ### |
| 93T449 | Soft Tissue | Liposarcoma | ### |
| SW1116 | Bowel | Colorectal Adenocarcinoma | ## |
| CHLA10 | Bone | Ewing Sarcoma | ### |
| SKN | Uterus | Uterine Sarcoma/Mesenchymal | ### |
| ICC12 | Biliary Tract | Intraductal Papillary Neoplasm | ### |
| | | of the Bile Duct | |
| BT12 | CNS/Brain | Embryonal Tumor | ### |
| HS683 | CNS/Brain | Diffuse Glioma | ### |
| TE8 | Esophagus/Stomach | Esophageal Squamous Cell | ## |
| | | Carcinoma | |
| T3M5 | Head and Neck | Head and Neck Squamous Cell | ### |
| | | Carcinoma | |
| T84 | Bowel | Colorectal Adenocarcinoma | ### |
| CADOES1 | Bone | Ewing Sarcoma | # |
| ICC8 | Biliary Tract | Intraductal Papillary Neoplasm | ### |
| | | of the Bile Duct | |
| HCC44 | Lung | Non-Small Cell Lung Cancer | ### |
| 8505C | Thyroid | Anaplastic Thyroid Cancer | ### |
| LNZ308 | CNS/Brain | Diffuse Glioma | ### |
| ICC9 | Biliary Tract | Intraductal Papillary Neoplasm | ### |
| | | of the Bile Duct | |
| NCIN87 | Esophagus/Stomach | Esophagogastric | ## |
| | | Adenocarcinoma | |
| A375 | Skin | Melanoma | ### |
| YSCCC | Biliary Tract | Intraductal Papillary Neoplasm | ## |
| | | of the Bile Duct | |
| T98G | CNS/Brain | Diffuse Glioma | ### |
| UACC62 | Skin | Melanoma | ### |
| SNU1076 | Head and Neck | Head and Neck Squamous Cell | ## |
| | | Carcinoma | |
| MDAMB435S | Skin | Melanoma | ### |
| LS | Peripheral Nervous | Neuroblastoma | ## |
| | System | | |
| SNB75 | CNS/Brain | Diffuse Glioma | ### |
| CORL88 | Lung | Lung Neuroendocrine Tumor | ### |
| RT112 | Bladder/Urinary Tract | Bladder Urothelial Carcinoma | # |
| DMS53 | Lung | Lung Neuroendocrine Tumor | ### |
| JR | Soft Tissue | Rhabdomyosarcoma | ### |
| SW1463 | Bowel | Colorectal Adenocarcinoma | ### |
| NCIH522 | Lung | Non-Small Cell Lung Cancer | ## |
| MFE319 | Uterus | Endometrial Carcinoma | ## |
| SKGT2 | Esophagus/Stomach | Esophagogastric | ## |
| | | Adenocarcinoma | |
| ECC2 | Biliary Tract | Intraductal Papillary Neoplasm | # |
| | | of the Bile Duct | |
| BICR6 | Head and Neck | Head and Neck Squamous Cell | ### |
| | | Carcinoma | |
| GIMEN | Peripheral Nervous | Neuroblastoma | ### |
| | System | | |
| COLO792 | Skin | Melanoma | ### |
| CAS1 | CNS/Brain | Diffuse Glioma | ### |
| JHH6 | Liver | Hepatocellular Carcinoma | ## |
| FADU | Head and Neck | Head and Neck Squamous Cell | ## |
| | | Carcinoma | |
| KE97 | Lymphoid | Mature B-Cell Neoplasms | ### |
| STM9101 | Kidney | Rhabdoid Cancer | ### |
| H157 | Head and Neck | Head and Neck Squamous Cell | ### |
| | | Carcinoma | |
| HS729 | Fibroblast | Non-Cancerous | ### |
| MCC26 | Skin | Merkel Cell Carcinoma | ### |
| LS513 | Bowel | Colorectal Adenocarcinoma | ### |
| RVH421 | Skin | Melanoma | ## |
| COV644 | Ovary/Fallopian Tube | Ovarian Epithelial Tumor | ## |
| ISTMES1 | Pleura | Pleural Mesothelioma | ### |
| SF539 | CNS/Brain | Diffuse Glioma | ### |
| HUO9 | Bone | Osteosarcoma | ### |
| CHLA57 | CNS/Brain | Embryonal Tumor | ## |
| KATOIII | Esophagus/Stomach | Esophagogastric | ### |
| | | Adenocarcinoma | |
| UACC257 | Skin | Melanoma | ## |
| 639V | Bladder/Urinary Tract | Urethral Cancer | ### |
| IPC298 | Skin | Melanoma | ## |
| HEC50B | Uterus | Endometrial Carcinoma | ## |
| NCIH1975 | Lung | Non-Small Cell Lung Cancer | ### |
| NCIH2171 | Lung | Lung Neuroendocrine Tumor | ### |
| RKO | Bowel | Colorectal Adenocarcinoma | ## |
| OVTOKO | Ovary/Fallopian Tube | Ovarian Epithelial Tumor | ## |
| G402 | Kidney | Renal Cell Carcinoma | ### |
| HCC1359 | Lung | Non-Small Cell Lung Cancer | ## |
| ALLSIL | Lymphoid | T-Lymphoblastic | ### |
| | | Leukemia/Lymphoma | |
| TE5 | Esophagus/Stomach | Esophageal Squamous Cell | ### |
| | | Carcinoma | |
| NCIH2110 | Lung | Non-Small Cell Lung Cancer | ## |
| EFM192A | Breast | Invasive Breast Carcinoma | ## |
| EW8 | Bone | Ewing Sarcoma | ## |
| AN3CA | Uterus | Endometrial Carcinoma | # |
| HEL | Myeloid | Acute Myeloid Leukemia | ### |
| UMUC6 | Bladder/Urinary Tract | Bladder Urothelial Carcinoma | ### |
| OCILY19 | Lymphoid | Mature B-Cell Neoplasms | ## |
| HPBALL | Lymphoid | T-Lymphoblastic | ## |
| | | Leukemia/Lymphoma | |
| SNU81 | Bowel | Colorectal Adenocarcinoma | ## |
| NCIH1792 | Lung | Non-Small Cell Lung Cancer | ### |
| ISHIKAWAHERAKLIO02ER | Uterus | Endometrial Carcinoma | # |
| YD10B | Head and Neck | Head and Neck Squamous Cell | ### |
| | | Carcinoma | |
| UMRC3 | Kidney | Renal Cell Carcinoma | ### |
| RMGI | Ovary/Fallopian Tube | Ovarian Epithelial Tumor | ### |
| JMURTK2 | Kidney | Rhabdoid Cancer | ## |
| 697 | Lymphoid | B-Lymphoblastic | ## |
| | | Leukemia/Lymphoma | |
| CAL33 | Head and Neck | Head and Neck Squamous Cell | ### |
| | | Carcinoma | |
| IGR39 | Skin | Melanoma | ## |
| M059K | CNS/Brain | Diffuse Glioma | ### |
| MDAMB453 | Breast | Invasive Breast Carcinoma | ## |
| JHUEM7 | Uterus | Endometrial Carcinoma | ## |
| KCL22 | Myeloid | Myeloproliferative Neoplasms | ## |
| EOL1 | Myeloid | Myeloproliferative Neoplasms | ### |
| MKN7 | Esophagus/Stomach | Esophagogastric | ### |
| | | Adenocarcinoma | |
| HCC1428 | Breast | Invasive Breast Carcinoma | ## |
| 42MGBA | CNS/Brain | Diffuse Glioma | ### |
| TE15 | Esophagus/Stomach | Esophageal Squamous Cell | ### |
| | | Carcinoma | |
| KARPAS620 | Lymphoid | Mature B-Cell Neoplasms | ### |
| MCC142 | Skin | Merkel Cell Carcinoma | ### |
| PK1 | Pancreas | Pancreatic Adenocarcinoma | ### |
| HCC1954 | Breast | Breast Ductal Carcinoma In Situ | ### |
| KYSE180 | Esophagus/Stomach | Esophageal Squamous Cell | ### |
| | | Carcinoma | |
| KS1 | CNS/Brain | Diffuse Glioma | ### |
| NCIH1793 | Lung | Non-Small Cell Lung Cancer | ### |
| 8305C | Thyroid | Anaplastic Thyroid Cancer | ### |
| A498 | Kidney | Renal Cell Carcinoma | ## |
| NCIH1651 | Lung | Non-Small Cell Lung Cancer | ### |
| JHOM1 | Ovary/Fallopian Tube | Ovarian Epithelial Tumor | ### |
| SH10TC | Esophagus/Stomach | Esophagogastric | ### |
| | | Adenocarcinoma | |
| C75 | Bowel | Colorectal Adenocarcinoma | # |
| WAOSEL | Lymphoid | Mature B-Cell Neoplasms | ## |
| SNU869 | Ampulla of Vater | Ampullary Carcinoma | ### |
| DV90 | Lung | Non-Small Cell Lung Cancer | ## |
| HT3 | Cervix | Cervical Adenocarcinoma | ### |
| JOPACA1 | Pancreas | Pancreatic Adenocarcinoma | ## |
| U2OS | Bone | Osteosarcoma | ### |
| OE21 | Esophagus/Stomach | Esophageal Squamous Cell | ### |
| | | Carcinoma | |
| COLO741 | Skin | Melanoma | ### |
| SCC3 | Lymphoid | Non-Hodgkin Lymphoma | ### |
| SF126 | CNS/Brain | Diffuse Glioma | ### |
| SQ1 | Lung | Non-Small Cell Lung Cancer | ### |
| MELHO | Skin | Melanoma | ### |
| KYSE520 | Esophagus/Stomach | Esophageal Squamous Cell | ## |
| | | Carcinoma | |
| KMS11 | Lymphoid | Mature B-Cell Neoplasms | ## |
| NCIH1703 | Lung | Non-Small Cell Lung Cancer | ## |
| UO31 | Kidney | Renal Cell Carcinoma | # |
| SCC9 | Head and Neck | Head and Neck Squamous Cell | ### |
| | | Carcinoma | |
| TOLEDO | Lymphoid | Mature B-Cell Neoplasms | ### |
| L82 | Lymphoid | Mature T and NK Neoplasms | ### |
| MELJUSO | Skin | Melanoma | ### |
| HCT15 | Bowel | Colorectal Adenocarcinoma | ## |
| KARPAS422 | Lymphoid | Mature B-Cell Neoplasms | ### |
| HEC116 | Uterus | Endometrial Carcinoma | ## |
| NCIH596 | Lung | Non-Small Cell Lung Cancer | ## |
| COV434 | Ovary/Fallopian Tube | Ovarian Epithelial Tumor | ## |
| TTC549 | Soft Tissue | Rhabdoid Cancer | ## |
| HT144 | Skin | Melanoma | ### |
| SNU761 | Liver | Hepatocellular Carcinoma | # |
| NCIH292 | Lung | Non-Small Cell Lung Cancer | ### |
| CORL279 | Lung | Lung Neuroendocrine Tumor | ### |
| EKVX | Lung | Non-Small Cell Lung Cancer | ### |
| SH4 | Skin | Melanoma | ### |
| RT4 | Bladder/Urinary Tract | Bladder Urothelial Carcinoma | ## |
| SNU685 | Uterus | Endometrial Carcinoma | ### |
| HCC1395 | Breast | Invasive Breast Carcinoma | ### |
| OCIP5X | Ovary/Fallopian Tube | Ovarian Epithelial Tumor | ### |
| A549 | Lung | Non-Small Cell Lung Cancer | ### |
| NCIH2444 | Lung | Non-Small Cell Lung Cancer | ## |
| SKNAS | Peripheral Nervous | Neuroblastoma | ## |
| | System | | |
| UBLC1 | Bladder/Urinary Tract | Bladder Urothelial Carcinoma | ### |
| SMSCTR | Soft Tissue | Rhabdomyosarcoma | # |
| DMS273 | Lung | Lung Neuroendocrine Tumor | ### |
| PECAPJ41CLONED2 | Head and Neck | Head and Neck Squamous Cell | ### |
| | | Carcinoma | |
| COLO320 | Bowel | Colorectal Adenocarcinoma | ### |
| KON | Head and Neck | Head and Neck Squamous Cell | ### |
| | | Carcinoma | |
| PANC1 | Pancreas | Pancreatic Adenocarcinoma | # |
| YD38 | Head and Neck | Head and Neck Squamous Cell | ### |
| | | Carcinoma | |
| JL1 | Pleura | Pleural Mesothelioma | ### |
| PL21 | Myeloid | Acute Myeloid Leukemia | ### |
| SKGII | Cervix | Cervical Squamous Cell | ## |
| | | Carcinoma | |
| GRANTA519 | Lymphoid | Mature B-Cell Neoplasms | ### |
| TTC709 | Soft Tissue | Rhabdoid Cancer | ## |
| RL | Lymphoid | Mature B-Cell Neoplasms | ### |
| C8166 | Lymphoid | T-Lymphoblastic | ### |
| | | Leukemia/Lymphoma | |
| NCIH2087 | Lung | Non-Small Cell Lung Cancer | # |
| MIAPACA2 | Pancreas | Pancreatic Adenocarcinoma | ## |
| NCIH322 | Lung | Non-Small Cell Lung Cancer | ### |
| HUG1N | Esophagus/Stomach | Esophagogastric | ### |
| | | Adenocarcinoma | |
| KNS81 | CNS/Brain | Diffuse Glioma | ### |
| A388 | Skin | Cutaneous Squamous Cell | ### |
| | | Carcinoma | |
| NUGC4 | Esophagus/Stomach | Esophagogastric | ## |
| | | Adenocarcinoma | |
| WM1799 | Skin | Melanoma | ### |
| 1618K | Testis | Non-Seminomatous Germ Cell | ### |
| | | Tumor | |
| RH30 | Soft Tissue | Rhabdomyosarcoma | ### |
| VMRCRCZ | Kidney | Renal Cell Carcinoma | ### |
| 786O | Kidney | Renal Cell Carcinoma | ## |
| TC106 | Bone | Ewing Sarcoma | ### |
| COV318 | Ovary/Fallopian Tube | Ovarian Epithelial Tumor | ## |
| KKU213 | Biliary Tract | Intraductal Papillary Neoplasm | ### |
| | | of the Bile Duct | |
| DOHH2 | Lymphoid | Mature B-Cell Neoplasms | ## |
| THP1 | Myeloid | Acute Myeloid Leukemia | ### |
| WERIRB1 | Eye | Retinoblastoma | ## |
| 9505BIK | Pancreas | Pancreatic Adenocarcinoma | ### |

### Example 46—Anti-Cancer Biological Activity Assay

The activity of compound I-1 and compound A1 was evaluated in the anti-cancer assay described herein below.

### Part I—Experimental Procedure

Compound I-1 and compound A1 were tested for anti-cancer activity against isogenic LN229 gliobastoma cells using a procedure based on that described in Example 43 above, except that the maximum concentration of compound tested was 200 μM.

| Compound | |
|---|---|
| No. | Chemical Structure |
| I-1 | 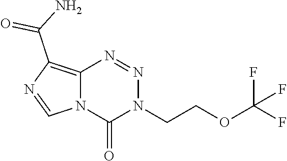 [SMILES: NC(=O)c1ncn2c(=O)n(CCOC(F)(F)F)nnc12] |
| A1 | 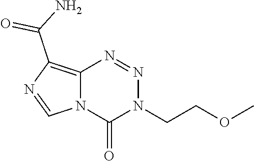 [SMILES: COCCn1nnc2c(C(N)=O)ncn2c1=O] |

### Part II—Results

Compound A1 had no detectable anti-cancer activity in this assay. In contrast, compound I-1 was able to cause the death of LN229 cells that were MGMT negative/MMR positive. Compound I-1 had an IC₅₀ less than 20 μM in the assay for causing the death of LN229 cells that were MGMT negative/MMR positive. Additionally, compound I-1 was able to cause the death of LN229 cells that were MGMT negative/MMR negative. Compound I-1 had an IC₅₀ less than 20 μM in the assay for causing the death of LN229 cells that were MGMT negative/MMR negative. This demonstrates the superior anti-cancer effects of compound I-1 compared to compound A1.

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. A compound represented by Formula I: 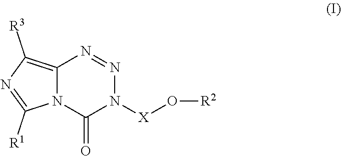
or a pharmaceutically acceptable salt thereof, wherein:
R¹ is hydrogen or C₁₋₄ alkyl;
R² is C₁₋₄ fluoroalkyl;
R³ is —C(O)N(R⁴)(R⁵), —CO₂R⁵, —C(O)SR⁴, —C(S)N(R⁴)(R⁵), —C(═NR⁷)OR⁴, —C(═NR⁷)SR⁴, —C(═NR⁷)N(R⁴)(R⁵), —C(O)-(halo), —C(O)—(C₁₋₄ alkyl), —CN, halo, or C₁₋₄ alkyl;
R⁴ is hydrogen, C₁₋₄ alkyl, or C₃₋₆ cycloalkyl;
R⁵ is hydrogen, C₁₋₄ alkyl, C₁₋₄ alkenyl, C₁₋₄ alkynyl, —(C₀₋₄ alkylene)-R⁶, —(C₁₋₄ alkylene)-C(O)—R⁶, —(C₁₋₄ alkylene)-C(O)—(C₁₋₄ alkyl), —(C₁₋₄ alkylene)-OR⁷, or —(C₁₋₄ alkylene)-N(R⁷)(R⁸); or R⁴ and R⁵ are taken together with the nitrogen atom to which they are attached to form a 3-7 membered heterocyclic ring containing the nitrogen atom and 0 or 1 additional heteroatom selected from nitrogen, oxygen, and sulfur, wherein the 3-7 membered heterocyclic ring is substituted with 0, 1, 2, or 3 occurrences of R⁹;
R⁶ is C₃₋₆ cycloalkyl, phenyl, or 5-6 membered heteroaryl; each of which is substituted with 0, 1, 2, or 3 occurrences of R⁹;
R⁷ and R⁸ are independently for each occurrence hydrogen, C₁₋₄ alkyl, or C₃₋₆ cycloalkyl; or R⁷ and R⁸ are taken together with the nitrogen atom to which they are attached to form a 3-7 membered heterocyclic ring containing the nitrogen atom;
R⁹ represents independently for each occurrence C₁₋₄ alkyl, C₃₋₆ cycloalkyl, halo, —OR⁷, or —N(R⁷)(R⁸); and
X is C₁₋₃ alkylene or C₁₋₃ deuteroalkylene.

2. (canceled)

3. The compound of claim 1, wherein X is —CH₂CH₂—.

4. (canceled)

5. (canceled)

6. The compound of claim 1, wherein R² is C₁₋₂ fluoroalkyl.

7. The compound of claim 1, wherein R² is trifluoromethyl.

8. The compound of claim 1, wherein R³ is —C(O)N(R⁴)(R⁵).

9-21. (canceled)

22. The compound of claim 1, wherein the compound is represented by Formula I-A: 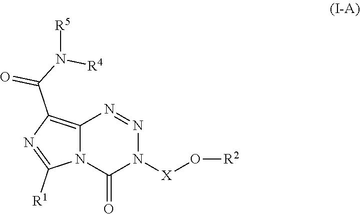
or a pharmaceutically acceptable salt thereof, wherein:
R¹ is hydrogen or methyl;
R² is C₁₋₂ fluoroalkyl;
R⁴ is hydrogen or C₁₋₄ alkyl;
R⁵ is hydrogen, C₁₋₄ alkyl, C₃₋₆ cycloalkyl, phenyl, or 5-6 membered heteroaryl; wherein the phenyl is substituted with 0 or 1 occurrence of R⁹; or R⁴ and R⁵ are taken together with the nitrogen atom to which they are attached to form a 3-7 membered saturated heterocyclic ring containing the nitrogen atom and 0 or 1 additional nitrogen atom; wherein the additional nitrogen atom is optionally substituted with C₁₋₄ alkyl;
R⁹ is C₁₋₄ alkyl, fluoro, chloro, —OH, or —NH₂; and
X is C₁₋₃ alkylene.

23-24. (canceled)

25. The compound of claim 22, wherein R² is trifluoromethyl.

26-28. (canceled)

29. The compound of claim 25, wherein X is —CH₂CH₂—.

30. The compound of claim 1, wherein the compound is represented by Formula I-aa: 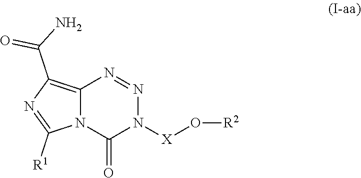
or a pharmaceutically acceptable salt thereof, wherein:
R¹ is hydrogen or methyl;
R² is C₁₋₂ fluoroalkyl; and
X is C₁₋₃ alkylene.

31. (canceled)

32. The compound of claim 30, wherein X is —CH₂CH₂—.

33. (canceled)

34. (canceled)

35. The compound of claim 30, wherein R² is trifluoromethyl.

36. (canceled)

37. (canceled)

38. A compound that is 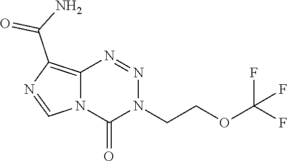 [SMILES: NC(=O)c1ncn2c(=O)n(CCOC(F)(F)F)nnc12]
or a pharmaceutically acceptable salt thereof.

39. The compound of claim 39, wherein the compound is

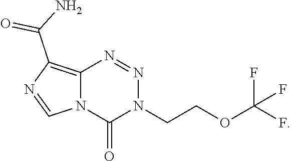 [SMILES: NC(=O)c1ncn2c(=O)n(CCOC(F)(F)F)nnc12]

40. The compound of claim 1, wherein the compound is a compound in Table 1 below, or a pharmaceutically acceptable salt thereof:
TABLE 1

| Compound | |
|---|---|
| No. | Chemical Structure |
| I-1 | 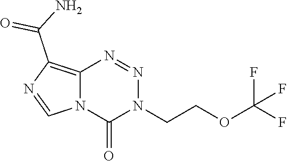 [SMILES: NC(=O)c1ncn2c(=O)n(CCOC(F)(F)F)nnc12] |
| I-2 | 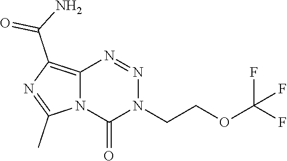 [SMILES: Cc1nc(C(N)=O)c2nnn(CCOC(F)(F)F)c(=O)n12] |
| I-3 | 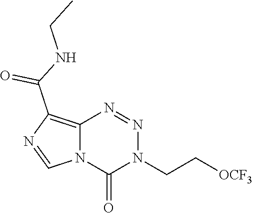 [SMILES: CCNC(=O)c1ncn2c(=O)n(CCOC(F)(F)F)nnc12] |
| I-4 | 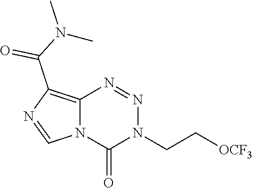 [SMILES: CN(C)C(=O)c1ncn2c(=O)n(CCOC(F)(F)F)nnc12] |
| I-5 | 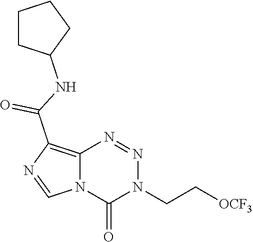 [SMILES: O=C(NC1CCCC1)c1ncn2c(=O)n(CCOC(F)(F)F)nnc12] |
| I-6 | 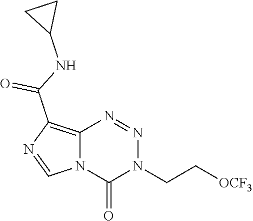 [SMILES: O=C(NC1CC1)c1ncn2c(=O)n(CCOC(F)(F)F)nnc12] |
| I-7 | 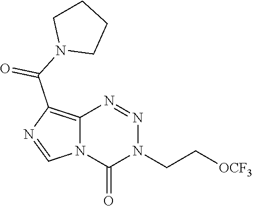 [SMILES: O=C(c1ncn2c(=O)n(CCOC(F)(F)F)nnc12)N1CCCC1] |
| I-8 | 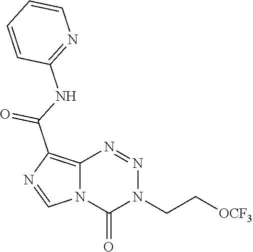 [SMILES: O=C(Nc1ccccn1)c1ncn2c(=O)n(CCOC(F)(F)F)nnc12] |
| I-9 | 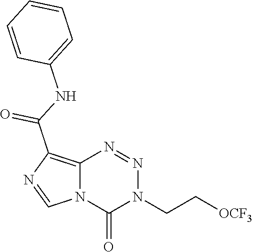 [SMILES: O=C(Nc1ccccc1)c1ncn2c(=O)n(CCOC(F)(F)F)nnc12] |
| I-10 | 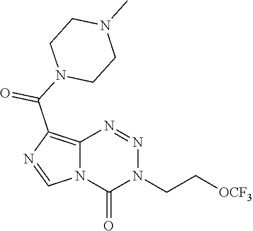 [SMILES: CN1CCN(C(=O)c2ncn3c(=O)n(CCOC(F)(F)F)nnc23)CC1] |
| I-11 | 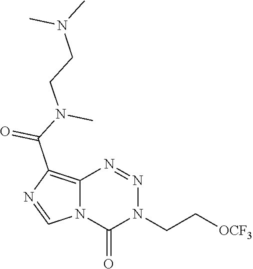 [SMILES: CN(C)CCN(C)C(=O)c1ncn2c(=O)n(CCOC(F)(F)F)nnc12] |
| I-12 | 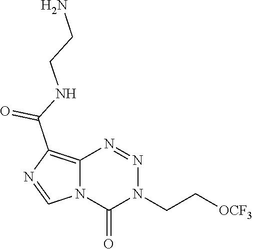 [SMILES: NCCNC(=O)c1ncn2c(=O)n(CCOC(F)(F)F)nnc12] |
| I-13 | 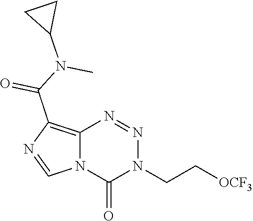 [SMILES: CN(C(=O)c1ncn2c(=O)n(CCOC(F)(F)F)nnc12)C1CC1] |
| I-14 | 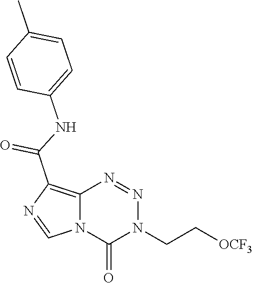 [SMILES: Cc1ccc(NC(=O)c2ncn3c(=O)n(CCOC(F)(F)F)nnc23)cc1] |
| I-15 | 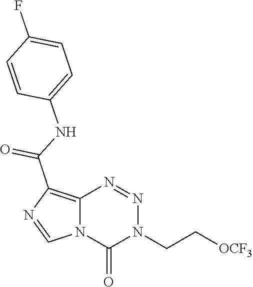 [SMILES: O=C(Nc1ccc(F)cc1)c1ncn2c(=O)n(CCOC(F)(F)F)nnc12] |
| I-16 | 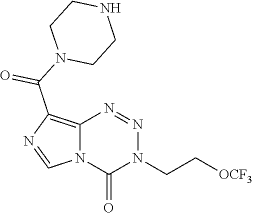 [SMILES: O=C(c1ncn2c(=O)n(CCOC(F)(F)F)nnc12)N1CCNCC1] |
| I-17 | 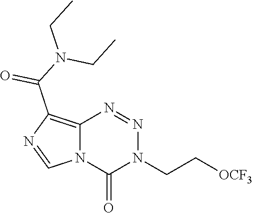 [SMILES: CCN(CC)C(=O)c1ncn2c(=O)n(CCOC(F)(F)F)nnc12] |
| I-18 | 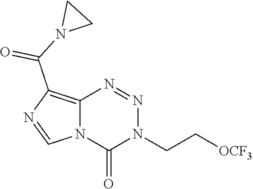 [SMILES: O=C(c1ncn2c(=O)n(CCOC(F)(F)F)nnc12)N1CC1] |
| I-19 | 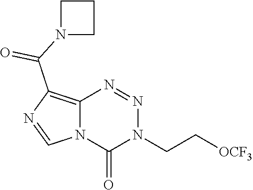 [SMILES: O=C(c1ncn2c(=O)n(CCOC(F)(F)F)nnc12)N1CCC1] |
| I-20 | 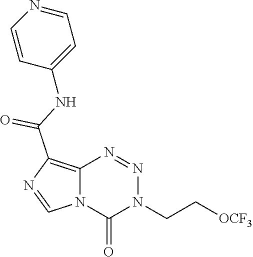 [SMILES: O=C(Nc1ccncc1)c1ncn2c(=O)n(CCOC(F)(F)F)nnc12] |
| I-21 | 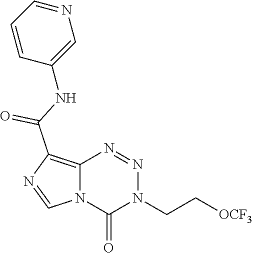 [SMILES: O=C(Nc1cccnc1)c1ncn2c(=O)n(CCOC(F)(F)F)nnc12] |
| I-22 | 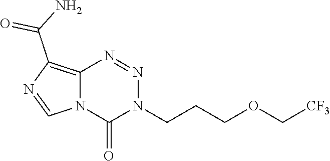 [SMILES: NC(=O)c1ncn2c(=O)n(CCCOCC(F)(F)F)nnc12] |
| I-23 | 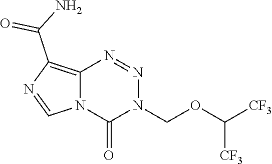 [SMILES: NC(=O)c1ncn2c(=O)n(COC(C(F)(F)F)C(F)(F)F)nnc12] |
| I-24 | 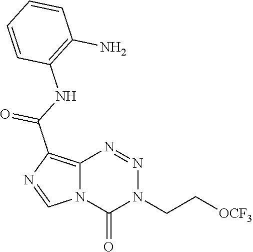 [SMILES: Nc1ccccc1NC(=O)c1ncn2c(=O)n(CCOC(F)(F)F)nnc12] |
| I-25 | 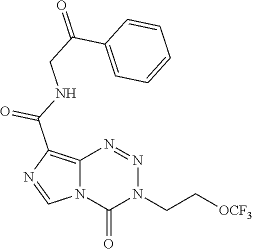 [SMILES: O=C(CNC(=O)c1ncn2c(=O)n(CCOC(F)(F)F)nnc12)c1ccccc1] |
| I-26 | 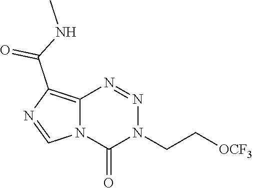 [SMILES: CNC(=O)c1ncn2c(=O)n(CCOC(F)(F)F)nnc12] |
| I-27 | 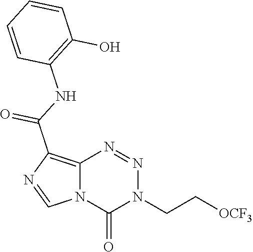 [SMILES: O=C(Nc1ccccc1O)c1ncn2c(=O)n(CCOC(F)(F)F)nnc12] |
| I-28 | 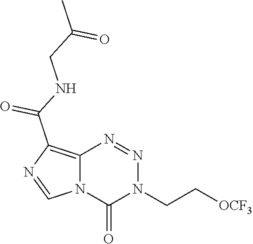 [SMILES: CC(=O)CNC(=O)c1ncn2c(=O)n(CCOC(F)(F)F)nnc12] |
| I-29 | 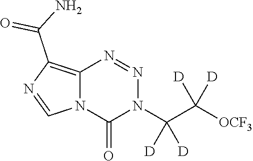 [SMILES: [2H]C([2H])(OC(F)(F)F)C([2H])([2H])n1nnc2c(C(N)=O)ncn2c1=O] |
| I-30 | 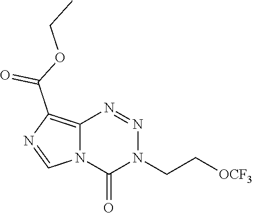 [SMILES: CCOC(=O)c1ncn2c(=O)n(CCOC(F)(F)F)nnc12] |
| I-31 | 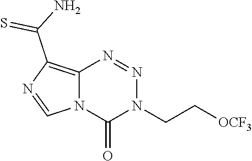 [SMILES: NC(=S)c1ncn2c(=O)n(CCOC(F)(F)F)nnc12] |
| I-32 | 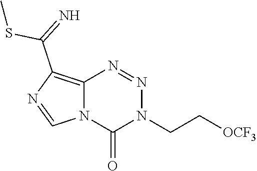 [SMILES: CSC(=N)c1ncn2c(=O)n(CCOC(F)(F)F)nnc12] |
| I-33 | 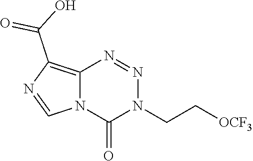 [SMILES: O=C(O)c1ncn2c(=O)n(CCOC(F)(F)F)nnc12] |
| I-34 | 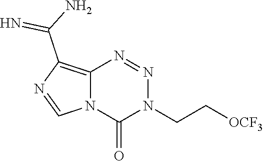 [SMILES: N=C(N)c1ncn2c(=O)n(CCOC(F)(F)F)nnc12] |
| I-35 | 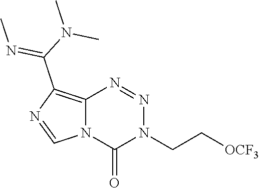 [SMILES: C/N=C(/c1ncn2c(=O)n(CCOC(F)(F)F)nnc12)N(C)C] |
| I-36 | 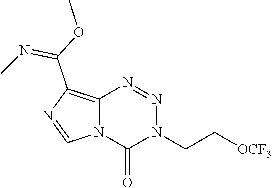 [SMILES: C/N=C(/OC)c1ncn2c(=O)n(CCOC(F)(F)F)nnc12] |
| I-37 | 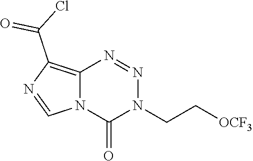 [SMILES: O=C(Cl)c1ncn2c(=O)n(CCOC(F)(F)F)nnc12] |
| I-38 | 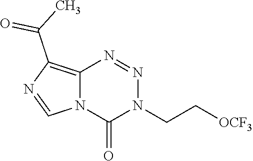 [SMILES: CC(=O)c1ncn2c(=O)n(CCOC(F)(F)F)nnc12] |
| I-39 | 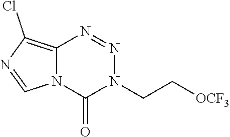 [SMILES: O=c1n(CCOC(F)(F)F)nnc2c(Cl)ncn12] |
| I-40 | 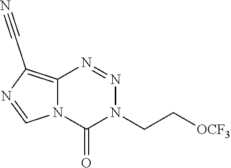 [SMILES: N#Cc1ncn2c(=O)n(CCOC(F)(F)F)nnc12] |
| I-41 | 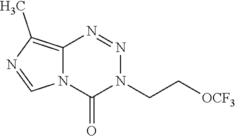 [SMILES: Cc1ncn2c(=O)n(CCOC(F)(F)F)nnc12] |
| I-42 | 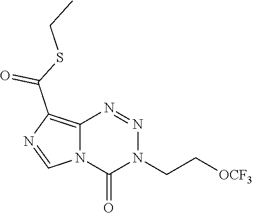 [SMILES: CCSC(=O)c1ncn2c(=O)n(CCOC(F)(F)F)nnc12] |
| I-43 | 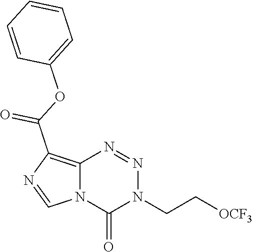 [SMILES: O=C(Oc1ccccc1)c1ncn2c(=O)n(CCOC(F)(F)F)nnc12] |
| I-44 | 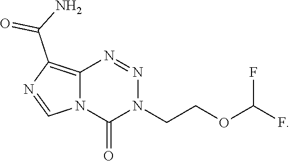 [SMILES: NC(=O)c1ncn2c(=O)n(CCOC(F)F)nnc12] |

41. A pharmaceutical composition comprising a compound of claim 1 and a pharmaceutically acceptable carrier.

42. (canceled)

43. A pharmaceutical composition comprising a compound of claim 38 and a pharmaceutically acceptable carrier.

44. A method of treating cancer, comprising administering to a subject in need thereof a therapeutically effective amount of a compound of claim 1 to treat the cancer.

45. A method of treating cancer, comprising administering to a subject in need thereof a therapeutically effective amount of a compound of claim 38 to treat the cancer.

46. A method of producing a DNA lesion in a subject, comprising administering to a subject an effective amount of a compound of claim 1 to produce a DNA lesion in the subject.

47. A method of producing a DNA lesion in a subject, comprising administering to a subject an effective amount of a compound of claim 38 to produce a DNA lesion in the subject.

48. The method of claim 46, wherein the subject has cancer.

49. The method of claim 48, wherein the cancer is ovarian cancer, uterine cancer, endometrial cancer, cervical cancer, prostate cancer, testicular cancer, breast cancer, brain cancer, lung cancer, oral cancer, esophageal cancer, head and neck cancer, stomach cancer, colon cancer, rectal cancer, skin cancer, sebaceous gland carcinoma, bile duct cancer, gallbladder cancer, liver cancer, pancreatic cancer, bladder cancer, urinary tract cancer, kidney cancer, eye cancer, thyroid cancer, lymphoma, leukemia, urothelial cancer, colorectal cancer, or glioblastoma multiforme.

50. The method of claim 48, wherein the cancer is a breast invasive carcinoma, colon adenocarcinoma, head and neck cancer, lung adenocarcinoma, rectal adenocarcinoma, acute myeloid leukemia, glioblastoma multiforme, brain lower grade glioma, colorectal cancer, or metastatic melanoma.

51. The method of claim 48, wherein the cancer is a glioblastoma multiforme.

52. The method of claim 51, wherein the cancer is MGMT deficient.

53. The method of claim 52, wherein the cancer is MMR deficient.

54. The method of claim 51, wherein the cancer is resistant to treatment using temozolomide.

55-158. (canceled)
